(19) United States
(12) Patent Application Publication
(10) Pub. No.: US 2024/0131032 A1
(43) Pub. Date: Apr. 25, 2024

(54) AN ACTIVITY-GUIDED MAP OF ELECTROPHILE-CYSTEINE INTERACTIONS IN PRIMARY HUMAN IMMUNE CELLS

(71) Applicant: The Scripps Research Institute, La Jolla, CA (US)

(72) Inventors: Benjamin CRAVATT, La Jolla, CA (US); Ekaterina VINOGRADOVA, San Diego, CA (US); Vincent CROWLEY, San Diego, CA (US); Xiaoyu ZHANG, San Diego, CA (US); Michael Andreas SCHAFROTH, San Diego, CA (US); Minoru YOKOYAMA, San Diego, CA (US); Dave REMILLARD, San Diego, CA (US); Bruno MELILLO, San Diego, CA (US); Stuart SCHREIBER, Boston, MA (US)

(21) Appl. No.: 17/768,616

(22) PCT Filed: Oct. 15, 2020
(86) PCT No.: PCT/US2020/055781

§ 371 Date: Apr. 13, 2022

Related U.S. Application Data
(60) Provisional application No. 62/916,170, filed on Oct. 16, 2019.
(60) Provisional application No. 62/916,736, filed on Oct. 17, 2019.
(60) Provisional application No. 63/039,387, filed on Jun. 15, 2020.

(51) Int. Cl. A61K 31/5377; A61K 31/167; A61K 31/357; A61K 31/437; A61K 31/444; A61K 31/4468; A61K 31/454; A61K 31/4545; A61K 31/47; A61K 31/496; A61K 31/506; A61K 31/517; A61K 31/55; A61K 39/39; A61P 35/00

(52) U.S. Cl. CPC A61K 31/5377; A61K 31/167; A61K 31/357; A61K 31/437; A61K 31/444; A61K 31/4468; A61K 31/454; A61K 31/4545; A61K 31/47; A61K 31/496; A61K 31/506; A61K 31/517; A61K 31/55; A61K 39/39; A61P 35/00; A61K 2039/55511

(57) ABSTRACT

Disclosed herein are methods, pharmaceutical compositions, and vaccines for modulating an immune response. Also disclosed herein are methods, pharmaceutical compositions, and vaccines for inducing an immune response.

### CROSS REFERENCE

This application claims the benefit of U.S. Application No. 62/916,170, filed Oct. 16, 2019, U.S. Application No. 62/916,736, filed Oct. 17, 2019, and U.S. Application No. 63/039,387, filed Jun. 15, 2020, which are hereby incorporated by reference in their entirety.

### BACKGROUND OF THE DISCLOSURE

The immune system is a complex network of responses and processes that protects an organism and enables the organism to fight against a foreign agent. In some instances, there are two types of immune response when presented with a foreign agent. In one instance, the immune system responds with a B cell mediated response (e.g., humoral response or antibody-mediated response) when foreign agents (e.g., antigens and/or pathogens) are present in the lymph or blood. In another instance, the immune system responds with a T cell mediated response (e.g., a cell-mediated response) when cells that display aberrant MHC markers are present. In some instances, both humoral response and cell-mediated response are triggered by a foreign agent when, e.g., both antigens and cells containing aberrant MHC markers are present.

### SUMMARY OF THE DISCLOSURE

In certain embodiments, disclosed herein include methods, pharmaceutical compositions, and vaccines for modulating an immune response. In some embodiments, included herein are methods of administrating a small molecule fragment described herein for modulating an immune response. In additional embodiments, described herein are pharmaceutical compositions and vaccines which comprise a small molecule fragment described herein for modulating an immune response.

Disclosed herein, in certain embodiments, is a method of modulating an immune response in a subject in need thereof, comprising administering to the subject a therapeutically effective amount of a small molecule fragment of Formula (I):

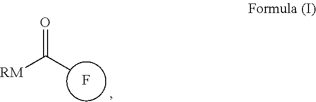

wherein:
RM is a reactive moiety selected from a Michael acceptor moiety, a leaving group moiety, or a moiety capable of forming a covalent bond with the thiol group of a cysteine residue; and
F is a small molecule fragment moiety.

In some embodiments, the small molecule fragment interacts with an endogenous cysteine-containing polypeptide expressed in the subject to form a cysteine-containing polypeptide-small molecule fragment adduct. In some embodiments, the small molecule fragment is covalently bond to a cysteine residue of the cysteine-containing polypeptide. In some embodiments, the cysteine-containing polypeptide-small molecule fragment adduct induces an immune response. In some embodiments, the cysteine-containing polypeptide-small molecule fragment adduct induces a humoral immune response. In some embodiments, the cysteine-containing polypeptide-small molecule fragment adduct induces a cell mediated immune response. In some embodiments, the cysteine-containing polypeptide-small molecule fragment adduct increases an immune response relative to a control. In some embodiments, the cysteine-containing polypeptide-small molecule fragment adduct increases a humoral immune response relative to a control. In some embodiments, the cysteine-containing polypeptide-small molecule fragment adduct increases a cell mediated immune response relative to a control. In some embodiments, the control is the level of an immune response in the subject prior to administration of the small molecule fragment. In some embodiments, the control is the level of an immune response in a subject who has not been exposed to the small molecule fragment. In some embodiments, the control is the level of a humoral immune response or a cell mediated immune response in the subject prior to administration of the small molecule fragment. In some embodiments, the control is the level of a humoral immune response or a cell mediated immune response in a subject who has not been exposed to the small molecule fragment. In some embodiments, the cysteine-containing polypeptide is overexpressed in a disease or condition. In some embodiments, the cysteine-containing polypeptide comprises one or more mutations. In some embodiments, the cysteine-containing polypeptide comprising one or more mutations is overexpressed in a disease or condition. In some embodiments, the disease or condition is cancer. In some embodiments, the cysteine-containing polypeptide is a cancer-associated protein. In some embodiments, the cysteine-containing polypeptide is overexpressed in a cancer. In some embodiments, the cysteine-containing polypeptide comprising one or more mutations is overexpressed in a cancer. In some embodiments, the cysteine-containing polypeptide is a non-denatured form of the polypeptide. In some embodiments, the cysteine-containing polypeptide comprises a biologically active cysteine site. In some embodiments, the biologically active cysteine site is a cysteine residue that is located about 10 Å or less to an active-site ligand or residue. In some embodiments, the cysteine residue that is located about 10 Å or less to the active-site ligand or residue is an active site cysteine. In some embodiments, the biologically active cysteine site is an active site cysteine. In some embodiments, the biologically active cysteine site is a cysteine residue that is located greater than 10 Å from an active-site ligand or residue. In some embodiments, the cysteine residue that is located greater than 10 Å from the active-site ligand or residue is a non-active site cysteine. In some embodiments, the biologically active cysteine site is a non-active site cysteine. In some embodiments, the cysteine-containing polypeptide is an enzyme, a transporter, a receptor, a channel protein, an adaptor protein, a chaperone, a signaling protein, a plasma protein, transcription related protein, translation related protein, mitochondrial protein, or cytoskeleton related protein. In some embodiments, the cysteine-containing polypeptide is an enzyme, a transporter, a receptor, a channel protein, an adaptor protein, a chaperone, a signaling protein, transcription related protein, or translation related protein. In some embodiments, the enzyme comprises kinases, proteases, or deubiquitinating enzymes. In some embodiments, the protease is a cysteine protease.

In some embodiments, the cysteine protease comprises caspases. In some embodiments, the signaling protein comprises vascular endothelial growth factor. In some embodiments, the signaling protein comprises a redox signaling protein. In some embodiments, the cysteine-containing polypeptide is about 20, 30, 35, 40, 45, 50, 55, 60, 65, 70, 75, 80, 85, 90, 95, 100 amino acid residues in length or more. In some embodiments, the Michael acceptor moiety comprises an alkene or an alkyne moiety. In some embodiments, the covalent bond is formed between a portion of the Michael acceptor moiety on the small molecule fragment and a portion of a cysteine residue of the cysteine-containing polypeptide. In some embodiments, F is obtained from a compound library. In some embodiments, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some embodiments, F is a small molecule fragment moiety illustrated in FIG. 1. In some embodiments, F further comprises a linker moiety that connects F to the carbonyl moiety. In some embodiments, the small molecule fragment is a small molecule fragment illustrated in FIGS. 2B and 4C. In some embodiments, the small molecule fragment has a molecular weight of about 150 Dalton or higher. In some embodiments, the small molecular fragment has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of the small molecular fragment is prior to enrichment with a halogen, a nonmetal, or a transition metal. In some embodiments, the small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher. In some embodiments, the method further comprises administering a cysteine-containing polypeptide-small molecule fragment adduct. In some embodiments, the cysteine-containing polypeptide is at most 50 amino acid residues in length. In some embodiments, the cysteine-containing polypeptide comprises an isolated and purified polypeptide comprising at least 80%, 85%, 90%, 95%, 96%, 97%, 98%, or 99% sequence identity to at least seven contiguous amino acids of an amino acid sequence selected from SEQs disclosed in the Examples. In some embodiments, the method further comprises administration of an adjuvant. In some embodiments, the small molecule fragment is formulated for parenteral, oral, or intranasal administration. In some embodiments, the subject is a human.

Disclosed herein, in certain embodiments, is a vaccine comprising a small molecule fragment of Formula (I):
wherein:

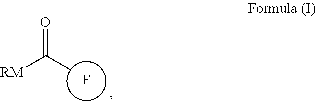

RM is a reactive moiety selected from a Michael acceptor moiety, a leaving group moiety, or a moiety capable of forming a covalent bond with the thiol group of a cysteine residue; and
F is a small molecule fragment moiety.

In some embodiments, the small molecule fragment interacts with a cysteine-containing polypeptide to form a cysteine-containing polypeptide-small molecule fragment adduct. In some embodiments, the small molecule fragment is covalently bond to a cysteine residue of the cysteine-containing polypeptide. In some embodiments, the cysteine-containing polypeptide is an endogenous cysteine-containing polypeptide expressed in a subject. In some embodiments, administration of the small molecule fragment induces an immune response. In some embodiments, administration of the small molecule fragment induces a humoral immune response. In some embodiments, administration of the small molecule fragment induces a cell mediated immune response. In some embodiments, administration of the small molecule fragment increases an immune response relative to a control. In some embodiments, administration of the small molecule fragment increases a humoral immune response relative to a control. In some embodiments, administration of the small molecule fragment increases a cell mediated immune response relative to a control. In some embodiments, the control is the level of an immune response in the subject prior to administration of the small molecule fragment. In some embodiments, the control is the level of an immune response in a subject who has not been exposed to the small molecule fragment. In some embodiments, the control is the level of a humoral immune response or a cell mediated immune response in the subject prior to administration of the small molecule fragment. In some embodiments, the control is the level of a humoral immune response or a cell mediated immune response in a subject who has not been exposed to the small molecule fragment. In some embodiments, the cysteine-containing polypeptide is overexpressed in a disease or condition. In some embodiments, the cysteine-containing polypeptide comprises one or more mutations. In some embodiments, the cysteine-containing polypeptide comprising one or more mutations is overexpressed in a disease or condition. In some embodiments, the disease or condition is cancer. In some embodiments, the cysteine-containing polypeptide is a cancer-associated protein. In some embodiments, the cysteine-containing polypeptide is overexpressed in a cancer. In some embodiments, the cysteine-containing polypeptide comprising one or more mutations is overexpressed in a cancer. In some embodiments, the cysteine-containing polypeptide is a non-denatured form of the polypeptide. In some embodiments, the cysteine-containing polypeptide is an enzyme, a transporter, a receptor, a channel protein, an adaptor protein, a chaperone, a signaling protein, a plasma protein, transcription related protein, translation related protein, mitochondrial protein, or cytoskeleton related protein. In some embodiments, the Michael acceptor moiety comprises an alkene or an alkyne moiety. In some embodiments, the covalent bond is formed between a portion of the Michael acceptor moiety on the small molecule fragment and a portion of a cysteine residue of the cysteine-containing polypeptide. In some embodiments, F is obtained from a compound library. In some embodiments, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some embodiments, F is a small molecule fragment moiety illustrated in FIGS. 2B and 4C. In some embodiments, F further comprises a linker moiety that connects F to the carbonyl moiety. In some embodiments, the small molecule fragment is a small molecule fragment illustrated in FIGS. 2B and 4C. In some embodiments, the small molecule fragment has a molecular weight of about 150 Dalton or higher. In some embodiments, the small molecular fragment has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of the small molecular fragment is prior to enrichment with a halogen, a nonmetal, or a transition metal. In some embodiments, the small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher. In some embodiments, the vaccine further comprises a cysteine-containing polypeptide-small molecule fragment adduct. In some embodiments, the cysteine-containing polypeptide comprises an isolated and purified polypeptide comprising at least 80%, 85%, 90%, 95%, 96%, 97%, 98%, or 99% sequence identity to at least seven contiguous amino acids of an amino acid sequence selected from SEQs disclosed in the Examples. In some embodiments, the vaccine further comprises an adjuvant. In some embodiments, the vaccine is formulated for parenteral, oral, or intranasal administration.

Disclosed herein, in certain embodiments, is a pharmaceutical composition comprising:
a) a cysteine-containing polypeptide covalently bond to a small molecule fragment, wherein the small molecule fragment is a small molecule fragment of Formula (I):

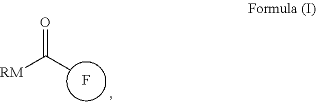

wherein:
RM is a reactive moiety selected from a Michael acceptor moiety, a leaving group moiety, or a moiety capable of forming a covalent bond with the thiol group of a cysteine residue; and
F is a small molecule fragment moiety; and
wherein the small molecule fragment is covalently bond to a cysteine residue of the cysteine-containing polypeptide; and
b) an excipient.

In some embodiments, the cysteine-containing polypeptide is a non-denatured form of the polypeptide. In some embodiments, the cysteine-containing polypeptide comprises a biologically active cysteine site. In some embodiments, the biologically active cysteine site is a cysteine residue that is located about 10 Å or less to an active-site ligand or residue. In some embodiments, the cysteine residue that is located about 10 Å or less to the active-site ligand or residue is an active site cysteine. In some embodiments, the biologically active cysteine site is an active site cysteine. In some embodiments, the biologically active cysteine site is a cysteine residue that is located greater than 10 Å from an active-site ligand or residue. In some embodiments, the cysteine residue that is located greater than 10 Å from the active-site ligand or residue is a non-active site cysteine. In some embodiments, the biologically active cysteine site is a non-active site cysteine. In some embodiments, the cysteine-containing polypeptide is an enzyme, a transporter, a receptor, a channel protein, an adaptor protein, a chaperone, a signaling protein, a plasma protein, transcription related protein, translation related protein, mitochondrial protein, or cytoskeleton related protein. In some embodiments, the cysteine-containing polypeptide is an enzyme, a transporter, a receptor, a channel protein, an adaptor protein, a chaperone, a signaling protein, transcription related protein, or translation related protein. In some embodiments, the enzyme comprises kinases, proteases, or deubiquitinating enzymes. In some embodiments, the protease is a cysteine protease. In some embodiments, the cysteine protease comprises caspases. In some embodiments, the signaling protein comprises vascular endothelial growth factor. In some embodiments, the signaling protein comprises a redox signaling protein. In some embodiments, the cysteine-containing polypeptide is about 20, 30, 35, 40, 45, 50, 55, 60, 65, 70, 75, 80, 85, 90, 95, 100 amino acid residues in length or more. In some embodiments, the cysteine-containing polypeptide comprises an isolated and purified protein. In some embodiments, the cysteine-containing polypeptide is at most 50 amino acid residues in length. In some embodiments, the cysteine-containing polypeptide comprises an isolated and purified polypeptide comprising at least 80% sequence identity to at least seven contiguous amino acids of an amino acid sequence selected from SEQs disclosed in the Examples. In some embodiments, the cysteine-containing polypeptide comprises an isolated and purified polypeptide comprising at least 85% sequence identity to at least seven contiguous amino acids of an amino acid sequence selected from SEQs disclosed in the Examples. In some embodiments, the cysteine-containing polypeptide comprises an isolated and purified polypeptide comprising at least 90% sequence identity to at least seven contiguous amino acids of an amino acid sequence selected from SEQs disclosed in the Examples. In some embodiments, the cysteine-containing polypeptide comprises an isolated and purified polypeptide comprising at least 95% sequence identity to at least seven contiguous amino acids of an amino acid sequence selected from SEQs disclosed in the Examples. In some embodiments, the cysteine-containing polypeptide comprises an isolated and purified polypeptide comprising at least 96% sequence identity to at least seven contiguous amino acids of an amino acid sequence selected from SEQs disclosed in the Examples. In some embodiments, the cysteine-containing polypeptide comprises an isolated and purified polypeptide comprising at least 97% sequence identity to at least seven contiguous amino acids of an amino acid sequence selected from SEQs disclosed in the Examples. In some embodiments, the cysteine-containing polypeptide comprises an isolated and purified polypeptide comprising at least 98% sequence identity to at least seven contiguous amino acids of an amino acid sequence selected from SEQs disclosed in the Examples. In some embodiments, the cysteine-containing polypeptide comprises an isolated and purified polypeptide comprising at least 99% sequence identity to at least seven contiguous amino acids of an amino acid sequence selected from SEQs disclosed in the Examples. In some embodiments, the cysteine-containing polypeptide comprises an isolated and purified polypeptide comprising 100% sequence identity to at least seven contiguous amino acids of an amino acid sequence selected from SEQs disclosed in the Examples. In some embodiments, the cysteine-containing polypeptide comprises an isolated and purified polypeptide consisting of 100% sequence identity to at least seven contiguous amino acids of an amino acid sequence selected from SEQs disclosed in the Examples. In some embodiments, the Michael acceptor moiety comprises an alkene or an alkyne moiety. In some embodiments, the covalent bond is formed between a portion of the Michael acceptor moiety on the small molecule fragment and a portion of a cysteine residue of the cysteine-containing polypeptide. In some embodiments, F is obtained from a compound library. In some embodiments, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some embodiments, F is a small molecule fragment moiety illustrated in FIGS. 2B and 4C. In some embodiments, F further comprises a linker moiety that connects F to the carbonyl moiety. In some embodiments, the small molecule fragment is a small molecule fragment illustrated in FIGS. 2B and 4C. In some embodiments, the small molecule fragment has a molecular weight of about 150 Dalton or higher. In some embodiments, the small molecular fragment has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of the small molecular fragment is prior to enrichment with a halogen, a nonmetal, or a transition metal. In some embodiments, the small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher. In some embodiments, the pharmaceutical composition is formulated for parenteral, oral, or intranasal administration.

Disclosed herein, in certain embodiments, is a vaccine comprising a pharmaceutical composition disclosed above. In some embodiments, the vaccine further comprises an adjuvant. In some embodiments, the vaccine is formulated for parenteral, oral, or intranasal administration.

Disclosed herein, in certain embodiments, is an isolated and purified antibody or its binding fragment thereof comprising a heavy chain CDR1, CDR2 and CDR3 sequence and a light chain CDR1, CDR2 and CDR3 sequence, wherein the heavy chain and light chain CDRs interact with a cysteine-containing polypeptide that is covalently bond to a small molecule fragment, wherein the small molecule fragment is a small molecule fragment of Formula (I):

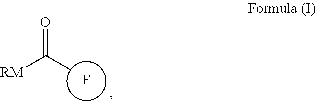

wherein:
RM is a reactive moiety selected from a Michael acceptor moiety, a leaving group moiety, or a moiety capable of forming a covalent bond with the thiol group of a cysteine residue; and
wherein the small molecule fragment is covalently bond to a cysteine residue of the cysteine-containing polypeptide.

In some embodiments, the antibody or its binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof.

Disclosed herein, in certain embodiments, is a kit comprising a pharmaceutical composition described above.

Disclosed herein, in certain embodiments, is a kit comprising an isolated and purified antibody or its binding fragment thereof disclosed above.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-1I provides an overview of chemical proteomic mapping of the cysteine reactivity changes of activated T cells. FIG. 1A is a diagram of experimental workflow for proteomic experiments measuring cysteine reactivity (TMT-ABPP) and protein expression (TMT-exp) in primary human T cells: 1) T cells were isolated from peripheral blood mononuclear cells (PBMCs) using negative selection EasySep Human T Cell Isolation Kit. PBMCs were isolated from blood of healthy donors over standard Lymphoprep gradient; 2) isolated T cells were activated in αCD3 and αCD28 pre-coated 6-well plates (3 days) and expanded by growing in RPMI media containing recombinant IL2 (10 U/mL) for 10-12 days, splitting the cells every 3-4 days; 3) control (expanded, but not activated) and activated T cells were processed for TMT-ABPP and TMT-exp analysis (see Experimental Methods); and 4) cysteine reactivity and protein expression changes were distinguished by integrating TMT-ABPP and TMT-exp data. FIG. 1B diagrams the overlap between proteins quantified by TMT-exp and immune-relevant proteins. Results were derived from two independent TMT-exp experiments (four biological replicates), where a protein was required to have two unique peptides per experiment. FIG. 1C shows protein expression differences between control and activated T cells. Results represent mean values from two independent TMT-exp experiments (four biological replicates). FIG. 1D shows representative protein expression differences between control and activated T cells, where results from both TMT-ABPP (black dots) and TMT-exp (green dots) concordantly support the expression changes. Horizontal lines mark average values for the indicated groups. FIG. 1E is a bar graph representation of the fraction of proteins with cysteine reactivity changes observed for proteins with the indicated numbers of quantified peptides in TMT-ABPP experiments. A cysteine was considered to show a reactivity change if the R value for its parent tryptic peptide differed more than two-fold from the protein expression value measured by TMT-exp (if quantified) and/or from the median R value of all quantified cysteines on that same protein measured by TMT-ABPP (for proteins with ≥5 quantified cysteines). Proteins with only 1 or 2 quantified cysteines in TMT-ABPP experiments were not interpreted for reactivity changes (gray bars). FIG. 1F-FIG. 1I are representative cysteine reactivity changes in activated human T cells organized by functional categories. Horizontal lines mark average values for the indicated groups. FIG. 1F shows reactivity changes in active-site cysteines in redox-related proteins; x-ray crystal structure of FAD bound to the active site of GSR (PDB: 1GRF) with the reactivity-changing cysteine C102 highlighted in blue. FIG. 1G shows reactivity changes in metal-binding cysteines; solution NMR structure of the EF-hand domain of LCP-1 (PDB: 5JOJ) bound to calcium ions with the reactivity-changing cysteine C42 highlighted in blue. LCP-1 α-helices undergoing most significant rearrangement upon calcium binding are highlighted in green. FIG. 1H shows reactivity changes in cysteines at DNA/RNA-binding sites; cryo-EM structure of human ribonuclease P RPP30 (PDB: 6AHU) bound to mature tRNA with the reactivity-changing cysteine C225 highlighted in blue. FIG. 1I shows reactivity changes in cysteines at cofactor/metabolite-binding sites; x-ray crystal structure of the human NADP(+)-dependent isocitrate dehydrogenase 1 (IDH1) in complex with NADP, isocitrate, and calcium (PDB: 1T0L) with the reactivity-changing cysteine C269 highlighted in blue.

FIG. 2A-2J provides an overview of chemical proteomic mapping of fragment electrophile-cysteine interactions and reactivity changes in human T cells. FIG. 2A is an experimental workflow for chemical proteomic experiments measuring scout fragment electrophile effects on cysteine reactivity in primary human T cells: 1) T cells isolated from human blood were lysed by probe sonication (2×8 pulses) and the soluble and particulate fractions separated by ultracentrifugation (100,000 g, 45 min) and treated with DMSO or scout fragments (KB02, KB05; 500 μM, 1 h); 2) fractions were then treated with a broadly cysteine-reactive iodoacetamide (IA) probe (IA-alkyne and IA-desthiobiotin (DTB); 100 μM, 1 h)) for isoTOP-ABPP and TMT-ABPP, respectively; 3) DMSO- and fragment-treated T cell proteomes were analyzed by isoTOP- and TMT-ABPP, where a cysteine was considered liganded if it displayed an R value (DMSO/scout fragment) of ≥5 (see Experimental Methods for more information). This protocol was applied to both control and activated T cells. FIG. 2B are structures of scout fragments KB02 and KB05. FIG. 2C-FIG. 2D are pie chart representations of cysteines (C) and proteins (D) liganded with scout fragments. Results were obtained by combining soluble and particulate proteomic fraction data for KB02 and KB05 treatments (500 μM, 1 h) of both control and activated T cells. R-values within each experimental treatment group were derived from 3-5 independent isoTOP-ABPP experiments and 4 independent TMT-ABPP experiments (6 TMT channels). A cysteine was required to be quantified in at least two experiments for each compound treatment or proteomic fraction group to be reported. FIG. 2E are bar graphs showing the total number (left) and percentage (right) of liganded cysteines per total number of cysteines quantified across the indicated reactivity ranges, where cysteine reactivity was determined by isoTOP-ABPP experiments performed with different concentrations of the IA-alkyne probe (10 and 100 μM), as described previously (Weerapana et al., 2010). FIG. 2F are bar graphs showing the total number (left) and percentage (right) of liganded proteins with expression or reactivity changes in activated T cells. FIG. 2G are bar graphs showing percent MS3-signal intensity for quantified peptides from PDCD1, revealing elevated expression of this protein in activated T cells and KB02-sensitivity for C93 in these cells. Results represent mean R-values derived from 1-4 independent TMT-ABPP experiments (6 TMT channels). FIG. 2H is a diagram showing the overlap of liganded proteins with immune-relevant proteins. FIG. 2I is a diagram showing the fraction of liganded proteins from total proteins with human genetics-based immune phenotypes. FIG. 2J is a diagram showing the fraction of liganded proteins from total proteins encoded by T cell proliferation genes.

FIG. 3A-3F provides an overview of the ligandable cysteines in immune-relevant targets. FIG. 3A is a diagram of TCR and NF-κB signaling pathways marking proteins that possess cysteines liganded by scout fragments (green) or elaborated electrophilic compounds (blue). FIG. 3B shows the physical location of ligandable cysteines in three-dimensional structures of immune-relevant kinases IKBKB (PDB: 4E3C) and CHUK (PDB: 5EBZ), including non-active site cysteines (C464 in IKBKB, C406 in CHUK). FIG. 3C is a pie chart showing the fractions of liganded transcription factors and adaptor proteins that are also immune-relevant (immune-enriched (blue) and/or have human genetics-based immune phenotypes (green, listed on the right)). FIG. 3D and FIG. 3E show the physical location of ligandable cysteines at sites of protein-protein interactions for MALT1 (C71; interaction partner BCL10; PDB: 6GK2) and IRF9 (C313 (mouse orthologue to human C319); interaction partner STAT2; PDB: 5OEN). FIG. 3F provides a ligandability analysis of Reactome pathways within Immune System (left, hierarchical level 3) grouped according to their parent nodes (blue shades, Immune System hierarchical level 2) or Signal Transduction (right, hierarchical level 2) categories.

FIG. 4A-4D is an overview of a multidimensional screen to identify elaborated electrophilic compounds that suppress T cell activation. FIG. 4A is a workflow for T-cell activation screen. Primary human T cells were treated with a focused library of elaborated electrophilic compounds (10 μM, structures of compounds in FIG. 12), a positive control immunosuppressive compound (DMF, 50 μM), or DMSO under TCR-stimulating conditions in 96-well plates pre-coated with 5 μg/mL αCD3 and 2 μg/mL αCD28 for 24 h. T cell activation was measured using a combination of markers, including IL2 and IFNγ secretion, as well as surface expression of CD25 and CD69. T cell viability was measured by flow cytometry using a Fixable Near-IR LIVE/DEAD™ Cell Stain. Compounds were considered as active hits if they reduced IL2 cytokine production by >65% with <15% reduction in T cell viability in the cytotoxicity assay compared to DMSO control. Representative primary screen results are shown for active hit compounds, inactive compounds, and cytotoxic compounds. FIG. 4B shows a pie chart of screening results for elaborated electrophilic compounds. FIG. 4C are structures of active hit compounds selected for follow-up studies, including two acrylamides (BPK-21, BPK-25), two chloroacetamides (EV-3, EV-93) and DMF as a positive control. FIG. 4D are line graphs showing T cell activation and cytotoxicity profiles for selected hit compounds. Data are presented as the mean percentage of DMSO-treated control cells±SEM; n=3/group.

FIG. 5A-5H is an overview of the cysteines liganded by active compounds in human T cells. FIG. 5A is a heatmap showing liganded cysteine profiles for active compounds in primary human T cells (treated with the indicated concentrations of compounds (μM) for 3 h followed by ABPP analysis). Cysteines quantified for at least two active compounds with R values ≥4 (DMSO/compound) for at least one of them are shown. Results were obtained by combining isoTOP-ABPP and TMT-ABPP data for both soluble and particulate proteomic fractions. R-values within each experimental treatment group were derived from 3-6 independent isoTOP-ABPP experiments and 2-3 independent TMT-ABPP experiments (6 TMT channels). A cysteine was required to be quantified in at least two experiments for each proteomic fraction. FIG. 5B is a heatmap showing cysteines liganded by active compounds that are found in immune-relevant proteins (immune-enriched and/or have human genetics-based immune phenotypes). FIG. 5C is a pie chart showing the distribution of protein classes containing cysteines liganded by active compounds. FIG. 5D-FIG. 5E are comparisons of cysteines liganded by active compounds versus scout fragments in human T cells, as displayed in correlation plot (D) and pie chart (E) analyses. Cysteines liganded by both active compounds and scout fragments, only by active compounds, and only by scout fragments shown in purple, red, and blue, respectively. FIG. 5F is a bar graph of the percent prediction success rate querying for pockets within the indicated distances from cysteines liganded by active compounds as measured in angstroms. FIG. 5G shows modeling of active compound interactions with C203 in the TLR domain of MYD88. Predicted pockets highlighted as green mesh. Docking of BPK-21 and BPK-25 interactions with C203 showing preferential liganding with BPK-25 due to predicted hydrogen bonds with E183 and R188 (bottom, left), which are not accessible in docked structure of BPK-21 (bottom, right). A second pocket containing C274, which is liganded by scout fragments is also shown. FIG. 5H shows modeling of active compound interactions with C342 in the helicase domain of ERCC3. Docking of BPK-21 and BPK-25 interactions with C342 showing preferential liganding with BPK-21 due to predicted hydrogen bonds with T469 and Q497 and π-π interaction with W469, which are less accessible in the docked structure of BPK-25 (FIG. 13B).

FIG. 6A-6K is a functional analysis of protein targets of active compounds in human T cells. FIG. 6A is a bar graph showing the effects of active compounds versus the GCLC inhibitor BSO on glutathione (GSH) content and T cell activation parameters. Data are presented as the mean percentage of DMSO-treated control cells I SEM; n=3/group. FIG. 6B shows effects of active compounds on NF-κB activity. Flow cytometry analysis of phosphorylation (S536) of p65 in T cells treated with active compounds at the indicated concentrations (24 h). Plots show p65 (pS536) content in DMSO versus compound-treated cells. Data are from a single experiment representative of at least two independent biological experiments. FIG. 6C shows effects of active compounds on NFAT activity. Jurkat-Lucia™ NFAT cells were stimulated with PMA (50 ng/mL) and ionomycin (3 μg/mL) in the presence of the indicated concentrations of active compounds and NFAT transcriptional activity was determined by the levels of Lucia luciferase measured with QUANTI-Luc™ detection reagent in comparison to DMSO-treated control cells. Data are presented as the mean percentage of DMSO-treated control cells±SEM; n=2-5/group. FIG. 6D shows the effect of genetic disruption of representative targets of active compound BPK-21 by CRISPR/Cas9 genome editing on T cell activation. Target disruption was considered to have an effect if T cell activation was suppressed >33% with a p value <0.01. Data are presented as the mean percentage of control guide sgRNA-treated control cells±SEM; n=6/group. **** p<0.0001. FIG. 6E shows the effect of genetic disruption of ERCC3 and BPK-21 treatment and that it produces similar degrees of blockade of T cell activation. Data are presented as the mean percentage of control sgRNA-treated control cells±SEM; n=6/group. **** p<0.0001. FIG. 6F is a heat map showing that active compound EV-3 engages C45 and C28 of BIRC2 and BIRC3. FIG. 6G are domain maps for BIRC2, BIRC3, and related protein XIAP, highlighting location of EV-3-sensitive cysteines in BIRC2/3. FIG. 6H shows the physical location of EV-3-sensitive cysteine C28 in structure of a BIRC3-TRAF2 protein complex (PDB: 3M0A). FIG. 6I-FIG. 6J show that EV-3 causes loss of BIRC2 and BIRC3 in human T cells. FIG. 6I, left panels, are western blots showing reductions in BIRC2 and BIRC3 content in human T cells treated with EV-3 (10 μM), but not other active compounds (DMF (50 μM), BPK-21 (20 μM), and BPK-25 (10 μM)). The BIR2 domain ligand AT406 (1 μM) was also included for comparison and found to cause loss of BIRC2, but not BIRC3. Right panels, western blots showing that the proteasome inhibitor MG132 (10 μM) blocks EV-3-induced loss of BIRC2 and BIRC3. All treatments were for 24 h. FIG. 6J is a bar graph representation of western blot data shown in (I), where protein band intensity calculations were made using ImageJ software and represent the mean percentage of DMSO treated control±SEM; n=2-5/datapoint. FIG. 6K is a bar graph showing the effect of genetic disruption of BIRC2, BIRC3, or BIRC2 and BIRC3, by CRISPR/Cas9 genome editing on T cell activation. Target disruption was considered to have an effect if T cell activation was suppressed >33% with a p value <0.01. EV-3 treatment in BIRC2/BIRC3-disrupted T cells is shown for comparison. Data are presented as the mean percentage of control guide sgRNA-treated control cells±SEM; n=6/group. **** p<0.0001.

FIG. 7A-7G is an overview of experiments demonstrating active compound BPK-25 promotes degradation of NuRD complex in human T cells. FIG. 7A is an experimental workflow for quantitative proteomic experiments evaluating protein abundance changes caused by active compound treatment in primary human T cells: 1) T cells were treated with hit compounds (DMF (50 μM), EV-3 (10 μM), BPK-21 (20 μM), BPK-25 (10 μM)) or DMSO control for 24 h; 2) cells were then processed and analyzed by TMT-based quantitative proteomics where a 50% reduction in average peptide signals for a protein were interpreted as a reduction in the quantity of that protein. FIG. 7B is a scatter plot representation of protein abundance changes caused by BPK-25 (10 μM, 24 h) in two independent replicate experiments, with decreases in NuRD complex components highlighted in red. FIG. 7C is a heatmap of top proteins with decreased abundance in BPK-25-treated T cells showing that the subset of these proteins in the NuRD complex (asterisks) were largely unaltered by other active compounds and blocked in their degradation by co-treatment with the proteasome inhibitor MG132. Additional NuRD complex members are also displayed, and most of these proteins showed evidence of reduced abundance (25-50%) in T cells treated with BPK-25. FIG. 7D shows mRNA measurements at 24, 6, and 3 hour time points and shows that active compound treatment did not, in general, reduce the mRNA content for NuRD complex members. mRNA quantities were measured by RNAseq. Data are presented as the mean percentage of DMSO-treated control cells±SEM; n=3/group. FIG. 7E is a bar graph of mRNA levels showing time-dependent reductions in NuRD complex members in human T cells treated with BPK-25 (10 μM). Left, representative western blots. Right, quantification of changes in protein. FIG. 7F is a bar graph representation of western blot data, where protein band intensity calculations were made using ImageJ software and represent the mean percentage of DMSO treated control±SD; n=2. FIG. 7G is a heat map showing the changes in cysteine reactivity for NuRD complex members in human T cells treated with BPK-25 and other active compounds.

FIG. 8A-8D is an overview of the chemical proteomic mapping of cysteine reactivity changes in activated T cells. FIG. 8A shows an extended experimental workflow for proteomic experiments measuring cysteine reactivity (TMT-ABPP) in primary human T cells. FIG. 8B shows an extended experimental workflow for proteomic experiments measuring protein expression (TMT-exp) in primary human T cells. FIG. 8A is a pie chart showing the fraction of proteins with human genetics-based immune phenotypes and immune-enriched protein expression from total proteins showing cysteine reactivity changes in activated T cells. FIG. 8B is a bar graph representation of proteins with cysteine reactivity changes organized by molecular function GO term enrichment. FIG. 8C is a graph showing the representative cysteine reactivity changes in activated human T cells for cysteines at protein-protein interaction (PPI) surfaces. FIG. 8D shows 3 dimensional models of complexed human proteins. Representative cysteine reactivity changes in activated human T cells for cysteines in cofactor/metabolite-binding sites; x-ray crystal structure of the active form of human origin recognition complex subunit 1 (ORC1) in complex with ATP and Mg2+(PDB: 5UJ7) with the reactivity-changing cysteine C506 highlighted in blue (top image); x-ray crystal structure of human 3′-phosphoadenosine-5′-phosphosulfate synthetase 1 (PAPSS1) in complex with ADP (PDB: 1X6V) with the reactivity-changing cysteine C165 highlighted in blue (upper middle image); x-ray crystal structure of human glucose-6-phosphate dehydrogenase (G6PD) in complex with NADP+(PDB: 2BH9) with the reactivity-changing cysteine C385 highlighted in blue (lower middle image); x-ray crystal structure human methylmalonyl-CoA mutase (MMUT) in complex with B12 (PDB: 2XIQ) with the reactivity-changing cysteine C742 highlighted in blue (bottom image).

FIG. 9A-9G is an overview of the chemical proteomic mapping of fragment electrophile-cysteine interactions in human T cells. FIG. 9A shows an extended experimental workflow for chemical proteomic experiments measuring scout fragment electrophile effects on cysteine reactivity in primary human T cells using isobaric tandem mass tags for mass differentiation and MS3-based quantification (TMT-ABPP). FIG. 9B is an extended experimental workflow for chemical proteomic experiments measuring scout fragment electrophile effects on cysteine reactivity in primary human T cells using clickable, TEV protease-sensitive, isotopically labeled tags for mass differentiation and MS1-based quantification (isoTOP-ABPP). FIG. 9C-FIG. 9D are comparisons of R-values from isoTOP-ABPP and TMT-ABPP experiments, as displayed in correlation plot (C) and bar graph (D) analyses. Results represent mean R-values derived from 3-5 independent isoTOP-ABPP experiments and 4 independent TMT-ABPP experiments (6 TMT channels) for each compound treatment and proteomic fraction (2-5 biological donors for each method). A cysteine was required to be quantified in at least two experiments for each compound treatment or proteomic fraction group to be reported. KB02-treated soluble proteome samples are used as an example for the correlation plot. For (C) and (D), data points with R(TMT-ABPP)≥5 are colored in blue with the exception of rare outlier cases that also exhibited R(isoTOP-ABPP)<5 and R(isoTOP-ABPP)<1/2R(TMT), which are colored in red. FIG. 9E is a bar graph showing the total number of quantified peptides in isoTOP-ABPP and TMT-ABPP experiments for soluble and particulate proteomic fractions of primary human T cells. Results represent a combination of data from KB02 and KB05 experiments (500 μM, 1 h) with both control and activated T cells. R-values within each experimental treatment group were derived from 3-5 independent isoTOP-ABPP experiments and 4 independent TMT-ABPP experiments (6 TMT channels). A cysteine was required to be quantified in at least two experiments for each compound treatment or proteomic fraction group to be reported. FIG. 9F shows on the left: MS1 signal intensities for USP16_C205 in KB02- and KB05-treated expanded T cell proteome; right: USP16_C205 reactivity change in activated human T cells. FIG. 9G is a bar graph showing the fraction of liganded proteins from total proteins found in previously described immune-enriched modules.

FIG. 10 is a diagram of TCR and NF-κB signaling pathways related to FIG. 3. The peptide quantification events in experiments with scout fragments and elaborated compounds with the following colors used to mark proteins with no (grey), one (yellow), two (brown), or >two (orange) quantified peptides.

FIG. 11A-11C show the number of quantified ligandable cysteines. FIG. 11A shows flow cytometry analyses of cell populations from FIG. 5. FIG. 11B is a bar graph showing the total number of quantified (black) and liganded (R≥4, red) cysteines in cells treated with active compounds. Results are obtained by combining isoTOP-ABPP and TMT-ABPP data for both soluble and particulate proteomic fractions. R-values within each experimental treatment group were derived from 3-6 independent isoTOP-ABPP experiments and 2-3 independent TMT-ABPP experiments (6 TMT channels). A cysteine was required to be quantified in at least two experiments for each proteomic fraction to be reported. FIG. 11C shows bar graphs showing the total number of liganded proteins as relates to the corresponding number of quantified (blue) and liganded (R≥4, red) cysteines per protein. Results are obtained by combining isoTOP-ABPP and TMT-ABPP data for both soluble and particulate proteomic fractions for each compound treatment. A cysteine was required to be quantified in at least two experiments for each proteomic fraction to be reported.

FIG. 12A-12F shows a functional analysis of protein targets of active compounds in human T cells, related to FIG. 5. FIG. 12A is a pie chart showing the fraction of protein targets of active compounds with available crystal structures containing the corresponding liganded cysteine residues. FIG. 12B is a computer-generated model of BPK-21 and BPK-25 interactions with C91 of stimulator of interferon genes protein (STING, or TMEM173). FIG. 12C is a graph of IRF response of THP1-Lucia™ ISG cells. THP-1 Lucia ISG cells were treated with DMSO or BPK-25 in the presence of viral dsDNA (2 μg/mL) for 24 h and the levels of IRF-induced Lucia luciferase were determined using QUANTI-Luc™. Cell viability was measured using CellTiter-Glo™ assay. Luminescence signals for test samples were normalized to DMSO-treated samples and reported as relative light units (RLU)±SEM; n=3/group. FIG. 12D is a bar graph showing the effect of BPK-25 treatment on gene expression related to TMEM173/STING pathway activation in PBMCs. PBMCs were treated with DMSO or BPK-25 (10 μM) for 5 h and stimulated with cGAMP (2 μM) for 2 h. Relative expression of IL-6, IL-1β, and IP10 (CXCL10) genes was measured by qPCR and normalized to actin. FIG. 12E are bar graphs showing the effect of BPK-25 treatment on secretion of cytokines related to TMEM173/STING pathway activation in PBMCs. PBMCs were treated with DMSO or BPK-25 (10 μM) for 5 h and stimulated with cGAMP (10 μM) for 20 hours. Cytokine levels (IFN-(3, IP10 (CXCL10), IL-6, MCP-1, TNFα) were measured using standard ELISA and Bio-Plex protocols (see Supplementary Methods for details). Data are presented as the mean percentage of DMSO-treated control cells±SEM; n=3/group. FIG. 12 F shows flow cytometry analysis of T cell activation following ERCC3 gene disruption by CRISPR/Cas9 genome editing. T cells were activated for 2 days prior to Cas9 RNP transfection and were then cultured in IL-2 containing RPMI media to return the cells to a quiescent state. Seven days post-transfection, T cells were stimulated overnight with αCD3 and αCD28 antibodies in the presence of DMSO or BPK-21 with the activation monitored by measuring CD25 and CD69 expression levels. The following FACS gating strategy was used: 1) Lymphocyte gating; 2-3) Doublet discrimination by plotting forward scatter height (x-axis) versus forward scatter width (y-axis) and side scatter height (x-axis) versus side scatter width (y-axis); 4) Live cells were selected by plotting APC-Cy7 channel (x-axis, area, logarithmic scale, eBioscience™ Fixable Viability Dye eFluor™ 780) versus side scatter area (y-axis) and gating on the negative cell population; 5) Transfected cells were selected by gating on FITC-positive cell population (GFP-positive cells). GFP-positive cells were further analyzed for CD25 and CD69 expression levels by measuring mean fluorescence intensity of the PE- (PE-CD25) and APC-channels (APC-CD69). Representative histograms showing CD25 levels in stimulated T cells in the presence of scrambled sgRNA control (light blue) or ERCC3 guide RNAs (green) in the presence of DMSO or BPK-21 (orange) are presented. Data is representative of a total of six replicate treatments.

FIG. 13A-13F shows a functional analysis of protein targets of active compounds in human T cells, related to FIG. 6. FIG. 13A shows MS1 signal intensities for BIRC3_C28, BIRC3_C164, and BIRC2_C45 in isoTOP-ABPP experiments of expanded T cells treated with EV-3 (10 μM, 3 h) or DMF (50 μM, 3 h). FIG. 13B—FIG. 13C show that EV-3 causes loss of BIRC2 and BIRC3 in human T cells. FIG. 13B shows a full western blot from FIG. 6I (right) showing reductions in BIRC2 and BIRC3 content in human T cells treated with EV-3 (1-10 μM, 24 h) and the blockade of EV-3-induced loss of BIRC2 and BIRC3 by co-treatment with the proteasome inhibitor MG132 (10 μM). FIG. 13C is a western blot showing time-dependent reductions in BIRC2 and BIRC3 content in human T cells treated with EV-3 (10 μM). FIG. 13D is a bar graph showing mRNA content for BIRC2 and BIRC3 in T cells treated with EV-3 (10 μM), DMF (50 μM), AT-406 (1 μM), BPK-25 (10 μM), or BPK-21 (20 μM). All treatments were done with T cells for 24 hours. Data are presented as the mean percentage of DMSO-treated control cells±SEM; n=3/group. FIG. 13F is a bar graph showing the effect of genetic disruption of representative targets of active compound EV-3 by CRISPR/Cas9 genome editing on T cell activation. Target disruption was considered to have an effect if T cell activation was suppressed >33% with a p value <0.01. Data are presented as the mean percentage of control guide sgRNA-treated control cells±SEM; n=3/group. **** p<0.0001. FIG. 13E shows flow cytometry analysis of T cell activation following BIRC2, BIRC3, or BIRC2 and BIRC3 gene disruption by CRISPR/Cas9 genome editing.

FIG. 14A-14B provides an overview of chemical proteomic mapping of the cysteine reactivity changes of activated T cells. FIG. 14A provides a GO-term enrichment analysis for proteins undergoing reactivity (top) or expression (bottom) changes in activated T cells. Top-10 enriched biological processes are shown for the expression changes group. Red bold font highlights immune-relevant pathways enriched in expression changes group and cell redox homeostasis pathway enriched in reactivity changes group. FIG. 14B provides an overview of chemical proteomic mapping of fragment electrophile-cysteine interactions and reactivity changes in human T cells. Shows the location of liganded cysteine C408 (blue) and pathogenic missense mutations (yellow—mutation of H112, which is within 5 Å of C408, red—other mutations) in a three-dimensional structure of adenosine deaminase 2, CECR1 (PDB: 3LGD).

FIG. 15A-15D is an overview of a multidimensional screen to identify elaborated electrophilic compounds that suppress T cell activation. FIGS. 15A and 15B show structures (A) and activity (B) of a set of four stereoisomeric probes, where one of the stereoisomers (EV-96) stereoselectively inhibited T-cell activation (B). In (A), the stereoisomeric relationships of compounds are shown in blue (diastereomers) and red (enantiomers). Red color in chemical structures indicates the acrylamide reactive group. In (B), T-cell activation (CD25) and cytotoxicity profiles are shown for the stereoisomeric probes (5 μM, 24 h treatment). Data are presented as the mean percentage of DMSO-treated control cells±SD; n=2-5/group. ***p<0.001 by two-tailed unpaired t test with Welch's correction compared to EV-97 treatment. T-cell activation (CD25) profiles for the enantiomeric probes EV-96 and EV-97 (24 h treatment). FIG. 15C shows data presented as the mean percentage of DMSO-treated control cells±SEM; n=4-5/group. **, p<0.01 by two-tailed unpaired t test with Welch's correction compared to 0.5 μM treatment groups. FIG. 15D shows T-cell activation and cytotoxicity profiles for the active enantiomer EV-96. Data are presented as the mean percentage of DMSO-treated control cells±SEM; n=3-5/group.

FIG. 16A-16J shows the mechanistic analysis of active compounds in human T cells. FIGS. 16A and 16B show effects of active compounds on immune-relevant NFκB and mTOR pathways, as determined by western blot analysis of phosphorylation of IκBa (S32/S36) and S6K (T389), respectively, in stimulated T cells treated with DMSO, active (EV-3 (10 μM), BPK-21 (20 μM), BPK-25 (10 EV-96 (5 μM), or control (EV-97 (5 μM)) compounds for 24 h. (A) Representative Western blots. (B) Quantitation of indicated signals for p-IκBα (S32/S36, top) and p-S6K (T389). Protein band intensity calculations were made using ImageJ software and are presented as the band intensity±SEM; n=5-8/group (p-S6K) or mean percentage of DMSO-treated control±SD; n=2-5/group (p-IκBa). *, p<0.05; **, p<0.01 by two-tailed unpaired t test with Welch's correction compared to DMSO(αCD3/αCD28) control. FIG. 16C Top: shows active compound EV-3 engages C45 and C28 of BIRC2 and BIRC3, respectively. Heat map showing cysteines liganded by active compounds in BIRC2 and BIRC3. Bottom: Domain maps highlighting location of EV-3-sensitive cysteines in BIRC2 and BIRC3. FIG. 16D shows the location of EV-3-sensitive cysteine C28 in structure of a BIRC3-TRAF2 protein complex (PDB: 3M0A). FIG. 16E shows that EV-3 causes loss of BIRC2 and BIRC3 in human T cells. Western blots showing reductions in BIRC2 and BIRC3 content in human T cells treated with EV-3 (10 μM), but not other active compounds (DMF (50 μM), BPK-21 (20 μM), and BPK-25 (10 μM)). The BIR3 domain ligand AT406 (1 μM) was also included for comparison and found to cause loss of BIRC2, but not BIRC3. Right panels: western blots showing that the proteasome inhibitor MG132 (10 μM) blocks EV-3-induced loss of BIRC2 and BIRC3. All treatments were for 24 h. FIGS. 16F-16G show impact of cysteine mutagenesis on EV-3-mediated degradation of BIRC2 and BIRC3. Plasmids expressing FLAG epitope-tagged versions of wild-type (WT) or the indicated cysteine-to-alanine mutants of BIRC2 (C45A) and BIRC3 (C28A) were co-transfected into primary human T cells with mCherry-expressing plasmid to control for transfection efficiency for 24 h. Cells were then treated with DMSO, EV-3 (10 μM), or AT-406 (1 μM) for 24 h and analyzed by anti-FLAG western blotting. (F) Representative Western blot showing reductions in WT-BIRC2 and BIRC3, but not cysteine mutants in EV-3-treated T cells. Note that AT406 still promotes the degradation of the BIRC2 C45A mutant because this compound does not engage C45. (G) Quantitation of western blot data, where protein band intensity calculations were made using ImageJ software and represent the mean percentage of DMSO treated control±SEM; n=3 independent experiments per group. *p<0.05; **p<0.01 by two-tailed unpaired t test with Welch's correction compared to respective DMSO treatments. FIG. 16H shows the effect of genetic disruption of BIRC2, BIRC3, or BIRC2 and BIRC3, by CRISPR/Cas9 genome editing on T cell activation. Target disruption was considered to have an effect on T-cell activation if suppression was >33% with a p value <0.01. EV-3 treatment in BIRC2/BIRC3-disrupted T cells is shown for comparison. Data are presented as the mean percentage of control guide sgRNA-treated control cells±SEM; n=6/group. **, p<0.01 by two-tailed unpaired t test with Welch's correction compared to control guides. FIG. 16I shows experimental workflow for quantitative proteomic experiments evaluating protein expression changes (TMT-exp experiments) caused by active compound treatment in primary human T cells: 1) T cells were treated with hit compounds (DMF (50 μM), EV-3 (10 1μM), BPK-21 (20 μM), BPK-25 (10 μM)) or DMSO control for 24 h; 2) cells were then processed and analyzed by TMT-exp, where a >50% reduction in average peptide signals for a protein was interpreted as a reduction in the quantity of that protein. FIG. 16J shows the volcano plot representation of protein expression changes caused by BPK-25 (101.1M, 24 h) with significant decreases in NuRD complex proteins highlighted in red. Fig. K shows T-cell activation and cytotoxicity profile of the pan-HDAC inhibitor vorinostat. Data are presented as the mean percentage of DMSO-treated control cells±SD; n=2-4/group.

FIG. 17A-17I shows that EV-96 stereoselectively engages and degrades immune kinases in T cells. FIG. 17A provides a heatmap showing cysteines that are engaged >50% by EV-96, EV-97, EV-98, and/or EV-99 (5 μM, 3 h). For inclusion in the heat map, cysteines were also required to show a concentration-dependent increase in engagement by the relevant stereoisomeric electrophile at 20 μM. FIG. 17B provides a western blot showing reduction in TEC protein content in human T cells treated with EV-96 (5 μM), but not EV-97 (5 μM of each compound, 24 h). FIG. 17C shows unenriched proteomic analysis (TMT-exp) comparing protein expression signals in DMSO-treated αCD3/CD28-stimulated (DMSO-stim)-versus-naïve control (DMSO-ctrl) T cells (y-axis) and EV-97-treated-versus-EV-96-treated stimulated T cells (x-axis). T cells were treated with DMSO or compounds (5 μM each) for 8 h. Red background denotes proteins with: i)>2-fold higher expression in stimulated T cells treated with EV-97 versus EV-96; and ii)<1.5 fold change in expression in DMSO-stim vs DMSO-ctrl T cells. The two proteins in this region are marked and colored green. Proteins showing >2-fold changes in expression in DMSO-stim vs DMSO-ctrl T cells were removed from the analysis. FIG. 17D provides protein sequences showing EV-96-liganded cysteine in TEC (C449) and its conservation in ITK (C442). FIGS. 17E-FIG. 17F provide western blot analysis (E) showing reductions in ITK protein (8 h) and PLCG1 phosphorylation (Y783, 24 h) in αCD3/CD28-stimulated (stim) T cells treated with EV-96, but not EV-97 (5 μM of each compound). (F) Quantitation of western blot data, where protein band intensity calculations were made using ImageJ software and represent the mean percentage of DMSO-treated control±SD; n=2-5 independent experiments per group. **p<0.01 by two-tailed unpaired t test with Welch's correction compared to EV-97 treatment. FIG. 17G provides a western blot analysis showing reductions in ITK protein in stimulated, but not control (naïve) human T cells treated with EV-96 (5 μM) and that co-treatment with the proteasome inhibitor MG132 (10 μM) blocks EV-96-induced reductions in ITK. FIG. 17H shows quantitation of unenriched proteomic (TMT-exp) data showing the effects of EV-96 and EV-97 (5 μM of each compound, 8 h) on ITK protein content in naïve control (ctrl) T cells versus αCD3/CD28-treated (stimulated, stim) T cells. Data are presented as the mean percentage of DMSO-treated control±SEM; n=4 independent experiments per group. ****p<0.0001 by two-tailed unpaired t test with Welch's correction compared to DMSO-treated stim control. FIG. 17I provides a western blot showing that pre-treatment with the ITK inhibitor PF-064655469 (1 h, 5 μM), which covalently modifies C442, blocks EV-96-induced degradation of ITK. PF-064655469 did not independently alter ITK protein in T cells.

FIG. 18A-18B provides for a chemical proteomic map of cysteine reactivity changes in activated T cells. FIG. 18A shows the principal component analysis (PCA) of protein expression profiles in naïve, expanded, and activated T cells. Different cell states are indicated by colors in the legend, with each independent replicate shown separately (4-8 replicates from 4 independent donors per group). Reactome pathways that were enriched (Benjamini-Hochberg corrected p-values <0.05) for each principal component as determined by Perseus. FIG. 18B shows representative cysteine reactivity changes in activated human T cells organized by functional categories. Dashed line marks unchanged R value for act vs cntrl cells, and horizontal black lines for each protein measurement mark average R value for the quantified peptides (excluding the reactivity-changing cysteine(s)) from that protein. Green dots represent act/ctrl expression value for proteins in TMT-exp experiments. Top: cysteines at DNA/RNA-binding sites; bottom: cryo-EM structure of human ribonuclease P RPP30 (PDB: 6AHU) bound to mature tRNA with the reactivity-changing cysteine C225 highlighted in blue.

FIG. 19A-19F provides for chemical proteomic map of fragment electrophile-cysteine interactions in human T cells. FIG. 19A provides a bar graph showing types of pathogenic mutations in liganded proteins with immune phenotypes (OMIM). FIG. 19B shows the fraction of proteins with pathogenic missense mutations and immune phenotypes (OMIM) for which crystal structures are available. Crystal structures that contain at least one liganded cysteine and pathogenic missense mutation are shown in green. MM—missense mutations. FIG. 19C provides a bar graph showing distance of pathogenic missense mutations from liganded cysteines. For cases with distances of <15 Å, the number of liganded cysteines is cumulatively as distance increases. FIG. 19D shows the location of the liganded cysteine C346 (blue) in the immune-relevant kinase ZAP70 and pathogenic missense mutations (yellow—mutation within 5 Å of C346; red—other mutations). PDB: 4K2R. FIG. 19E shows the fraction of liganded proteins from total proteins encoded by T cell proliferation genes. FIG. 19F shows the fraction of liganded proteins from total proteins found in previously described immune-enriched modules.

FIG. 20A-20C provides for an analysis of cysteine ligandability in immune signaling pathways. Related to FIG. 3. FIG. 20A shows cysteine ligandability analysis of the enriched GO terms for proteins undergoing reactivity (top) or expression (bottom) changes in activated T cells. Enriched terms were passed through REVIGO, which identifies redundant terms and chooses representative terms for each group. Red sub-bars on right graphs represent both percentage and total number of proteins in each GO-term category having one or more liganded cysteines. Top-10 enriched biological processes are shown for the expression changes group. FIG. 20B provides a Venn diagram showing overlap between proteins quantified by TMT-exp and proteins liganded with scout fragments. FIG. 20C shows the location of the liganded cysteine C313 in IRF9 (mouse orthologous residue to human C319) at the site of its protein-protein interaction with STAT2 (PDB: 5OEN).

FIG. 21A-21B provides for an analysis of active compound effects in human T cells. FIG. 21A shows concentration-dependent effects of EV-96 and EV-97 on T-cell activation parameters (left graphs: IL-2 and IFN-γ) and cytotoxicity (right graph) (24 h treatment). Data are presented as the mean percentage of DMSO-treated control cells±SEM; n=4-5/group. *, p<0.05; **, p<0.01 by two-tailed unpaired t test with Welch's correction compared to EV-97 (0.5 μM) treatment group. FIG. 21B provides a bar graph showing the total number of quantified (black) and liganded (R≥4, red) cysteines in cells treated with active compounds. Results are obtained by combining isoTOP-ABPP and TMT-ABPP data for both soluble and particulate proteomic fractions. R-values within each experimental treatment group were derived from 3-6 independent isoTOP-ABPP experiments and 2-3 independent TMT-ABPP experiments (4-6 TMT channels).

FIG. 22A-22G provides for a functional analysis of protein targets of active compounds in human T cells. FIG. 22A and FIG. 22B show the effects of active compounds on NFAT pathway activation. FIG. 22A shows Western blot analysis of NFATc2 content and phosphorylation in T cells treated with active (EV-3 (10 μM), BPK-21 (20 μM), BPK-25 (10 μM) (EV-96 (5 μM)) and control (EV-97 (5 μM)) compounds for 4 h in stimulated T cells. The anti-NFATc2 antibody recognizes both phosphorylated (upper band) and dephosphorylated (lower band) NFATc2. FIG. 22B shows a bar graph representation of western blot data showing compound effects on p-NFATc2 (green) and NFATc2 (red) content. Protein band intensity calculations were made using ImageJ software and represent the mean percentage of corresponding DMSO treated control cells±SD; n=2/group. p-NFATc2 levels were normalized using a mean (p-NFATc2/NFATc2) ratio for DMSO-treated controls to account for the differences between p-NFATc2 and NFATc2 in DMSO (stim) control. Dashed line shows 50% of mean signal intensities for dephosphorylated NFATc2 in DMSO (stim) control. FIG. 22C and FIG. 22D show the effects of active compounds on MAPK pathway. FIG. 22C shows Western blot data showing compound effects on p-ERK1/2 (T202/Y204) content. FIG. 22D shows a bar graph representation of western blot data showing the compound effects on p-ERK1/2 (T202/Y204) content. All treatments were done for 24 h. Protein band intensity calculations were made using ImageJ software and represent the mean percentage of DMSO treated control cells±SD; n=3-5/group. FIG. 22E shows the structure of immunosuppressive natural product triptolide (left) and MS1 signal intensities (right) for ERCC3_C342 in isoTOP-ABPP experiments of expanded T cells treated with BPK-21 (20 μM, 3 h), BPK-25 (10 μM, 3 h), or triptolide (0.2 μM, 3 h. FIG. 22F shows a T cell activation and cytotoxicity profile of the immunosuppressive natural product triptolide. Data are presented as the mean percentage of DMSO-treated control cells±SEM; n=3/group. FIG. 22G shows the efficiency of CRISPR/Cas9 genome editing. Left: western blot analysis showing ERCC3 protein content after CRISPR/Cas9 genome editing using control or ERCC3 sgRNA and sorting for GFP-positive T cells. Right: Quantitation of western blot data, where protein band intensity calculations were made using ImageJ software and represent the mean percentage of control guide sgRNA-treated cells±SD; n=2/group.

FIG. 23A-23C provides for a functional analysis of protein targets of active compounds in human T cells. FIG. 23A shows quantification of BPK-25 concentration-dependent changes in protein expression. Western blot protein band intensity calculations were made using ImageJ software and represent the mean percentage of DMSO treated control±SD; n=2. FIG. 23B shows that BPK-25-ctrl, a non-electrophilic propanamide analogue of BPK-25, does not inhibit T-cell activation. Left: Structure of BPK-25. Middle: Structure of BPK-25-ctrl. Right: T-cell activation (CD25) and cytotoxicity profiles for BPK-25 and BPK-25-ctrl (10 μM of each compound, 24 h). Data are presented as the mean percentage of DMSO-treated control cells±SD; n=2/group. FIG. 23C provides Western blot data showing that BPK-25-ctrl, does not alter protein content for NuRD complex members.

FIG. 24A-24I provides for a characterization of protein targets of EV-96 in human T cells. FIG. 24A Left: provides a bar graph showing fraction of total targets that show enantioselective (ES) engagement with one of the stereoisomeric compounds EV-96, EV-97, EV-98, and EV-99 at either 5 μM or 20 μM test concentrations in T cells (3 h). A target was considered enantioselective if it was engaged by one of the stereoisomers with an R value ≥4 and showed a reduction in engagement by at least 50% with the corresponding enantiomeric compound. Middle: Bar graph showing the percentage of enantioselective targets that were also liganded by scout fragments. Data for enantioselective (ES) and non-enantioselective (other) targets are shown separately. Right: Fraction of enantioselective targets that are immune-relevant (immune-enriched (blue) and/or have human genetics-based immune phenotypes (green)). FIG. 24B shows enantioselective engagement of C449 of TEC kinase by EV-96. Bar graphs showing percent reductions in IA-DTB labeling of C449 of TEC in T cells treated with the indicated concentrations of stereoisomeric electrophiles (3 h treatment; left: 5 μM, right: 20 μM), as quantified by TMT-ABPP experiments. Results represent mean values±SD; n=2-4/group. FIG. 24C provide western blot results showing enantioselective reductions in ITK protein and PLCG1 phosphorylation (Y783) in stimulated T cells treated with EV-96 (5 μM) compared to DMSO- or EV-97 (5 μM), EV-98 (5 μM), and EV-99 (5 μM)-treated T cells, measured at the indicated time points. Results are from one experiment representative of 2-5 independent experiments. FIG. 24D provides quantitation of western blot data showing effects of EV-96 and EV-97 on ITK protein content in naïve control (ctrl) versus αCD3/CD28-treated (stimulated, stim) T cells and the blockade of EV-96-induced loss of ITK by co-treatment with the proteasome inhibitor MG132 (10 μM). Cells were treated with compounds (5 μM each) or DMSO control for 8 h. Protein band intensity calculations were made using ImageJ software and represent the mean percentage of DMSO-treated control±SD; n=2-5 independent experiments per group. **p<0.01 by two-tailed unpaired t test with Welch's correction compared to DMSO-treated stim control. FIG. 24E provides a western blot showing time-dependent decrease in ITK protein content in EV-96-treated αCD3/CD28-stimulated (Stim), but not expanded control (Ctrl) T cells. Cells treated with DMSO or EV-96 (5 μM) for 1-8 h. Results are from a single experiment representative of 2-4 independent experiments. FIG. 24F shoes that EV-96 does not block the kinase activity of recombinant, purified ITK protein, as measured by the ADP-Glo™ Kinase Assay (Promega) following manufacturer's instructions. *PF—PF-06465469, an irreversible ITK inhibitor that reacts with C442. FIG. 24G provides western blot results showing that EV-96 (5 μM, 2-8 h) does not impair the phosphorylation of SLP-76 (S376) in stimulated (Stim) T cells. FIG. 24G shows that EV-96-ctrl, a non-electrophilic propanamide analogue of EV-96, does not inhibit T-cell activation. Left: Structure of EV-96-ctrl. Right: T-cell activation and cytotoxicity profiles for EV-96 and EV-96-ctrl (5 μM, 24 h). Data are presented as the mean percentage of DMSO-treated control cells±SD; n=2-5/group. FIG. 24H provides western blot results showing that EV-96-ctrl does not alter ITK protein content in stimulated T cells. Cells were treated with compounds (5 μM each) or DMSO for 8 h. Results are from one experiment representative of 2 independent experiments. FIG. 24I provides western blot results showing that pre-treatment (1 h) with EV-97 (5 μM) does not block EV-96-dependent decreases in ITK protein content in stimulated T cells. After 1 h pre-treatment, cells were treated with EV-96 (5 μM) or DMSO for 8 h. FIG. 24I shows the structure of PF-06465469, an irreversible ITK inhibitor that reacts with C442 (left) and the corresponding crystal structure of a PF-06465469-ITK complex showing covalent modification of C442 (red).

### DETAILED DESCRIPTION OF THE DISCLOSURE

Cysteine containing proteins encompass a large repertoire of proteins that participate in numerous cellular functions such as mitogenesis, proliferation, apoptosis, gene regulation, and proteolysis. These proteins include enzymes, transporters, receptors, channel proteins, adaptor proteins, chaperones, signaling proteins, plasma proteins, transcription related proteins, translation related proteins, mitochondrial proteins, or cytoskeleton related proteins. Dysregulated expression of a cysteine containing protein, in many cases, is associated with or modulates a disease, for example, such as cancer.

In some instances, small molecule compounds are capable of eliciting an immune response. In some instances, these small molecule compounds are referred to as haptens. In some cases, a hapten is a non-immunogenic compound but becomes immunogenic when it interacts with a carrier molecule such as a protein. For example, upon administration of a small molecule hapten, the hapten forms an adduct with a protein of interest in a process refers to as haptenization. In some cases, the protein-hapten adduct becomes antigenically active and enables priming of T cells and B cells, thereby directing immune response to a cell that expresses the protein of interest.

In some embodiments, disclosed herein are small molecule fragments that elicit an immune response upon interaction with cysteine-containing proteins (or cysteine-containing polypeptides). In some instances, also disclosed herein includes use of a small molecule fragment described herein to elicit or modulate an immune response in a subject. In such instances, the small molecule fragment forms an adduct with an endogenous cysteine-containing protein, and subsequently directs immune response to the cell that expresses the endogenous cysteine-containing protein. In some instances, the cell that expresses the endogenous cysteine-containing protein is a disease cell (e.g., a cancerous cell). In some instances, the endogenous cysteine-containing protein is present only in a diseased cell (e.g., a cancerous cell). In other instances, the endogenous cysteine-containing protein is overexpressed in a diseased cell (e.g., a cancerous cell) and/or comprises one or more mutations in a diseased cell (e.g., a cancerous cell).

In some embodiments, also disclosed herein are vaccines and pharmaceutical compositions that comprise one or more small molecule fragments described herein. In some instances, additionally descried herein are vaccines and pharmaceutical compositions that comprise one or more cysteine-containing polypeptide-small molecule fragment adducts or antibodies that recognize a cysteine-containing polypeptide-small molecule fragment adduct described herein.

In additional embodiments, described herein include kits for use with any of the methods, vaccines, and pharmaceutical compositions disclosed herein.

### Small Molecule Fragments

In some embodiments, described herein include pharmaceutical compositions, vaccines, and methods of use of a small molecule fragment. In some embodiments, a small molecule fragment described herein comprises a non-naturally occurring molecule. In some instances, the non-naturally occurring molecule does not include a natural and/or non-natural peptide fragment, or a small molecule that is produced naturally within the body of a mammal.

In some embodiments, a small molecule fragment described herein comprises a molecule weight of about 100 Dalton or higher. In some embodiments, a small molecule fragment comprises a molecule weight of about 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 550, 600, 650, 700, 750, 800, 850, 900, 950, 1000 Dalton, or higher. In some instances, the molecule weight of a small molecule fragment is between about 150 and about 500, about 150 and about 450, abut 150 and about 440, about 150 and about 430, about 150 and about 400, about 150 and about 350, about 150 and about 300, about 150 and about 250, about 170 and about 500, about 180 and about 450, about 190 and about 400, about 200 and about 350, about 130 and about 300, or about 120 and about 250 Dalton.

In some embodiments, the molecule weight of a small molecule fragment described herein is the molecule weight prior to enrichment with one or more elements selected from a halogen, a nonmetal, a transition metal, or a combination thereof. In some embodiments, the molecule weight of a small molecule fragment described herein is the molecule weight prior to enrichment with a halogen. In some embodiments, the molecule weight of a small molecule fragment described herein is the molecule weight prior to enrichment with a nonmetal. In some embodiments, the molecule weight of a small molecule fragment described herein is the molecule weight prior to enrichment with a transition metal.

In some embodiments, a small molecule fragment described herein comprises micromolar or millimolar binding affinity. In some instances, a small molecule fragment comprises a binding affinity of about 104, 10 μM, 100 μM, 50004, 1 mM, 10 mM, or higher.

In some embodiments, a small molecule fragment described herein has a high ligand efficiency (LE). Ligand efficiency is the measurement of the binding energy per atom of a ligand to its binding partner. In some instances, the ligand efficiency is defined as the ratio of the Gibbs free energy (AG) to the number of non-hydrogen atoms of the compound (N):

LE=(ΔG)/N.

In some cases, LE is also arranged as:

LE=1.4(−log IC₅₀)/N.

In some instances, the LE score is about 0.3 kcal mol⁻¹ HA⁻¹, about 0.35 kcal mol⁻¹ HA⁻¹, about 0.4 kcal mol⁻¹ HA⁻¹, or higher.

In some embodiments, a small molecule fragment described herein is designed based on the Rule of 3. In some embodiments, the Rule of 3 comprises a non-polar solvent-polar solvent (e.g. octanol-water) partition coefficient log P of about 3 or less, a molecular mass of about 300 Daltons or less, about 3 hydrogen bond donors or less, about 3 hydrogen bond acceptors or less, and about 3 rotatable bonds or less.

In some embodiments, a small molecule fragment described herein comprises three cyclic rings or less.

In some embodiments, a small molecule fragment described herein binds to a cysteine residue of a polypeptide that is about 20 amino acid residues in length or more. In some instances, a small molecule fragment described herein binds to a cysteine residue of a polypeptide that is about 30, 35, 40, 45, 50, 55, 60, 65, 70, 75, 80, 85, 90, 95, 100 amino acid residues in length or more.

In some embodiments, a small molecule fragment described herein further comprises pharmacokinetic parameters that are unsuitable as a therapeutic agent for administration without further optimization of the small molecule fragments. In some instances, the pharmacokinetic parameters that are suitable as a therapeutic agent comprise parameters in accordance with FDA guideline, or in accordance with a guideline from an equivalent Food and Drug Administration outside of the United States. In some instances, the pharmacokinetic parameters comprise the peak plasma concentration (Cmax), the lowest concentration of a therapeutic agent (Cmin), volume of distribution, time to reach Cmax, elimination half-life, clearance, and the life. In some embodiments, the pharmacokinetic parameters of the small molecule fragments are outside of the parameters set by the FDA guideline, or by an equivalent Food and Drug Administration outside of the United States. In some instances, a skilled artisan understands in view of the pharmacokinetic parameters of the small molecule fragments described herein that these small molecule fragments are unsuited as therapeutic agents without further optimization.

In some embodiments, a small molecule fragment described herein comprises a reactive moiety which forms a covalent interaction with the thiol group of a cysteine residue of a cysteine-containing protein, and an affinity handle moiety.

In some instances, a small molecule fragment described herein is a small molecule fragment of Formula (I):

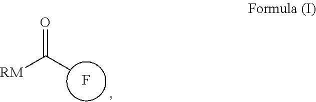

wherein:
RM is a reactive moiety selected from a Michael acceptor moiety, a leaving group moiety, or a moiety capable of forming a covalent bond with the thiol group of a cysteine residue; and
F is a small molecule fragment moiety.

In some instances, the Michael acceptor moiety comprises an alkene or an alkyne moiety. In some cases, F is obtained from a compound library. In some cases, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library.

In some embodiments, a small molecule fragment of Formula (I) selectively interact with one or more protein variants. In some instances, for example, a small molecule fragment of Formula (I) interacts or binds to the wild-type protein but does not bind to a mutant form of the protein. Conversely, in some instances, a small molecule fragment of Formula (I) interacts or binds to one specific protein mutant but does not interact with either the wild-type or the same protein comprising a different mutation. As used herein, the term “variant” comprises mutations within the protein sequence, additions or deletions of the protein sequence, and/or termini truncations. As used herein, the term “variant” comprises a protein having different conformations, for example, an active conformation or an inactive conformation. In some instances, a small molecule fragment of Formula (I) interacts with about 1, 2, 3, 4, 5, or more different variants of a protein of interest. In additional instances, a small molecule fragment of Formula (I) interacts with about 1 variant of a protein of interest. In additional instances, a small molecule fragment of Formula (I) interacts with about 2 variants of a protein of interest. In additional instances, a small molecule fragment of Formula (I) interacts with about 3 variants of a protein of interest. In additional instances, a small molecule fragment of Formula (I) interacts with about 4 variants of a protein of interest. In additional instances, a small molecule fragment of Formula (I) interacts with about 5 variants of a protein of interest.

In some embodiments, a small molecule fragment of Formula (I) does not contain a second binding site. In some instances, a small molecule fragment moiety does not bind to the protein. In some cases, a small molecule fragment moiety does not covalently bind to the protein. In some instances, a small molecule fragment moiety does not interact with a secondary binding site on the protein. In some instances, the secondary binding site is an active site such as an ATP binding site. In some cases, the active site is at least about 10, 15, 20, 25, 35, 40 Å, or more away from the biologically active cysteine residue. In some instances, the small molecule fragment moiety does not interact with an active site such as an ATP binding site.

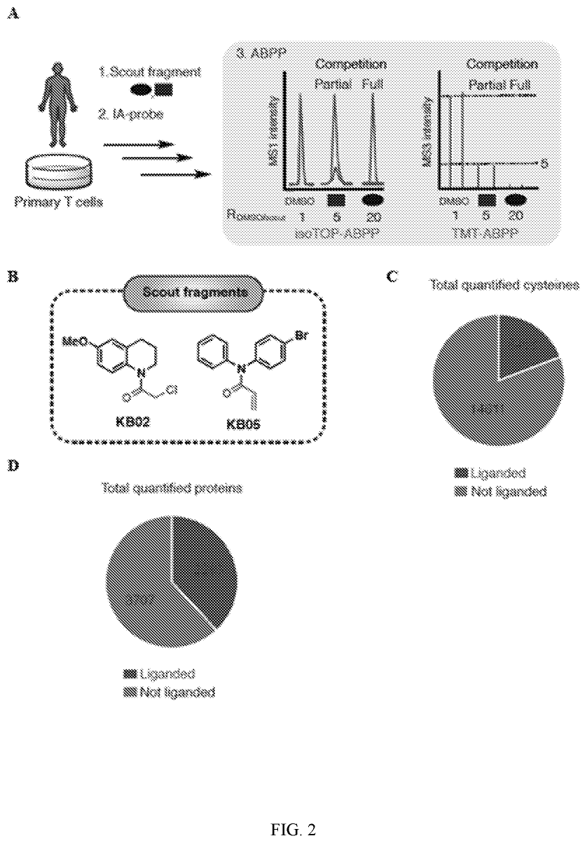

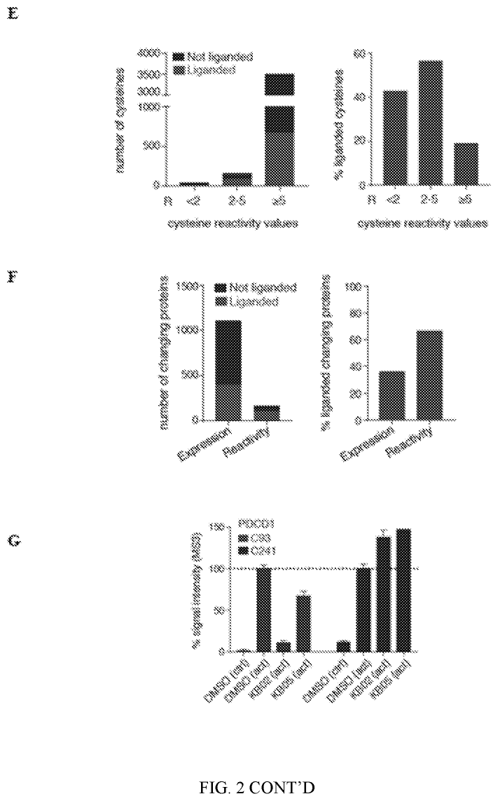

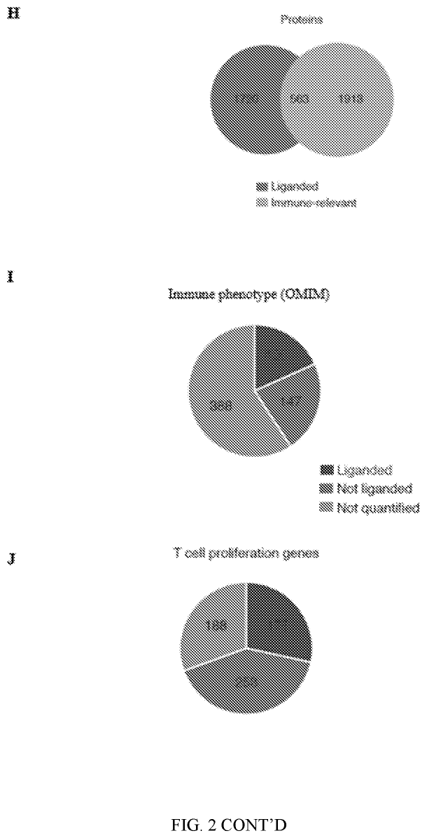

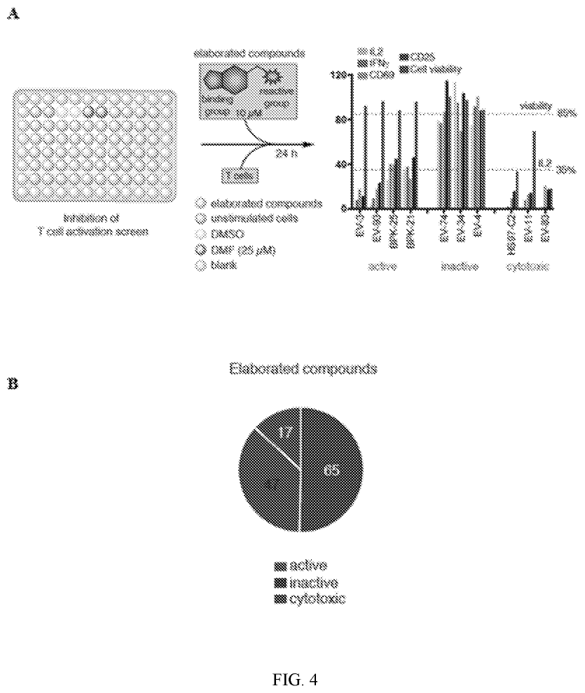

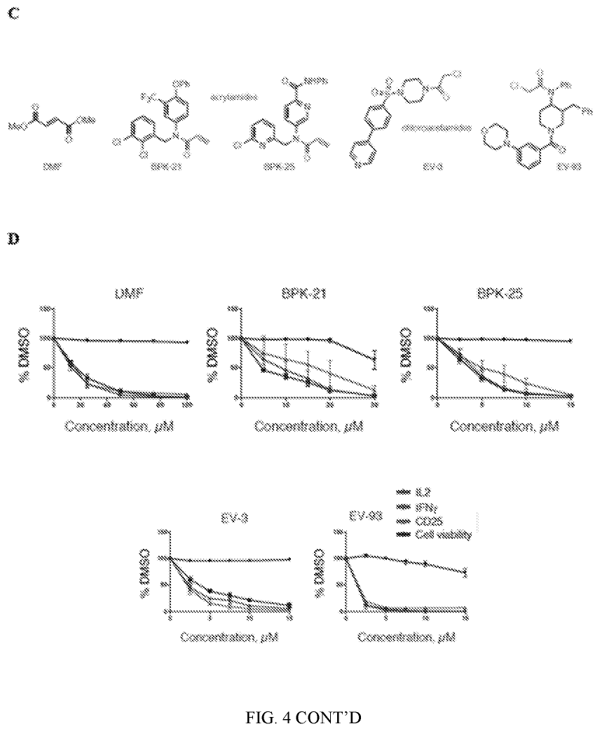

In some instances, F is a small molecule fragment moiety illustrated in FIGS. 2B and 4C. In some cases, F further comprises a linker moiety that connects F to the carbonyl moiety. In some cases, the small molecule fragment is a small molecule fragment illustrated in FIGS. 2B and 4C.

In some instances, F is a small molecule fragment moiety selected from: N-(4-bromophenyl)-N-phenylacrylamide, N-(1-benzoylpiperidin-4-yl)-2-chloro-N-phenylacetamide, 1-(4-benzylpiperidin-1-yl)-2-chloroethan-1-one, N-(2-(1H-indol-3-yl)ethyl)-2-chloroacetamide, N-(3,5-bis(trifluoromethyl)phenyl)acrylamide, N-(4-phenoxy-3-(trifluoromethyl)phenyl)-N-(pyridin-3-ylmethyl)acrylamide, N-(3,5-bis(trifluoromethyl)phenyl)acetamide, 2-chloro-1-(4-(hydroxydiphenylmethyl)piperidin-1-yl)ethan-1-one, (E)-3-(3,5-bis(trifluoromethyl)phenyl)-2-cyanoacrylamide, N-(3,5-bis(trifluoromethyl)phenyl)-2-bromopropanamide, N-(3,5-bis(trifluoromethyl)phenyl)-2-chloropropanamide, N-(3,5-bis(trifluoromethyl)phenyl)-N-(pyridin-3-ylmethyl)acrylamide, 3-(2-chloroacetamido)-5-(trifluoromethyl)benzoic acid, 1-(4-(5-fluorobenzisoxazol-3-yl)piperidin-1-yl)prop-2-en-1-one, tert-butyl 4-(4-acrylamido-2,6-difluorophenyl)piperazine-1-carboxylate, N-(4-bromo-2,5-dimethylphenyl)acrylamide, 2-Chloroacetamido-2-deoxy-α/β-D-glucopyrano se, 2-chloro-1-(2-methyl-3,4-dihydroquinolin-1(2H)-yl)ethan-1-one, N-cyclohexyl-N-phenylacrylamide, 1-(5-bromoindolin-1-yl)prop-2-en-1-one, N-(1-benzylpiperidin-4-yl)-N-phenylacrylamide, 2-chloro-N-(2-methyl-5-(trifluoromethyl)phenyl)acetamide, 1-(5-bromoindolin-1-yl)-2-chloroethan-1-one, 2-chloro-N-(quinolin-5-yl)acetamide, 1-(4-benzylpiperidin-1-yl)prop-2-en-1-one, 2-chloro-N-((3-hydroxy-5-(hydroxymethyl)-2-methylpyridin-4-yl)methyl)acetamide, or 1-(6,7-dimethoxy-3,4-dihydroisoquinolin-2(1H)-yl)prop-2-en-1-one.

In some embodiments, the small molecule fragment of Formula (I) comprise a molecule weight of about 100, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 550, 600, 650, 700, 750, 800, 850, 900, 950, 1000 Dalton, or higher. In some instances, the molecule weight of the small molecule fragment of Formula (I) is between about 150 and about 500, about 150 and about 450, abut 150 and about 440, about 150 and about 430, about 150 and about 400, about 150 and about 350, about 150 and about 300, about 150 and about 250, about 170 and about 500, about 180 and about 450, about 190 and about 400, about 200 and about 350, about 130 and about 300, or about 120 and about 250 Dalton.

In some embodiments, the molecule weight of the small molecule fragment of Formula (I) is the molecule weight prior to enrichment with one or more elements selected from a halogen, a nonmetal, a transition metal, or a combination thereof. In some embodiments, the molecule weight of the small molecule fragment of Formula (I) is the molecule weight prior to enrichment with a halogen. In some embodiments, the molecule weight of the small molecule fragment of Formula (I) is the molecule weight prior to enrichment with a nonmetal. In some embodiments, the molecule weight of the small molecule fragment of Formula (I) is the molecule weight prior to enrichment with a transition metal.

In some instances, the small molecule fragment of Formula (I) comprises micromolar or millimolar binding affinity. In some instances, the small molecule fragment of Formula (I) comprises a binding affinity of about 1 μM, 10 μM, 100 μM, 500 μM, 1 mM, 10 mM, or higher.

In some cases, the small molecule fragment of Formula (I) has a LE score about 0.3 kcal mol⁻¹ HA⁻¹, about 0.35 kcal mol⁻¹ HA⁻¹, about 0.4 kcal mol⁻¹ HA⁻¹, or higher.

In some embodiments, the small molecule fragment of Formula (I) follows the design parameters of Rule of 3. In some instances, the small molecule fragment of Formula (I) has a non-polar solvent-polar solvent (e.g. octanol-water) partition coefficient log P of about 3 or less, a molecular mass of about 300 Daltons or less, about 3 hydrogen bond donors or less, about 3 hydrogen bond acceptors or less, and about 3 rotatable bonds or less.

In some embodiments, the small molecule fragment of Formula (I) comprises three cyclic rings or less.

In some embodiments, the small molecule fragment of Formula (I) binds to a cysteine residue of a polypeptide (e.g., a cysteine-containing protein) that is about 20 amino acid residues in length or more. In some instances, the small molecule fragments described herein binds to a cysteine residue of a polypeptide (e.g., a cysteine-containing protein) that is about 30, 35, 40, 45, 50, 55, 60, 65, 70, 75, 80, 85, 90, 95, 100 amino acid residues in length or more.

In some instances, the small molecule fragment of Formula (I) has pharmacokinetic parameters outside of the parameters set by the FDA guideline, or by an equivalent Food and Drug Administration outside of the United States. In some instances, a skilled artisan understands in view of the pharmacokinetic parameters of the small molecule fragment of Formula (I) described herein that these small molecule fragments are unsuited as a therapeutic agent without further optimization.

### Cysteine-Containing Proteins

In some embodiments, disclosed herein include a cysteine-containing polypeptide. In some instances, the cysteine-containing polypeptide is about 20, 30, 35, 40, 45, 50, 55, 60, 65, 70, 75, 80, 85, 90, 95, 100 amino acid residues in length or more. In some instances, the cysteine-containing polypeptide is a cysteine-containing protein or its fragment thereof. In some instances, the cysteine-containing protein is a soluble protein or its fragment thereof, or a membrane protein or its fragment thereof. In some instances, the cysteine-containing protein is involved in one or more of a biological process such as protein transport, lipid metabolism, apoptosis, transcription, electron transport, mRNA processing, or host-virus interaction. In some instances, the cysteine-containing protein is associated with one or more of diseases such as cancer or one or more disorders or conditions such as immune, metabolic, developmental, reproductive, neurological, psychiatric, renal, cardiovascular, or hematological disorders or conditions.

In some embodiments, the cysteine-containing protein comprises a biologically active cysteine residue. In some embodiments, the cysteine-containing protein comprises one or more cysteines in which at least one cysteine is a biologically active cysteine residue. In some cases, the biologically active cysteine site is a cysteine residue that is located about 10 Å or less to an active-site ligand or residue. In some cases, the cysteine residue that is located about 10 Å or less to the active-site ligand or residue is an active site cysteine. In other cases, the biologically active cysteine site is a cysteine residue that is located greater than 10 Å from an active-site ligand or residue. In some instances, the cysteine residue is located greater than 12 Å, 15 Å, 20 Å, 25 Å, 30 Å, 35 Å, 40 Å, 45 Å, or greater than 50 Å from an active-site ligand or residue. In some cases, the cysteine residue that is located greater than 10 Å from the active-site ligand or residue is a non-active site cysteine. In additional cases, the cysteine-containing protein exists in an active form, or in a pro-active form.

In some embodiments, the cysteine-containing protein comprises one or more functions of an enzyme, a transporter, a receptor, a channel protein, an adaptor protein, a chaperone, a signaling protein, a plasma protein, transcription related protein, translation related protein, mitochondrial protein, or cytoskeleton related protein. In some embodiments, the cysteine-containing protein is an enzyme, a transporter, a receptor, a channel protein, an adaptor protein, a chaperone, a signaling protein, a plasma protein, transcription related protein, translation related protein, mitochondrial protein, or cytoskeleton related protein. In some instances, the cysteine-containing protein has an uncategorized function.

In some embodiments, the cysteine-containing protein is an enzyme. An enzyme is a protein molecule that accelerates or catalyzes chemical reaction. In some embodiments, non-limiting examples of enzymes include kinases, proteases, or deubiquitinating enzymes.

In some instances, exemplary kinases include tyrosine kinases such as the TEC family of kinases such as Tec, Bruton's tyrosine kinase (Btk), interleukin-2-indicible T-cell kinase (Itk) (or Emt/Tsk), Bmx, and Txk/Rlk; spleen tyrosine kinase (Syk) family such as SYK and Zeta-chain-associated protein kinase 70 (ZAP-70); Src kinases such as Src, Yes, Fyn, Fgr, Lck, Hck, Blk, Lyn, and Frk; JAK kinases such as Janus kinase 1 (JAK1), Janus kinase 2 (JAK2), Janus kinase 3 (JAK3), and Tyrosine kinase 2 (TYK2); or ErbB family of kinases such as Her1 (EGFR, ErbB1), Her2 (Neu, ErbB2), Her3 (ErbB3), and Her4 (ErbB4).

In some embodiments, the cysteine-containing protein is a protease. In some embodiments, the protease is a cysteine protease. In some cases, the cysteine protease is a caspase. In some instances, the caspase is an initiator (apical) caspase. In some instances, the caspase is an effector (executioner) caspase. Exemplary caspase includes CASP2, CASP8, CASP9, CASP10, CASP3, CASP6, CASP7, CASP4, and CASP5. In some instances, the cysteine protease is a cathepsin. Exemplary cathepsin includes Cathepsin B, Cathepsin C, CathepsinF, Cathepsin H, Cathepsin K, Cathepsin L1, Cathepsin L2, Cathepsin O, Cathepsin S, Cathepsin W, or Cathepsin Z.

In some embodiments, the cysteine-containing protein is a deubiquitinating enzyme (DUB). In some embodiments, exemplary deubiquitinating enzymes include cysteine proteases DUBs or metalloproteases. Exemplary cysteine protease DUBs include ubiquitin-specific protease (USP/UBP) such as USP1, USP2, USP3, USP4, USP5, USP6, USP7, USP5, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, or USP46; ovarian tumor (OTU) proteases such as OTUB1 and OTUB2; Machado-Josephin domain (MJD) proteases such as ATXN3 and ATXN3L; and ubiquitin C-terminal hydrolase (UCH) proteases such as BAP1, UCHL1, UCHL3, and UCHL5. Exemplary metalloproteases include the Jab1/Mov34/Mpr1 Pad1 N-terminal+(MPN+) (JAMM) domain proteases.

In some embodiments, exemplary cysteine-containing proteins as enzymes include, but are not limited to, Glyceraldehyde-3-phosphate dehydrogenase (GAPDH), Protein arginine N-methyltransferase 1 (PRMT1), Peptidyl-prolyl cis-trans isomerase NIMA-interaction (PIN1), Acetyl-CoA acetyltransferase (mitochondrial) (ACAT1), Glutathione S-transferase P (GSTP1), Elongation factor 2 (EEF2), Glutathione S-transferase omega-1 (GST01), Acetyl-CoA acetyltransferase (mitochondrial) (ACAT1), Protein disulfide-isomerase A4 (PDIA4), Prostaglandin E synthase 3 (PTGES3), Adenosine kinase (ADK), Elongation factor 2 (EEF2), Isoamyl acetate-hydrolyzing esterase 1 homolog (IAH1), Peroxiredoxin-5 (mitochondrial) (PRDX5), Inosine-5-monophosphate dehydrogenase 2 (IMPDH2), 3-hydroxyacyl-CoA dehydrogenase type-2 (HSD17B10), Omega-amidase NIT2 (NIT2), Aldose reductase (AKR1B1), Monofunctional C1-tetrahydrofolate synthase (mitochondrial) (MTHFD1L), Protein disulfide-isomerase A6 (PDIA6), Pyruvate kinase isozymes M1/M2 (PKM), 6-phosphogluconolactonase (PGLS), Acetyl-CoA acetyltransferase (mitochondrial) (ACAT1), ER01-like protein alpha (ERO1L), Thioredoxin domain-containing protein 17 (TXNDC17), Protein disulfide-isomerase A4 (PDIA4), Protein disulfide-isomerase A3 (PDIA3), 3-ketoacyl-CoA thiolase (mitochondrial) (ACAA2), Dynamin-2 (DNM2), DNA replication licensing factor MCM3 (MCM3), Serine—tRNA ligase (cytoplasmic) (SARS), Fatty acid synthase (FASN), Acetyl-CoA acetyltransferase (mitochondrial) (ACAT1), Protein disulfide-isomerase (P4HB), Deoxycytidine kinase (DCK), Eukaryotic translation initiation factor 3 subunit (EIF3F), Protein disulfide-isomerase A6 (PDIA6), UDP-N-acetylglucosamine-peptide N-acetylglucosamine (OGT), Ketosamine-3-kinase (FN3KRP), Protein DJ-1 (PARK7), Phosphoglycolate phosphatase (PGP), DNA replication licensing factor MCM6 (MCM6), Fructose-2,6-bisphosphatase TIGAR (TIGAR), Cleavage and polyadenylation specificity factor subunit (CPSF3), Ubiquitin-conjugating enzyme E2 L3 (UBE2L3), Alanine—tRNA ligase, cytoplasmic (AARS), Mannose-1-phosphate guanyltransferase alpha (GMPPA), C-1-tetrahydrofolate synthase (cytoplasmic) (MTHED1), Dynamin-1-like protein (DNM1L), Protein disulfide-isomerase A3 (PDIA3), Aspartyl aminopeptidase (DNPEP), Acetyl-CoA acetyltransferase (cytosolic) (ACAT2), Thioredoxin domain-containing protein 5 (TXNDC5), Thymidine kinase (cytosolic) (TK1), Inosine-5-monophosphate dehydrogenase 2 (IMPDH2), Ubiquitin carboxyl-terminal hydrolase isozyme L3 (UCHL3), Integrin-linked protein kinase (ILK), Cyclin-dependent kinase 2 (CDK2), Histone acetyltransferase type B catalytic subunit (HAT1), Enoyl-CoA delta isomerase 2 (mitochondrial) (ECI2), C-1-tetrahydrofolate synthase (cytoplasmic) (MTHFD1), Deoxycytidine kinase (DCK), Ubiquitin-like modifier-activating enzyme 6 (UBA6), Protein-L-isoaspartate(D-aspartate) O-methyltransferase (PCMT1), Monofunctional C1-tetrahydrofolate synthase (mitochondrial) (MTHFD1L), Thymidylate kinase (DTYMK), Protein ETHE1 (mitochondrial) (ETHE1), Arginine—tRNA ligase (cytoplasmic) (RARS), NEDD8-activating enzyme E1 catalytic subunit (UBA3), Dual specificity mitogen-activated protein kinase (MAP2K3), Ubiquitin-conjugating enzyme E2S (UBE2S), Amidophosphoribosyltransferase (PPAT), Succinate-semialdehyde dehydrogenase (mitochondrial) (ALDH5A1), CAD, Phosphoenolpyruvate carboxykinase (PCK2), 6-phosphofructokinase type C (PFKP), Acyl-CoA synthetase family member 2 (mitochondrial) (ACSF2), Multifunctional protein ADE2 (PAICS), Desumoylating isopeptidase 1 (DESI1), 6-phosphofructokinase type C (PFKP), V-type proton ATPase catalytic subunit A (ATP6V1A), 3-ketoacyl-CoA thiolase (peroxisomal) (ACAA1), Galactokinase (GALK1), Thymidine kinase (cytosolic) (TK1), ATPase WRNIP1 (WRNIP1), Phosphoribosylformylglycinamidine synthase (PFAS), V-type proton ATPase catalytic subunit A (ATP6V1A), Thioredoxin domain-containing protein 5 (TXNDC5), 4-trimethylaminobutyraldehyde dehydrogenase (ALDH9A1), Dual specificity mitogen-activated protein kinase (MAP2K4), Calcineurin-like phosphoesterase domain-containing (CPPED1), Dual specificity protein phosphatase 12 (DUSP12), Phosphoribosylformylglycinamidine synthase (PFAS), Diphosphomevalonate decarboxylase (MVD), D-3-phosphoglycerate dehydrogenase (PHGDH), Cell cycle checkpoint control protein RAD9 Å (RAD9A), Peroxiredoxin-1 (PRDX1), Sorbitol dehydrogenase (SORD), Peroxiredoxin-4 (PRDX4), AMP deaminase 2 (AMPD2), Isocitrate dehydrogenase (IDH1), Pyruvate carboxylase (mitochondrial) (PC), Integrin-linked kinase-associated serine/threonine (ILKAP), Methylmalonate-semialdehyde dehydrogenase (ALDH6A1), 26S proteasome non-ATPase regulatory subunit 14 (PSMD14), Thymidylate kinase (DTYMK), 6-phosphofructo-2-kinase/fructose-2,6-bisphosphata (PFKFB2), Peroxiredoxin-5 (mitochondrial) (PRDX5), PDP1, Cathepsin B (CTSB), Transmembrane protease serine 12 (TMPRSS12), UDP-glucose 6-dehydrogenase (UGDH), Histidine triad nucleotide-binding protein 1 (HINT1), E3 ubiquitin-protein ligase UBR5 (UBR5), SAM domain and HD domain-containing protein 1 (SAMHD1), Probable tRNA threonylcarbamoyladenosine biosynthesis (OSGEP), Methylated-DNA—protein-cysteine methyltransferase (MGMT), Fatty acid synthase (FASN), Adenosine deaminase (ADA), Cyclin-dependent kinase 19 (CDK19), Serine/threonine-protein kinase 38 (STK38), Mitogen-activated protein kinase 9 (MAPK9), tRNA (adenine(58)-N(1))-methyltransferase catalytic (TRMT61A), Glyoxylate reductase/hydroxypyruvate reductase (GRHPR), Aldehyde dehydrogenase (mitochondrial) (ALDH2), Mitochondrial-processing peptidase subunit beta (PMPCB), 3-ketoacyl-CoA thiolase, peroxisomal (ACAA1), Lysophosphatidic acid phosphatase type 6 (ACP6), Ubiquitin/ISG15-conjugating enzyme E2 L6 (UBE2L6), Caspase-8 (CASP8), 2,5-phosphodiesterase 12 (PDE12), Thioredoxin domain-containing protein 12 (TXNDC12), Nitrilase homolog 1 (NIT1), ERO1-like protein alpha (ERO1L), SUMO-activating enzyme subunit 1 (SAE1), Leucine—tRNA ligase (cytoplasmic) (LARS), Protein-glutamine gamma-glutamyltransferase 2 (TGM2), Probable DNA dC-dU-editing enzyme APOBEC-3C (APOBEC3C), Double-stranded RNA-specific adenosine deaminase (ADAR), Isocitrate dehydrogenase (IDH2), Methylcrotonoyl-CoA carboxylase beta chain (mitochondrial) (MCCC2), Uridine phosphorylase 1 (UPP1), Glycogen phosphorylase (brain form) (PYGB), E3 ubiquitin-protein ligase UBR5 (UBR5), Procollagen-lysine,2-oxoglutarate 5-dioxygenase 1 (PLOD1), Ubiquitin carboxyl-terminal hydrolase 48 (USP48), Aconitate hydratase (mitochondrial) (ACO2), GMP reductase 2 (GMPR2), Pyrroline-5-carboxylate reductase 1 (mitochondrial) (PYCR1), Cathepsin Z (CTSZ), E3 ubiquitin-protein ligase UBR2 (UBR2), Cysteine protease ATG4B (ATG4B), Serine/threonine-protein kinase Nek9 (NEK9), Lysine-specific demethylase 4B (KDM4B), Insulin-degrading enzyme (IDE), Dipeptidyl peptidase 9 (DPP9), Decaprenyl-diphosphate synthase subunit 2 (PDSS2), TFIIH basal transcription factor complex helicase (ERCC3), Methionine-R-sulfoxide reductase B2 (mitochondrial) (MSRB2), E3 ubiquitin-protein ligase BRE1B (RNF40), Thymidylate synthase (TYMS), Cyclin-dependent kinase 5 (CDK5), Bifunctional 3-phosphoadenosine 5-phosphosulfate (PAPS S2), Short/branched chain specific acyl-CoA dehydrogenase (ACADSB), Cathepsin D (CTSD), E3 ubiquitin-protein ligase HUWE1 (HUWE1), Calpain-2 catalytic subunit (CAPN2), Dual specificity mitogen-activated protein kinase (MAP2K7), Mitogen-activated protein kinase MLT (MLTK), Bleomycin hydrolase (BLMH), Probable ATP-dependent RNA helicase DDX59 (DDX59), Cystathionine gamma-lyase (CTH), S-adenosylmethionine synthase isoform type-2 (MAT2A), 6-phosphofructokinase type C (PFKP), Cytidine deaminase (CDA), DNA-directed RNA polymerase II subunit RPB2 (POLR2B), Protein disulfide-isomerase (P4HB), Procollagen-lysine,2-oxoglutarate 5-dioxygenase 3 (PLOD3), Nucleoside diphosphate-linked moiety X motif 8 (mitochondrial) (NUDT8), E3 ubiquitin-protein ligase HUWE1 (HUWE1), Methylated-DNA—protein-cysteine methyltransferase (MGMT), Nitrilase homolog 1 (N1T1), Interferon regulatory factor 2-binding protein 1 (IRF2BP1), Ubiquitin carboxyl-terminal hydrolase 16 (USP16), Glycylpeptide N-tetradecanoyltransferase 2 (NMT2), Cyclin-dependent kinase inhibitor 3 (CDKN3), Hydroxysteroid dehydrogenase-like protein 2 (HSDL2), Serine/threonine-protein kinase VRK1 (VRK1), Serine/threonine-protein kinase A-Raf (ARAF), ATP-citrate synthase (ACLY), Probable ribonuclease ZC3H12D (ZC3H12D), Peripheral plasma membrane protein CASK (CASK), DNA polymerase epsilon subunit 3 (POLE3), Aldehyde dehydrogenase X (mitochondrial) (ALDH1B1), UDP-N-acetylglucosamine transferase subunit ALG13 (ALG13), Protein disulfide-isomerase A4 (PDIA4), DNA polymerase alpha catalytic subunit (POLA1), Ethylmalonyl-CoA decarboxylase (ECHDC1), Protein-tyrosine kinase 2-beta (PTK2B), E3 SUMO-protein ligase RanBP2 (RANBP2), Legumain (LGMN), Non-specific lipid transfer protein (SCP2), Long-chain-fatty-acid—CoA ligase 4 (ACSL4), Dual specificity protein phosphatase 12 (DUSP12), Oxidoreductase HTATIP2 (HTATIP2), Serine/threonine-protein kinase MRCK beta (CDC42BPB), Histone-lysine N-methyltransferase EZH2 (EZH2), Non-specific lipid-transfer protein (SCP2), Dual specificity mitogen-activated protein kinase (MAP2K7), Ubiquitin carboxyl-terminal hydrolase 28 (USP28), 6-phosphofructokinase (liver type) (PFKL), SWI/SNF-related matrix-associated actin-dependent (SMARCAD1), Protein phosphatase methylesterase 1 (PPME1), DNA replication licensing factor MCM5 (MCM5), 6-phosphofructo-2-kinase/fructose-2,6-bisphosphata (PFKFB4), Dehydrogenase/reductase SDR family member 11 (DHRS11), Pyroglutamyl-peptidase 1 (PGPEP1), Probable E3 ubiquitin-protein ligase (MYCBP2), DNA fragmentation factor subunit beta (DFFB), Deubiquitinating protein VCIP135 (VCPIP1), Putative transferase CAF17 (mitochondrial) (IBA57), Calpain-7 (CAPN7), GDP-L-fucose synthase (TSTA3), Protein disulfide-isomerase A4 (PDIA4, Probable ATP-dependent RNA helicase (DDX59), RNA exonuclease 4 (REXO4), PDK1, E3 SUMO-protein ligase (PIAS4), DNA (cytosine-5)-methyltransferase 1 (DNMT1), Alpha-aminoadipic semialdehyde dehydrogenase (ALDH7A1), Hydroxymethylglutaryl-CoA synthase (cytoplasmic) (HMGCS1), E3 ubiquitin-protein ligase (SMURF2), Aldehyde dehydrogenase X (mitochondrial) (ALDH1B1), Tyrosine-protein kinase (BTK), DNA repair protein RAD50 (RAD50), ATP-binding domain-containing protein 4 (ATPBD4), Nucleoside diphosphate kinase 3 (NME3), Interleukin-1 receptor-associated kinase 1 (IRAK1), Ribonuclease P/MRP protein subunit POPS (POPS), Peptide-N(4)-(N-acetyl-beta-glucosaminyl)asparagin (NGLY1), Caspase-2 (CASP2), Ribosomal protein S6 kinase alpha-3 (RPS6KA3), E3 ubiquitin-protein ligase UBR1 (UBR1), Serine/threonine-protein kinase Chk2 (CHEK2), Phosphatidylinositol 3,4,5-trisphosphate 5-phospha (INPPL1), Histone acetyltransferase p300 (EP300), Creatine kinase U-type (mitochondrial) (CKMT1B), E3 ubiquitin-protein ligase TRIM33 (TRIM33), Cancer-related nucleoside triphosphatase (NTPCR), Aconitate hydratase (mitochondrial) (ACO2), Ubiquitin carboxyl-terminal hydrolase 34 (USP34), Probable E3 ubiquitin-protein ligase HERC4 (HERC4), E3 ubiquitin-protein ligase HECTD1 (HECTD1), Peroxisomal 2,4-dienoyl-CoA reductase (DECR2), Helicase ARIP4 (RAD54L2), Ubiquitin-like modifier-activating enzyme 7 (UBA7), ER degradation-enhancing alpha-mannosidase-like 3 (EDEM3), Ubiquitin-conjugating enzyme E20 (UBE2O), Dual specificity mitogen-activated protein kinase (MAP2K7), Myotubularin-related protein 1 (MTMR1), Calcium-dependent phospholipase A2 (PLA2G5), Mitotic checkpoint serine/threonine-protein kinase (BUB1B), Putative transferase CAF17 (mitochondrial) (IBA57), Tyrosine-protein kinase ZAP-70 (ZAP70), E3 ubiquitin-protein ligase pellino homolog 1 (PELI1), Neuropathy target esterase (PNPLA6), Ribosomal protein S6 kinase alpha-3 (RPS6KA3), N6-adenosine-methyltransferase 70 kDa subunit (METTL3), Fructosamine-3-kinase (FN3K), Ubiquitin carboxyl-terminal hydrolase 22 (USP22), Rab3 GTPase-activating protein catalytic subunit (RAB3GAP1), Caspase-5 (CASP5), L-2-hydroxyglutarate dehydrogenase (mitochondrial) (L2HGDH), Saccharopine dehydrogenase-like oxidoreductase (SCCPDH), FLAD1 FAD synthase, Lysine-specific demethylase 3 Å (KDM3A), or Ubiquitin carboxyl-terminal hydrolase 34 (USP34).

In some embodiments, the cysteine-containing protein is a signaling protein. In some instances, exemplary signaling protein includes vascular endothelial growth factor (VEGF) proteins or proteins involved in redox signaling. Exemplary VEGF proteins include VEGF-A, VEGF-B, VEGF-C, VEGF-D, and PGF. Exemplary proteins involved in redox signaling include redox-regulatory protein FAM213A.

In some embodiments, the cysteine-containing protein is a transcription factor or regulator. Exemplary cysteine-containing proteins as transcription factors and regulators include, but are not limited to, 40S ribosomal protein S3 (RPS3), Basic leucine zipper and W2 domain-containing protein (BZW1), Poly(rC)-binding protein 1 (PCBP1), 40S ribosomal protein S11 (RPS11), 40S ribosomal protein S4, X isoform (RPS4X), Signal recognition particle 9 kDa protein (SRP9), Non-POU domain-containing octamer-binding protein (NONO), N-alpha-acetyltransferase 15, NatA auxiliary subunit (NAA15), Cleavage stimulation factor subunit 2 (CSTF2), Lamina-associated polypeptide 2, isoform alpha (TMPO), Heterogeneous nuclear ribonucleoprotein R (HNRNPR), MMS19 nucleotide excision repair protein homolog (MMS19), SWI/SNF complex subunit SMARCC2 (SMARCC2), Enhancer of mRNA-decapping protein 3 (EDC3), H/ACA ribonucleoprotein complex subunit 2 (NHP2), WW domain-containing adapter protein with coiled-c (WAC), N-alpha-acetyltransferase 15 NatA auxiliary subunit (NAA15), 40S ribosomal protein S11 (RPS11), Signal transducer and activator of transcription 1 (STAT1), Mediator of RNA polymerase II transcription subunit (MED15), Lamina-associated polypeptide 2 (isoform alpha) (TMPO), MMS19 nucleotide excision repair protein homolog (MMS19), DNA mismatch repair protein Msh2 (MSH2), Recombining binding protein suppressor of hairless (RBPJ), Mediator of RNA polymerase II transcription subunit (MED17), Heterogeneous nuclear ribonucleoprotein U (HNRNPU), Transcription initiation factor IIA subunit 2 (GTF2A2), Chromatin accessibility complex protein 1 (CHRAC1), CDKN2A-interacting protein (CDKN2AIP), Zinc finger protein 217 (ZNF217), Signal transducer and activator of transcription 3 (STAT3), WD repeat and HMG-box DNA-binding protein 1 (WDHD1), Lamina-associated polypeptide 2 (isoform alpha) (TMPO), Lamina-associated polypeptide 2 (isoforms beta/gam) (TMPO), Interferon regulatory factor 4 (IRF4), Protein flightless-1 homolog (FLII), Heterogeneous nuclear ribonucleoprotein F (HNRNPF), Nucleus accumbens-associated protein 1 (NACC1), Transcription elongation regulator 1 (TCERG1), Protein HEXIM1 (HEXIM1), Enhancer of mRNA-decapping protein (EDC3), Zinc finger protein Aiolos (IKZF3), Transcription elongation factor SPT5 (SUPT5H), Forkhead box protein K1 (FOXK1), LIM domain-containing protein 1 (LIMD1), MMS19 nucleotide excision repair protein homolog (MMS19), Elongator complex protein 4 (ELP4), Ankyrin repeat and KH domain-containing protein 1 (ANKHD1), PML, Nuclear factor NF-kappa-B p100 subunit (NFκB2), Heterogeneous nuclear ribonucleoprotein L-like (HNRPLL), CCR4-NOT transcription complex subunit 3 (CNOT3), Constitutive coactivator of PPAR-gamma-like protein (FAM120A), Mediator of RNA polymerase II transcription subunit (MED15), 60S ribosomal protein L7 (RPL7), Interferon regulatory factor 8 (IRF8), COUP transcription factor 2 (NR2F2), Mediator of RNA polymerase II transcription subunit (MEDI), tRNA (uracil-5-)-methyltransferase homolog A (TRMT2A), Transcription factor p65 (RELA), Exosome complex component RRP42 (EXOSC7), General transcription factor 3C polypeptide 1 (GTF3C1), Mothers against decapentaplegic homolog 2 (SMAD2), Ankyrin repeat domain-containing protein 17 (ANKRD17), MMS19 nucleotide excision repair protein homolog (MMS19), Death domain-associated protein 6 (DAXX), Zinc finger protein 318 (ZNF318), Thioredoxin-interacting protein (TXNIP), Glucocorticoid receptor (NR3C1), Iron-responsive element-binding protein 2 (IREB2), Zinc finger protein 295 (ZNF295), Polycomb protein SUZ12 (SUZ12), Cleavage stimulation factor subunit 2 tau variant (CSTF2T), C-myc promoter-binding protein (DENND4A), Pinin (PNN), Mediator of RNA polymerase II transcription subunit (MEDS), POU domain, class 2, transcription factor 2 (POU2F2), Enhancer of mRNA-decapping protein 3 (EDC3), A-kinase anchor protein 1 (mitochondrial) (AKAP1), Transcription factor RelB (RELB), RNA polymerase II-associated protein 1 (RPAP1), Zinc finger protein 346 (ZNF346), Chromosome-associated kinesin KIF4 Å (KIF4A), Mediator of RNA polymerase II transcription subunit (MED12), Protein NPAT (NPAT), Leucine-rich PPR motif-containing protein (mitochondrial) (LRPPRC), AT-hook DNA-binding motif-containing protein 1 (AHDC1), Mediator of RNA polymerase II transcription subunit (MED12), Bromodomain-containing protein 8 (BRD8), Trinucleotide repeat-containing gene 6B protein (TNRC6B), Aryl hydrocarbon receptor nuclear translocator (ARNT), Activating transcription factor 7-interacting protein (ATF7IP), Glucocorticoid receptor (NR3C1), Chromosome transmission fidelity protein 18 homolog (CHTF18), or C-myc promoter-binding protein (DENND4A).

In some embodiments, the cysteine-containing protein is a channel, transporter or receptor. Exemplary cysteine-containing proteins as channels, transporters, or receptors include, but are not limited to, Chloride intracellular channel protein 4 (CLIC4), Exportin-1 (XPO1), Thioredoxin (TXN), Protein SEC13 homolog (SEC13), Chloride intracellular channel protein 1 (CLIC1), Guanine nucleotide-binding protein subunit beta-2 (GNB2L1), Sorting nexin-6 (SNX6), Conserved oligomeric Golgi complex subunit 3 (COG3), Nuclear cap-binding protein subunit 1 (NCBP1), Cytoplasmic dynein 1 light intermediate chain 1 (DYNC1LI1), MOB-like protein phocein (MOB4), Programmed cell death 6-interacting protein (PDCD6IP), Glutaredoxin-1 (GLRX), ATP synthase subunit alpha (mitochondrial) (ATP5A1), Treacle protein (TCOF1), Dynactin subunit 1 (DCTN1), Importin-7 (IP07), Exportin-2 (CSE1L), ATP synthase subunit gamma (mitochondrial) (ATP5C1), Trafficking protein particle complex subunit 5 (TRAPPC5), Thioredoxin mitochondrial (TXN2), THO complex subunit 6 homolog (THOC6), Exportin-1 (XP01), Nuclear pore complex protein Nup50 (NUP50), Treacle protein (TCOF1), Nuclear pore complex protein Nup93 (NUP93), Nuclear pore glycoprotein p62 (NUP62), Cytoplasmic dynein 1 heavy chain 1 (DYNC1H1), Thioredoxin-like protein 1 (TXNL1), Nuclear pore complex protein Nup214 (NUP214), Protein lin-7 homolog C (LIN7C), ADP-ribosylation factor-binding protein GGA2 (GGA2), Trafficking protein particle complex subunit 4 (TRAPPC4), Protein quaking (QKI), Perilipin-3 (PLIN3), Copper transport protein ATOX1 (ATOX1), Unconventional myosin-Ic (MYO1C), Nucleoporin NUP53 (NUP35), Vacuolar protein sorting-associated protein 18 homolog (VPS18), Dedicator of cytokinesis protein 7 (DOCK7), Nucleoporin p54 (NUP54), Ras-related GTP-binding protein C (RRAGC), Arf-GAP with Rho-GAP domain (ANK repeat and PH domain) (ARAP1), Exportin-5 (XP05), Kinectin (KTN1), Chloride intracellular channel protein 6 (CLIC6), Voltage-gated potassium channel subunit beta-2 (KCNAB2), Exportin-5 (XP05), Ras-related GTP-binding protein C (RRAGC), Ribosome-binding protein 1 (RRBP1), Acyl-CoA-binding domain-containing protein 6 (ACBD6), Chloride intracellular channel protein 5 (CLIC5), Pleckstrin homology domain-containing family A member (PLEKHA2), ADP-ribosylation factor-like protein 3 (ARL3), Protein transport protein Sec24C (SEC24C), Voltage-dependent anion-selective channel protein (VDAC3), Programmed cell death 6-interacting protein (PDCD6IP), Chloride intracellular channel protein 3 (CLIC3), Multivesicular body subunit 12 Å (FAM125A), Eukaryotic translation initiation factor 4E transporter (EIF4ENIF1), NmrA-like family domain-containing protein 1 (NMRAL1), Nuclear pore complex protein Nup98-Nup96 (NUP98), Conserved oligomeric Golgi complex subunit 1 (COG1), Importin-4 (IP04), Pleckstrin homology domain-containing family A member (PLEKHA2), Cytoplasmic dynein 1 heavy chain 1 (DYNC1H1), DENN domain-containing protein 1C (DENND1C), Cytoplasmic dynein 1 heavy chain 1 (DYNC1H1), Protein ELYS (AHCTF1), Trafficking protein particle complex subunit 1 (TRAPPC1), Guanine nucleotide-binding protein-like 3 (GNL3), or Importin-13 (IPO13).

In some embodiments, the cysteine-containing protein is a chaperone. Exemplary cysteine-containing proteins as chaperones include, but are not limited to, 60 kDa heat shock protein (mitochondrial) (HSPD1), T-complex protein 1 subunit eta (CCT7), T-complex protein 1 subunit epsilon (CCT5), Heat shock 70 kDa protein 4 (HSPA4), GrpE protein homolog 1 (mitochondrial) (GRPEL1), Tubulin-specific chaperone E (TBCE), Protein unc-45 homolog A (UNC45A), Serpin H1 (SERPINH1), Tubulin-specific chaperone D (TBCD), Peroxisomal biogenesis factor 19 (PEX19), BAG family molecular chaperone regulator 5 (BAGS), T-complex protein 1 subunit theta (CCT8), Protein canopy homolog 3 (CNPY3), DnaJ homolog subfamily C member 10 (DNAJC10), ATP-dependent Clp protease ATP-binding subunit clp (CLPX), or Midasin (MDN1).

In some embodiments, the cysteine-containing protein is an adapter, scaffolding or modulator protein. Exemplary cysteine-containing proteins as adapter, scaffolding, or modulator proteins include, but are not limited to, Proteasome activator complex subunit 1 (PSME1), TIP41-like protein (TIPRL), Crk-like protein (CRKL), Cofilin-1 (CFL1), Condensin complex subunit 1 (NCAPD2), Translational activator GCN1 (GCN1L1), Serine/threonine-protein phosphatase 2 Å 56 kDa regulatory (PPP2R5D), UPF0539 protein C7orf59 (C7orf59), Protein diaphanous homolog 1 (DIAPH1), Protein asunder homolog (Asun), Ras GTPase-activating-like protein IQGAP1 (IQGAP1), Sister chromatid cohesion protein PDSS homolog A (PDSSA), Reticulon-4 (RTN4), Proteasome activator complex subunit 4 (PSME4), Condensin complex subunit 2 (NCAPH), Sister chromatid cohesion protein PDSS homolog A (PDSSA), cAMP-dependent protein kinase type I-alpha regulatory (PRKAR1A), Host cell factor 1 (HCFC1), Serine/threonine-protein phosphatase 4 regulatory (PPP4R2), Apoptotic chromatin condensation inducer in the nucleus (ACINI), BRISC and BRCA1-A complex member 1 (BABAM1), Interferon-induced protein with tetratricopeptide (IFIT3), Ras association domain-containing protein 2 (RASSF2), Hsp70-binding protein 1 (HSPBP1), TBC1 domain family member 15 (TBC1D15), Dynamin-binding protein (DNMBP), Condensin complex subunit 1 (NCAPD2), Beta-2-syntrophin (SNTB2), Disks large homolog 1 (DLG1), TBC1 domain family member 13 (TBC1D13), Formin-binding protein 1-like (FNBP1L), Translational activator GCN1 (GCN1L1), GRB2-related adapter protein (GRAP), G2/mitotic-specific cyclin-B1 (CCNB1), Myotubularin-related protein 12 (MTMR12), Protein FADD (FADD), Translational activator GCN1 (GCN1L1), Wings apart-like protein homolog (WAPAL), cAMP-dependent protein kinase type II-beta regulatory (PRKAR2B), Malcavernin (CCM2), MPP1 55 kDa erythrocyte membrane protein, Actin filament-associated protein 1 (AFAP1), Tensin-3 (TNS3), tRNA methyltransferase 112 homolog (TRMT112), Symplekin (SYMPK), TBC1 domain family member 2 Å (TBC1D2), ATR-interacting protein (ATRIP), Ataxin-10 (ATXN10), Succinate dehydrogenase assembly factor 2 (mitochondrial) (SDHAF2), Formin-binding protein 1 (FNBP1), Myotubularin-related protein 12 (MTMR12), Interferon-induced protein with tetratricopeptide (IFIT3), Protein CBFA2T2 (CBFA2T2), Neutrophil cytosol factor 1 (NCF1), or Protein syndesmos (NUDT16L1).

### Polypeptides Comprising a Cysteine Interacting Site

In some embodiments, a cysteine-containing polypeptide comprises a polypeptide that is at most 50 amino acid residues in length. In some embodiments, a cysteine-containing polypeptide comprises an isolated and purified polypeptide comprising at least 70% sequence identity to at least seven contiguous amino acids of an amino acid sequence selected from SEQs disclosed in the Examples. In some embodiments, a cysteine-containing polypeptide comprises an isolated and purified polypeptide comprising at least 75% sequence identity to at least seven contiguous amino acids of an amino acid sequence selected from SEQs disclosed in the Examples. In some embodiments, a cysteine-containing polypeptide comprises an isolated and purified polypeptide comprising at least 80% sequence identity to at least seven contiguous amino acids of an amino acid sequence selected from SEQs disclosed in the Examples. In some embodiments, a cysteine-containing polypeptide comprises an isolated and purified polypeptide comprising at least 85% sequence identity to at least seven contiguous amino acids of an amino acid sequence selected from SEQs disclosed in the Examples. In some embodiments, a cysteine-containing polypeptide comprises an isolated and purified polypeptide comprising at least 90% sequence identity to at least seven contiguous amino acids of an amino acid sequence selected from SEQs disclosed in the Examples. In some embodiments, a cysteine-containing polypeptide comprises an isolated and purified polypeptide comprising at least 91% sequence identity to at least seven contiguous amino acids of an amino acid sequence selected from SEQs disclosed in the Examples. In some embodiments, a cysteine-containing polypeptide comprises an isolated and purified polypeptide comprising at least 92% sequence identity to at least seven contiguous amino acids of an amino acid sequence selected from SEQs disclosed in the Examples. In some embodiments, a cysteine-containing polypeptide comprises an isolated and purified polypeptide comprising at least 93% sequence identity to at least seven contiguous amino acids of an amino acid sequence selected from SEQs disclosed in the Examples. In some embodiments, a cysteine-containing polypeptide comprises an isolated and purified polypeptide comprising at least 94% sequence identity to at least seven contiguous amino acids of an amino acid sequence selected from SEQs disclosed in the Examples. In some embodiments, a cysteine-containing polypeptide comprises an isolated and purified polypeptide comprising at least 95% sequence identity to at least seven contiguous amino acids of an amino acid sequence selected from SEQs disclosed in the Examples. In some embodiments, a cysteine-containing polypeptide comprises an isolated and purified polypeptide comprising at least 96% sequence identity to at least seven contiguous amino acids of an amino acid sequence selected from SEQs disclosed in the Examples. In some embodiments, a cysteine-containing polypeptide comprises an isolated and purified polypeptide comprising at least 97% sequence identity to at least seven contiguous amino acids of an amino acid sequence selected from SEQs disclosed in the Examples. In some embodiments, a cysteine-containing polypeptide comprises an isolated and purified polypeptide comprising at least 98% sequence identity to at least seven contiguous amino acids of an amino acid sequence selected from SEQs disclosed in the Examples. In some embodiments, a cysteine-containing polypeptide comprises an isolated and purified polypeptide comprising at least 99% sequence identity to at least seven contiguous amino acids of an amino acid sequence selected from SEQs disclosed in the Examples. In some embodiments, a cysteine-containing polypeptide comprises an isolated and purified polypeptide comprising 100% sequence identity to at least seven contiguous amino acids of an amino acid sequence selected from SEQs disclosed in the Examples. In some embodiments, a cysteine-containing polypeptide comprises an isolated and purified polypeptide consisting of 100% sequence identity to at least seven contiguous amino acids of an amino acid sequence selected from SEQs disclosed in the Examples.

As used herein, a polypeptide includes natural amino acids, unnatural amino acids, or a combination thereof. In some instances, an amino acid residue refers to a molecule containing both an amino group and a carboxyl group. Suitable amino acids include, without limitation, both the D- and L-isomers of the naturally-occurring amino acids, as well as non-naturally occurring amino acids prepared by organic synthesis or other metabolic routes. The term amino acid, as used herein, includes, without limitation, α-amino acids, natural amino acids, non-natural amino acids, and amino acid analogs.

The term “α-amino acid” refers to a molecule containing both an amino group and a carboxyl group bound to a carbon which is designated the α-carbon.

The term “β-amino acid” refers to a molecule containing both an amino group and a carboxyl group in a β configuration.

“Naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

“Hydrophobic amino acids” include small hydrophobic amino acids and large hydrophobic amino acids. “Small hydrophobic amino acid” are glycine, alanine, proline, and analogs thereof “Large hydrophobic amino acids” are valine, leucine, isoleucine, phenylalanine, methionine, tryptophan, and analogs thereof. “Polar amino acids” are serine, threonine, asparagine, glutamine, cysteine, tyrosine, and analogs thereof “Charged amino acids” are lysine, arginine, histidine, aspartate, glutamate, and analogs thereof.

The term “amino acid analog” refers to a molecule which is structurally similar to an amino acid and which is substituted for an amino acid in the formation of a peptidomimetic macrocycle Amino acid analogs include, without limitation, β-amino acids and amino acids where the amino or carboxy group is substituted by a similarly reactive group (e.g., substitution of the primary amine with a secondary or tertiary amine, or substitution of the carboxy group with an ester).

The term “non-natural amino acid” refers to an amino acid which is not one of the twenty amino acids commonly found in peptides synthesized in nature, and known by the one letter abbreviations A, R, N, C, D, Q, E, G, H, I, L, K, M, F, P, S, T, W, Y and V.

In some instances, amino acid analogs include β-amino acid analogs. Examples of β-amino acid analogs include, but are not limited to, the following: cyclic β-amino acid analogs; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl) butyric acid; (S)-3-amino-4-(4-cyanophenyl)-butyric acid; (S)-3-amino-4-(4-fluorophenyl) butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid 6-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; NS-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; 0-t-butyl-L-β-homotyrosine; 2-aminocyclopentane carboxylic acid; and 2-aminocyclohexane carboxylic acid.

In some instances, amino acid analogs include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanin; β-cyclohexyl-D-alanine; β-cyclohexyl-L-alanine; β-cyclopenten-1-yl-alanine; β-cyclopentyl-alanine; β-cyclopropyl-L-Ala-OH·dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-β-thienyl)glycine; D-2-aminocaproic acid; D-2-indanylglycine; D-allylglycine-dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine·dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

In some instances, amino acid analogs include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃)—OH; Nδ-benzyloxycarbonyl-L-omithine; Nω-nitro-D-arginine; Nω-nitro-L-arginine; α-methyl-ornithine; 2,6-diaminoheptane dioic acid; L-ornithine; (Nδ-1-(4,4-dimethyl-2,6-dioxo-cyclohex-1-ylidene)ethyl)-D-omithine; (Nδ-1-(4,4-dimethyl-2,6-dioxo-cyclohex-1-ylidene)ethyl)-L-ornithine; (Nδ-4-methyltrityl)-D-omithine; (Nδ-4-methyltrityl)-L-ornithine; D-omithine; L-ornithine; Arg(Me)(Pbf)-OH; Arg(Me)₂-OH (asymmetrical); Arg(Me)2-OH (symmetrical); Lys(ivDde)-OH; Lys(Me)2-OH·HCl; Lys(Me3)-OH chloride; Nω-nitro-D-arginine; and Nω-nitro-L-arginine.

In some instances, amino acid analogs include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)-OH; and pyroglutamic acid.

In some instances, amino acid analogs include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridypethyl]-DL-penicillamine, [2-(4-pyridypethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

In some instances, amino acid analogs include analogs of phenylalanine and tyrosine. Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

In some instances, amino acid analogs include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

In some instances, amino acid analogs include analogs of serine and threonine. Examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

In some instances, amino acid analogs include analogs of tryptophan. Examples of amino acid analogs of tryptophan include, but are not limited to, the following: α-methyl-tryptophan; β-(3-benzothienyl)-D-alanine; β-(3-benzothienyl)-L-al anine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some instances, amino acid analogs are racemic. In some instances, the D isomer of the amino acid analog is used. In some cases, the L isomer of the amino acid analog is used. In some instances, the amino acid analog comprises chiral centers that are in the R or S configuration. Sometimes, the amino group(s) of a β-amino acid analog is substituted with a protecting group, e.g., tert-butyloxycarbonyl (BOC group), 9-fluorenylmethyloxycarbonyl (FMOC), tosyl, and the like. Sometimes, the carboxylic acid functional group of a β-amino acid analog is protected, e.g., as its ester derivative. In some cases, the salt of the amino acid analog is used.

### Cysteine-Containing Polypeptide Production

In some embodiments, a cysteine-containing polypeptide described above is generated recombinantly or is synthesized chemically. In some instances, a cysteine-containing polypeptide described above is generated recombinantly, for example, by a host cell system or in a cell-free system. In some instances, a cysteine-containing polypeptide described above is synthesized chemically.

In some embodiments, a cysteine-containing polypeptide described above is generated recombinantly by a host cell system. Exemplary host cell systems include eukaryotic cell system (e.g., mammalian cell, insect cells, yeast cells or plant cell) or a prokaryotic cell system (e.g., gram-positive bacterium or a gram-negative bacterium).

In some embodiments, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293 Å cell line, 293FT cell line, 293F cells, 293H cells, A549 cells, MDCK cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some embodiments, a eukaryotic host cell is an insect host cell. Exemplary insect host cell include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris yeast strains such as GS115, KM71H, SMD1168, SMD1168H, and X-33, and Saccharomyces cerevisiae yeast strain such as INVScl.

In some embodiments, a eukaryotic host cell is a plant host cell. In some instances, the plant cells comprises a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Mach1™, DH10B™, TOP10, DH5α, DH10Bac™, OmniMax™, MegaX™, DH12S™ INV110, TOP10F′, INVαF, TOP10/P3, ccdB Survival, PIR1, PIR2, Stb12™, Stb13™, or Stb14™.

In some instances, suitable vectors for the production of a cysteine-containing polypeptide include any suitable vectors derived from either eukaryotic or prokaryotic sources. Exemplary vectors include vectors from bacteria (e.g., E. coli), insects, yeast (e.g., Pichia pastoris), algae, or mammalian source. Bacterial vectors include, for example, pACYC177, pASK75, pBAD vector series, pBADM vector series, pET vector series, pE™ vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBacl, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is used for the production of a cysteine-containing polypeptide. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, or HeLa cells. Exemplary cell-free systems include E. coli S30 Extract system, E. coli T7 S30 system, or PURExpress®.

### Methods of Use

In some embodiments, disclosed herein include methods of modulating an immune response in a subject. In some embodiments, disclosed herein is a method of modulating an immune response in a subject, which comprises administering to the subject a therapeutically effective amount of a small molecule fragment of Formula (I):
wherein:

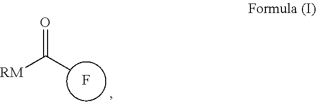

RM is a reactive moiety selected from a Michael acceptor moiety, a leaving group moiety, or a moiety capable of forming a covalent bond with the thiol group of a cysteine residue; and
F is a small molecule fragment moiety.

In some embodiments, the small molecule fragment interacts with an endogenous cysteine-containing polypeptide expressed in the subject to form a cysteine-containing polypeptide-small molecule fragment adduct. In some instances, the cysteine-containing polypeptide-small molecule fragment adduct comprises a covalent bonding. In some cases, the cysteine-containing polypeptide-small molecule fragment adduct comprises an irreversible bonding. In other cases, the cysteine-containing polypeptide-small molecule fragment adduct comprises a reversible bonding. In some instances, an endogenous cysteine-containing polypeptide is a polypeptide that is expressed or present in a cell of interest (e.g., a diseased cell such as a cancerous cell). In some instances, an endogenous cysteine-containing polypeptide is a polypeptide that is overexpressed in a cell of interest (e.g., a diseased cell such as a cancerous cell). In some instances, an endogenous cysteine-containing polypeptide is a polypeptide that harbors one or more mutations in a cell of interest (e.g., a diseased cell such as a cancerous cell). In some instances, a mutation comprises a missense mutation, an insertion, or a deletion. In some instances, a mutation comprises a truncation, for example, a truncation at the N-terminus or the C-terminus of the polypeptide. In additional instances, an endogenous cysteine-containing polypeptide is a polypeptide that has an altered conformation in a cell of interest (e.g., a diseased cell such as a cancerous cell) relative to the conformation of the wild-type polypeptide.

In some instances, a cysteine-containing polypeptide-small molecule fragment adduct induces an immune response. In some cases, the immune response is a humoral immune response. In other cases, the immune response is a cell mediated immune response. In some instances, the cysteine-containing polypeptide-small molecule fragment adduct induces a humoral immune response. In some instances, a cysteine-containing polypeptide-small molecule fragment adduct induces a cell mediated immune response. In additional instances, a cysteine-containing polypeptide-small molecule fragment adduct induces a humoral immune response and a cell mediated immune response. In some instances, humoral immunity (or antibody-mediated beta cellularis immune system) is the production of antibody and its accessory processes such as Th2 activation, cytokine production, germinal center formation, isotype switching, affinity maturation, and memory cell generation. In some instances, humoral immunity is mediated by macromolecules in the extracellular fluids. In some cases, cell mediated immunity comprises activation of phagocytes, antigen-specific cytotoxic T-lymphocytes, and release of cytokines in response to an antigen. In some cases, cell mediated immunity differs from humoral immunity in that it does not involve production of antibody.

In some embodiments, a cysteine-containing polypeptide-small molecule fragment adduct increases an immune response relative to a control. In some cases, a cysteine-containing polypeptide-small molecule fragment adduct increases a humoral immune response relative to a control. In additional cases, a cysteine-containing polypeptide-small molecule fragment adduct increases a cell mediated immune response relative to a control. In additional cases, a cysteine-containing polypeptide-small molecule fragment adduct increases a humoral immune response and a cell mediated immune response relative to a control.

In some cases, a control is the level of an immune response in the subject prior to administration of the small molecule fragment or is the level of an immune response in a subject who has not been exposed to the small molecule fragment. In some cases, a control is the level of a humoral immune response or a cell mediated immune response in the subject prior to administration of the small molecule fragment or is the level of a humoral immune response or a cell mediated immune response in a subject who has not been exposed to the small molecule fragment.

In some instances, a cysteine-containing polypeptide-small molecule fragment adduct modulates an immune response. In some cases, the immune response is a humoral immune response. In other cases, the immune response is a cell mediated immune response. In some instances, the cysteine-containing polypeptide-small molecule fragment adduct modulates a humoral immune response. In some instances, a cysteine-containing polypeptide-small molecule fragment adduct modulates a cell mediated immune response. In additional instances, a cysteine-containing polypeptide-small molecule fragment adduct modulates a humoral immune response and a cell mediated immune response.

In some instances, a cysteine-containing polypeptide is a non-denatured form of the polypeptide.

In some instances, a cysteine-containing polypeptide comprises a biologically active cysteine site. As described above and elsewhere herein, in some cases, a biologically active cysteine site is a cysteine residue that is located about 10 Å or less to an active-site ligand or residue. In other cases, a biologically active cysteine site is a cysteine residue that is located greater than 10 Å from an active-site ligand or residue. In some cases, the cysteine residue that is located greater than 10 Å from the active-site ligand or residue is a non-active site cysteine.

Further as described elsewhere herein, a cysteine-containing polypeptide comprises, in some instances, an enzyme, a transporter, a receptor, a channel protein, an adaptor protein, a chaperone, a signaling protein, a plasma protein, transcription related protein, translation related protein, mitochondrial protein, or cytoskeleton related protein. In some cases, the cysteine-containing polypeptide comprises an enzyme, a transporter, a receptor, a channel protein, an adaptor protein, a chaperone, a signaling protein, transcription related protein, or translation related protein.

In some embodiments, a cysteine-containing polypeptide is about 20, 30, 35, 40, 45, 50, 55, 60, 65, 70, 75, 80, 85, 90, 95, 100, 150, 200, 250, 300, 350, 400, 450, 500, 600, 700, 800, 900, 1000, 1100, 1200, 1500, 2000, 2100, 2200, 2500 amino acid residues in length or more. In some cases, a cysteine-containing polypeptide is about 20 amino acid residues in length or more. In some cases, a cysteine-containing polypeptide is about 60 amino acid residues in length or more. In some cases, a cysteine-containing polypeptide is about 70 amino acid residues in length or more. In some cases, a cysteine-containing polypeptide is about 80 amino acid residues in length or more. In some cases, a cysteine-containing polypeptide is about 90 amino acid residues in length or more. In some cases, a cysteine-containing polypeptide is about 100 amino acid residues in length or more. In some cases, a cysteine-containing polypeptide is about 150 amino acid residues in length or more. In some cases, a cysteine-containing polypeptide is about 200 amino acid residues in length or more. In some cases, a cysteine-containing polypeptide is about 300 amino acid residues in length or more. In some cases, a cysteine-containing polypeptide is about 400 amino acid residues in length or more. In some cases, a cysteine-containing polypeptide is about 500 amino acid residues in length or more. In some cases, a cysteine-containing polypeptide is about 800 amino acid residues in length or more. In some cases, a cysteine-containing polypeptide is about 1000 amino acid residues in length or more. In some cases, a cysteine-containing polypeptide is about 1500 amino acid residues in length or more.

In some embodiments, as described above, a small molecule fragment comprises a Michael acceptor moiety which comprises an alkene or an alkyne moiety. In some instances, a covalent bond is formed between a portion of the Michael acceptor moiety on the small molecule fragment and a portion of a cysteine residue of the cysteine-containing polypeptide.

In some instances, a small molecule fragment comprises a small molecule fragment moiety F which is obtained from a compound library. In some instances, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some cases, F is a small molecule fragment moiety illustrated in FIGS. 2B and 4C. In some cases, the small molecule fragment is a small molecule fragment illustrated in FIGS. 2B and 4C.

In some instances, a small molecule fragment has a molecular weight of about 150 Dalton or higher. In some cases, a small molecular fragment has a molecular weight of about 175, 200, 225, 250, 275, 300, 350, 400, 450, 500, 550, 600, 650, 700, 750, 800, 850, 900, 950, 1000 Dalton, or higher. In some cases, a molecular weight of the small molecular fragment is prior to enrichment with a halogen, a nonmetal, or a transition metal. In some cases, a small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher.

In some instances, the method further comprises administration of a cysteine-containing polypeptide-small molecule fragment adduct. In some instances, the cysteine-containing polypeptide comprises an isolated and purified polypeptide comprising at least 80%, 85%, 90%, 95%, 96%, 97%, 98%, or 99% sequence identity to at least seven contiguous amino acids of an amino acid sequence selected from SEQs disclosed in the Examples. In some cases, the cysteine-containing polypeptide-small molecule fragment adduct further enhances or increases an immune response. In some instances, an enhancement or an increase of the immune response is relative to a level of the immune response prior to administration of the cysteine-containing polypeptide-small molecule fragment adduct.

In some cases, the method further comprises administration of an adjuvant.

In some cases, the small molecule fragment is formulated for parenteral, oral, or intranasal administration.

### Diseases or Indications

In some embodiments, disclosed herein include a method of administrating a small molecule fragment to a subject in which the small molecule fragment interacts with an endogenous cysteine-containing polypeptide expressed in the subject to form a cysteine-containing polypeptide-small molecule fragment adduct. In some embodiments, the cysteine-containing polypeptide is overexpressed in a disease or condition. In some cases, the overexpressed cysteine-containing polypeptide comprises one or more mutations. In some cases, the cysteine-containing polypeptide comprising one or more mutations is overexpressed in a disease or condition.

In some instances, the disease or condition is cancer. In some cases, the cysteine-containing polypeptide is a cancer-associated protein. In some cases, the cysteine-containing polypeptide is overexpressed in a cancer. In some cases, the cysteine-containing polypeptide comprising one or more mutations is overexpressed in a cancer. In some instances, a mutation comprises a missense mutation, an insertion, or a deletion. In some instances, a mutation comprises a truncation at a terminus of a protein. In some instances, a mutation alters the conformation of a protein relative to the conformation of its wild-type protein. In additional instances, a mutation does not alter the conformation of a protein.

In some instances, a cancer is a solid tumor. In some instances, a cancer is a hematologic malignancy. In some instances, a cancer is a relapsed or refractory cancer, or a metastatic cancer. In some instances, a solid tumor is a relapsed or refractory solid tumor, or a metastatic solid tumor. In some cases, a hematologic malignancy is a relapsed or refractory hematologic malignancy, or a metastatic hematologic malignancy.

In some embodiments, a cancer is a solid tumor. Exemplary solid tumor includes, but is not limited to, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer or vulvar cancer.

In some embodiments, a cysteine-containing polypeptide described herein that is overexpressed and/or comprises one or more mutations is present in a solid tumor. In some cases, a cysteine-containing polypeptide described herein that is overexpressed and/or comprises one or more mutations is present in metastatic solid tumor. In some cases, a cysteine-containing polypeptide described herein that is overexpressed and/or comprises one or more mutations is present in a relapsed or refractory solid tumor. In some instances, a small molecule fragment described herein interacts with a cysteine-containing polypeptide that is present, overexpressed, and/or comprises a mutation in a solid tumor.

In some instances, a cancer is a hematologic malignancy. In some instances, a hematologic malignancy is a leukemia, a lymphoma, a myeloma, a non-Hodgkin's lymphoma, or a Hodgkin's lymphoma. In some instances, a hematologic malignancy comprises chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, a cysteine-containing polypeptide described herein that is overexpressed and/or comprises one or more mutations is present in a hematologic malignancy. In some cases, a cysteine-containing polypeptide described herein that is overexpressed and/or comprises one or more mutations is present in metastatic hematologic malignancy. In some cases, a cysteine-containing polypeptide described herein that is overexpressed and/or comprises one or more mutations is present in a relapsed or refractory hematologic malignancy. In some instances, a small molecule fragment described herein interacts with a cysteine-containing polypeptide that is present, overexpressed, and/or comprises a mutation in a hematologic malignancy.

### Vaccines

In some embodiments, disclosed herein include vaccines and vaccine formulations that comprises a small molecule fragment described herein, an antibody that recognizes a cysteine-containing polypeptide-small molecule fragment adduct described herein, or a cysteine-containing polypeptide-small molecule fragment adduct described herein. In some embodiments, disclosed herein is a vaccine that comprises a small molecule fragment described herein. In some embodiments, disclosed herein is a vaccine that comprises an antibody that recognizes a cysteine-containing polypeptide-small molecule fragment adduct described herein. In some embodiments, disclosed herein is a vaccine that comprises a cysteine-containing polypeptide-small molecule fragment adduct described herein.

In some instances, a cysteine-containing polypeptide is an enzyme, a transporter, a receptor, a channel protein, an adaptor protein, a chaperone, a signaling protein, a plasma protein, transcription related protein, translation related protein, mitochondrial protein, or cytoskeleton related protein. In some cases, the cysteine-containing polypeptide is an enzyme, a transporter, a receptor, a channel protein, an adaptor protein, a chaperone, a signaling protein, transcription related protein, or translation related protein.

In some embodiments, a small molecule fragment comprises a Michael acceptor moiety which comprises an alkene or an alkyne moiety. In some instances, a covalent bond is formed between a portion of the Michael acceptor moiety on the small molecule fragment and a portion of a cysteine residue of the cysteine-containing polypeptide.

In some instances, a small molecule fragment comprises a small molecule fragment moiety F which is obtained from a compound library. In some instances, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library.

In some instances, a small molecule fragment has a molecular weight of about 150 Dalton or higher. In some cases, a small molecular fragment has a molecular weight of about 175, 200, 225, 250, 275, 300, 350, 400, 450, 500, 550, 600, 650, 700, 750, 800, 850, 900, 950, 1000 Dalton, or higher. In some cases, the molecular weight of the small molecular fragment is prior to enrichment with a halogen, a nonmetal, or a transition metal. In some cases, the small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher.

In some instances, a vaccine is formulated in a conventional manner using one or more physiologically acceptable carriers including excipients and auxiliaries which facilitate processing of the active agents into preparations which is used pharmaceutically. Proper formulation is dependent upon the route of administration chosen. Any of the well-known techniques, carriers, and excipients are used as suitable and as understood in the art.

In some instances, a vaccine is further formulated with a cysteine-containing polypeptide-small molecule fragment adduct. In some instances, a cysteine-containing polypeptide-small molecule fragment adduct enhances an immune response. In some instances, the cysteine-containing polypeptide comprises an isolated and purified polypeptide comprising at least 80%, 85%, 90%, 95%, 96%, 97%, 98%, or 99% sequence identity to at least seven contiguous amino acids of an amino acid sequence selected from SEQs disclosed in the Examples.

In some embodiments, disclosed herein is a vaccine or comprising an antibody or its binding fragment thereof that recognizes a derivative of a cysteine-containing protein having the structure of Formula (I),

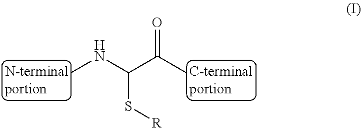

wherein,
   the derivation occurs at a cysteine residue;
   R is selected from:

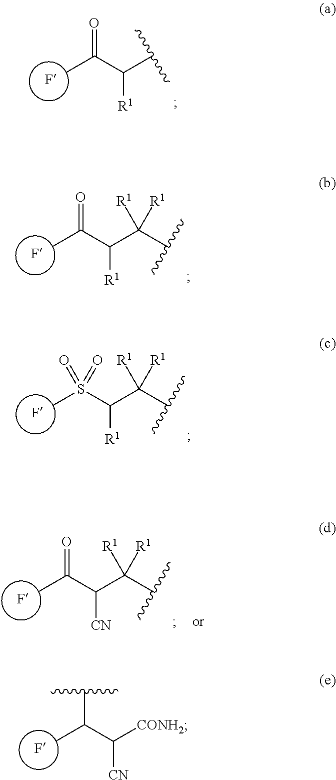

wherein,
R¹ is H, C1-C3 alkyl, or aryl; and
F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIGS. 2B and 4C.

### Adjuvant

In some embodiments, the pharmaceutical composition and/or the vaccine further comprises an adjuvant. In some instances, an adjuvant enhances the immune response (humoral and/or cellular) elicited in a subject receiving the pharmaceutical composition and/or the vaccine. In some instances, an adjuvant elicits a Th1-type response. In other instances, an adjuvant elicits a Th2-type response. In some instances, a Th1-type response is characterized by the production of cytokines such as IFN-γ as opposed to a Th2-type response which is characterized by the production of cytokines such as IL-4, IL-5 and IL-10.

In some embodiments, an adjuvant comprises a stimulatory molecule such as a cytokine. Non-limiting examples of cytokines include: CCL20, α-interferon(IFN-α), β-interferon (IFN-β), γ-interferon, platelet derived growth factor (PDGF), TNFα, TNFp, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, IL-28, MHC, CD80, CD86, IL-1, IL-2, IL-4, IL-5, IL-6, IL-10, IL-18, MCP-1, MIP-la, MIP-1-, IL-8, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DRS, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-I, JNK, interferon response genes, NFkB, Bax, TRAIL, TRAILrec, TRAILrecDRC5, TRAIL-R3, 1′RAIL-R4, RANK, RANK LIGAND, Ox40, Ox40 LIGAND, NKG2D, MICA, MICB, NKG2A, NKG2B, NKG2C, NKG2E, NKG2F, TAPI, and TAP2.

Additional adjuvants include, for example: MCP-1, MIP-la, MIP-lp, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

In some embodiments, an adjuvant is a modulator of a toll like receptor. Examples of modulators of toll-like receptors include TLR-9 agonists and are not limited to small molecule modulators of toll-like receptors such as Imiquimod. Other examples of adjuvants that are used in combination with a vaccine described herein include and are not limited to saponin, CpG ODN and the like.

Sometimes, an adjuvant is selected from bacteria toxoids, polyoxypropylene-polyoxyethylene block polymers, aluminum salts, liposomes, CpG polymers, oil-in-water emulsions, or a combination thereof.

In some embodiments, an adjuvant is a lipid-based adjuvant, such as MPLA and MDP. In some instances, monophosphoryl lipid A (MPLA) is an adjuvant that causes increased presentation of liposomal antigen to specific T Lymphocytes. In some cases, a muramyl dipeptide (MDP) is used as a suitable adjuvant in conjunction with the vaccine formulations described herein.

In some embodiments, an adjuvant is an oil-in-water emulsion. The oil-in-water emulsion suitable for use with a vaccine described herein include, for example, at least one oil and at least one surfactant, with the oil(s) and surfactant(s) being biodegradable (metabolisable) and biocompatible. In some instances, the oil droplets in the emulsion is less than 5 μm in diameter, or have a sub-micron diameter, with these small sizes being achieved with a microfluidiser to provide stable emulsions. Droplets with a size less than 220 nm are optionally subjected to filter sterilization.

In some instances, oils used include such as those from an animal (such as fish) or vegetable source. Sources for vegetable oils include, for example, nuts, seeds and grains. Peanut oil, soybean oil, coconut oil, and olive oil, exemplify the nut oils. Jojoba oil is used e.g. obtained from the jojoba bean. Seed oils include safflower oil, cottonseed oil, sunflower seed oil, sesame seed oil, etc. The grain group include: corn oil and oils of other cereal grains such as wheat, oats, rye, rice, teff, triticale, and the like. 6-10 carbon fatty acid esters of glycerol and 1,2-propanediol, while not occurring naturally in seed oils, can be prepared by hydrolysis, separation and esterification of the appropriate materials starting from the nut and seed oils. Fats and oils from mammalian milk are optionally metabolizable and are therefore used in with the vaccines described herein. The procedures for separation, purification, saponification and other means necessary for obtaining pure oils from animal sources are well known in the art. Fish contain metabolizable oils which are readily recovered. For example, cod liver oil, shark liver oils, and whale oil such as spermaceti can exemplify several of the fish oils which can be used herein. A number of branched chain oils can be synthesized biochemically in 5-carbon isoprene units and can be generally referred to as terpenoids. Shark liver oil contains a branched, unsaturated terpenoid known as squalene, 2,6,10,15,19,23-hexamethyl-2,6,10,14,18,22-tetracosahexaene. Squalane, the saturated analog to squalene, can also be used. Fish oils, including squalene and squalane, can be readily available from commercial sources or can be obtained by methods known in the art.

Other useful oils include tocopherols, for use in elderly patients (e.g. aged 60 years or older) due to vitamin E been reported to have a positive effect on the immune response in this patient group. Further, tocopherols have antioxidant properties that, for example, help to stabilize the emulsions. Various tocopherols exist (α, β, γ, δ, ϵ or ξ) but α is usually used. An example of α-tocopherol is DL-α-tocopherol. α-tocopherol succinate can be compatible with HW vaccines and can be a useful preservative as an alternative to mercurial compounds.

Mixtures of oils are used e.g. squalene and α-tocopherol. In some instances, an oil content in the range of 2-20% (by volume) is used.

In some instances, surfactants are classified by their ‘14LB’ (hydrophile/lipophile balance). In some cases, surfactants have a HLB of at least 10, at least 15, and/or at least 16. Surfactants can include, but are not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants can be used herein.

Mixtures of surfactants are used e.g. Tween 80/Span 85 mixtures. A combination of a polyoxyethylene sorbitan ester and an octoxynol are also suitable. Another combination comprises, for example, laureth 9 plus a polyoxyethylene sorbitan ester and/or an octoxynol.

The amounts of surfactants (% by weight) include, for example, polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

### Carriers and Excipients

In some instances, a vaccine further includes carriers and excipients (including but not limited to buffers, carbohydrates, mannitol, proteins, polypeptides or amino acids such as glycine, antioxidants, bacteriostats, chelating agents, suspending agents, thickening agents and/or preservatives), water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In another instances, the pharmaceutical preparation is substantially free of preservatives. In other instances, the pharmaceutical preparation can contain at least one preservative. General methodology on pharmaceutical dosage forms is found in Ansel et al., Pharmaceutical Dosage Forms and Drug Delivery Systems (Lippencott Williams & Wilkins, Baltimore Md. (1999)). It will be recognized that, while any suitable carrier known to those of ordinary skill in the art can be employed to administer the pharmaceutical compositions described herein, the type of carrier will vary depending on the mode of administration.

In some instances, a pharmaceutical composition of the vaccine is encapsulated within liposomes using well-known technology. Biodegradable microspheres can also be employed as carriers for the pharmaceutical compositions described herein. Suitable biodegradable microspheres are disclosed, for example, in U.S. Pat. Nos. 4,897,268; 5,075,109, 5,928,647; 5,811,128; 5,820,883; 5,853,763; 5,814,344 and 5,942,252.

In some cases, a pharmaceutical composition is administered in liposomes or microspheres (or microparticles). Methods for preparing liposomes and microspheres for administration to a patient are well known to those of skill in the art. U.S. Pat. No. 4,789,734, the contents of which are hereby incorporated by reference, describes methods for encapsulating biological materials in liposomes. Essentially, the material is dissolved in an aqueous solution, the appropriate phospholipids and lipids added, along with surfactants if required, and the material dialyzed or sonicated, as necessary. A review of known methods is provided by G. Gregoriadis, Chapter 14. “Liposomes,” Drug Carriers in Biology and Medicine, pp. 2.sup.87-341 (Academic Press, 1979).

Microspheres formed of polymers or proteins are well known to those skilled in the art and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, and kin, TIPS 19:15-57 (1998), the contents of which are hereby incorporated by reference.

In some cases, a vaccine includes preservatives such as thiomersal or 2-phenoxyethanol. In some instances, the vaccine is substantially free from (e.g. <10 μg/ml) mercurial material e.g. thiomersal-free. α-Tocopherol succinate may be used as an alternative to mercurial compounds.

For controlling the tonicity, a physiological salt such as sodium salt are optionally included in the vaccine. Other salts include potassium chloride, potassium dihydrogen phosphate, disodium phosphate, and/or magnesium chloride, or the like.

In some instances, a vaccine has an osmolality of between 200 mOsm/kg and 400 mOsm/kg, between 240-360 mOsm/kg, or within the range of 290-310 mOsm/kg.

In some cases, a vaccine comprises one or more buffers, such as a Tris buffer; a borate buffer; a succinate buffer; a histidine buffer (particularly with an aluminum hydroxide adjuvant); or a citrate buffer. Buffers, in some cases, are included in the 5-20 mM range.

In some cases, the pH of the vaccine is between about 5.0 and about 8.5, between about 6.0 and about 8.0, between about 6.5 and about 7.5, or between about 7.0 and about 7.8.

In some instances, a vaccine is sterile. In some cases, the vaccine is non-pyrogenic e.g. containing <1 EU (endotoxin unit, a standard measure) per dose, and can be <0.1 EU per dose.

In some instances, a vaccine includes detergent e.g. a polyoxyethylene sorbitan ester surfactant (known as ‘Tweens’), an octoxynol (such as octoxynol-9 (Triton X-100) or t-octylphenoxypolyethoxyethanol), a cetyl trimethyl ammonium bromide (‘CTAB’), or sodium deoxycholate, particularly for a split or surface antigen vaccine. The detergent can be present only at trace amounts. Thus, the vaccine can include less than 1 mg/ml of each of octoxynol-10 and polysorbate 80. Other residual components in trace amounts can be antibiotics (e.g. neomycin, kanamycin, polymyxin B).

In some instances, a vaccine is formulated as a sterile solution or suspension, in suitable vehicles, well known in the art. The pharmaceutical compositions can be sterilized by conventional, well-known sterilization techniques, or can be sterile filtered. The resulting aqueous solutions can be packaged for use as is, or lyophilized, the lyophilized preparation being combined with a sterile solution prior to administration. Suitable formulations and additional carriers are described in Remington “The Science and Practice of Pharmacy” (20^{th} Ed., Lippincott Williams & Wilkins, Baltimore Md.), the teachings of which are incorporated by reference in their entirety herein.

In some instances, a vaccine is formulated with one or more pharmaceutically acceptable salts. Pharmaceutically acceptable salts can include those of the inorganic ions, such as, for example, sodium, potassium, calcium, magnesium ions, and the like: Such salts can include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the agent(s) contain a carboxy group or other acidic group, it can be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine, triethanolamine, and the like.

Pharmaceutical compositions comprising an active, agent such as small molecule fragment and/or a cysteine-containing polypeptide-small molecule fragment adduct described herein, in combination with one or more adjuvants can be formulated to comprise certain molar ratios. For example, molar ratios of about 99:1 to about 1:99 of an active agent such as a peptide, a nucleic acid, an antibody or fragments thereof, and/or an APC described herein, in combination with one or more adjuvants can be used. In some instances, the range of molar ratios of an active agent such as a peptide, a nucleic acid, an antibody or fragments thereof, and/or an APC described herein, in combination with one or more adjuvants can be selected from about 80:20 to about 20:80; about 75:25 to about 25:75, about 70:30 to about 30:70, about 66:33 to about 33:66, about 60:40 to about 40:60; about 50:50, and about 90:10 to about 10:90. The molar ratio of an active agent such as a peptide, a nucleic acid, an antibody or fragments thereof, and/or an APC described herein, in combination with one or more adjuvants can be about 1:9, and in some cases can be about 1:1. The active agent such as a peptide, a nucleic acid, an antibody or fragments thereof, and/or an APC described herein, in combination with one or more adjuvants can be formulated together, in the same dosage unit e.g., in one vial, suppository, tablet, capsule, an aerosol spray; or each agent, form, and/or compound can be formulated in separate units, e.g., two vials, suppositories, tablets, two capsules, a tablet and a vial, an aerosol spray, and the like.

### Methods of Generating an Antibody

In some embodiments, a method of generating or raising an antibody or its binding fragment thereof comprises inoculating a mammal (e.g., a mouse, rat or rabbit) with a small molecule fragment composition described herein. In some instances, the small molecule fragment is a small molecule fragment of Formula (I). In some instances, the method further comprises harvesting and purifying an antibody against the small molecule fragment composition.

In some embodiments, a method of generating or raising an antibody or its binding fragment thereof comprises inoculating a mammal (e.g., a mouse, rat or rabbit) with a cysteine-containing polypeptide-small molecule fragment adduct described herein. In some instances, the cysteine-containing polypeptide-small molecule fragment adduct is a purified cysteine-containing polypeptide-small molecule fragment adduct. In some instances, the cysteine-containing polypeptide an isolated and purified polypeptide comprising at least 70%, 80%, 85%, 90%, 95%, 96%, 97%, 98%, 99% or more sequence identity to at least seven contiguous amino acids of an amino acid sequence selected from SEQs disclosed in the Examples. In some instances, the method further comprises harvesting and purifying an antibody against the cysteine-containing polypeptide-small molecule fragment adduct.

In some instances, a method of generating or raising an antibody or its binding fragment thereof comprises inoculating a mammal (e.g., a mouse, rat or rabbit) with a cultured cell expressing a cysteine-containing polypeptide and further administrating a small molecule fragment described herein to generate a cysteine-containing polypeptide-small molecule fragment adduct. In some instances, the cysteine-containing polypeptide is an isolated and purified polypeptide comprising at least 70%, 80%, 85%, 90%, 95%, 96%, 97%, 98%, 99% or more sequence identity to at least seven contiguous amino acids of an amino acid sequence selected from SEQs disclosed in the Examples. In some instances, the method further comprises harvesting and purifying an antibody against the cultured cell expressing a cysteine-containing polypeptide and further incubated with a small molecule fragment described herein.

In some instances, a method of generating or raising an antibody or its binding fragment thereof comprises inoculating a mammal (e.g., a mouse, rat or rabbit) with dendritic-cell derived exosomes. In some instances, a dendritic-cell derived exosome comprises an antigen (e.g., a cysteine-containing polypeptide-small molecule fragment adduct) which then incudes activation of the antigen-specific B-cell antibody response. In some cases, the dendritic-cell derived exosome comprises a cysteine-containing polypeptide-small molecule fragment antigen. In some cases, a method of generating or raising an antibody or its binding fragment thereof comprises inoculating a mammal (e.g., a mouse, rat or rabbit) with dendritic-cell derived exosomes comprising a cysteine-containing polypeptide-small molecule fragment antigen. In some instances, the method further comprises harvesting and purifying an antibody against the dendritic-cell derived exosomes.

### Vaccine Formulations

In some embodiments, a vaccine described herein, in combination with one or more adjuvants is formulated in conventional manner using one or more physiologically acceptable carriers, comprising excipients, diluents, and/or auxiliaries, e.g., which facilitate processing of the active agents into preparations that can be administered. Proper formulation depends at least in part upon the route of administration chosen. The agent(s) described herein can be delivered to a patient using a number of routes or modes of administration, including oral, buccal, topical, rectal, transdermal, transmucosal, subcutaneous, intravenous, and intramuscular applications, as well as by inhalation.

In some instances, the active agents are formulated for parenteral administration (e.g., by injection, for example bolus injection or continuous infusion) and can be presented. In unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol.

For injectable formulations, the vehicle can be chosen from those known in art to be suitable, including aqueous solutions or oil suspensions, or emulsions, with sesame oil, corn oil, cottonseed oil, or peanut oil, as risen as elixirs, mannitol, dextrose, or a sterile aqueous solution, and similar pharmaceutical vehicles. The formulation can also comprise polymer compositions which are biocompatible, biodegradable, such as poly(lactic-co-glycolic)acid. These materials can be made into micro or nanospheres loaded with drug and further coated or derivatized to provide superior sustained release performance. Vehicles suitable for periocular or intraocular injection include, for example, suspensions of therapeutic agent in injection grade water, liposomes and vehicles suitable for lipophilic substances. Other vehicles for periocular or intraocular injection are well known in the art.

When administration is by injection, the active agent is sometimes formulated in aqueous solutions, specifically in physiologically compatible buffers such as Hanks solution, Ringer's solution, or physiological saline buffer. The solution can contain formulatory agents such as suspending, stabilizing and/or dispersing agents. Alternatively, the active compound can be in powder form for constitution with a suitable vehicle, e.g., sterile pyrogen-free water, before use. In another embodiment, the pharmaceutical composition does not comprise an adjuvant or any other substance added to enhance the immune response stimulated by the peptide. In another embodiment, the pharmaceutical composition comprises a substance that inhibits an immune response to the peptide. Methods of formulation are known in the art, for example, as disclosed in Remington's Pharmaceutical Sciences, latest edition, Mack Publishing Co., Easton P.

For oral administration, the active agent is sometimes formulated readily by combining the active agent with pharmaceutically acceptable carriers well known in the art. Such carriers enable the agents of the disclosure to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. A solid carrier can be one or more substances which can also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Generally, the active agents can be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

In some instances, the vaccine is formulated into aerosol solutions, suspensions or dry powders. The aerosol can be administered through the respiratory system or nasal passages. For example, one skilled in the art will recognize that a composition of the present disclosure can be suspended or dissolved in an appropriate carrier, e.g., a pharmaceutically acceptable propellant, and administered directly into the lungs using a nasal spray or inhalant. For example, an aerosol formulation comprising a transporter, carrier, or ion channel inhibitor can be dissolved, suspended or emulsified in a propellant or a mixture of solvent and propellant, e.g., for administration as a nasal spray or inhalant. Aerosol formulations can contain any acceptable propellant under pressure, such as a cosmetically or dermatologically or pharmaceutically acceptable propellant, as conventionally used in the art.

An aerosol formulation for nasal administration is generally an aqueous solution designed to be administered to the nasal passages in drops or sprays. Nasal solutions can be similar to nasal secretions in that they are generally isotonic and slightly buffered to maintain a pH of about 5.5 to about 6.5, although pH values outside of this range can additionally be used. Antimicrobial agents or preservatives can also be included in the formulation.

In some instances, an aerosol formulation for inhalations and inhalants are designed so that the agent or combination of agents is carried into the respiratory tree of the subject when administered by the nasal or oral respiratory route. Inhalation solutions can be administered, for example, by a nebulizer. Inhalations or insufflations, comprising finely powdered or liquid drugs, can be delivered to the respiratory system as a pharmaceutical aerosol of a solution or suspension of the agent or combination of agents in a propellant, e.g., to aid in disbursement. Propellants can be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants can include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Halocarbon propellants are described in Johnson, U.S. Pat. No. 5,376,359, issued Dec. 27, 1994; Byron et al., U.S. Pat. No. 5,190,029, issued Mar. 2, 1993; and Purewal et al., U.S. Pat. No. 5,776,434, issued Jul. 7, 199S. Hydrocarbon propellants useful in the disclosure include, for example, propane, isobutane, α-butane, pentane, isopentane and neopentane. A blend of hydrocarbons can also be used as a propellant. Ether propellants include, for example, dimethyl ether as well as the ethers. An aerosol formulation in some instances also comprises more than one propellant. For example, the aerosol formulation can comprise more than one propellant from the same class, such as two or more fluorocarbons; or more than one, more than two, more than three propellants from different classes, such as a fluorohydrocarbon and a hydrocarbon. In some instances, vaccines are also dispensed with a compressed gas, e.g., an inert gas such as carbon dioxide, nitrous oxide or nitrogen.

Aerosol formulations can also include other components, for example, ethanol, isopropanol, propylene glycol, as well as surfactants or other components such as oils and detergents. These components can serve to stabilize the formulation and/or lubricate valve components.

In some instances, the aerosol formulation is packaged under pressure and is formulated as an aerosol using solutions, suspensions, emulsions, powders and semisolid preparations. For example, a solution aerosol formulation can comprise a solution of an agent of the disclosure such as a transporter, carrier, or ion channel inhibitor in (substantially) pure propellant or as a mixture of propellant and solvent. The solvent can be used to dissolve the agent and/or retard the evaporation of the propellant. Solvents can include, for example, water, ethanol and glycols. Any combination of suitable solvents can be use, optionally combined with preservatives, antioxidants, and/or other aerosol components.

In some instances, an aerosol formulation is a dispersion or suspension. A suspension aerosol formulation can comprise a suspension of an agent or combination of agents of the instant disclosure, e.g., a transporter, carrier, or ion channel inhibitor, and a dispersing agent. Dispersing/agents can include, for example, sorbitan trioleate, oleyl alcohol, oleic acid, lecithin and corn oil. A suspension aerosol formulation can also include lubricants, preservatives, antioxidant, and/or other aerosol components.

In some cases, an aerosol formulation is formulated as an emulsion. An emulsion aerosol formulation can include, for example, an alcohol such as ethanol, a surfactant, water and a propellant, as well as an agent or combination of agents of the disclosure, e.g., a transporter, carrier, or ion channel. The surfactant used can be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane,

### Vaccine Dosages, Routes of Administration and Therapeutic Regimens

In some instances, a vaccine is delivered via a variety of routes. Exemplary delivery routes include oral (including buccal and sub-lingual), rectal, nasal, topical, transdermal patch, pulmonary, vaginal, suppository, or parenteral (including intramuscular, intraarterial, intrathecal, intradermal, intraperitoneal, subcutaneous and intravenous) administration or in a form suitable for administration by aerosolization, inhalation or insufflation. General information on drug delivery systems can be found in Ansel et al., Pharmaceutical Dosage Forms and Drug Delivery Systems (Lippencott Williams & Wilkins, Baltimore Md. (1999). The vaccine described herein can be administered to muscle, or can be administered via intradermal or subcutaneous injections, or transdermally, such as by iontophoresis. Epidermal administration of the vaccine can be employed.

In some instances, the vaccine is formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

In some cases, the vaccine is a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

In some instances, the vaccine includes material for a single immunization, or may include material for multiple immunizations (i.e. a ‘multidose’ kit). The inclusion of a preservative is preferred in multidose arrangements. As an alternative (or in addition) to including a preservative in multidose compositions, the compositions can be contained in a container having an aseptic adaptor for removal of material.

In some instances, the vaccine is administered in a dosage volume of about 0.5 mL, although a half dose (i.e. about 0.25 mL) can be administered to children. Sometimes the vaccine can be administered in a higher dose e.g. about 1 ml.

In some instances, the vaccine is administered as a 1, 2, 3, 4, 5, or more dose-course regimen. Sometimes, the vaccine is administered as a 2, 3, or 4 dose-course regimen. Sometimes the vaccine is administered as a 2 dose-course regimen.

In some instances, the administration of the first dose and second dose of the 2 dose-course regimen are separated by about 0 day, 1 day, 2 days, 5 days, 7 days, 14 days, 21 days, 30 days, 2 months, 4 months, 6 months, 9 months, 1 year, 1.5 years, 2 years, 3 years, 4 years, or more.

In some instances, the vaccine described herein is administered every 1, 2, 3, 4, 5, 6, 7, 8, 9, 10, or more years. Sometimes, the vaccine described herein is administered every 2, 3, 4, 5, 6, 7, or more years. Sometimes, the vaccine described herein is administered every 4, 5, 6, 7, or more years. Sometimes, the vaccine described herein is administered once.

The dosage examples are not limiting and are only used to exemplify particular dosing regiments for administering a vaccine described herein. The effective amount for use in humans can be determined from animal models. For example, a dose for humans can be formulated to achieve circulating, liver, topical and/or gastrointestinal concentrations that have been found to be effective in animals. Based on animal data, and other types of similar data, those skilled in the art can determine the effective amounts of a vaccine composition appropriate for humans.

The effective amount when referring to an agent or combination of agents will generally mean the dose ranges, modes of administration, formulations, etc., that have been recommended or approved by any of the various regulatory or advisory organizations in the medical or pharmaceutical arts (e.g., FDA, AMA) or by the manufacturer or supplier.

In some instances, the vaccine is administered before, during, or after the onset of a symptom associated with a disease or condition (e.g., a cancer). Exemplary symptoms can include fever, cough, sore throat, runny and/or stuffy nose, headaches, chills, fatigue, nausea, vomiting, diarrhea, pain, or a combination thereof. In some instances, a vaccine is administered for treatment of a cancer. In some cases, a vaccine is administered for prevention, such as a prophylactic treatment of a cancer. In some cases, a vaccine is administered to illicit an immune response from a patient.

In some aspects, a vaccine and kit described herein are stored at between 2° C. and 8° C. In some instances, a vaccine is not stored frozen. In some instances, a vaccine is stored in temperatures of such as at −20° C. or −80° C. In some instances, a vaccine is stored away from sunlight.

### Pharmaceutical Compositions and Formulations

In some embodiments, disclosed herein include pharmaceutical composition and formulations comprising a small molecule fragment of Formula (I). In some instances, also described herein include pharmaceutical composition and formulations comprising a cysteine-containing polypeptide-small molecule fragment adduct. In other instances, the cysteine-containing polypeptide is an isolated and purified polypeptide comprising at least 70%, 80%, 85%, 90%, 95%, 96%, 97%, 98%, 99% or more sequence identity to at least seven contiguous amino acids of an amino acid sequence selected from SEQs disclosed in the Examples. In some embodiments, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular) administration. In other instances, the pharmaceutical composition describe herein is formulated for oral administration. In still other instances, the pharmaceutical composition describe herein is formulated for intranasal administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Therapeutic Regimens for a Pharmaceutical Composition

In some embodiments, a pharmaceutical composition described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include a small molecule fragment disclosed herein or an antibody that recognizes a cysteine-containing polypeptide-small molecule fragment adduct described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

“Antibodies” and “immunoglobulins” (Igs) are glycoproteins having the same structural characteristics. The terms are used synonymously. In some instances, the antigen specificity of the immunoglobulin is known.

The term “antibody” is used in the broadest sense and covers fully assembled antibodies, antibody fragments that can bind antigen (e.g., Fab, F(ab′)₂, Fv, single chain antibodies, diabodies, antibody chimeras, hybrid antibodies, bispecific antibodies, humanized antibodies, and the like), and recombinant peptides comprising the forgoing.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (Vu) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular toxicity, initiation of complement dependent cytotoxicity, and mast cell degranulation.

The term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Antibody fragments” comprise a portion of an intact antibody, preferably the antigen-binding or variable region of the intact antibody. Examples of antibody fragments include Fab, Fab, F(ab′)2, and Fv fragments; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H1}) of the heavy chain. Fab fragments differ from Fab′ fragments by the addition of a few residues at the carboxy terminus of the heavy chain C_{H1} domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. It is understood that the alkyl group is acyclic. In some instances, the alkyl group is branched or unbranched. In some instances, the alkyl group is also substituted or unsubstituted. For example, the alkyl group is substituted with one or more groups including, but not limited to, alkyl, cycloalkyl, alkoxy, amino, ether, halide, hydroxy, nitro, silyl, sulfo-oxo, or thiol. A “lower alkyl” group is an alkyl group containing from one to six (e.g., from one to four) carbon atoms. In some instances, the term alkyl group is also a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

The term “aryl” as used herein is a group that contains any carbon-based aromatic group including, but not limited to, benzene, naphthalene, phenyl, biphenyl, anthracene, and the like. The aryl group can be substituted or unsubstituted. In some instances, the aryl group is substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol. The term “biaryl” is a specific type of aryl group and is included in the definition of “aryl.” In addition, the aryl group is optionally a single ring structure or comprise multiple ring structures that are either fused ring structures or attached via one or more bridging groups such as a carbon-carbon bond. For example, biaryl refers to two aryl groups that are bound together via a fused ring structure, as in naphthalene, or are attached via one or more carbon-carbon bonds, as in biphenyl.

### EXAMPLES

These examples are provided for illustrative purposes only and not to limit the scope of the claims provided herein.

### Example 1

### General Synthetic Methods

Chemicals and reagents were purchased from a variety of vendors, including Sigma Aldrich, Acros, Fisher, Fluka, Santa Cruz, CombiBlocks, BioBlocks, and Matrix Scientific, and were used without further purification, unless noted otherwise. Anhydrous solvents were obtained as commercially available pre-dried, oxygen-free formulations. Flash chromatography was carried out using 230-400 mesh silica gel. Preparative thin layer chromotography (PTLC) was carried out using glass backed PTLC plates 500-2000 inn thickness (Analtech). All reactions were monitored by thin layer chromatography carried out on 0.25 mm E. Merck silica gel plates (60E-254) and visualized with UV light, or by ninhydrin, ethanolic phosphomolybdic acid, iodine, p-anisaldehyde or potassium permanganate stain. NMR spectra were recorded on Varian INOVA-400, Bruker DRX-600 or Bruker DRX-500 spectrometers in the indicated solvent. Multiplicities are reported with the following abbreviations: s singlet; d doublet; t triplet; q quartet; p pentet; m multiplet; br broad. Chemical shifts were reported in ppm relative to TMS and J values were reported in Hz. Mass spectrometry data were collected on a HP1100 single-quadrupole instrument (ESI; low resolution) or an Agilent ESI-TOF instrument (HRMS).

In some embodiments, General Procedure A was used for the synthesis of one or more of the small molecule fragments and/or cysteine-reactive probes described herein. The amine was dissolved in anhydrous CH₂Cl₂ (0.2 M) and cooled to 0° C. To this, anhydrous pyridine (1.5 equiv.) was added in one portion, then chloroacetyl chloride (1.5 equiv.) dropwise and the reaction was monitored by TLC until complete disappearance of starting material and conversion to product was detected (typically 1 h). If the reaction did not proceed to completion, additional aliquots of pyridine (0.5 equiv.) and chloroacetyl chloride (0.5 equiv.) were added. The reaction was quenched with H₂O (1 mL), diluted with CH₂Cl₂ (20 mL), and washed twice with saturated NaHCO₃ (100 mL). The organic layer was concentrated in vacuo and purified by preparatory thin layer or flash column chromatography to afford the desired product. In some embodiments, General Procedure A1 is similar to General Procedure A except triethylamine (3 equiv.) was used instead of pyridine. In some embodiments, General Procedure A2 is similar to General Procedure A except N-methylmorpholine (3 equiv.) was used instead of pyridine.

In some embodiments, General Procedure B was used for the synthesis of one or more of the small molecule fragments and/or cysteine-reactive probes described herein. The amine was dissolved in anhydrous CH₂Cl₂ (0.2 M) and cooled to 0° C. To this, triethylamine (TEA, 1.5 equiv.), was added in one portion, then acryloyl chloride (1.5 equiv.) dropwise, and the reaction was monitored by TLC until complete disappearance of starting material and conversion to product was detected (typically 1 h). If the reaction did not proceed to completion, additional aliquots of TEA (0.5 equiv.) and acryloyl chloride (0.5 equiv.) were added. The reaction was quenched with H₂O (1 mL), diluted with CH₂Cl₂ (20 mL), and washed twice with saturated NaHCO₃ (100 mL). The organic layer was passed through a plug of silica, after which, the eluant was concentrated in vacuo and purified by preparatory thin layer or flash column chromatography to afford the desired product.

In some embodiments, General Procedure C was used for the synthesis of one or more of the small molecule fragments and/or cysteine-reactive probes described herein. Acryloyl chloride (80.4 μL, 1.0 mmol, 2 equiv.) was dissolved in anhydrous CH₂Cl₂ (4 mL) and cooled to 0° C. A solution of the amine (0.5 mmol, 1 equiv.) and N-methylmorpholine (0.16 mL, 1.5 mmol, 3 equiv.) in CH₂Cl₂ (2 mL) was then added dropwise. The reaction was stirred for 1 hr at 0° C. then allowed to warm up to room temperature slowly. After TLC analysis showed disappearance of starting material, or 6 h, whichever was sooner, the reaction was quenched with saturated aqueous NaHCO₃ (5 mL) and extracted with CH₂Cl₂ (3×10 mL). The combined organic layers were dried over anhydrous Na₂SO₄, concentrated in vacuo, and the residue obtained was purified by preparatory thin layer chromatography to afford the desired product.

### Synthesis of Probes and Fragments

### Purchased Fragments

The following electrophilic fragments were purchased from the indicated vendors. 2 (Santa Cruz Biotechnology sc-345083), 3 (Key Organics JS-092C), 4 (Sigma Aldrich T142433-10 mg), 6 (Toronto Research Chemicals M320600), 8 (Alfa Aesar H33763), 10 (Santa Cruz Biotechnology sc-345060), 11 (Santa Cruz Biotechnology sc-354895), 12 (Santa Cruz Biotechnology sc-354966), 21 (Santa Cruz Biotechnology, sc-279681), 22 (Sigma Aldrich 699357-5G), 26 (Sigma Aldrich T109959), 27 (Santa Cruz Biotechnology sc-342184), 28 (Santa Cruz Biotechnology sc-335173), 29 (Santa Cruz Biotechnology sc-348978), 30 (Santa Cruz Biotechnology sc-355362), 32 (Santa Cruz Biotechnology sc-354613), 33 (Sigma Aldrich R996505), 34 (Santa Cruz Biotechnology sc-355477), 35 (Santa Cruz Biotechnology sc-328985), 41 (Sigma Aldrich L469769), 42 (Sigma Aldrich R901946), 43 (Santa Cruz Biotechnology sc-307626), 52 (Enamine, EN300-08075), 55 (Santa Cruz Biotechnology sc-354880), 57 (VWR 100268-442), 58 (Enzo Life Sciences ALX-430-142-M005), 62 (WuXi Apptec).

### Synthesis of Isotopically-Labeled TEV-Tags:

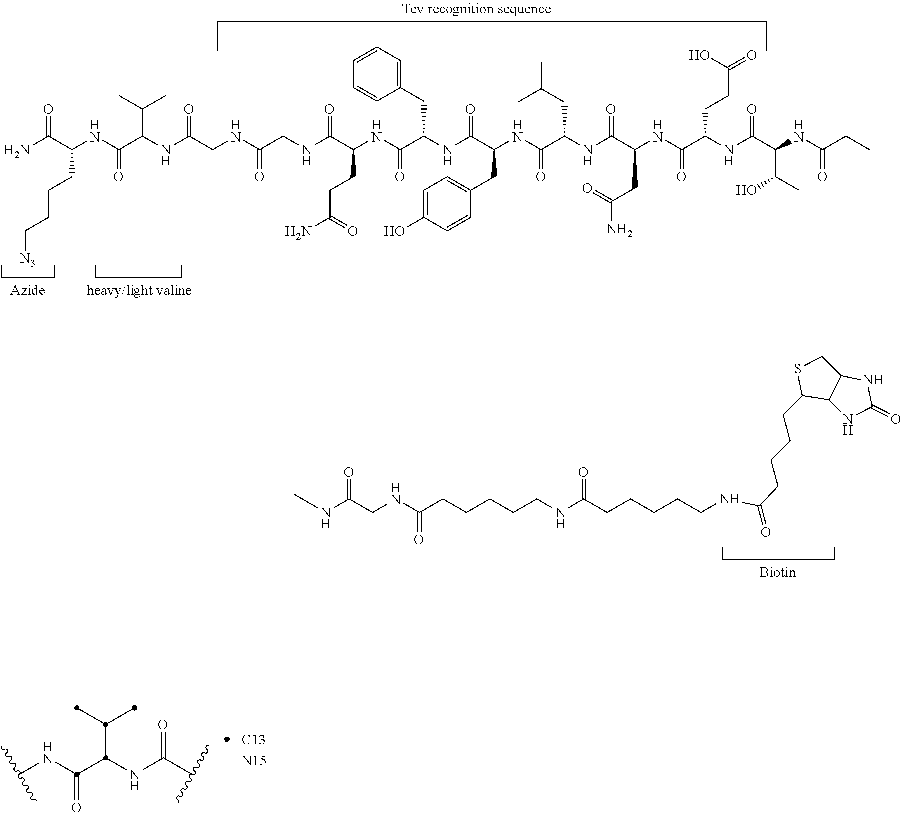

Isotopically-labeled heavy and light tags were synthesized with minor modifications to the procedure reported in Weerapana et al. Nat Protoc 2:1414-1425 (2007) and Weerapana et al. Nature 468:790-795 (2010). Fmoc-Rink-Amide-MBHA resin (EMD Biosciences; 0.5 M, 830 mg, 0.6 mmol/g loading) was deprotected with 4-methylpiperidine in DMF (50% v/v, 2×5 mL, 1 min). Fmoc-Lys(N₃)-OH (Anaspec) (500 mg, 1.26 mmol, 1.26 equiv.) was coupled to the resin overnight at room temperature with DIEA (113 μl) and 2-(6-chloro-1H-benzo tri azole-1-yl)-1,1,3,3-tetramethylaminium hexafluorophosphate (HCTU; 1.3 mL of 0.5 M stock in DMF) followed by a second overnight coupling with Fmoc-Lys(N₃)-OH (500 mg, 1.26 mmol, 1.26 equiv.), DIEA (113 μl), O-(7-azabenzotriazol-1-yl)-N,N,N′,N′-tetramethyluronium hexafluorophosphate (HATU; 1.3 mL of 0.5 M stock in DMF). Unmodified resin was then capped (2×30 min) with Ac₂O (400 μL) and DIEA (700 μl) in DMF after which the resin was washed with DMF (2×1 min). Deprotection with 4-methylpiperidine in DMF (50% v/v, 2×5 mL, 1 min) and coupling cycles (4 equiv. Fmoc-protected amino acid (EMD biosciences) in DMF) with HCTU (2 mL, 0.5 M in DMF) and DIEA (347.7 μL) were then repeated for the remaining amino acids. For the heavy TEV-tag, Fmoc-Valine-OH (¹³C₅C₁₅H₂₁¹⁵NO₄, ¹³C₅, 97-99%, ¹⁵N, 97-99%, Cambridge Isotope Laboratories, Inc.) was used. Reactions were monitored by ninhydrin stain and dual couplings were used for all steps that did not go to completion. Biotin (0.24 g, 2 equiv.) was coupled for two days at room temperature with NHS (0.1 g, 2 equiv.), DIC (0.16 g, 2 equiv.) and DIEA (0.175 g, 2 equiv.). The resin was then washed with DMF (5 mL, 2×1 min) followed by 1:1 CH₂Cl₂:MeOH (5 mL, 2×1 min), dried under a stream of nitrogen and transferred to a round-bottom flask. The peptides were cleaved for 90 minutes from the resin by treatment with 95:2.5:2.5 trifluoroacetic acid: water:triisopropylsilane. The resin was removed by filtration and the remaining solution was triturated with cold ether to provide either the light or heavy TEV-tag as a white solid. HPLC-MS revealed only minor impurities and the compounds were used without further purification. HRMS-ESI (m/z): calculated for C₈₃H₁₂₈N₂₃O₂₃S [M+H]: (Light-TEV-Tag) 1846.9268; found: 1846.9187; calculated for C₂₈¹³C₅H₁₂₈N₂₂¹⁵NO₂₃S [M+H]: (Heavy-TEV-Tag): 1852.9237; found: 1852.9309.

### Synthesis of Probes and Fragments

### Synthesis of 1

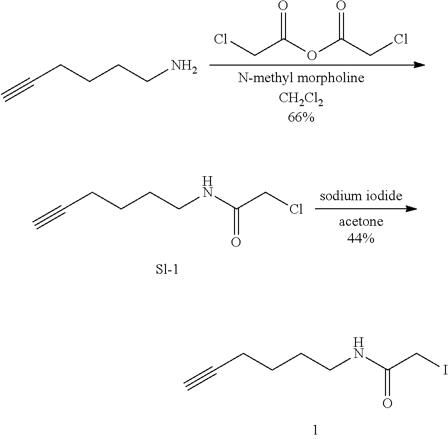

### N-(hex-5-yn-1-yl)-2-chloroacetamide (SI-1)

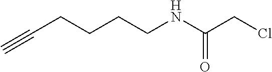

To a solution of 5-hexynylamine (63 mg, 0.65 mmol, 1.0 equiv.) in CH₂Cl₂ (3.2 mL, 0.2 M) at 0° C. was added N-methylmorpholine (2154, 3 equiv.) followed by chloroacetic anhydride portionwise (222 mg, 2 equiv.). The reaction was allowed to come to room temperature and then stirred overnight. The reaction was then diluted with ether (50 mL), washed with 1 M HCl, 1 M NaOH, then brine (20 mL each). The combined organic layers were dried over magnesium sulfate and concentrated to yield chloroacetamide SI-1 (74 mg, 66%). ¹H NMR (400 MHz, Chloroform-d) δ 6.79 (s, 1H), 4.09 (d, J=1.1 Hz, 2H), 3.34 (q, J=6.8 Hz, 2H), 2.23 (td, J=6.9, 2.7 Hz, 2H), 1.98 (t, J=2.7 Hz, 1H), 1.75-1.62 (m, 4H), 1.62-1.51 (m, 2H).

### N-(hex-5-yn-1-yl)-2-iodoacetamide (1)

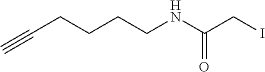

To a solution of chloroacetamide SI-1 (36.1 mg, 0.2 mmol) in acetone (1 mL, 0.2 M) was added sodium iodide (47 mg, 1.5 equiv.) and the reaction was stirred overnight. The next day the reaction was filtered through a plug of silica eluting with 20% ethyl acetate in hexanes, and the filtrate was concentrated to yield a 10:1 mixture of the desired iodoacetamide 1 and starting material. This mixture was re-subjected to the reaction conditions for one further day, at which point complete conversion was observed. The product was purified by silica gel chromatography, utilizing a gradient of 5 to 10 to 15 to 20% ethyl acetate in hexanes to yield the desired product (24 mg, 44%). In some embodiments, the reaction is performed with 2.5 equiv. of sodium iodide, in which case re-subjection is not necessary, and purification by PTLC is accomplished in 30% EtOAc/hexanes as eluent. ¹H NMR (500 MHz, Chloroform-d) δ 6.16 (s, 1H), 3.69 (s, 2H), 3.30 (q, J=6.8 Hz, 2H), 2.23 (td, J=6.8, 2.6 Hz, 2H), 1.97 (t, J=2.6 Hz, 1H), 1.75-1.61 (m, 2H), 1.61-1.52 (m, 2H).

### N-(4-bromophenyl)-N-phenylacrylamide (5)

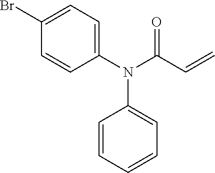 [SMILES: C=CC(=O)N(c1ccccc1)c1ccc(Br)cc1]

The title compound was synthesized according to General Procedure C from 4-bromophenylaniline (18.9 mg, 0.0762 mmol, 1 equiv.). Purification of the crude product by prep. TLC (30% EtOAc/hexanes) provided the title compound as a white solid (12.5 mg, 54%). ¹H NMR (500 MHz, Chloroform-d) δ 7.47 (d, J=8.2 Hz, 2H), 7.39 (t, J=7.6 Hz, 2H), 7.32 (d, J=7.4 Hz, 1H), 7.21 (d, J=7.7 Hz, 2H), 7.12 (d, J=8.2 Hz, 2H), 6.48 (d, J=16.7 Hz, 1H), 6.17 (dd, J=16.8, 10.3 Hz, 1H), 5.65 (d, J=10.3 Hz, 1H); HRMS-ESI (m/z) calculated for C₁₅H₁₃BrNO [M+H]: 302.0175; found: 302.0176.

### Synthesis of 7

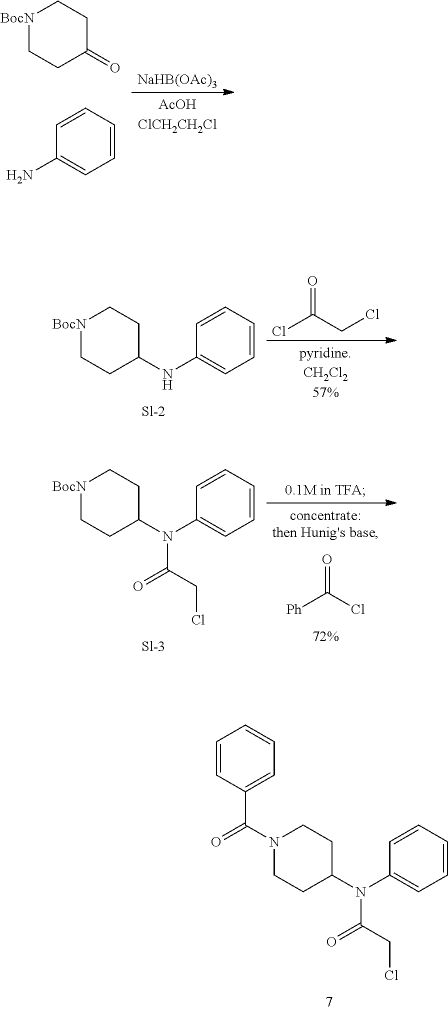

### tert-butyl 4-(phenylamino)piperidine-1-carboxylate (SI-2)

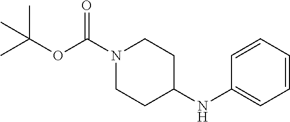 [SMILES: CC(C)(C)OC(=O)N1CCC(Nc2ccccc2)CC1]

SI-2 was prepared according to Thoma et al, J. Med. Chem. 47:1939-1955 (2004). ¹H NMR (400 MHz, Chloroform-d) δ 7.24-7.12 (m, 2H), 6.75-6.68 (m, 1H), 6.66-6.58 (m, 2H), 3.88-3.81 (m, 1H), 3.44 (tt, J=10.4, 3.9 Hz, 2H), 3.00-2.88 (m, 2H), 2.10-1.99 (m, 2H), 1.48 (bs 9H), 1.41-1.27 (m, 2H).

### tert-butyl 4-(2-chloro-N-phenylacetamido)piperidine-1-carboxylate (SI-3)

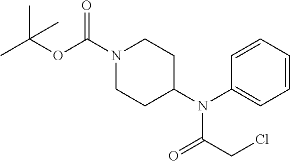 [SMILES: CC(C)(C)OC(=O)N1CCC(N(C(=O)CCl)c2ccccc2)CC1]

To a solution of aniline SI-2 (65 mg, 0.24 mmol) at 0° C. in CH₂Cl₂ (0.6 mL) was added pyridine (38 μL, 2 equiv.) followed by chloroacetyl chloride (37.4 μL, 2.0 equiv.) in CH₂Cl₂ (0.6 mL). The resulting solution was allowed to warm to room temperature and stirred overnight. The solution was then quenched with saturated aqueous sodium bicarbonate, extracted with Et₂O (3×10 mL). The combined organic layers were dried over magnesium sulfate, filtered and concentrated to give an off-white solid, which was used without further purification (47 mg, 57%). ¹H NMR (400 MHz, Chloroform-d) δ 7.47-7.38 (m, 3H), 7.18-7.03 (m, 2H), 4.75-4.63 (m, 1H), 4.07 (s, 2H), 3.68 (s, 2H), 2.76 (s, 2H), 1.84-1.69 (m, 2H), 1.35 (s, 9H), 1.27-1.12 (m, 2H).

### N-(1-benzoylpiperidin-4-yl)-2-chloro-N-phenylacetamide (7)

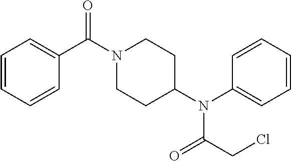 [SMILES: O=C(c1ccccc1)N1CCC(N(C(=O)CCl)c2ccccc2)CC1]

To neat SI-3 (47 mg, 0.128 mmol) was added trifluoroacetic acid (0.7 mL, final 0.2 M). The resulting solution was concentrated under a stream of nitrogen until no further evaporation was observed, providing the deprotected amine as its trifluoroacetate salt. This viscous gum was then treated with triethylamine in ethyl acetate (10% v/v, 2 mL; solution smokes upon addition). The resulting solution was concentrated to afford the free base, which contained only triethylammonium trifluoroacetate and the free amine by proton NMR. A stock solution was prepared by dissolving the resulting gum in CH₂Cl₂ (1.2 mL, ˜0.1 M final).

The deprotected amine (0.3 mL of stock solution, 0.0319 mmol) was treated with Hunig's base (17.5 μL, 3 equiv.) and benzoyl chloride (7.6 μL, 2.0 equiv.). This solution was stirred overnight, quenched with saturated aqueous sodium bicarbonate, extracted with Et₂O (3×10 mL). The resulting solution was dried over magnesium sulfate, filtered and concentrated. The resulting oil was purified by silica gel chromatography (20% EtOAc/hexanes) to afford chloroacetamide 7 as a white solid (8.6 mg, 75%). ¹H NMR (500 MHz, Chloroform-d) δ 7.55 (dd, J=5.5, 3.0 Hz, 3H), 7.50-7.32 (m, 5H), 7.21 (s, 2H), 4.92 (tt, J=12.3, 4.0 Hz, 1H), 4.87 (s, 1H), 3.87 (s, 1H), 3.78 (s, 2H), 3.21 (s, 1H), 2.97-2.90 (m, 1H), 2.01 (s, 1H), 1.90 (s, 1H), 1.45 (s, 1H), 1.36-1.26 (m, 1H); HRMS-ESI (m/z) calculated for C₂₀H₂₂ClN₂O₂ [M+H]: 357.1364; found: 357.1362.

### 1-(4-benzylpiperidin-1-yl)-2-chloroethan-1-one (9)

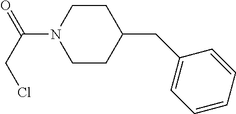 [SMILES: O=C(CCl)N1CCC(Cc2ccccc2)CC1]

Following General Procedure A, starting from 4-benzylpiperidine (840 mg, 5.2 mmol, 1 equiv.), the desired compound was obtained after column chromatography as a yellow oil (1 g, 81%). Spectroscopic data matches those reported previously reported in Papadopoulou et al. J Med. Chem. 55:5554-5565 (2012). ¹H NMR (500 MHz, Chloroform-d) δ 7.42-7.14 (m, 5H), 4.61 (d, J=13.4 Hz, 1H), 4.14 (q, J=21.9, 11.5 Hz, 2H), 3.89 (d, J=13.5, 1H), 3.11 (td, J=13.1, 2.7 Hz, 1H), 2.69-2.57 (m, 3H), 1.92-1.75 (m, 3H), 1.40-1.21 (m, 2H); HRMS-ESI (m/z) calculated for C₁₄H₁₉ClNO [M+H]: 252.115; found: 252.115.

### N-(2-(1H-indol-3-yl)ethyl)-2-chloroacetamide (13)

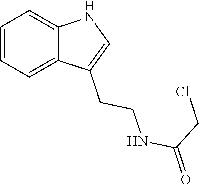 [SMILES: O=C(CCl)NCCc1c[nH]c2ccccc12]

Following General Procedure A, starting from tryptamine (400 mg, 2.5 mmol, 1 equiv.), the desired compound was obtained after column chromatography as a brownish solid (460 mg, 77%). ¹H NMR (500 MHz, Chloroform-d) δ 8.55 (s, 1H), 7.70 (d, J=7.9 Hz, 1H), 7.45 (d, J=8.1 Hz, 1H), 7.30 (t, J=7.5 Hz, 1H), 7.23 (t, J=7.4 Hz, 1H), 7.10 (s, 1H), 6.84 (s, 1H), 4.08 (s, 2H), 3.72 (q, J=6.4 Hz, 2H), 3.10 (t, J=6.8 Hz, 2H); HRMS-ESI (m/z) calculated for C₁₂H₁₄ClN₂O₂ [M+H]: 237.0789; found: 237.0791.

### N-(3,5-bis(trifluoromethyl)phenyl)acrylamide (14)

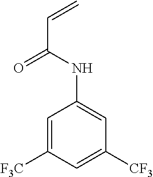 [SMILES: C=CC(=O)Nc1cc(C(F)(F)F)cc(C(F)(F)F)c1]

Following General Procedure B, starting from 3,5-bis(trifluoromethyl)aniline (1.16 g, 5 mmol, 1 equiv.), the desired compound was obtained after column chromatography as a white solid (1.05 g, 74%). ¹H NMR (500 MHz, Chloroform-d) δ 8.33 (s, 1H), 8.18 (s, 2H), 7.68 (s, 1H), 6.57 (d, J=17.5 Hz, 1H), 6.38 (dd, J=16.9, 10.3 Hz, 1H), 5.93 (d, J=12.5 Hz, 1H); HRMS-ESI (m/z) calculated for C₁₁H₈F₆NO₂ [M+H]: 284.0505; found: 284.0504.

### N-(4-phenoxy-3-(trifluoromethyl)phenyl)-N-(pyridin-3-ylmethyl)acrylamide (15)

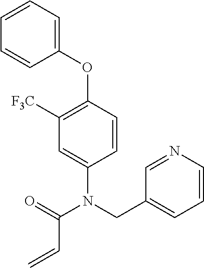 [SMILES: C=CC(=O)N(Cc1cccnc1)c1ccc(Oc2ccccc2)c(C(F)(F)F)c1]

4-phenoxy-3-(trifluoromethyl)aniline (260 mg, 1 mmol, 1 equiv.) (Combi-Blocks) was dissolved in TFA (5 mL). Following the reductive amination protocol reported by Boros et al. J. Org. Chem 74:3587-3590 (2009), the reaction mixture was cooled to 0° C. and to this sodium triacetoxyborohydride (STAB) (270 mg, 1.3 mmol, 1.3 equiv.) was added. 3-pyridinecarboxaldehyde (200 mg, 2 mmol, 2 equiv.) was dissolved in CH₂Cl₂ (5 mL) and slowly added to the reaction mixture. Upon complete conversion to product, the reaction was diluted with CH₂Cl₂ (20 mL) and washed with saturated sodium bicarbonate solution (3×20 mL) and the organic layer was dried then concentrated under reduced pressure. Without further purification the crude material was dissolved in anhydrous CH₂Cl₂ and subjected to General Procedure B. The resulting crude was purified by prep. TLC to give a white solid (31 mg, 10%). ¹H NMR (500 MHz, Chloroform-d) δ 8.52 (d, J=3.5 Hz, 1H), 8.39 (s, 1H), 7.68 (d, J=7.8 Hz, 1H), 7.40 (t, J=7.7 Hz, 2H), 7.34 (s, 1H), 7.28-7.18 (m, 2H), 7.07 (d, J=8.2 Hz, 2H), 6.98 (d, J=7.5 Hz, 1H), 6.82 (d, J=8.8 Hz, 1H), 6.46 (d, J=16.8 Hz, 1H), 6.01 (dd, J=16.2, 10.7 Hz, 1H), 5.64 (d, J=10.3 Hz, 1H), 4.96 (s, 2H). HRMS-ESI (m/z) calculated for C₂₂H₁₈F₃N₂O₂ [M+H]: 399.1315; found: 399.1315.

### Iodoacetamide-rhodamine (16)

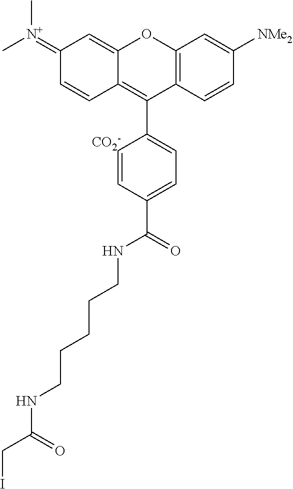

5-(and-6)-((N-(5-aminopentyl)amino)carbonyl)tetramethylrhodamine (tetramethylrhodamine cadaverine) mixed isomers (60 mg, 0.12 mmol, 1 equiv.) were dissolved in anhydrous DMF (500 μL) with sonication. To this was added DIPEA (60 μL, 0.34 mmol, 3 equiv.) and chloroacetyl chloride (10 μL, 0.13 mmol, 1 equiv., diluted 1:10 in DMF) and the reaction was stirred at room temperature for 20 min until complete conversion to the product was detected by TLC. The DMF was removed under a stream of nitrogen and the reaction mixture was separated by PTLC in MeOH:CH₂Cl₂:TEA (15:85:0.001). The chloroacetamide rhodamine was then eluted in MeOH:CH₂Cl₂ (15:85), concentrated under reduced pressure and redissolved in acetone (500 μL). NaI (150 mg, 1 mmol, 10 equiv.) was added to this and the reaction was stirred for 20 min at 50° C. until complete conversion to product was detected and the crude reaction mixture was purified by reverse phase HPLC on a C18 column and concentrated to yield the title compound as a purple solid that is a mixture of 5 and 6 carboxamide tetramethylrhodamine isomers (ratio ˜6:1) (10 mg, 12%). ¹H NMR (600 MHz, Methanol-d₄) δ 8.87 (t, J=4.8 Hz, 0.14H), 8.80-8.71 (m, 1H), 8.41 (dd, J=8.2, 1.1 Hz, 0.86H), 8.35 (br s, 1H), 8.27 (dt, J=7.9, 1.5 Hz, 0.164H), 8.20 (dt, J=8.2, 1.5 Hz, 0.86H), 7.81 (s, 0.86H), 7.53 (d, J=7.8 Hz, 0.14H), 7.18-7.11 (m, 2H), 7.07 (d, J=9.5 Hz, 2H), 7.00 (s, 2H), 3.68-3.62 (m, 2H), 3.46-3.37 (m, 2H), 3.31 (s, 12H, obscured by solvent) 3.21-3.12 (m, 2H), 1.81-1.21 (m, 6H); HRMS-ESI (m/z) calculated for C₃₂H₃₆IN₄O₅ [M+H]: 683.1725; found: 683.1716.

### N-(3,5-bis(trifluoromethyl)phenyl)acetamide (17)

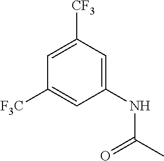 [SMILES: CC(=O)Nc1cc(C(F)(F)F)cc(C(F)(F)F)c1]

Following General Procedure A, starting with 3,5-bis(trifluoromethyl)aniline (327 mg, 1.42 mmol, 1 equiv.) and acetic anhydride (200 μL, 3 mmol, 2 equiv.), the title compound was obtained after PTLC as a white solid (302 mg, 78%). ¹H NMR (500 MHz, Chloroform-d) δ 8.10 (s, 2H), 7.72 (s, 1H), 7.68 (s, 1H), 2.32 (d, J=0.9 Hz, 3H). HRMS-ESI (m/z) calculated for C₁₁H₈F₆NO₂ [M+H]: 284.0505; found: 284.0504.

### Synthesis of 18 and 19

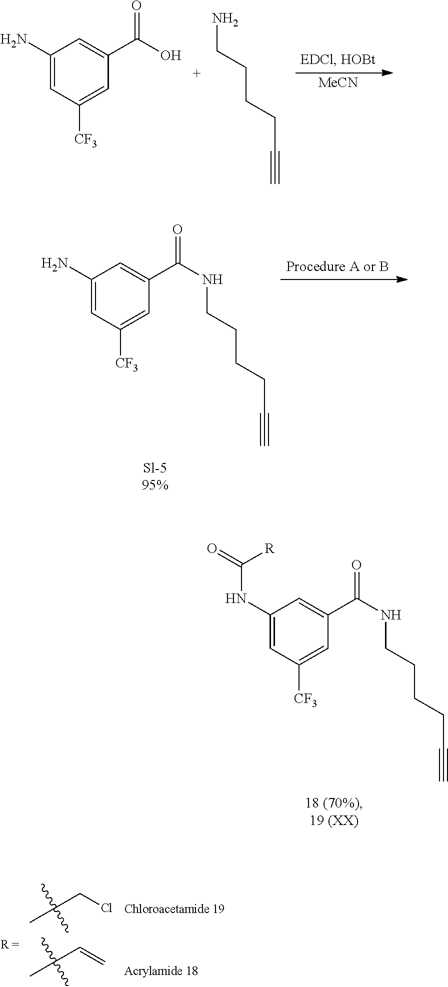

### 3-amino-N-(hex-5-yn-1-yl)-5-(trifluoromethyl)benzamide (SI-5)

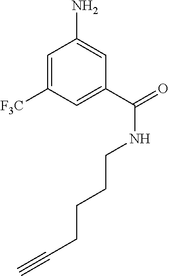 [SMILES: C#CCCCCNC(=O)c1cc(N)cc(C(F)(F)F)c1]

To a solution of 3-amino-5-(trifluoromethyl)benzoic acid (74 mg, 0.36 mmol) in acetonitrile (3.6 mL, 0.1 M final) was added EDCI (83 mg, 1.2 equiv.) followed by hex-5-ynamine (35 mg, 1.0 equiv.) followed by 1-hydroxybenzotriazole hydrate (HOBt, 66.3 mg, 1.2 equiv.) and the resulting solution was stirred overnight. The reaction was diluted with ethyl acetate, washed with 1 M HCl twice and then brine. The organic layer was dried over magnesium sulfate and concentrated to yield aniline SI-5 (97.4 mg, 95%) as a white solid. NMR (400 MHz, Chloroform-d) δ 7.29-7.22 (m, 2H), 6.98 (t, J=1.8 Hz, 1H), 6.38 (t, J=5.5 Hz, 1H), 4.08 (s, 2H), 3.46 (td, J=7.1, 5.7 Hz, 2H), 2.25 (td, J=6.9, 2.6 Hz, 2H), 1.99 (t, J=2.7 Hz, 1H), 1.81-1.55 (m, 4H).

### 3-acrylamido-N-(hex-5-yn-1-yl)-5-(trifluoromethyl)benzamide (18)

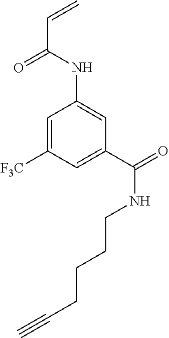 [SMILES: C#CCCCCNC(=O)c1cc(NC(=O)C=C)cc(C(F)(F)F)c1]

Following General Procedure B, starting with SI-5 (42 mg, 0.15 mmol, 1 equiv.), the title compound was obtained after column chromatography as a white solid (34 mg, 70%). ¹H NMR (500 MHz, Chloroform-d) δ 8.94 (s, 1H), 8.24 (d, J=11.9 Hz, 2H), 7.71 (s, 1H), 6.87 (t, J=5.7 Hz, 1H), 6.55 (dd, J=17.4, 0.7 Hz, 1H), 6.43 (dd, J=16.9, 10.1 Hz, 1H), 5.88 (dd, J=10.1, 1.3 Hz, 1H), 3.56 (q, J=6.7 Hz, 2H), 2.33 (td, J=6.9, 2.7 Hz, 2H), 2.06 (t, J=2.7 Hz, 1H), 1.87 (p, J=7.3 Hz, 2H), 1.69 (p, J=7.8 Hz, 2H); HRMS-ESI (m/z) calculated for C₁₇H₁₈F₃N₂O₂ [M+H]: 339.1314; found 339.1313.

### 3-acrylamido-N-(hex-5-yn-1-yl)-5-(trifluoromethyl)benzamide (19)

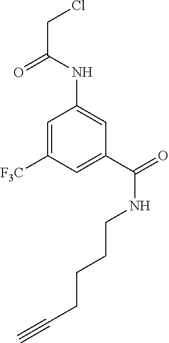 [SMILES: C#CCCCCNC(=O)c1cc(NC(=O)CCl)cc(C(F)(F)F)c1]

Synthesized according to General Procedure A2, starting from SI-5. ¹H NMR (600 MHz, Chloroform-d) δ 8.57 (s, 1H), 8.16 (t, J=1.8 Hz, 1H), 8.05 (t, J=1.8 Hz, 1H), 7.79 (d, J=2.0 Hz, 1H), 6.38 (d, J=6.1 Hz, 1H), 4.23 (s, 2H), 3.51 (td, J=7.1, 5.7 Hz, 2H), 2.27 (td, J=6.9, 2.7 Hz, 2H), 2.00 (t, J=2.6 Hz, 1H), 1.82-1.74 (m, 2H), 1.71-1.59 (m, 2H); HRMS-ESI (m/z) calculated for C₁₆H₁₇ClF₃N₂O₂ [M+H]: 361.0925; found: 361.0925.

### 2-chloro-1-(4-(hydroxydiphenylmethyl)piperidin-1-yl)ethan-1-one (20)

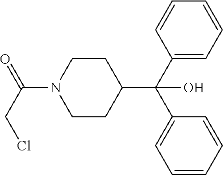 [SMILES: O=C(CCl)N1CCC(C(O)(c2ccccc2)c2ccccc2)CC1]

Following General Procedure A, starting with α,α-diphenyl-4-piperidinomethanol (800 mg, 3 mmol, 1 equiv.), the title compound was obtained after column chromatography as a white solid (637 mg, 61%). ¹H NMR (500 MHz, Chloroform-d) δ 7.56 (d, J=7.6 Hz, 4H), 7.39 (q, J=7.1 Hz, 4H), 7.28 (q, J=6.8 Hz, 2H), 4.66 (d, J=13.3 Hz, 1H), 4.07 (dd, J=12.2, 4.2 Hz, 2H), 3.91 (d, J=13.4 Hz, 1H), 3.18 (t, J=12.9 Hz, 1H), 2.77-2.62 (m, 3H), 1.67 (t, J=12.5 Hz, 2H), 1.56 (q, J=11.8 Hz, 1H), 1.44 (q, J=12.4, 11.8 Hz, 1H); HRMS-ESI (m/z) calculated for C₂₀H₂₃ClNO₂ [M+H]: 344.1412; found: 344.1412.

### (E)-3-(3,5-bis(trifluoromethyl)phenyl)-2-cyanoacrylamide (23)

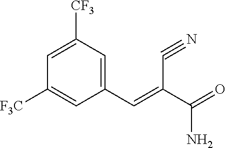 [SMILES: N#C/C(=C\c1cc(C(F)(F)F)cc(C(F)(F)F)c1)C(N)=O]

3,5-bis(trifluoromethyl)benzaldehyde (880 mg, 3.6 mmol, 1 equiv.) and 2-cyanoacetamide (460 mg, 5.5 mmol, 1.5 equiv.) were dissolved in MeOH (10 mL). To this was added piperidine (214 mg, 0.7 equiv.) and the reaction was stirred at room temperature for 30 minutes at which point starting material was consumed. After addition of an equivalent volume of water (10 mL), the precipitate was collected by filtration and washed with water/methanol (1:1) to yield the title compound as a white solid (534 mg, 47%); ¹H NMR (400 MHz, Acetone-d₆) δ 8.78 (s, 2H), 8.61 (s, 1H), 8.41 (s, 1H), 7.57 (s, 1H), 7.42 (s, 1H); HRMS-ESI (m/z) calculated for C₁₂H₇F₆N₂O₂ [M+H]: 309.0457; found: 309.0459.

### N-(3,5-bis(trifluoromethyl)phenyl)-2-bromopropanamide (24)

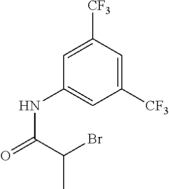 [SMILES: CC(Br)C(=O)Nc1cc(C(F)(F)F)cc(C(F)(F)F)c1]

Following General Procedure A1, starting with 3,5-bis(trifluoromethyl)aniline (250 mg, 1.1 mmol, 1 equiv.) and 2-bromopropionyl chloride (200 μL, 2 mmol, 1.8 equiv.) the title compound was obtained by PTLC as a white solid (130 mg, 35%). ¹H NMR (500 MHz, Chloroform-d) δ 8.34 (s, 1H), 8.06 (s, 2H), 7.66 (s, 1H), 4.58 (q, J=7.0 Hz, 1H), 1.98 (d, J=7.0 Hz, 3H); HRMS-ESI (m/z) calculated for C₁₁H₇BrF₆NO [M−H]: 361.9621; found: 361.9623.

### N-(3,5-bis(trifluoromethyl)phenyl)-2-chloropropanamide (25)

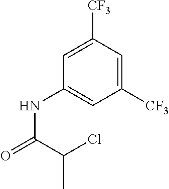 [SMILES: CC(Cl)C(=O)Nc1cc(C(F)(F)F)cc(C(F)(F)F)c1]

Following General Procedure A1, starting with 3,5-bis(trifluoromethyl)aniline (327 mg, 1.42 mmol, 1 equiv.) and 2-chloropropionyl chloride (200 μL, 2 mmol, 1.8 equiv.) the title compound was obtained by PTLC as a white solid (250 mg, 55%). ¹H NMR (500 MHz, Chloroform-d) δ 8.61 (s, 1H), 8.16 (s, 2H), 7.75 (s, 1H), 4.67 (q, J=7.1 Hz, 1H), 1.93 (d, J=7.1 Hz, 3H). HRMS-ESI (m/z) calculated for C₁₁H₇ClF₆NO [M−H]: 318.0126; found: 318.0126.

### N-(3,5-bis(trifluoromethyl)phenyl)-N-(pyridin-3-ylmethyl)acglamide (31)

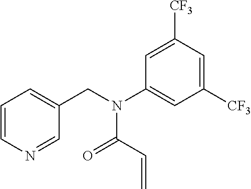 [SMILES: C=CC(=O)N(Cc1cccnc1)c1cc(C(F)(F)F)cc(C(F)(F)F)c1]

3,5-bis(trifluoromethyl)aniline (350 mg, 1.6 mmol, 1 equiv.) was dissolved in TFA (5 mL). The reaction mixture was cooled to 0° C. and to this sodium triacetoxyborohydride (STAB) (400 mg, 2 mmol, 1.3 equiv.) was added. 3-pyridinecarboxaldehyde (244 mg, 1.5 mmol, 1 equiv.) was dissolved in CH₂Cl₂ (5 mL) and slowly added to the reaction mixture dropwise over 10 minutes. Upon complete conversion to product, the reaction mixture was diluted with CH₂Cl₂ (20 mL) and washed with saturated sodium bicarbonate solution (3×20 mL) and the organic layer was dried then concentrated under reduced pressure. Without further purification the crude material was dissolved in anhydrous CH₂Cl₂ and subjected to General Procedure B. The resulting crude was purified by PTLC to give a white solid (10 mg, 2%). NMR (500 MHz, Chloroform-d) δ 8.63 (d, J=3.8 Hz, 1H), 8.49 (s, 1H), 7.93 (s, 1H), 7.70 (d, J=7.7 Hz, 1H), 7.55 (s, 2H), 7.35 (dd, J=7.6, 5.3 Hz, 1H), 6.60 (dd, J=16.6, 1.6 Hz, 1H), 6.02 (dd, J=16.9, 10.2 Hz, 1H), 5.79 (dd, J=10.3, 1.6 Hz, 1H), 5.11 (s, 2H). HRMS-ESI (m/z) calculated for C₁₇H₁₃F₆N₂O [M+H]: 375.0927; found: 375.0928.

### 3-(2-chloroacetamido)-5-(trifluoromethyl)benzoic acid (36)

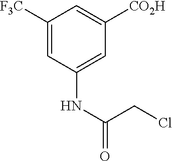 [SMILES: O=C(CCl)Nc1cc(C(=O)O)cc(C(F)(F)F)c1]

To a solution of 3-amino-5-(trifluoromethyl)benzoic acid (500 mg, 2.44 mmol) in 1.5 mL of dimethylacetamide (1.6 M) at 0° C. was added chloroacetyl chloride (214 μL, 2.69 mmol, 1.1 equiv.). The resulting solution was warmed to ambient temperature and stirred for 20 minutes, at which point ethyl acetate (40 mL) and water (30 mL) were added. The pH of the aqueous layer was adjusted to pH 10 via addition of 1 N NaOH, and the phases were separated. The aqueous layer was washed with 40 mL of ethyl acetate, then acidified by adding 1 N HCl. The product was extracted with ethyl acetate (40 mL), and the organic layer was washed with 1M HCl (2×40 mL), brine (40 mL), dried over magnesium sulfate and concentrated to provide the desired product (456 mg, 66%). NMR (500 MHz, Chloroform-d) δ 8.31 (s, 1H), 8.27 (s, 1H), 8.14 (s, 1H), 4.13 (s, 2H); HRMS-ESI (m/z) calculated for C₁₀H₈ClF₃NO₃ [M+H]: 282.0139; found: 282.0141.

### 1-(4-(5-fluorobenzisoxazol-3-yl)piperidin-1-yl)prop-2-en-1-one (37)

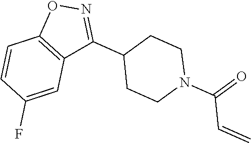 [SMILES: C=CC(=O)N1CCC(c2noc3ccc(F)cc23)CC1]

The title compound was obtained starting from 6-fluoro-3(4-piperidinyl)-1,2-benzisoxazole hydrochloride (53 mg, 0.2 mmol, 1 equiv.) according to General Procedure C as a colorless oil (49.1 mg, 87%). ¹H NMR (400 MHz, Chloroform-d) δ 7.64 (dd, J=8.7, 5.1 Hz, 1H), 7.27 (dd, J=8.4, 2.3 Hz, 1H), 7.08 (td, J=8.9, 2.1 Hz, 1H), 6.64 (dd, J=16.8, 10.6 Hz, 1H), 6.32 (dd, J=16.9, 1.9 Hz, 1H), 5.73 (dd, J=10.6, 1.9 Hz, 1H), 4.70 (d, J=13.4 Hz, 1H), 4.15 (d, J=12.4 Hz, 1H), 3.53-3.13 (m, 2H), 2.99 (t, J=13.1 Hz, 1H), 2.25-2.07 (m, 2H), 2.00 (ddd, J=23.1, 14.2, 7.8 Hz, 2H); HRMS-ESI (m/z) calculated for C₁₅H₁₆FN₂O [M+H]: 275.119; found: 275.119.

### tert-butyl 4-(4-acrylamido-2,6-difluorophenyl)piperazine-1-carboxylate (38)

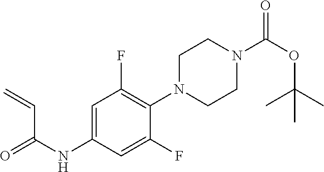 [SMILES: C=CC(=O)Nc1cc(F)c(N2CCN(C(=O)OC(C)(C)C)CC2)c(F)c1]

The title compound was obtained starting from tert-Butyl 4-(4-amino-2,6-difluorophenyl)piperazine-1-carboxylate according to General Procedure B. ¹H NMR (400 MHz, Chloroform-d) δ 8.12 (s, 1H), 7.13 (d, J=10.4 Hz, 2H), 6.36 (d, J=16.9 Hz, 1H), 6.19 (dd, J=16.8, 10.2 Hz, 1H), 5.70 (d, J=10.2 Hz, 1H), 3.45 (t, J=4.7 Hz, 4H), 3.00 (t, J=3.7 Hz, 4H), 1.41 (s, 9H); HRMS-ESI (m/z) calculated for C₈H₂₄F₂N₃O₃ [M+H]: 368.178; found: 368.178.

### N-(4-bromo-2,5-dimethylphenyl)acrylamide (40)

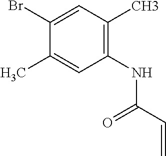

Following General Procedure B, starting from 4-bromo-2,5-dimethylaniline (900 mg, 4.5 mmol, 1 equiv.), the title compound was obtained after column chromatography and recrystallization from cold CH₂Cl₂ as a white solid (611 mg, 40%). ¹H NMR (500 MHz, Chloroform-d) δ 7.87 (s, 1H), 7.43 (s, 1H), 7.16 (s, 1H), 6.50 (d, J=16.7 Hz, 1H), 6.35 (dd, J=16.4, 10.3 Hz, 1H), 5.86 (d, J=10.3 Hz, 1H), 2.42 (s, 3H), 2.28 (s, 3H); HRMS-ESI (m/z) calculated for C₁₁H₁₃BrNO [M+H]: 254.0175; found: 254.0175.

### 2-Chloroacetamido-2-deoxy-α/β-D-glucopyranose (44)

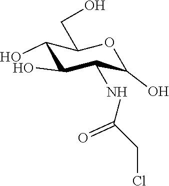

To a stirred solution of hexosamine hydrochloride (590 mg, 3.39 mmol, 1 equiv.) in anhydrous MeOH (200 mL) at room temperature was added sodium metal (60 mg, 2.6 mmol, 0.78 equiv.), TEA (400 μL, 5.7 mmol, 1.8 equiv.). Chloroacetic anhydride (1 g, 5.9 mmol, 1 equiv.) was then added and the mixture stirred for 6 h, monitoring for completeness by TLC. After which, the reaction mixture was concentrated in vacuo. The crude product then was purified by two rounds of column chromatography to afford the pure title product as a white solid (610 mg, 72%). ¹H NMR (500 MHz, Methanol-d₄) δ 5.20 (d, J=3.7 Hz, 1Hα), 4.75 (d, J=8.3 Hz, 1H(3), 4.19 (dd, J=20.2, 13.9 Hz, 2H), 4.19 (d, J=12.6 Hz, 1H), 3.95 (dd, J=10.6, 3.5 Hz, 1Hα), 3.83 (m, 3Hα, 3H(3), 3.74 (d, J=5.1 Hz, 1H₁₃), 3.70 (dd, J=11.4, 8.9 Hz, 1H(3), 3.60 (dd, J=10.7, 9.5 Hz, 1H(3), 3.46 (t, J=9.3 Hz, 1H), 3.42 (t, J=10.0 Hz, 1H₁₃); HRMS-ESI (m/z) calculated for C₈H₁₅ClNO₆ [M+H]: 256.0582; found: 256.0582.

### 2-chloro-1-(2-methyl-3,4-dihydroquinolin-1(2H)-yl)ethan-1-one (45)

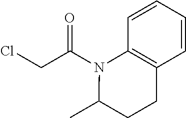

Chloroacetyl chloride (80.4 μL, 0.9 mmol, 1.7 equiv.) was dissolved in anhydrous CH₂Cl₂ (3 mL) and cooled to 0° C. A solution of 2-methyl-1,2,3,4-tetrahydroquinoline (80.1 mg, 0.544 mmol, 1 equiv.) and N-methylmorpholine (0.11 mL, 1.0 mmol, 1.8 equiv.) in CH₂Cl₂ (2 mL) was then added dropwise. After 6 h, the reaction was quenched with saturated aqueous NaHCO₃ (5 mL) and extracted with CH₂Cl₂ (3×10 mL). The combined organic layers were dried over anhydrous Na₂SO₄ and concentrated under reduced pressure. The resultant residue was purified by prep. TLC (30% EtOAc/hexanes), providing the title compound as an off-white solid (108.8 mg, 89%). NMR (400 MHz, chloroform-d) δ 7.30-7.13 (m, 4H), 4.86-4.75 (m, 1H), 4.20 (d, J=12.5 Hz, 1H), 4.09 (d, J=12.5 Hz, 1H), 2.69-2.58 (m, 1H), 2.59-2.46 (m, 1H), 2.46-2.31 (m, 1H), 1.36-1.29 (m, 1H), 1.15 (d, J=6.5 Hz, 3H); HRMS-ESI (m/z) calculated for C₁₂H₁₅ClNO [M+H]: 224.0837; found: 224.0836.

### N-cyclohexyl-N-phenylacrylamide (46)

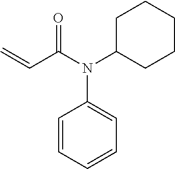 [SMILES: C=CC(=O)N(c1ccccc1)C1CCCCC1]

The title compound was synthesized according to General Procedure C from N-cyclohexylaniline (89.5 mg, 0.511 mmol, 1 equiv.). Purification of the crude product by flash column chromatography (10-20% EtOAc/hexanes) then prep. TLC (30% EtOAc/hexanes) provided the title compound as an off-white solid (53.1 mg, 45%). NMR (400 MHz, chloroform-d) δ 7.42-7.33 (m, 3H), 7.10-7.06 (m, 2H), 6.31 (dd, J=16.7, 2.1 Hz, 1H), 5.77 (dd, J=16.7, 10.3 Hz, 1H), 5.41 (dd, J=10.4, 2.1 Hz, 1H), 4.65 (tt, J=12.2, 3.7 Hz, 1H), 1.85 (dt, J=11.2, 1.8 Hz, 2H), 1.75-1.68 (m, 2H), 1.61-1.53 (m, 1H), 1.40 (qt, J=13.3, 3.6 Hz, 2H), 1.07 (qd, J=12.4, 3.6 Hz, 2H), 0.91 (qt, J=13.1, 3.8 Hz, 1H); HRMS-ESI (m/z) calculated for C₁₅H₂₀NO [M+H]: 230.1539; found: 230.1539.

### 1-(5-bromoindolin-1-yl)prop-2-en-1-one (47)

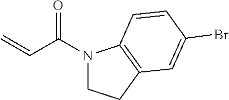

The title compound was synthesized according to General Procedure C from 5-bromoindoline (41.7 mg, 0.211 mmol, 1 equiv.), acryloyl chloride (32 μL, 0.40 mmol, 1.9 equiv.), and changing the base to pyridine (32 IA, 0.40 mmol, 1.9 equiv.). Purification of the crude product by re-precipitation from EtOAc provided the title compound as a white solid (67.8 mg, 64%). NMR (400 MHz, chloroform-d) δ 8.16 (d, J=8.6 Hz, 1H), 7.33-7.25 (m, 2H), 6.60-6.42 (m, 2H), 5.84-5.76 (m, 1H), 4.15 (t, J=8.6 Hz, 2H), 3.17 (t, J=8.6 Hz, 2H); HRMS-ESI (m/z) calculated for C₁₁H₁₁BrNO [M+H]: 252.0018; found: 252.0017.

### N-(1-benzylpiperidin-4-yl)-N-phenylacrylamide (48)

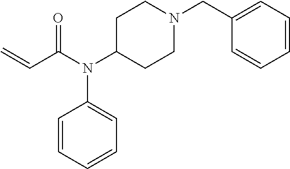 [SMILES: C=CC(=O)N(c1ccccc1)C1CCN(Cc2ccccc2)CC1]

The title compound was synthesized according to General Procedure C from 1-benzyl-N-phenylpiperidin-4-amine (30.0 mg, 0.113 mmol, 1 equiv.), acryloyl chloride (17 μL, 0.21 mmol, 1.9 equiv.), and changing the base to pyridine (17 μL, 0.21 mmol, 1.9 equiv.). Purification of the crude product by prep. TLC provided the title compound as a white solid (22.5 mg, 64%). ¹H NMR (400 MHz, chloroform-d) δ 7.62-7.56 (m, 2H), 7.43-7.36 (m, 6H), 7.05 (d, J=6.2 Hz, 2H), 6.29 (dd, J=16.8, 2.1 Hz, 1H), 5.79 (dd, J=16.8, 10.3 Hz, 1H), 5.46 (dd, J=10.3, 2.1 Hz, 1H), 4.81-4.70 (m, 1H), 4.09 (s, 2H), 3.41 (d, J=12.0 Hz, 2H), 2.82 (q, J=11.5 Hz, 2H), 2.21 (q, J=11.9 Hz, 2H), 1.94 (d, J=14.2 Hz, 2H); HRMS-ESI (m/z) calculated for C₂₁H₂₅N₂O [M+H]: 321.1961; found: 321.1962.

### 2-chloro-N-(2-methyl-5-(trifluoromethyl)phenyl)acetamide (49)

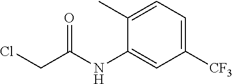 [SMILES: Cc1ccc(C(F)(F)F)cc1NC(=O)CCl]

The title compound was synthesized according to General Procedure A1 from 2-methyl-5-(trifluoromethyl)aniline (35.0 mg, 0.2 mmol, 1 equiv.). Purification of the crude product by prep. TLC (20% EtOAc/hexanes) provided the title compound as a white solid (48.2 mg, 95%). ¹H NMR (600 MHz, chloroform-d) δ 8.31 (s, 1H), 8.25 (d, J=1.9 Hz, 1H), 7.37 (dd, J=7.9, 1.8 Hz, 1H), 7.32 (d, J=7.9 Hz, 1H), 4.25 (s, 2H), 2.36 (s, 3H); HRMS-ESI calculated for C₁₀H₁₀ClF₃NO [M+H]: 252.0397; found: 252.0397.

### 1-(5-bromoindolin-1-yl)-2-chloroethan-1-one (50)

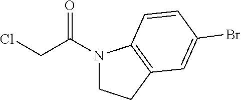 [SMILES: O=C(CCl)N1CCc2cc(Br)ccc21]

The title compound was synthesized according to General Procedure A1 from 5-bromoindoline (39.6 mg, 0.2 mmol, 1 equiv.). Purification of the crude product by prep. TLC (25% EtOAc/hexanes) provided the title compound as an off-white solid (48.6 mg, 89%). NMR (600 MHz, CDCl₃) δ 8.07 (d, J=8.4 Hz, 1H), 7.32 (d, J=8.8 Hz, 2H), 4.17 (t, J=8.6 Hz, 2H), 4.14 (s, 2H), 3.22 (t, J=8.4 Hz, 2H); HRMS-ESI (m/z) calculated for C₁₀H₁₀BrClNO [M+H]: 273.9629; found: 273.9629.

### 2-chloro-N-(quinolin-5-yl)acetamide (51)

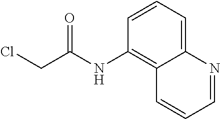 [SMILES: O=C(CCl)Nc1cccc2ncccc12]

To a stirring suspension of 5-aminoquinoline (28.8 mg, 0.2 mmol, 1 equiv.) and potassium carbonate (82.9 mg, 0.6 mmol, 3 equiv.) in anhydrous CH₂Cl₂ (3 mL) at 0° C. was added chloroacetyl chloride (24 μL, 1.5 equiv.). The reaction was allowed to slowly warm up to room temperature. After 3 hours, the mixture was filtered, washed with EtOAc (10 mL) and CH₂Cl₂ (10 mL). The solid cake was then eluted with MeOH (20 mL) and the filtrate concentrated in vacuo. The residue was taken up in 10% MeOH/CH₂Cl₂ and passed through a pad of silica to provide the title compound as an off-white solid (42.6 mg, 82%). ¹H NMR (500 MHz, CDCl₃) δ 8.96 (d, J=2.5 Hz, 1H), 8.71 (s, 1H), 8.20 (d, J=8.6 Hz, 1H), 8.04 (d, J=8.5 Hz, 1H), 7.94 (d, J=7.5 Hz, 1H), 7.74 (t, J=8.0 Hz, 1H), 7.48 (dd, J=8.5, 4.2 Hz, 1H), 4.35 (s, 2H); HRMS-ESI (m/z) calculated for C₁₁H₉ClN₂O [M+H]: 221.0476; found: 221.0477.

### 1-(4-benzylpiperidin-1-yl)prop-2-en-1-one (53)

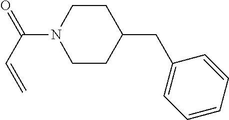 [SMILES: C=CC(=O)N1CCC(Cc2ccccc2)CC1]

Following General Procedure B, starting from 4-benzylpiperidine (1 g, 5.7 mmol, 1 equiv.), the title compound was obtained after column chromatography as a yellow oil (748 mg, 57%). ¹H NMR (500 MHz, Chloroform-d) δ 7.36 (t, J=7.4 Hz, 2H), 7.28 (t, J=7.4 Hz, 1H), 7.20 (d, J=7.1 Hz, 2H), 6.64 (dd, J=16.8, 10.6 Hz, 1H), 6.32 (dd, J=16.8, 1.9 Hz, 1H), 5.72 (dd, J=10.6, 1.9 Hz, 1H), 4.72 (d, J=12.7 Hz, 1H), 4.03 (d, J=13.0 Hz, 1H), 3.05 (t, J=12.7 Hz, 1H), 2.70-2.59 (m, 3H), 1.86 (ddp, J=14.6, 7.2, 3.5 Hz, 1H), 1.77 (m, 2H), 1.37-1.18 (m, 2H); HRMS-ESI (m/z) calculated for C₅H₂₀ClNO [M+H]: 230.1539; found: 230.1539.

### 2-chloro-N-((3-hydroxy-5-(hydroxymethyl)-2-methylpyridin-4-yl)methyl)acetamide (54)

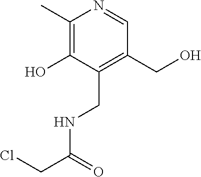 [SMILES: Cc1ncc(CO)c(CNC(=O)CCl)c1O]

To a stirred solution of pyridoxamine hydrochloride (150 mg, 0.64 mmol, 1 equiv.) in anhydrous MeOH (20 mL) at room temperature was added sodium metal (30 mg, 1.5 mmol, 2.3 equiv.), TEA (100 μL, 1 mmol, 1.6 equiv.). Chloroacetic anhydride (390 mg, 2.29 mmol, 3.5 equiv.) was added and the mixture stirred for 6 h, monitoring for completeness by TLC. After which, the reaction mixture was concentrated in vacuo. The crude product then was the purified by prep. TLC to afford the title compound as a white solid (46 mg, 30%). ¹H NMR (500 MHz, Methanol-d₄) δ 7.97 (s, 1H), 4.81 (s, 2H), 4.61 (s, 2H), 4.17 (s, 3H), 4.06 (s, 1H), 3.35 (s, 1H), 2.52 (s, 3H); HRMS-ESI (m/z) calculated for C₁₀H₁₄ClN₂O₃ [M+H]: 245.0687; found: 245.0688.

### 1-(6,7-dimethoxy-3,4-dihydroisoquinolin-2(1H)-yl)prop-2-en-1-one (56)

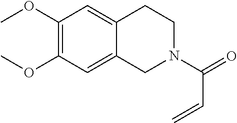 [SMILES: C=CC(=O)N1CCc2cc(OC)c(OC)cc2C1]

To a stirring suspension of the 6,7-dimethoxy-3,4-dihydroisoquinoline (1 g, 5.2 mmol, 1 equiv.) and TEA (1800 μL, 12.6 mmol, 2.5 equiv.) in anhydrous THF (10 mL) at 0° C. was added acryloyl chloride (1320 μL, 13.2 mmol, 2.6 equiv.) and the reaction was allowed to slowly warm up to room temperature. After 2 hours, the mixture was diluted with CH₂Cl₂ (2×50 mL) and washed with saturated brine (2×50 mL) and the combined organics were concentrated in vacuo. The residue was taken up in 10% MeOH/CH₂Cl₂ and purified by column chromatography to afford the title compound as a white solid (700 mg, 54%, mixture of E/Z isomers). ¹H NMR (500 MHz, Chloroform-d) δ 6.63 (m, 3H), 6.29 (d, J=16.8 Hz, 1H), 5.69 (dd, J=10.6, 1.8 Hz, 1H), 4.69 (s, 1H [major]), 4.63 (s, 0.8H [minor]), 3.82 (s, 7H), 3.73 (t, J=5.6 Hz, 1H), 2.84-2.77 (m, 2H); HRMS-ESI (m/z) calculated for C₁₄H₁₈NO₃ [M+H]: 248.128; found: 248.1281.

### 2-chloro-N-(1-(3-ethynylbenzoyl)piperidin-4-yl)-N-phenylacetamide (61)

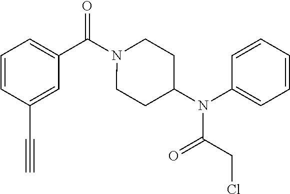 [SMILES: C#Cc1cccc(C(=O)N2CCC(N(C(=O)CCl)c3ccccc3)CC2)c1]

To an excess of neat SI-3 was added 0.7 mL of trifluoroacetic acid (0.2 M). The resulting solution was concentrated under a stream of nitrogen until no further evaporation was observed, providing the deprotected amine as its trifluoroacetate salt. The triflouroacetate amine salt (90.6 mg, 0.25 mmol) was taken up in DMF (0.5 mL, 0.5 M) and the resulting solution was cooled to 0° C. 3-ethynyl benzoic acid (44 mg, 1.2 equiv.), HATU (113 mg, 1.2 equiv.), and Hunig's base (86 μL, 2 equiv.) were sequentially added. The reaction was stirred for 2 hours at 0° C., diluted with Et₂O, and then washed with 1 M HCl. The organic layer was dried over magnesium sulfate, concentrated, and purified by flash chromatography (gradient from 40 to 70% ethyl acetate in hexanes) to provide the title compound (87 mg, 92%). ¹H NMR (400 MHz, Chloroform-d) δ 7.51 (dd, J=9.5, 5.4 Hz, 4H), 7.43 (d, J=1.9 Hz, 1H), 7.39-7.25 (m, 2H), 7.14 (d, J=10.4 Hz, 2H), 4.86 (tt, J=15.1, 5.3 Hz, 2H), 3.72 (s, 3H), 3.19 (d, J=14.0 Hz, 1H), 3.11 (s, 1H), 2.86 (s, 1H), 1.90 (d, J=36.6 Hz, 2H), 1.38 (s, 1H), 1.24 (d, J=19.9 Hz, 1H); HRMS-ESI (m/z) calculated for C₂₂H₂₂ClN₂O₂ [M+H]: 381.1364; found: 381.1363.

The synthesis of the compound fragments from Table S12 was performed according the procedures of the above examples.

“An activity-guided map of electrophile-cysteine interactions in primary human immune cells”, Ekaterina Vinogradova, et al., bioRxiv 808113; doi: https://doi.org/10.1101/808113 (accessible on Oct. 17, 2019) and is herein incorporated by reference in its entirety.

TABLE S12
Chemical structures screened

| Compound | Structure |
|---|---|
| EV-1 | 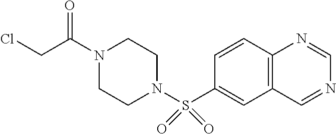 [SMILES: O=C(CCl)N1CCN(S(=O)(=O)c2ccc3ncncc3c2)CC1] |
| EV-2 | 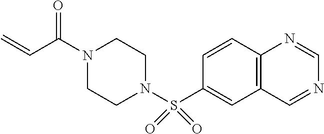 [SMILES: C=CC(=O)N1CCN(S(=O)(=O)c2ccc3ncncc3c2)CC1] |
| EV-3 | 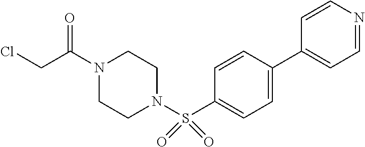 [SMILES: O=C(CCl)N1CCN(S(=O)(=O)c2ccc(-c3ccncc3)cc2)CC1] |
| EV-4 | 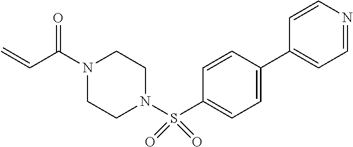 [SMILES: C=CC(=O)N1CCN(S(=O)(=O)c2ccc(-c3ccncc3)cc2)CC1] |
| EV-5 | 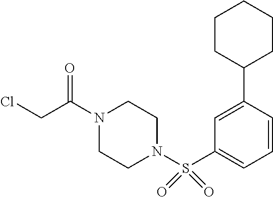 [SMILES: O=C(CCl)N1CCN(S(=O)(=O)c2cccc(C3CCCCC3)c2)CC1] |
| EV-6 | 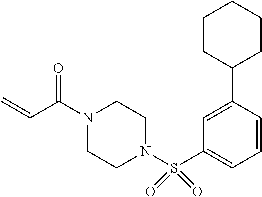 [SMILES: C=CC(=O)N1CCN(S(=O)(=O)c2cccc(C3CCCCC3)c2)CC1] |
| EV-7 | 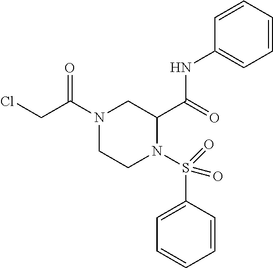 [SMILES: O=C(Nc1ccccc1)C1CN(C(=O)CCl)CCN1S(=O)(=O)c1ccccc1] |
| EV-8 | 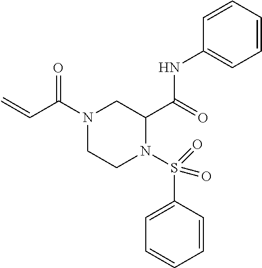 [SMILES: C=CC(=O)N1CCN(S(=O)(=O)c2ccccc2)C(C(=O)Nc2ccccc2)C1] |
| EV-9 | 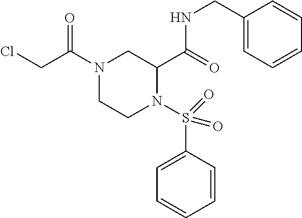 [SMILES: O=C(NCc1ccccc1)C1CN(C(=O)CCl)CCN1S(=O)(=O)c1ccccc1] |
| EV-10 | 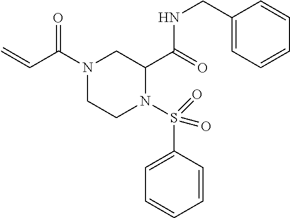 [SMILES: C=CC(=O)N1CCN(S(=O)(=O)c2ccccc2)C(C(=O)NCc2ccccc2)C1] |
| EV-11 | 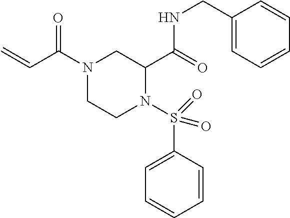 [SMILES: C=CC(=O)N1CCN(S(=O)(=O)c2ccccc2)C(C(=O)NCc2ccccc2)C1] |
| EV-12 | 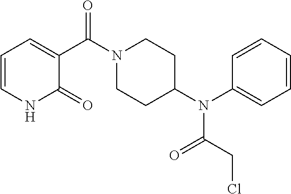 [SMILES: O=C(c1ccc[nH]c1=O)N1CCC(N(C(=O)CCl)c2ccccc2)CC1] |
| EV-13 | 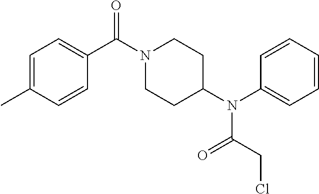 [SMILES: Cc1ccc(C(=O)N2CCC(N(C(=O)CCl)c3ccccc3)CC2)cc1] |
| EV-14 | 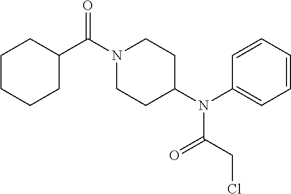 [SMILES: O=C(C1CCCCC1)N1CCC(N(C(=O)CCl)c2ccccc2)CC1] |
| EV-15 | 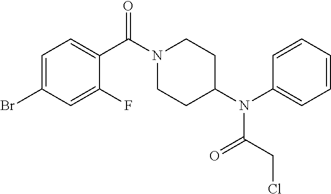 [SMILES: O=C(c1ccc(Br)cc1F)N1CCC(N(C(=O)CCl)c2ccccc2)CC1] |
| EV-16 | 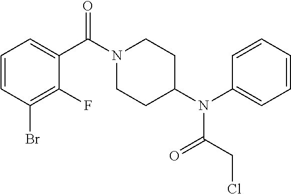 [SMILES: O=C(c1cccc(Br)c1F)N1CCC(N(C(=O)CCl)c2ccccc2)CC1] |
| EV-17 | 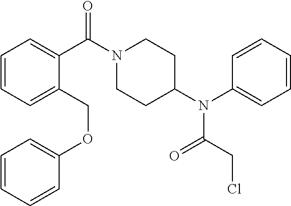 [SMILES: O=C(c1ccccc1COc1ccccc1)N1CCC(N(C(=O)CCl)c2ccccc2)CC1] |
| EV-18 | 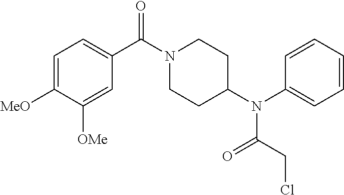 [SMILES: COc1ccc(C(=O)N2CCC(N(C(=O)CCl)c3ccccc3)CC2)cc1OC] |
| EV-19 | 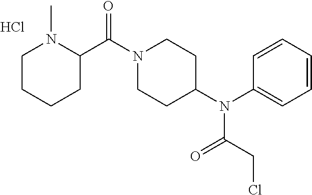 [SMILES: Cl ; CN1CCCCC1C(=O)N1CCC(N(C(=O)CCl)c2ccccc2)CC1] |
| EV-20 | 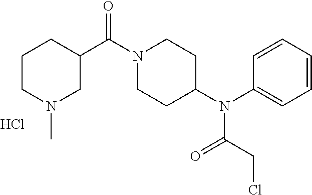 [SMILES: Cl ; CN1CCCC(C(=O)N2CCC(N(C(=O)CCl)c3ccccc3)CC2)C1] |
| EV-21 | 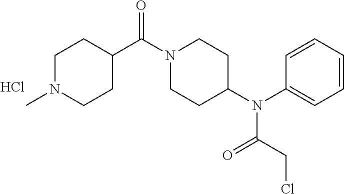 [SMILES: Cl ; CN1CCC(C(=O)N2CCC(N(C(=O)CCl)c3ccccc3)CC2)CC1] |
| EV-22 | 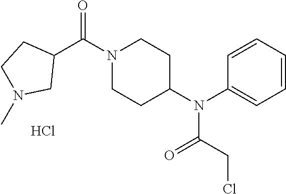 [SMILES: CN1CCC(C(=O)N2CCC(N(C(=O)CCl)c3ccccc3)CC2)C1 ; Cl] |
| EV-23 | 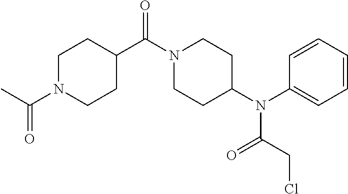 [SMILES: CC(=O)N1CCC(C(=O)N2CCC(N(C(=O)CCl)c3ccccc3)CC2)CC1] |
| EV-24 | 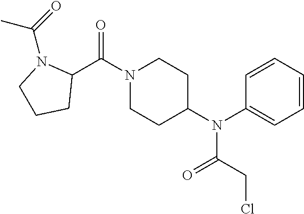 [SMILES: CC(=O)N1CCCC1C(=O)N1CCC(N(C(=O)CCl)c2ccccc2)CC1] |
| EV-25 | 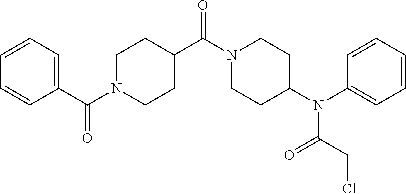 [SMILES: O=C(c1ccccc1)N1CCC(C(=O)N2CCC(N(C(=O)CCl)c3ccccc3)CC2)CC1] |
| EV-26 | 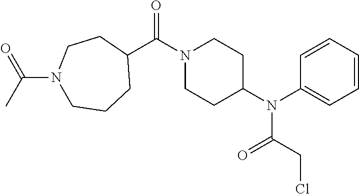 [SMILES: CC(=O)N1CCCC(C(=O)N2CCC(N(C(=O)CCl)c3ccccc3)CC2)CC1] |
| EV-27 | 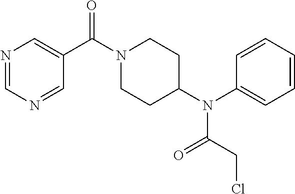 [SMILES: O=C(c1cncnc1)N1CCC(N(C(=O)CCl)c2ccccc2)CC1] |
| EV-28 | 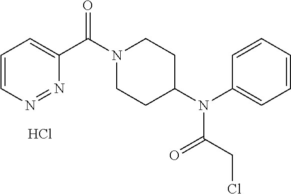 [SMILES: O=C(c1cccnn1)N1CCC(N(C(=O)CCl)c2ccccc2)CC1 ; Cl] |
| EV-29 | 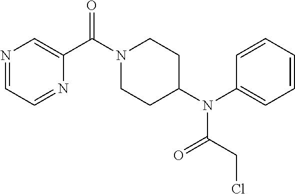 [SMILES: O=C(c1cnccn1)N1CCC(N(C(=O)CCl)c2ccccc2)CC1] |
| EV-30 | 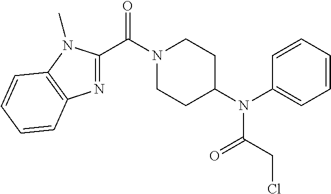 [SMILES: Cn1c(C(=O)N2CCC(N(C(=O)CCl)c3ccccc3)CC2)nc2ccccc21] |
| EV-31 | 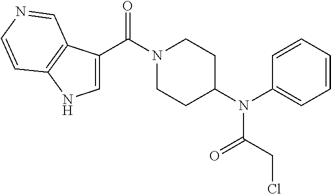 [SMILES: O=C(c1c[nH]c2ccncc12)N1CCC(N(C(=O)CCl)c2ccccc2)CC1] |
| EV-32 | 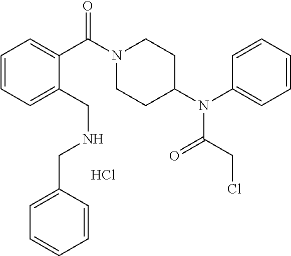 [SMILES: O=C(c1ccccc1CNCc1ccccc1)N1CCC(N(C(=O)CCl)c2ccccc2)CC1 ; Cl] |
| EV-33 | 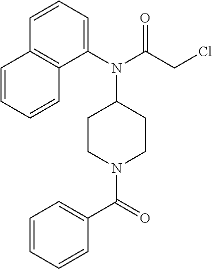 [SMILES: O=C(c1ccccc1)N1CCC(N(C(=O)CCl)c2cccc3ccccc23)CC1] |
| EV-34 | 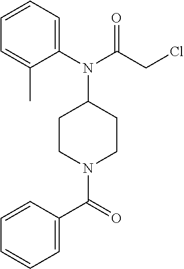 [SMILES: Cc1ccccc1N(C(=O)CCl)C1CCN(C(=O)c2ccccc2)CC1] |
| EV-35 | 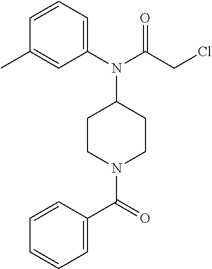 [SMILES: Cc1cccc(N(C(=O)CCl)C2CCN(C(=O)c3ccccc3)CC2)c1] |
| EV-36 | 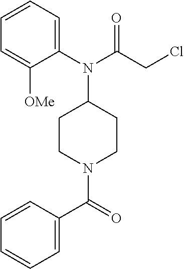 [SMILES: COc1ccccc1N(C(=O)CCl)C1CCN(C(=O)c2ccccc2)CC1] |
| EV-37 | 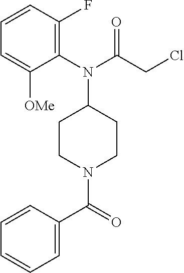 [SMILES: COc1cccc(F)c1N(C(=O)CCl)C1CCN(C(=O)c2ccccc2)CC1] |
| EV-38 | 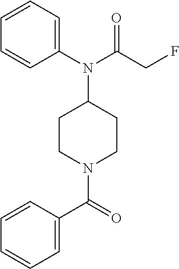 [SMILES: O=C(c1ccccc1)N1CCC(N(C(=O)CF)c2ccccc2)CC1] |
| EV-39 | 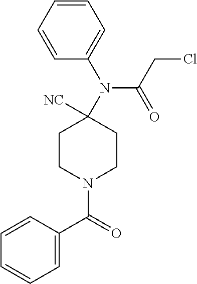 [SMILES: N#CC1(N(C(=O)CCl)c2ccccc2)CCN(C(=O)c2ccccc2)CC1] |
| EV-40 | 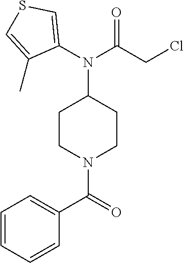 [SMILES: Cc1cscc1N(C(=O)CCl)C1CCN(C(=O)c2ccccc2)CC1] |
| EV-41 | 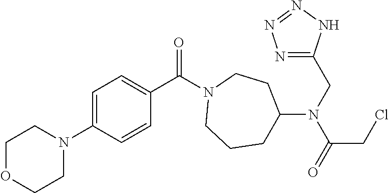 [SMILES: O=C(c1ccc(N2CCOCC2)cc1)N1CCCC(N(Cc2nnn[nH]2)C(=O)CCl)CC1] |
| EV-42 | 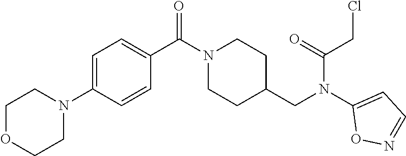 [SMILES: O=C(c1ccc(N2CCOCC2)cc1)N1CCC(CN(C(=O)CCl)c2ccno2)CC1] |
| EV-43 | 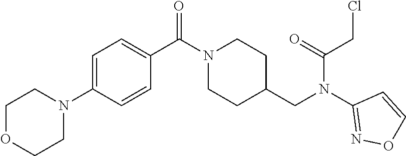 [SMILES: O=C(c1ccc(N2CCOCC2)cc1)N1CCC(CN(C(=O)CCl)c2ccon2)CC1] |
| EV-44 | 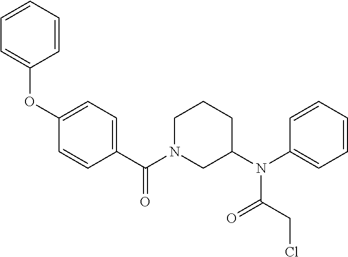 [SMILES: O=C(c1ccc(Oc2ccccc2)cc1)N1CCCC(N(C(=O)CCl)c2ccccc2)C1] |
| EV-45 | 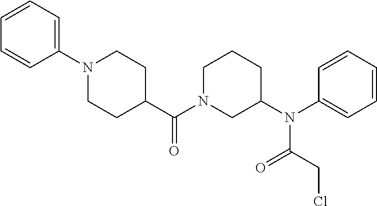 [SMILES: O=C(C1CCN(c2ccccc2)CC1)N1CCCC(N(C(=O)CCl)c2ccccc2)C1] |
| EV-46 | 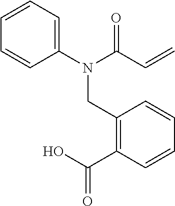 [SMILES: C=CC(=O)N(Cc1ccccc1C(=O)O)c1ccccc1] |
| EV-47 | 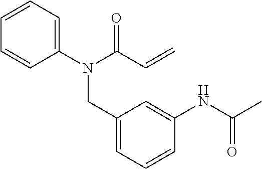 [SMILES: C=CC(=O)N(Cc1cccc(NC(C)=O)c1)c1ccccc1] |
| EV-48 | 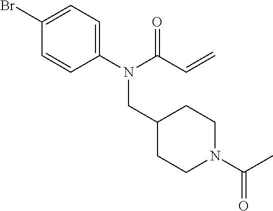 [SMILES: C=CC(=O)N(CC1CCN(C(C)=O)CC1)c1ccc(Br)cc1] |
| EV-49 | 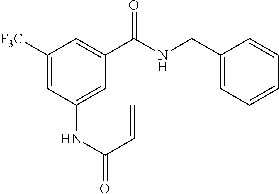 [SMILES: C=CC(=O)Nc1cc(C(=O)NCc2ccccc2)cc(C(F)(F)F)c1] |
| EV-50 | 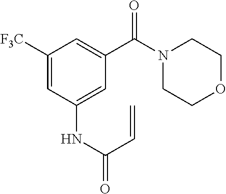 [SMILES: C=CC(=O)Nc1cc(C(=O)N2CCOCC2)cc(C(F)(F)F)c1] |
| EV-51 | 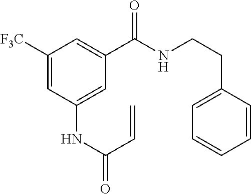 [SMILES: C=CC(=O)Nc1cc(C(=O)NCCc2ccccc2)cc(C(F)(F)F)c1] |
| EV-52 | 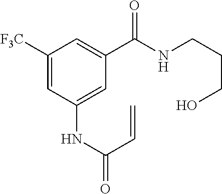 [SMILES: C=CC(=O)Nc1cc(C(=O)NCCCO)cc(C(F)(F)F)c1] |
| EV-53 | 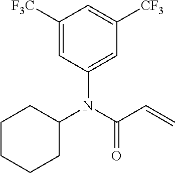 [SMILES: C=CC(=O)N(c1cc(C(F)(F)F)cc(C(F)(F)F)c1)C1CCCCC1] |
| EV-54 | 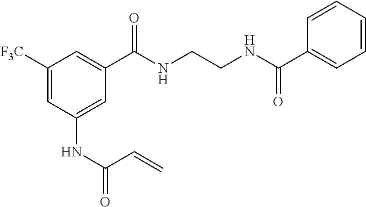 [SMILES: C=CC(=O)Nc1cc(C(=O)NCCNC(=O)c2ccccc2)cc(C(F)(F)F)c1] |
| EV-55 | 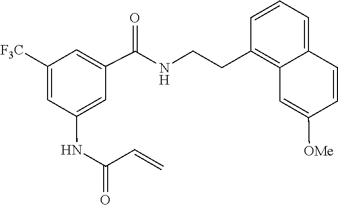 [SMILES: C=CC(=O)Nc1cc(C(=O)NCCc2cccc3ccc(OC)cc23)cc(C(F)(F)F)c1] |
| EV-56 | 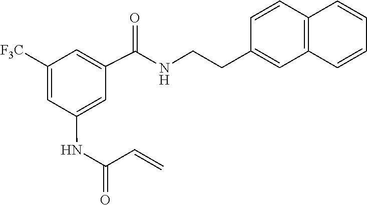 [SMILES: C=CC(=O)Nc1cc(C(=O)NCCc2ccc3ccccc3c2)cc(C(F)(F)F)c1] |
| EV-57 | 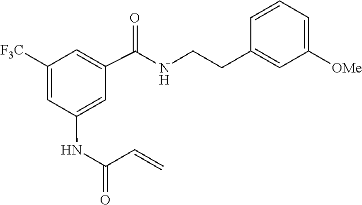 [SMILES: C=CC(=O)Nc1cc(C(=O)NCCc2cccc(OC)c2)cc(C(F)(F)F)c1] |
| EV-58 | 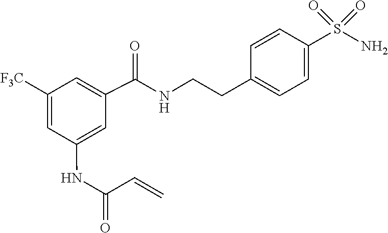 [SMILES: C=CC(=O)Nc1cc(C(=O)NCCc2ccc(S(N)(=O)=O)cc2)cc(C(F)(F)F)c1] |
| EV-59 | 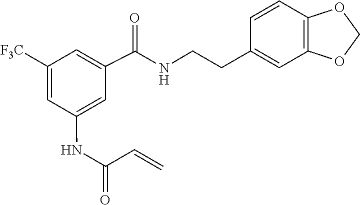 [SMILES: C=CC(=O)Nc1cc(C(=O)NCCc2ccc3c(c2)OCO3)cc(C(F)(F)F)c1] |
| EV-60 | 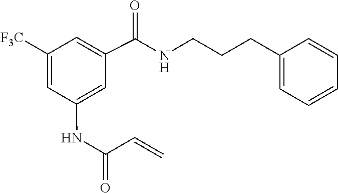 [SMILES: C=CC(=O)Nc1cc(C(=O)NCCCc2ccccc2)cc(C(F)(F)F)c1] |
| EV-61 | 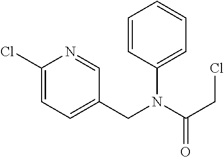 [SMILES: O=C(CCl)N(Cc1ccc(Cl)nc1)c1ccccc1] |
| EV-62 | 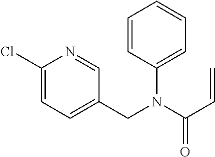 [SMILES: C=CC(=O)N(Cc1ccc(Cl)nc1)c1ccccc1] |
| EV-63 | 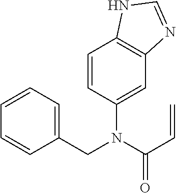 [SMILES: C=CC(=O)N(Cc1ccccc1)c1ccc2[nH]cnc2c1] |
| EV-64 | 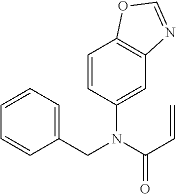 [SMILES: C=CC(=O)N(Cc1ccccc1)c1ccc2ocnc2c1] |
| EV-65 | 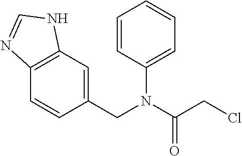 [SMILES: O=C(CCl)N(Cc1ccc2nc[nH]c2c1)c1ccccc1] |
| EV-66 | 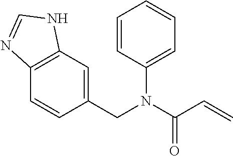 [SMILES: C=CC(=O)N(Cc1ccc2nc[nH]c2c1)c1ccccc1] |
| EV-67 | 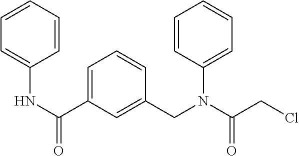 [SMILES: O=C(Nc1ccccc1)c1cccc(CN(C(=O)CCl)c2ccccc2)c1] |
| EV-68 | 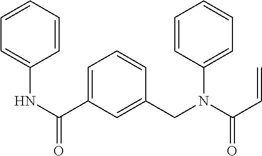 [SMILES: C=CC(=O)N(Cc1cccc(C(=O)Nc2ccccc2)c1)c1ccccc1] |
| EV-69 | 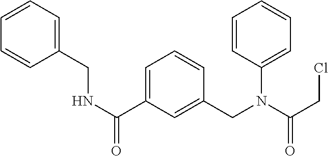 [SMILES: O=C(NCc1ccccc1)c1cccc(CN(C(=O)CCl)c2ccccc2)c1] |
| EV-70 | 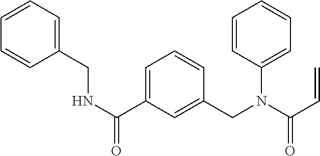 [SMILES: C=CC(=O)N(Cc1cccc(C(=O)NCc2ccccc2)c1)c1ccccc1] |
| EV-71 | 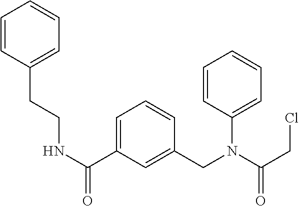 [SMILES: O=C(NCCc1ccccc1)c1cccc(CN(C(=O)CCl)c2ccccc2)c1] |
| EV-72 | 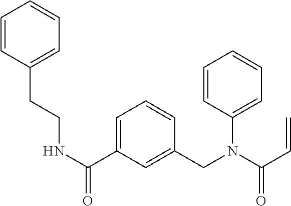 [SMILES: C=CC(=O)N(Cc1cccc(C(=O)NCCc2ccccc2)c1)c1ccccc1] |
| EV-73 | 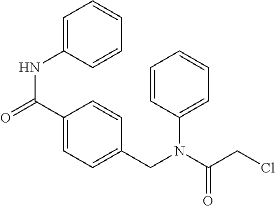 [SMILES: O=C(Nc1ccccc1)c1ccc(CN(C(=O)CCl)c2ccccc2)cc1] |
| EV-74 | 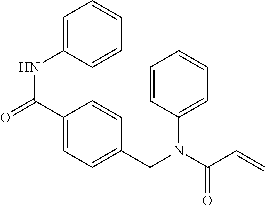 [SMILES: C=CC(=O)N(Cc1ccc(C(=O)Nc2ccccc2)cc1)c1ccccc1] |
| EV-75 | 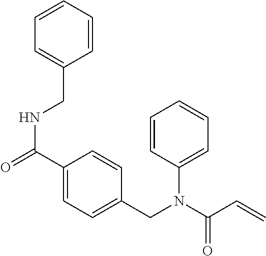 [SMILES: C=CC(=O)N(Cc1ccc(C(=O)NCc2ccccc2)cc1)c1ccccc1] |
| EV-76 | 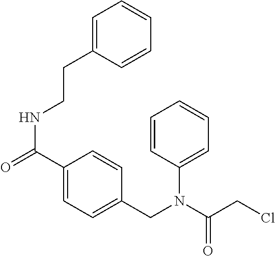 [SMILES: O=C(NCCc1ccccc1)c1ccc(CN(C(=O)CCl)c2ccccc2)cc1] |
| EV-77 | 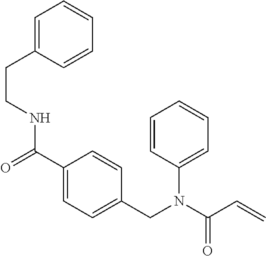 [SMILES: C=CC(=O)N(Cc1ccc(C(=O)NCCc2ccccc2)cc1)c1ccccc1] |
| EV-78 | 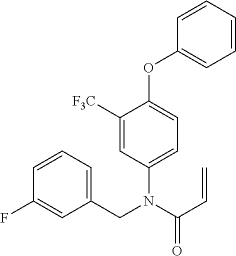 [SMILES: C=CC(=O)N(Cc1cccc(F)c1)c1ccc(Oc2ccccc2)c(C(F)(F)F)c1] |
| EV-79 | 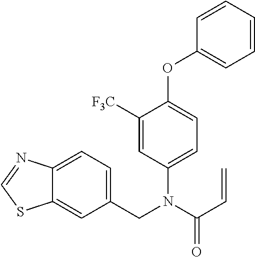 [SMILES: C=CC(=O)N(Cc1ccc2ncsc2c1)c1ccc(Oc2ccccc2)c(C(F)(F)F)c1] |
| EV-80 | 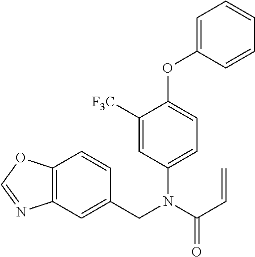 [SMILES: C=CC(=O)N(Cc1ccc2ocnc2c1)c1ccc(Oc2ccccc2)c(C(F)(F)F)c1] |
| EV-81 | 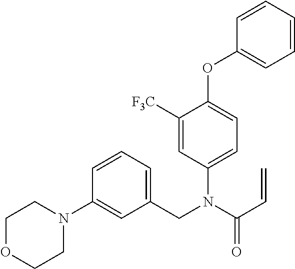 [SMILES: C=CC(=O)N(Cc1cccc(N2CCOCC2)c1)c1ccc(Oc2ccccc2)c(C(F)(F)F)c1] |
| EV-82 | 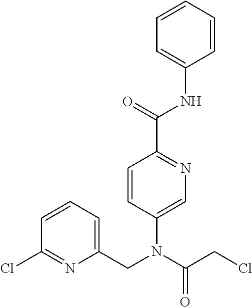 [SMILES: O=C(Nc1ccccc1)c1ccc(N(Cc2cccc(Cl)n2)C(=O)CCl)cn1] |
| EV-83 | 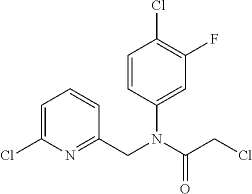 [SMILES: O=C(CCl)N(Cc1cccc(Cl)n1)c1ccc(Cl)c(F)c1] |
| EV-84 | 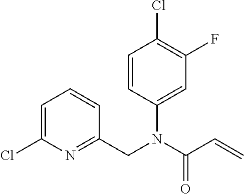 [SMILES: C=CC(=O)N(Cc1cccc(Cl)n1)c1ccc(Cl)c(F)c1] |
| EV-85 | 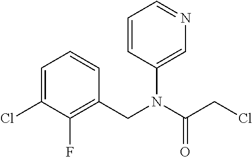 [SMILES: O=C(CCl)N(Cc1cccc(Cl)c1F)c1cccnc1] |
| EV-86 | 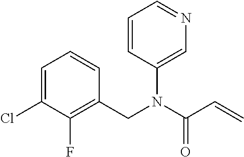 [SMILES: C=CC(=O)N(Cc1cccc(Cl)c1F)c1cccnc1] |
| EV-87 | 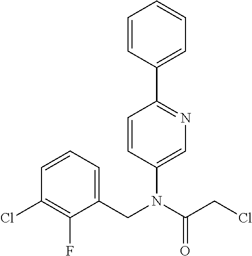 [SMILES: O=C(CCl)N(Cc1cccc(Cl)c1F)c1ccc(-c2ccccc2)nc1] |
| EV-88 | 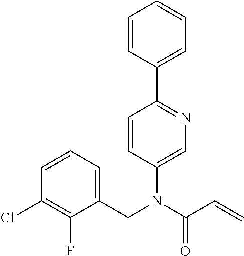 [SMILES: C=CC(=O)N(Cc1cccc(Cl)c1F)c1ccc(-c2ccccc2)nc1] |
| EV-89 | 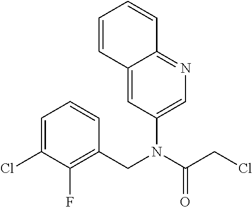 [SMILES: O=C(CCl)N(Cc1cccc(Cl)c1F)c1cnc2ccccc2c1] |
| EV-90 | 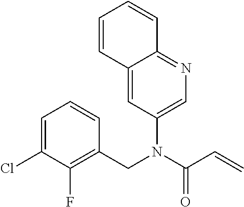 [SMILES: C=CC(=O)N(Cc1cccc(Cl)c1F)c1cnc2ccccc2c1] |
| EV-91 | 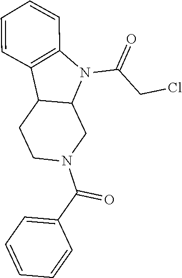 [SMILES: O=C(c1ccccc1)N1CCC2c3ccccc3N(C(=O)CCl)C2C1] |
| EV-92 | 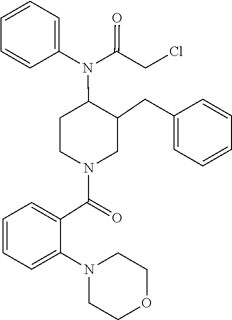 [SMILES: O=C(c1ccccc1N1CCOCC1)N1CCC(N(C(=O)CCl)c2ccccc2)C(Cc2ccccc2)C1] |
| EV-93 | 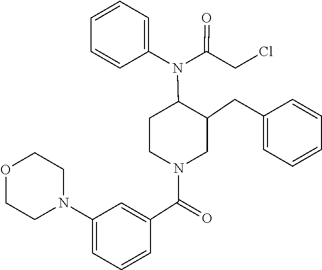 [SMILES: O=C(c1cccc(N2CCOCC2)c1)N1CCC(N(C(=O)CCl)c2ccccc2)C(Cc2ccccc2)C1] |
| EV-94 | 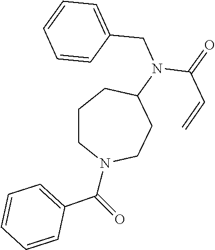 [SMILES: C=CC(=O)N(Cc1ccccc1)C1CCCN(C(=O)c2ccccc2)CC1] |
| EV-95 | 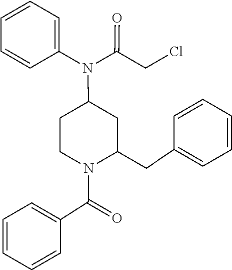 [SMILES: O=C(c1ccccc1)N1CCC(N(C(=O)CCl)c2ccccc2)CC1Cc1ccccc1] |
| BPK-5 | 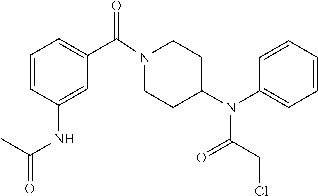 [SMILES: CC(=O)Nc1cccc(C(=O)N2CCC(N(C(=O)CCl)c3ccccc3)CC2)c1] |
| BPK-7 | 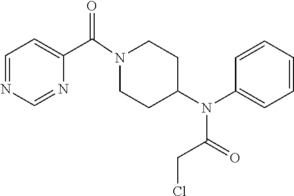 [SMILES: O=C(c1ccncn1)N1CCC(N(C(=O)CCl)c2ccccc2)CC1] |
| BPK-8 | 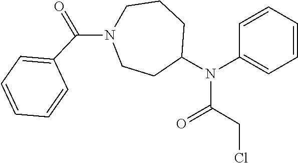 [SMILES: O=C(c1ccccc1)N1CCCC(N(C(=O)CCl)c2ccccc2)CC1] |
| BPK-9 | 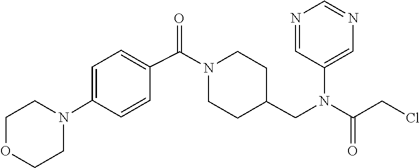 [SMILES: O=C(c1ccc(N2CCOCC2)cc1)N1CCC(CN(C(=O)CCl)c2cncnc2)CC1] |
| BPL-10 | 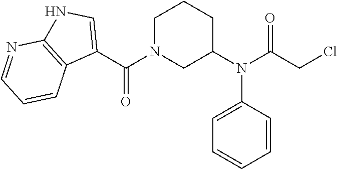 [SMILES: O=C(c1c[nH]c2ncccc12)N1CCCC(N(C(=O)CCl)c2ccccc2)C1] |
| BPK-11 | 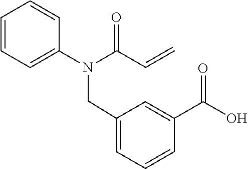 [SMILES: C=CC(=O)N(Cc1cccc(C(=O)O)c1)c1ccccc1] |
| BPK-16 | 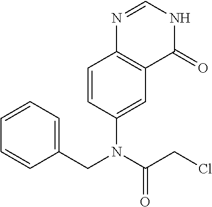 [SMILES: O=C(CCl)N(Cc1ccccc1)c1ccc2nc[nH]c(=O)c2c1] |
| BPK-18 | 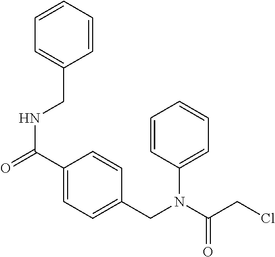 [SMILES: O=C(NCc1ccccc1)c1ccc(CN(C(=O)CCl)c2ccccc2)cc1] |
| BPK-19 | 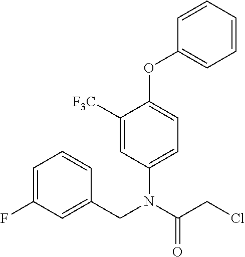 [SMILES: O=C(CCl)N(Cc1cccc(F)c1)c1ccc(Oc2ccccc2)c(C(F)(F)F)c1] |
| BPK-20 | 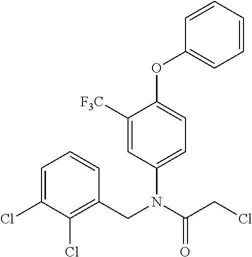 [SMILES: O=C(CCl)N(Cc1cccc(Cl)c1Cl)c1ccc(Oc2ccccc2)c(C(F)(F)F)c1] |
| BPK-21 | 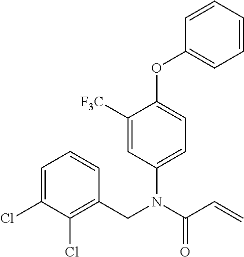 [SMILES: C=CC(=O)N(Cc1cccc(Cl)c1Cl)c1ccc(Oc2ccccc2)c(C(F)(F)F)c1] |
| BPK-22 | 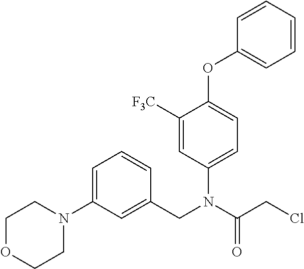 [SMILES: O=C(CCl)N(Cc1cccc(N2CCOCC2)c1)c1ccc(Oc2ccccc2)c(C(F)(F)F)c1] |
| BPK-25 | 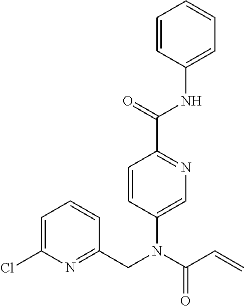 [SMILES: C=CC(=O)N(Cc1cccc(Cl)n1)c1ccc(C(=O)Nc2ccccc2)nc1] |
| BPK-29 | 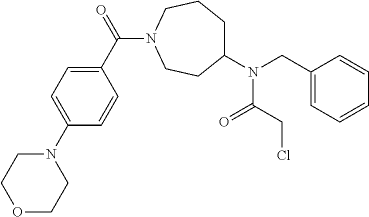 [SMILES: O=C(c1ccc(N2CCOCC2)cc1)N1CCCC(N(Cc2ccccc2)C(=O)CCl)CC1] |
| BPK-30 | 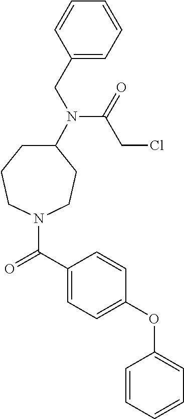 [SMILES: O=C(c1ccc(Oc2ccccc2)cc1)N1CCCC(N(Cc2ccccc2)C(=O)CCl)CC1] |
| BPK-31 | 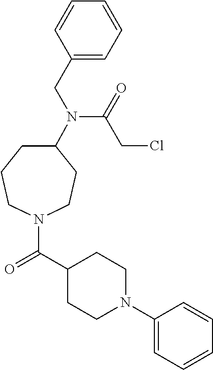 [SMILES: O=C(C1CCN(c2ccccc2)CC1)N1CCCC(N(Cc2ccccc2)C(=O)CCl)CC1] |
| BPK-34 | 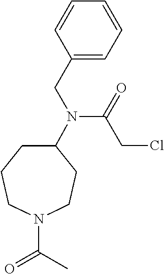 [SMILES: CC(=O)N1CCCC(N(Cc2ccccc2)C(=O)CCl)CC1] |
| HS58A-C2 | 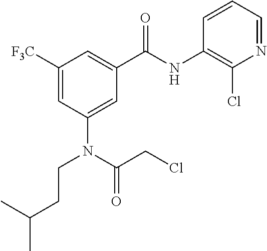 [SMILES: CC(C)CCN(C(=O)CCl)c1cc(C(=O)Nc2cccnc2Cl)cc(C(F)(F)F)c1] |
| HS77 | 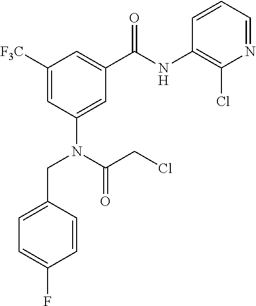 [SMILES: O=C(Nc1cccnc1Cl)c1cc(N(Cc2ccc(F)cc2)C(=O)CCl)cc(C(F)(F)F)c1] |
| HS81C | 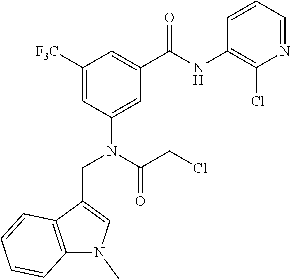 [SMILES: Cn1cc(CN(C(=O)CCl)c2cc(C(=O)Nc3cccnc3Cl)cc(C(F)(F)F)c2)c2ccccc21] |
| HS81e | 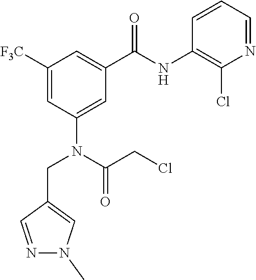 [SMILES: Cn1cc(CN(C(=O)CCl)c2cc(C(=O)Nc3cccnc3Cl)cc(C(F)(F)F)c2)cn1] |
| HS92 | 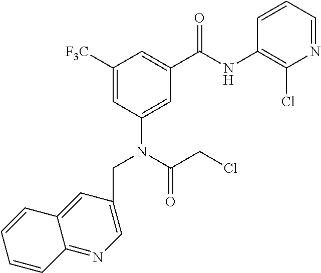 [SMILES: O=C(Nc1cccnc1Cl)c1cc(N(Cc2cnc3ccccc3c2)C(=O)CCl)cc(C(F)(F)F)c1] |
| HS95 | 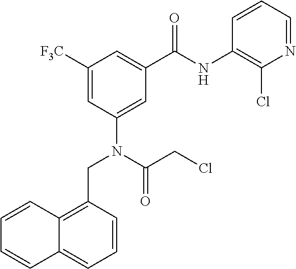 [SMILES: O=C(Nc1cccnc1Cl)c1cc(N(Cc2cccc3ccccc23)C(=O)CCl)cc(C(F)(F)F)c1] |
| HS96 | 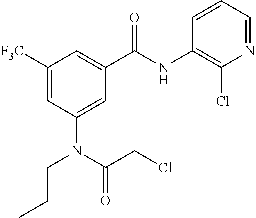 [SMILES: CCCN(C(=O)CCl)c1cc(C(=O)Nc2cccnc2Cl)cc(C(F)(F)F)c1] |
| HS97 | 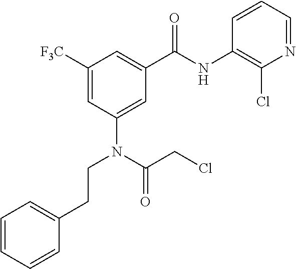 [SMILES: O=C(Nc1cccnc1Cl)c1cc(N(CCc2ccccc2)C(=O)CCl)cc(C(F)(F)F)c1] |
| HS98 | 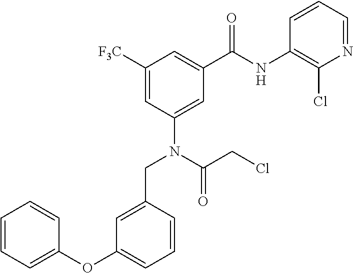 [SMILES: O=C(Nc1cccnc1Cl)c1cc(N(Cc2cccc(Oc3ccccc3)c2)C(=O)CCl)cc(C(F)(F)F)c1] |
| HS125 | 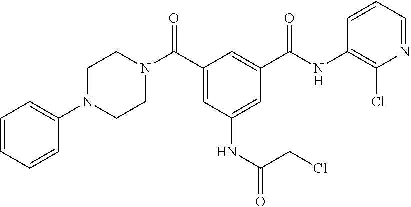 [SMILES: O=C(CCl)Nc1cc(C(=O)Nc2cccnc2Cl)cc(C(=O)N2CCN(c3ccccc3)CC2)c1] |
| HS126 | 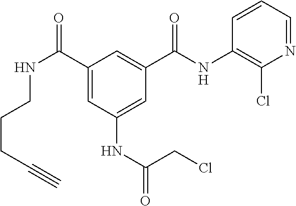 [SMILES: C#CCCCNC(=O)c1cc(NC(=O)CCl)cc(C(=O)Nc2cccnc2Cl)c1] |
| HS145 | 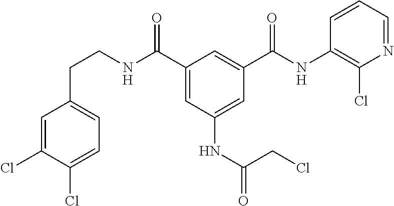 [SMILES: O=C(CCl)Nc1cc(C(=O)NCCc2ccc(Cl)c(Cl)c2)cc(C(=O)Nc2cccnc2Cl)c1] |
| HS175 | 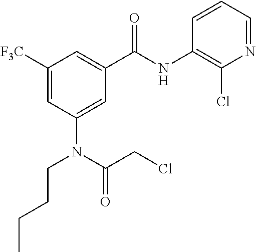 [SMILES: CCCCN(C(=O)CCl)c1cc(C(=O)Nc2cccnc2Cl)cc(C(F)(F)F)c1] |
| HS177 | 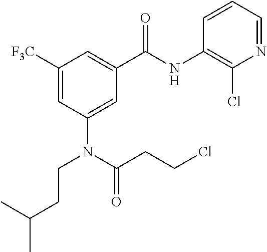 [SMILES: CC(C)CCN(C(=O)CCCl)c1cc(C(=O)Nc2cccnc2Cl)cc(C(F)(F)F)c1] |
| HS178 | 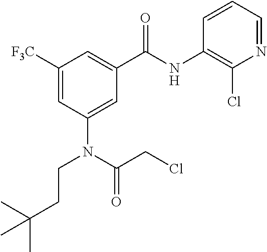 [SMILES: CC(C)(C)CCN(C(=O)CCl)c1cc(C(=O)Nc2cccnc2Cl)cc(C(F)(F)F)c1] |
| RS004 | 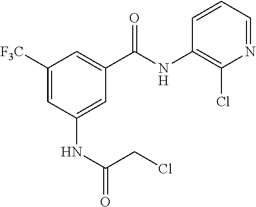 |
| KB63 | 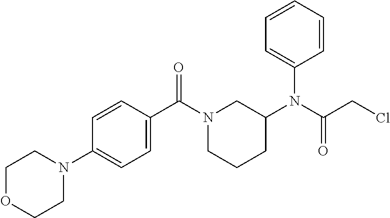 [SMILES: O=C(c1ccc(N2CCOCC2)cc1)N1CCCC(N(C(=O)CCl)c2ccccc2)C1] |

### Example 2

Animal Treatment

Female DBA/1 mice (7-10 week of age) are purchased from The Jackson Laboratory (Bar Harbor, ME), and are kept for 1 week before treatments. The animal facilities are certified by the Association for Assessment and Accreditation of Laboratory Animal Care. An illustrative compound from FIG. 4, compound A, is used for this study. The animals are injected i.p. with about 50 mg/kg of compound A (dissolved in phosphate-buffered saline) or vehicle four times weekly for 3 weeks. Four days after the last dose, mice are sacrificed, and splenocytes and lymph node cells are isolated for ex vivo T-cell proliferation assays.

Lymph Node and Splenic T-Cell Proliferation Assay

Splenocytes and lymph node cells obtained from the Animal Treatment study are separately pooled from three to five mice, and single-cell suspension are prepared. The cells (about 1×10⁶ cells/well) are stimulated with 10 μg/ml of compound A, and then incubated for 4 days in a 96-well plate in DMEM containing 10% fetal calf serum (FCS). During the last 16 hours, the cells are pulsed with [ 3H]thymidine (0.5 μCi/well), and T-cell proliferation is determined by thymidine uptake. In the lymph node proliferation assay, serum-free X-VIVO medium is used.

Electrophoresis Analysis

Splenocytes and lymph node cells obtained from the Animal Treatment study are separately pooled and centrifuged to collect the respective cell pellet. The cell pellet is subsequently lysed and resolved on a 10-12% polyacrylamide gel. Protein bands are subsequently visualized by silver staining.

### Example 3

Tumor Cell Lines and Mice

Six to eight-week female C57BL and C3H mice are purchased (Charles River Laboratories, Wilmington, MA). The animal facilities are certified by the Association for Assessment and Accreditation of Laboratory Animal Care.

ID8 is a clone of the MOSEC ovarian carcinoma of C57BL/6 origin. SW1 is a clone derived from the K1735 melanoma of C₃H origin.

In Vivo Studies

In experiments with the ID8 ovarian carcinoma, mice (5 or 10/group) are transplanted i.p. with 3×10⁶ cells. Either 10 or 15 days later, they are injected i.p. with compound A or vehicle, which is repeated weekly for a total of 3 times. Mice are monitored daily for tumor growth, including swollen bellies indicating that they have developed ascites, and for evidence of toxicity. Tumor growth is recorded using a digital caliper. The survival of each mouse is further recorded and overall survival is calculated as mean±standard error of mean (M±SEM).

In experiments with the SW1 melanoma, 5×10⁵ cells are transplanted s.c. on the right flank, When the mice have developed tumors of about 4-5 mm in mean diameter, they are randomized into treatment group and control group; with either compound A or vehicle injected i.p., respectively, at weekly intervals for a total of 3 times. Mice are monitored daily for evidence of toxicity. Tumor diameters are measured twice/week using a digital caliper and tumor surfaces are calculated. Overall survival is also recorded.

### EXAMPLE

Phase 1 Clinical Trial

Purpose: this clinical trial is to assess the safety and tolerability of administration of compound A in combination with low-dose cytokines (IL-2 and IFN-alpha) in patients with metastatic or refractory cancer.
Study Type: Interventional
Study Design: Allocation: Non-Randomized
Intervention Model: Single Group Assignment
Masking: Open Label
Primary Purpose: Treatment
Primary Outcome Measures:
   Safety [Time Frame: Initial dose of study therapy through 30 days post last dose of study therapy]
   Tolerability [Time Frame: Initial dose of study therapy through 30 days post last dose of study therapy]
   Anti-tumor Activity [Time Frame: From initial dose of study therapy to disease progression]
Eligibility
Ages Eligible for Study: 18 Years and older (Adult, Senior)
Sexes Eligible for Study: All
Accepts Healthy Volunteers: No

Criteria

Inclusion Criteria:
Have a histologically confirmed diagnosis of metastatic or refractory cancer for which there are no effective standard therapeutic options available;
Have signed an Institutional Review Board (IRB) approved informed consent form (ICF) prior to performing any study evaluation/procedures;
Be > or =18 years of age and women must either be 1) not of childbearing potential or 2) have a negative serum pregnancy test within 7 days prior to commencing treatment. Patients are considered not of childbearing potential if they are surgically sterile (they have undergone a hysterectomy, bilateral tubal ligation or bilateral oophorectomy) or they are postmenopausal (12 consecutive months of amenorrhea [lack of menstruation]);
(If applicable) Have completed prior cytotoxic chemotherapy, radiotherapy or immunotherapy or experimental therapy > or =30 days prior to the study enrollment, and recovered form associated toxicities;
Have an Eastern Cooperative Oncology Group (ECOG) score of < or =2, and an anticipated life expectancy of at least 6 months;
Have adequate hematologic function, as defined by an absolute or calculated neutrophil count > or =1500/microL, platelet count > or =100000/microL, lymphocyte count > or =500/microL, and hemoglobin level > or =10 g/dL. Patients may not receive prophylactic transfusion in order to qualify for trial eligibility;
Have adequate renal function, as defined by a documented serum creatinine of < or =2.0 mg/dL. Greater than “1+” proteinuria will require microscope evaluation and the results discussed with the medical monitor prior to patient enrollment; or if serum creatine is >2.0, patient must have an actual or calculated 24-hour creatinine clearance of >60 mL/min and no obvious evidence of concurrent medullary cystic disease or obstructive uropathy;
Have adequate hepatic function, as defined by a total bilirubin level < or =1.5×upper limit of normal (ULN) and alkaline phosphatase, aspartate transaminase (AST), and alanine transaminase (ALT) levels < or =2.5×ULN. If alkaline phosphatase is outside of these parameters and is due to bone metastases (as verified by the assessment of isoenzymes), then the patient is eligible.

Exclusion Criteria:
Have a history of severe hypersensitivity (grade 3-4 allergic reaction) to fluorescein or any drug, radiologic contrast agent, insect bite, food, cytokines, or any other agent; or have received fluorescein within 30 days of the study;
Have medical conditions that preclude the use of IL-2 or IFN-alpha. These conditions include but are not limited to, diabetes mellitus with a history of progression to diabetic ketoacidosis, history of severe coagulation disorder, psoriasis, sarcoidosis, retinal hemorrhage, symptomatic pulmonary disease, heart failure (> or =New York Heart Association NYHA class II), or transplant requiring immunosuppressive therapy;
Be pregnant or breast-feeding;
Be currently receiving an experimental drug, or used an experimental device within 30 days of study entry;
Be currently undergoing chemotherapy, anticancer hormonal therapy, and/or therapy with immunosuppressant agents;
Have any concomitant malignancy with the exception of basal cell or squamous cell carcinoma of skin;
Have radiographically documented evidence of current brain metastases, a history of stem cell transplant, immunodeficiency, and/or a medical or psychiatric illness (that in the investigator's opinion, would prevent adequate compliance with study therapy or evaluation of the endpoints).

### Example 5

### Methods Details

Isolation of Peripheral Blood Mononuclear Cells (PBMC) and T Cells

All studies with primary human cells were performed with samples from human volunteers followed by protocols approved by The Scripps Research Institute Institutional Review Board. Blood from healthy donors (age 18 to 65) was obtained after informed donor consent. Peripheral blood mononuclear cells (PBMCs) were isolated over Lymphoprep (STEMCELL Technologies) gradient using slightly modified manufacturer's instructions. Briefly, 25 mL of freshly isolated blood was layered on top of 12.5 mL of Lymphoprep in a 50 mL Falcon tube minimizing mixing of blood with Lymphoprep. The tubes were centrifuged at room temperature (931 g, 20 min, 23° C.) with break off and the plasma and Lymphoprep layers containing PBMCs were transferred to new 50 mL Falcon tubes with a 2:1 dilution with PBS. The cells were pelleted (524 g, 8 min, 4° C.) and washed with PBS once. T cells were isolated from fresh PBMCs using EasySep Human T Cell Isolation Kit (STEMCELL Technologies, negative selection) according to manufacturer's instructions.

T Cell Activation for Mass-Spectrometry Analysis

Non-tissue culture treated 6-well plates were pre-coated with αCD3 (5 μg/mL, BioXCell) and αCD28 antibodies (2 μg/mL, BioXCell) in PBS (2 mL/well) and kept at 4° C. overnight. The next day, the plates were transferred to a 37° C. incubator for 1 h and washed with PBS (2×5 mL/well). Freshly isolated T cells were resuspended in RPMI media supplemented with 10% FBS, L-glutamine (2 mM), penicillin (100 U/mL), and streptomycin (100 μg/mL) at 1×10\hat{ }6 cells/mL, plated into the pre-coated 6-well plates (6-10 mL/well) and kept at 37° C. in a 5% CO₂ incubator for 3 days. Following this incubation period, the cells were combined in 50 mL Falcon tubes, pelleted (524 g, 5 min, 4° C.), and washed with PBS (10 mL). The cells were then transferred into Eppendorf tubes in 1 mL of PBS, pelleted, flash-frozen, and kept at −80° C. until further analysis.

T Cell Expansion for Mass-Spectrometry Analysis (Control T Cells)

A non-tissue culture treated 6-well plate was pre-coated with αCD3 (1.5 μg/mL) antibody in PBS (3 mL/well) and kept at 4° C. overnight. The next day, the plates were transferred to a 37° C. incubator for 1 h and washed with PBS (2×5 mL/well). Freshly isolated T cells were re-suspended in RPMI media (10% FBS, L-glutamine (2 mM), penicillin (100 U/mL), streptomycin (100 μg/mL)), containing αCD28 antibody (1 μg/mL) at 1×10⁶ cells/mL, plated into the pre-coated 6-well plate (6-10 mL/well) and kept at 37° C. in a 5% CO₂ incubator for 3 days. Following this incubation period, the cells were combined in 50 mL Falcon tubes, pelleted (524 g, 5 min, 4° C.), and washed with PBS (10 mL). The cells were then re-suspended in RPMI media containing recombinant IL2 (10 U/mL) and kept at 37° C. in a 5% CO₂ incubator for 10-12 days, splitting the cells every 3-4 days to keep cell density below 2×10⁶ cells/mL. After this time, the cells were pelleted (524 g, 5 min, 4° C.), washed with PBS (10 mL) and either re-suspended in fresh RPMI media for in situ treatments or flash-frozen and kept at −80° C. until further analysis (in vitro treatments).

In Situ Labeling with Cysteine-Reactive Electrophiles

Activated or expanded T cells were re-suspended in RPMI media supplemented with 10% FBS, L-glutamine (2 mM), penicillin (100 U/mL), and streptomycin (100 μg/mL) at 2×10⁶ cells/mL. The compounds were added to cells as 1000×DMSO stocks and mixed well with the media by pipetting up and down after addition. The cells were kept at 37° C. in 5% CO₂ containing incubators for 3 h (or otherwise specified times), then pelleted by centrifugation (524 g, 5 min, 4° C.), washed with cold PBS (10 mL) and transferred to Eppendorf tubes (1 mL PBS). The cells were pelleted again (524 g, 5 min, 4° C.), flash-frozen, and kept at −80° C. until further analysis.

Multidimensional Screen for Inhibition of T Cell Activation

Non-tissue culture treated 96-well plates were pre-coated with αCD3 (5 μg/mL) and αCD28 antibodies (2 μg/mL) in PBS (100 μL/well) and left at 4° C. overnight. Freshly isolated T cells were re-suspended in RPMI media supplemented with 10% FBS, L-glutamine (2 mM), penicillin (100 U/mL), and streptomycin (100 mg/mL) at 2×10⁶ cells/mL. Compound stocks (200×) in DMSO were diluted to 2× stocks in the working RPMI media in another 96-well plate. The pre-coated 96-well treatment plates were washed with PBS (2×2000 μL), T cells (100 μL/well, 2×10⁵ cells/well) were then added to the wells, followed by the addition of 2× compound stocks in RPMI media (100 μL). The outer wells of the plates were filled with media without cells to avoid the edge effect in the assay. The treatment was done overnight (24 h) at 37° C. in a 5% CO₂ containing incubator. Following the treatment, the cells were transferred to a U-bottom 96-well plate and harvested by centrifugation (600 g, 3 min, 4° C.). The supernatants were kept and stored at −80° C. for further cytokine analysis, while the cells were washed with PBS (2×150 μL) prior to staining for flow cytometry analysis.

Flow Cytometry Analysis

Following the PBS washes, the cells were stained with fixable near-IR LIVE/DEAD cell stain (Invitrogen) according to manufacturer's instructions. Briefly, one vial of near-IR LIVE/DEAD dye was resuspended in DMSO (50 μL) and diluted with PBS (1:1000). The diluted stain was added to each well (200 μL) and the cells were incubated for 30 min at room temperature in the dark. After this time, the cells were pelleted (600 g, 3 min, 4° C.), washed once with PBS (200 μL/well) and incubated with a freshly made cocktail of antibodies for the appropriate cell surface markers diluted in PBS containing 2% FBS (1:400 antibody dilution). The corresponding data in FIG. 4D is presented as the mean percentage of DMSO treated control±SEM, n=3/group.

Measurement of Phospho-NF-κB p65 (Ser536) Levels

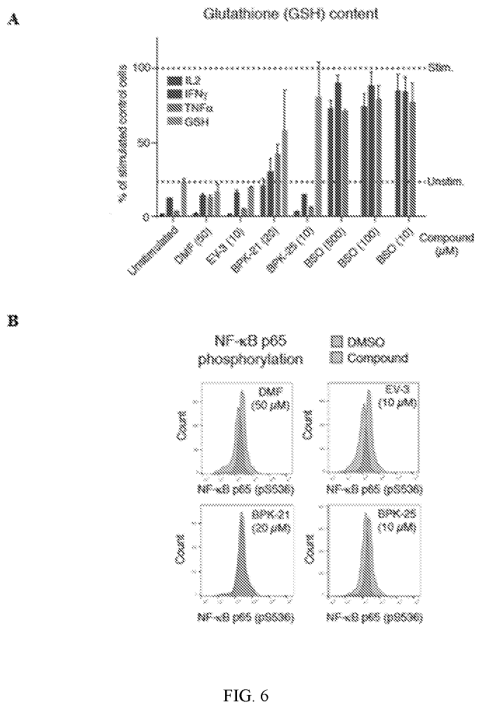

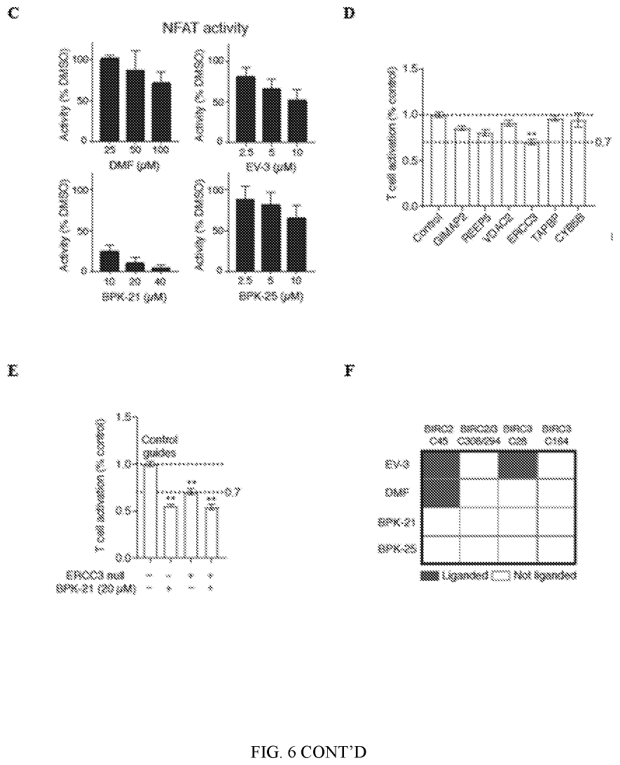

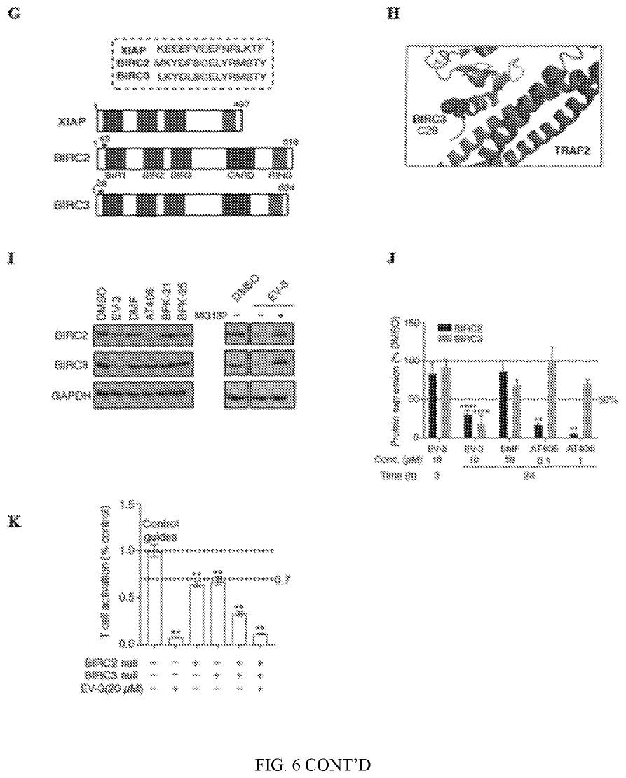

Freshly isolated T cells (2×10⁵ cells/well) were harvested and stimulated as described before in a 96-well plate in the presence of DMSO or compounds of interest. Following the overnight treatment, the cells were pelleted in a U-bottom plate, harvested by centrifugation (600 g, 3 min, 4° C.), washed with PBS, and stained with near-IR LIVE/DEAD dye as described above. After the staining, intracellular phospho-NF-κB p65 (Ser536) levels were measured using PE conjugate of phospho-NF-κB p65 (Ser536) (93H1) rabbit antibody (Cell Signaling Technology) according to manufacturer's instructions. Briefly, the cells were washed with PBS and fixed with 4% PFA in PBS (100 μL, 15 min, rt). The cells were washed with PBS again (2×150 μL), placed on ice and permeabilized with 90% MeOH (100 μL/well, slow addition with gentle mixing by pipetting up and down). Following a 30 min incubation on ice, the plate was sealed and stored at −20° C. overnight. The following day, the cells were thawed on ice, washed with PBS (150 μL×2), and stained with PE conjugate of phospho-NF-κB p65 (Ser536) (93H1) rabbit antibody (50 μL, 1:100 dilution in incubation buffer (1% FBS in PBS)) for 1 h at rt in the dark. The cells were then washed with incubation buffer (150 μL×2) and resuspended in PBS for further flow cytometry analysis. Data in FIG. 6B are from a single experiment representative of at least two independent biological experiments.

Measurement of Intracellular Glutathione Levels

Intracellular glutathione levels were determined using GSH-Glo glutathione assay (Promega Corporation) according to manufacturer's instructions. Briefly, freshly isolated T cells were treated with compounds or DMSO overnight under TCR-stimulating conditions (96-well plate, 1×10\hat{ }5 cells/well) at 37° C. in 5% CO₂ containing incubator, then transferred to a U-shape bottom 96-well plate and pelleted (600 g, 3 min, 4° C.). The supernatants were kept and stored at −80° C. for cytokine analysis. The cells were washed with PBS (2×150 μL) and re-suspended in 50 μL of PBS. An aliquot of treated cells (25 μL) was then added to an equal volume of 2×GSH reaction buffer containing Glutathione S-transferase and Luciferin-NT substrate (1:50 dilution in GSH-Glo Reaction Buffer). The reaction was incubated for 30 min at rt, after which Luciferin Detection Reagent (in reconstitution buffer with esterase, 25 μL/well) was added, and the plate was incubated for an additional 15 min and luminescence was read using a CLARIOstar (BMG Labtech) plate reader. The corresponding data in FIG. 6A is presented as the mean percentage of DMSO-treated control±SEM, n=3/group.

DuoSet ELISA Quantification of Secreted Cytokines (IL2, IFNγ, TNFα)

The levels of secreted IL2, IFNγ and TNFα after incubating T cells in the presence of DMSO or electrophilic compounds under TCR-stimulating conditions were measured using DuoSet ELISA cytokine kits (R&D Systems) in clear microplates (R&D Systems) according to manufacturer's instructions and read using a CLARIOstar (BMG Labtech) plate reader (450 nm). All cytokine concentrations were calculated according to the standard curve generated for each experiment. The corresponding data in FIGS. 4D and 6A is presented as the mean percentage of DMSO-treated control±SEM, n=3/group.

NFAT (Nuclear Factor of Activated T Cells) Luciferase Reporter Assay

NFAT activity was measured using the Jurkat-Lucia NFAT reporter cell line (Invitrogen) according to manufacturer's procedure. Briefly, Jurkat-Lucia NFAT cells were cultured at 37° C. in 5% CO₂ containing incubator in manufacturer-recommended growth medium (RPMI, 2 mM L-glutamine, 25 mM HEPES, 10% heat-inactivated fetal bovine serum (FBS, 30 min at 56° C.), 100 lag/mL Normocin, Pen-Strep (50 U/mL-50 μg/mL)) keeping cell density below 2×10⁶ cells/mL. To maintain selection pressure, Zeocin (100 μg/mL) was added to the growth medium every other passage and the cell passage number was kept less than 10. On the day of compound treatment, the cells were pelleted (300 g, 5 min) and resuspended at 2.2×10⁵ cells/mL in fresh, pre-warmed test medium (RPMI, 2 mM L-glutamine, 25 mM HEPES, 10% heat-inactivated FBS, Pen-Strep (100 U/mL-100 lag/mL) without Normocin). Cell suspension (180 μL, 4×10⁵ cells/well) was then added to the test plate containing stimulating solution (20 PMA (50 ng/mL) and ionomycin (3 μg/mL) in growth media) and test compounds (2 μL, 100× stock in DMSO) or DMSO, and the plate was kept at 37° C. in a 5% CO₂ containing incubator for 24 h. To evaluate expression of the luciferase reporter, 50 μL of Quanti-luc (Invivogen) detection reagent was combined with 20 μL of cell suspension from each well in a new 96-well white (opaque) plate and the luminescence was read using a CLARIOstar microplate reader (BMG Labtech). The corresponding data in FIG. 6C is presented as the mean percentage of DMSO treated control±SD or SEM, n=2-5/group.

ISRE-Luciferase and CellTiter Glo Assays

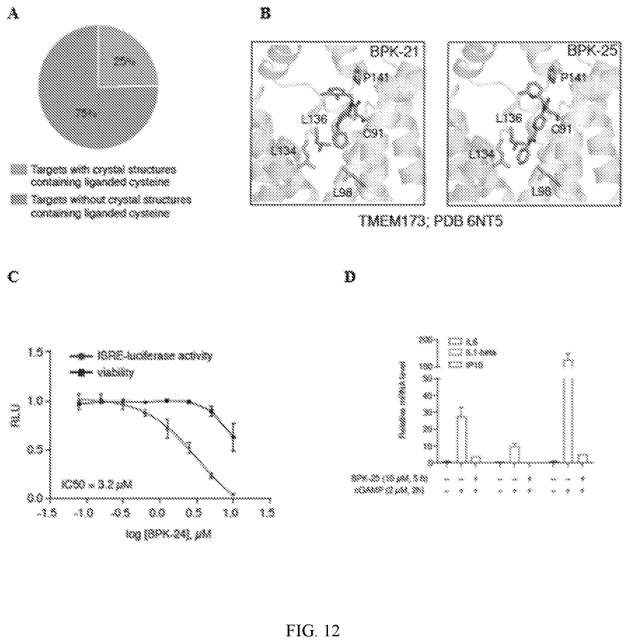

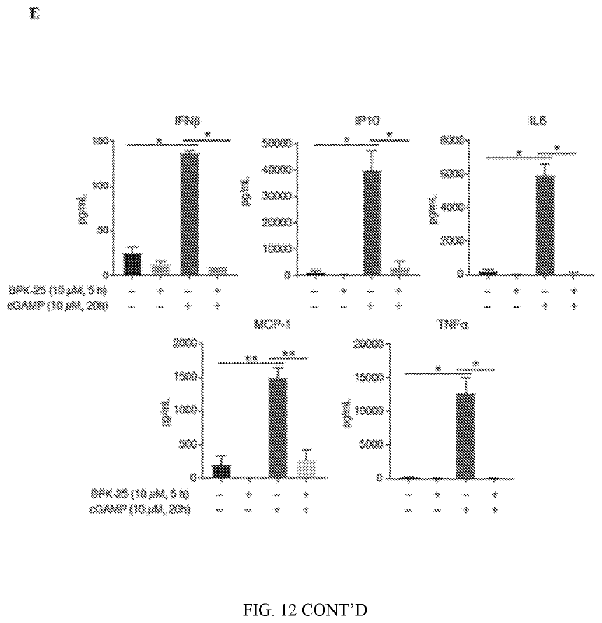

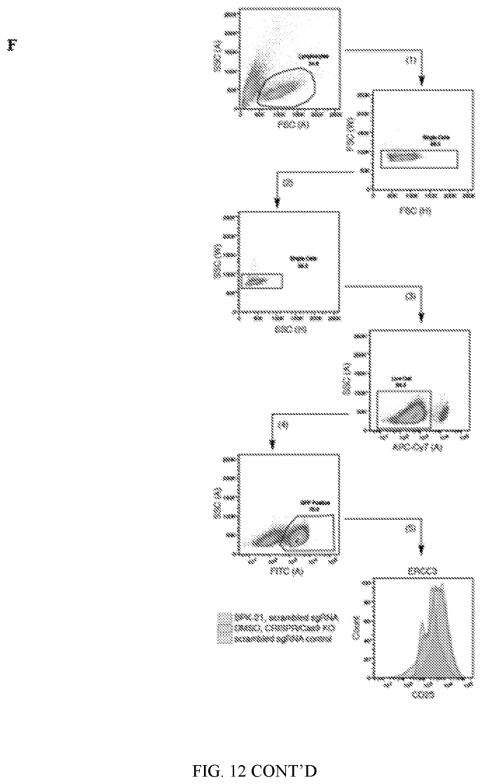

THP-1 Lucia ISG cells were resuspended in low-serum growth media (2% FBS) at a density of 5×10⁵ cells/mL and treated with BPK-25 or vehicle (DMSO) in the presence of viral dsDNA (2 μg/mL). 50 μL of cells were seeded into each well of a 384-well white greiner plates and incubated for 24 h. To evaluate expression of the luciferase reporter, 30 μL of Quanti-luc (Invivogen) detection reagent was added to each well and luminescence was read using an Envision plate reader (Perkin Elmer) set with an integration time of 0.1 seconds. To evaluate cell viability, 30 μL of CellTiter-Glo (Promega) reagent was added to each well and each plate was read using the same instrument settings utilized for the luciferase assay. For each cell type and assay, luminescence signals for test article samples were normalized to vehicle-treated samples and reported as relative light units (RLU). The corresponding data in FIG. 12C is presented as the mean percentage of DMSO-treated control±SEM, n=3/group.

Bio-Plex Quantification of Secreted Cytokines

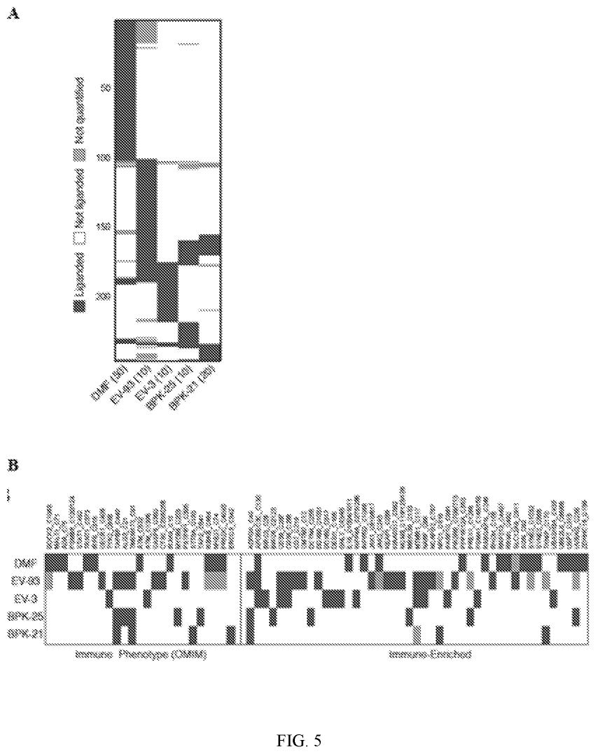

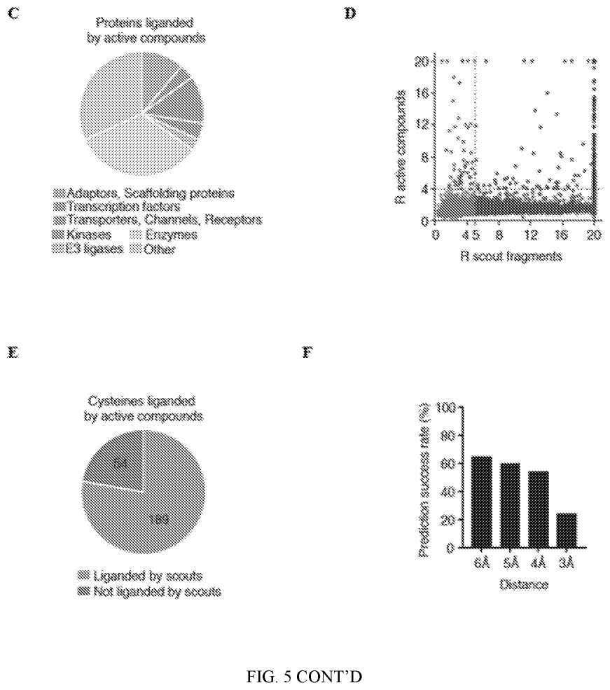

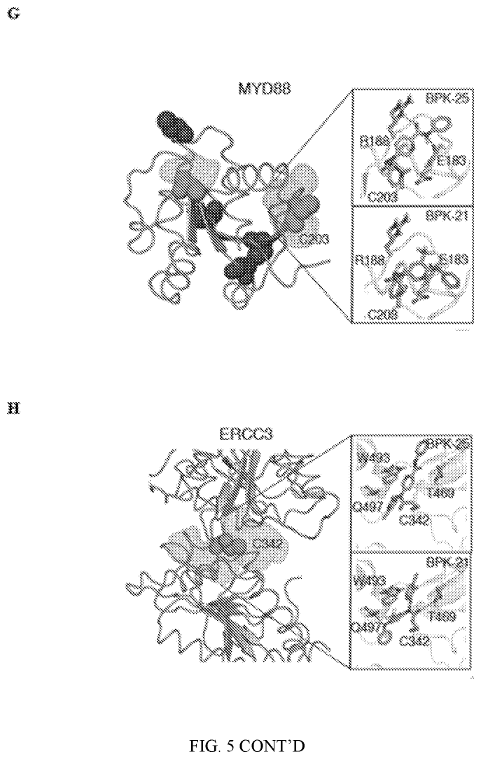

Freshly isolated PBMCs (4×10⁶ cells/mL, 1 mL/well), were treated with BPK-25 (10 μM) or vehicle (DMSO) for 6 h in a 24-well plate, after which cGAMP (10 μM) was added to the wells and the cells were incubated for additional 20 h. Following this treatment, the cells were transferred to 1.5 mL Eppendorf tubes and harvested by centrifugation (600 g, 8 min, 4° C.). The supernatants were saved (−80° C.) and used for further cytokine analysis using Bio-Plex Pro Human Cytokine assay (Bio-Rad) according to manufacturer's instructions. Bio-Plex Assay is a multiplex flow immunoassay that simultaneously detects and identifies cytokines based on fluorescent dye-labeled 6.5 μm magnetic beads in a single reaction. When run on the Bioplex 200 system, 50 IA of supernatant was mixed with 50 μL of beads and quantified against human cytokines standard curves. The corresponding data in FIG. 5SE is presented as the mean percentage of DMSO-treated control±SEM, n=3/group.

ELISA quantification of secreted IFN-β

Concentrations of IFN-β were determined with VeriKine-HS human IFN-β serum ELISA kit (PBL Assay Science) according to manufacturer's instructions. All concentrations of IFN-β were calculated according to the standard curve generated for each experiment. The corresponding data in FIG. 5SE is presented as the mean percentage of DMSO-treated control±SD, n=2/group.

Western Blot Analysis

Western blot analysis was performed on freshly isolated or expanded T cells. For Western blot protein degradation analysis, primary human T cells (2×10⁷ cells/treatment) were re-suspended in RPMI media at 2×10⁶ cells/mL and treated with the compounds or DMSO at 37° C. in a CO₂ containing incubator for 24 h (or otherwise indicated times). Following this incubation period, the cells were pelleted (600 g, 5 min, 4° C.), washed with PBS (10 mL), transferred to 1.5 mL Eppendorf tubes and flash-frozen until further analysis. On the day of the analysis, the cell pellets were thawed on ice, re-suspended in cold PBS and lysed by sonication with probe sonicator (2×8 pulses). Protein concentrations for all the samples were adjusted to 1 mg/mL, 4× loading buffer was added (10 μL to 30 μL of proteome), and the samples were heated at 95° C. for 5 min. The proteins were resolved using SDS-PAGE (10% acrylamide gel) and transferred to 0.45 μM nitrocellulose membranes (GE Healthcare). The membrane was blocked with 5% milk in Tris-buffered saline with tween (TBST) buffer (0.1% Tween 20, 20 mM Tris-HCl 7.6, 150 mM NaCl) at rt for 1 h (or at 4° C. overnight), washed 3 times with TB ST, and incubated with primary antibodies in 5% BSA in TBST at 4° C. overnight. Following another TBST wash (3 times), the membrane was incubated with secondary antibody (1:5,000 in 5% milk in TBST) at 4° C. overnight. The membrane was washed with TBST (3 times), developed with ECL western blotting detection reagent kit (Thermo Scientific) and recorded on CL-X Posure film (Thermo Scientific). Relative band intensities were quantified using ImageJ software. (Rasband, W. S., ImageJ, U. S. National Institutes of Health, Bethesda, Maryland, USA, https://imagej.nih.gov/ij/, 1997-2018.)

Western Blot Analysis of Chromatin-Bound Proteins

BPK-25 (10 μM) treated expanded T cells were washed with PBS before permeabilization by rotation at 4° C. for 10 min with cytoplasm lysis buffer (10 mM sodium phosphate pH 7.4, 25 mM KCl, 1.5 mM MgCl2, 10% glycerol, and 0.025% NP40 supplemented with 1×HALT protease inhibitor cocktail (Thermo Scientific)). Nuclei were pelleted (500 g, 5 min), and washed with cytoplasm lysis buffer without detergent, before being lysed by gentle sonication (Branson Sonifier 250) in cell lysis buffer (10 mM sodium phosphate pH 7.4, 25 mM KCl, 1.5 mM MgCl2, 10% glycerol, and 1% NP40, 0.1% SDS supplemented with 1×HALT, and 1× Benzoase (Pierce)) and rotated for 2 h at 4° C. Insoluble material was precipitated by centrifugation (12,000 g, 10 mM) and the protein concentration of nuclear extracts was measured using standard BCA assay (Thermo Scientific) and normalized. Electrophoretic separation was performed on Novex 4-20% Tris-Glycine Mini Gels (Invitrogen) using the Novex Wedgewell system, and transferred to 0.45 μM Nitrocellulose membranes (GE Healthcare). Primary antibodies were applied overnight at 4° C. in 5% BSA/TBST. Blots were imaged using fluorescence-labeled secondary antibodies (LI-COR) on the Odyssey CLx Imager. Relative band intensities were quantified using ImageJ software.

Gene Expression (qPCR) Analysis

Total RNA from compound or DMSO treated T cells (1.5×10⁷ cells/group) was isolated using RNeasy Mini Kit (Qiagen) according to manufacturer's protocol. RNA concentration was determined using NanoDrop and adjusted to 1 μg RNA in 15 μL RNAse free water for the reverse transcription reaction. cDNA amplification was done using iScript Reverse Transcription Supermix kit (BioRad) according to manufacturer's instructions. The following PCR settings were used for the reverse transcription reaction: 5 min at 25° C. (priming), 20 min at 46° C. (Reverse transcription), 1 min at 95° C. (RT inactivation), hold at 4° C. qPCR analysis was performed on ABI Real Time PCR System (Applied Biosystems) with the SYBR green Mastermix (Applied Biosystems). Relative gene expression was normalized to actin.

qPCR primers used (5′ to 3′):

| | |
|---|---|
| actin-fwd | AGAGCTACGAGCTGCCTGAC |
| actin-rev | AGCACTGTGTTGGCGTACAG |
| BIRC2-fwd | AGCACGATCTTGTCAGATTGG |
| BIRC2-rev | GGCGGGGAAAGTTGAATATGTA |
| BIRC3-fwd | AAGCTACCTCTCAGCCTACTTT |
| BIRC3-rev | CCACTGTTTTCTGTACCCGGA |
| IL6-fwd | AATTCGGTACATCCTCGACGG |
| IL6-rev | GGTTGTTTTCTGCCAGTGCC |
| IL1-beta-fwd | ACAGATGAAGTGCTCCTTCCA |
| IL1-beta-rev | GTCGGAGATTCGTAGCTGGAT |
| CXCL10-fwd | CCAGAATCGAAGGCCATCAA |
| CXCL10-rev | CATTTCCTTGCTAACTGCTTTCAG |

RNA Sequencing

Total RNA from compound or DMSO treated T cells (1.5×10⁷ cells/group) was isolated using RNeasy Mini Kit (Qiagen) using RNAse free DNAse set (Qiagen) for on column DNA digestion according to manufacturer's protocol and stored at −80° C. until further analysis.

RNA quality was assessed using TapeStation 4200 and RNA-Seq libraries were prepared using the TruSeq stranded mRNA Sample Preparation Kit v2 according to Illumina protocols. Multiplexed libraries were validated using TapeStation 4200, normalized, pooled and quantified by qPCR for sequencing. High-throughput sequencing was performed on the NextSeq 500 system (Illumina). Image analysis and base calling were done with Illumina CASAVA-1.8.2.

Sequenced reads were quality-tested using FASTQC Andrews S. (2010). FastQC: a quality control tool for high throughput sequence data. Available online at: http//www.bioinformatics.babraham.ac.uk/projects/fastqc and aligned to the hg19 human genome using the STAR (Dobin et al., 2013) version 2.5.3a. Mapping was carried out using default parameters (up to 10 mismatches per read, and up to 9 multi-mapping locations per read). The genome index was constructed using the gene annotation supplied with the hg19 Illumina iGenomes (iGenomes online. Illumina. 2015. http://support.illumina.com/sequencing/sequencing_software/igenome.html) collection and sjdbOverhang value of 100. Homer (Heinz et al., 2010) v4.10.4 was used to calculate the fragments per kilobase per million mapped reads (FPKM) normalized gene expression across all exons with the top expressed isoform reported.

isoTOP ABPP Sample Preparation

Activated or expanded primary human T cells were re-suspended in RPMI (1×10⁶ cells/mL) containing 10% fetal bovine serum (FBS), penicillin (100 U/mL), streptomycin (100 mg/mL), and L-glutamine (2 mM). The cells were treated with DMSO or compounds for 3 h, pelleted (524 g, 5 min), washed with PBS, and lysed by sonication (2×8 pulses). Soluble and particulate proteomic fractions were separated by ultracentrifugation (100,000 g, 45 min), and protein concentration was normalized to 1.7 mg/mL using a standard DC protein assay (Bio-Rad). The resulting proteomes were analyzed by competitive isotopic Tandem Orthogonal Proteolysis Activity-Based Protein Profiling (isoTOP-ABPP).

IA-Alkyne Labeling and Click Chemistry

Samples (500 μL, 1.7 mg/mL) were treated with iodoacetamide alkyne (IA-alkyne, 5 μL of 10 mM stock in DMSO, final concentration: 100 μM) for 1 h at ambient temperature. Modified proteins were then conjugated to isotopically labeled, TEV-cleavable biotin tags (TEV-tags) using copper-catalyzed azide-alkyne cycloaddition reaction (CuAAC). Reagents for the CuAAC reaction were pre-mixed prior to their addition to the proteome samples. TEV tags (light or heavy, 10 μL of 5 mM stocks in DMSO, final concentration=100 μM), tris(benzyltriazolylmethyl)amine ligand (TBTA; 30 μL of 1.7 mM stock in DMSO:t-butanol 1:4, final concentration=100 μM), tris(2-carboxyethyl)phosphine hydrochloride (TCEP; 10 μL of fresh 50 mM stock in water, final concentration=1 mM), and Cu(OAc)₂ (10 μL of 50 mM stock in water, final concentration=1 mM) were combined in an Eppendorf tube, vortexed and added to the proteomes (55 μL/sample). “Heavy” CuAAC reaction mixture was added to the DMSO treated control samples and “light”—to compound-treated samples. The reaction was allowed to proceed at rt for 1 h, “heavy” and “light” samples were combined pairwise in 15 mL conical Falcon tubes kept on ice containing 4 mL of cold methanol (pre-chilled at −80° C.), 1 mL CHCl₃, and 1 mL H₂O. Eppendorf tubes from the reaction mixtures were washed with additional H₂O (1 mL each) and the washes were added to the same Falcon tube (final ratios MeOH:CHCl₃:H₂O=4:1:4). Following centrifugation (5,000 g, 10 min, 4° C.), a protein disk formed at the interface of CHCl₃ and aqueous layers. Both layers were aspirated without perturbing the disk, which was resuspended in cold MeOH (2 mL) and CHCl₃ (1 mL) by vortexing. The proteins were pelleted (5,000 g, 10 min, 4° C.), and the resulting pellets were solubilized in 1.2% SDS in PBS (1 mL) with sonication and heating (95° C., 5 min).

isoTOPABPP Sample Streptavidin Enrichment

Once solubilized, the samples were diluted with PBS (4 mL) and streptavidin-agarose beads were added for the enrichment (final SDS concentration: 0.2% in PBS). The beads (100 μL of a 50% slurry per sample) were washed with PBS (2×10 mL) and resuspended in 1 mL of PBS per sample prior to addition. The final mixture was rotated for 3 h at rt. Following this enrichment step, the beads were pelleted by centrifugation (2,000 g, 2 min) and extensively washed to remove non-specifically binding proteins (2×10 mL 0.2% SDS in PBS, 2×10 mL PBS, and 2×10 mL H₂O).

isoTOPABPP Sample Trypsin and TEV Digestion

After the last wash, the beads were transferred to new Eppendorf tubes in water (2×0.5 mL), pelleted (4,000 g, 3 min), and resuspended in 6M urea in PBS (0.5 mL). DTT (25 μL of a fresh 200 mM stock in water, final concentration −10 mM) was added and the beads were incubated at 65° C. for 15 min. Iodoacetamide (25 μL of a 400 mM stock in water, final concentration −20 mM) was then added and the samples were incubated in the dark at 37° C. with shaking for 30 min. Following this incubation, the mixture was diluted with PBS (900 μL), the beads were pelleted by centrifugation and resuspended in 2M urea in PBS (200 μL). Trypsin (Promega, sequencing grade; 2 μg in 6 μL of trypsin buffer containing 1 mM CaCl₂)) was added to the mixture and the digestion was allowed to proceed overnight at 37° C. with shaking. The beads were pelleted (2,000 g, 2 min) and the tryptic digest was aspirated. The beads were then extensively washed (3×1 mL PBS, 3×1 mL H₂O), transferred to a new Eppendorf tube in H₂O (2×0.5 mL), washed with TEV buffer (200 μL, 50 mM Tris, pH 8, 0.5 mM EDTA, 1 mM DTT), and resuspended in TEV buffer (140 μL). TEV protease (4 μL, 80 μM) was then added and the beads were incubated at 30° C. overnight with rotation. Following the overnight digestion, the beads were pelleted by centrifugation (2,000 g, 2 min) and the TEV digest was separated from the beads using Micro Bio-Spin coulumns (Bio-rad) with centrifugation (800 g, 0.5 min) and an additional wash (100 μL H₂O). The samples were then acidified by the addition of 0.1% FA (14 μL, final concentration: 5% v/v) and stored at −80° C. prior to analysis.

isoTOPABPP Liquid-Chromatography-Mass-Spectrometry (LC-MS/MS) Analysis

Samples were pressure-loaded onto a 250 μm (inner diameter) fused silica capillary columns packed with C₁₈ resin (Aqua 5 Phenomenex) and analyzed by multidimensional liquid chromatography tandem mass-spectrometry (MudPIT) using an LTQ-Velos Orbitrap mass spectrometer (Thermo Scientific) coupled to an Agilent 1200-series quaternary pump. The peptides were eluted onto a biphasic column with a 5 μm tip (100 μm fused silica, packed with C₁₈ (10 cm) and bulk strong cation exchange resin (3 cm, SCX, Phenomenex) in a 5-step MudPIT experiment, using 0%, 30%, 60%, 90%, and 100% salt bumps of 500 mM aqueous ammonium acetate and a 5%-100% gradient of buffer B in buffer A (buffer A: 95% water, 5% CH₃CN, 0.1% FA; buffer B: 5% water, 95% CH 3 CN, 0.1% FA) as previously described (Weerapana et al., 2007). Data were collected in data-dependent acquisition mode with dynamic exclusion enabled (20 s, repeat of 2). One full MS (MS1) scan (400-1800 m/z) was followed by 30 MS2 scans (ITMS) of the nth most abundant ions.

isoTOP ABPP Peptide Identification

The MS2 spectra data were extracted from the raw file using RAW Converter (version 1.1.0.22; available at http://fields.scripps.edu/rawconv/), uploaded to Integrated Proteomics Pipeline (IP2), and searched using the ProLuCID algorithm (publicly available at http://fields.scripps.edu/downloads.php) using a reverse concatenated, non-redundant variant of the Human UniProt database (release-2012_11). Cysteine residues were searched with a static modification for carboxyamidomethylation (+57.02146) and up to one differential modification for either the light or heavy TEV tags (+464.28595 or +470.29976 respectively). Peptides were required to have at least one tryptic terminus and to contain the TEV modification. ProLuCID data was filtered through DTASelect (version 2.0) to achieve a peptide false-positive rate below 1%.

isoTOP ABPP R Value Calculation and Data Processing

The heavy/light isoTOP-ABPP ratios (R values) for each unique peptide (DMSO/compound treated) were quantified with in-house CIMAGE software (Weerapana et al., 2010) using default parameters (3 MS1 acquisitions per peak and signal to noise threshold set to 2.5). Site-specific engagement of cysteine residues was assessed by blockade of IA-alkyne probe labeling. A maximal ratio of 20 was assigned for peptides that showed a ≥95% reduction in MS1 peak area in the compound treated proteome (light TEV tag) compared to the control DMSO-treated proteome (heavy TEV tag). Ratios for unique peptide sequences were calculated for each experiment; overlapping peptides with the same modified cysteine (e.g., different charge states, elution times or tryptic termini) were grouped together and the median ratio was reported as the final ratio (R). Additionally, ratios for peptide sequences containing multiple cysteines were grouped together. When aggregating data across experimental replicates, the mean of each experimental median R was reported. The peptide ratios reported by CIMAGE were further filtered to ensure the removal or correction of low-quality ratios in each individual dataset. The quality filters applied were the following: removal of half-tryptic peptides, removal of peptides with more than one tryptic miscleaved, removal of peptides with R=20 and only a single MS2 event triggered during the elution of the parent ion, removal of non-unique peptides. Further filtering was then performed as described below for each experiment type.

Combining Data Across Experimental Groups

To combine data across replicates from different experiment groups (e. g., broad ligandability and elaborated fragment data or hyper-reactivity) or different experiment types (e. g., TMT and isoTOP), identifiers consisting of the Uniprot accession concatenated with the tryptic sequence associated with the particular peptide were used. Peptides that contained the same modified cysteine or where multiple cysteines were modified on that peptide were combined. When data from an experiment group associated with a miscleaved peptide sequence was combined with data from another group which contained a non miscleaved variant of the same peptide, all data was reported under the fully tryptic identifier, unless the non miscleaved variant introduced an additional cysteine, in which case the data was not merged.

Filtering of Broad Ligandability (Scout Fragment) Data

All peptides with R=20 were manually reviewed. Peptides with R=20 were discarded if the ratio set contained a single 20, and the minimum ratio in the set was less than 4. If the ratio set contained two or more 20 values and the minimum ratio in the set was less than 2, these 20 values were also discarded. This filter was applied on R values within a single experiment and when aggregating data from replicate experiments.

When aggregating data from replicate experiments, for peptides that had standard deviations greater than 60% of the mean, the lowest ratio of that set was reported, unless the minimum ratio of the set was ≥4, in which case the average ratio was reported. Individual peptide sequences were required to have been quantified (R≠0) in at least two replicates per condition. Peptides were considered liganded if they had a final value of R≥5.

Filtering of Elaborated Compound Data

All peptides with R=20 were manually reviewed. Within individual replicates, peptides with R=20 were discarded if the ratio set contained a single 20, and the minimum ratio in the set was less than 4. When aggregating ratios across replicates, peptides with R=20 were discarded if the ratio set contained a single 20, and the minimum ratio in the set was less than 3. Individual peptide sequences were required to have been quantified (R≠0) in at least two replicates per condition, unless they had R≥4 in both particulate and soluble conditions for a given compound.

During manual review of the data, some peptides were exempted from specific filters due to additional evidence of their validity. BIRC2 (C45) R=20 and CASP2 (C366, C370) R=20 values derived from DMF datasets values derived from DMF datasets were exempted from applied 20-filters as the same residues were convincingly liganded in the TMT datasets.

Peptides were considered liganded if they had a final value of R≥4.

Filtering and Processing of Hyper-Reactivity Data

Peptides with R=20 were discarded if the ratio set contained a single 20, and the minimum ratio in the set was less than 4. This filter was applied on R values within a single experiment and when aggregating data from replicate experiments.

Data from these experiments was separated according to activation state and the minimal ratio between soluble and particulate fractions for each state was reported for each peptide.

TMT-ABPP Sample Preparation and IA-DTB Labeling

Samples (500 μL, 1.7 mg/mL) were treated with iodoacetamide desthiobiotin (IA-DTB, 5 μL of 10 mM stock in DMSO, final concentration: 100 μM) for 1 h at ambient temperature. Ice-cold MeOH (500 μL) and CHCl₃ (200 μL) were then added, the mixture was vortexed and centrifuged (10,000 g, 10 min, 4° C.) to afford a protein disc at the interface of CHCl₃ and aqueous layers. Both layers were aspirated without perturbing the disk, which was re-suspended in cold methanol (500 μL) and CHCl₃ (200 μL) by sonication. The proteins were pelleted (10,000 g, 10 min, 4° C.), and the resulting pellets were re-suspended in a buffer (90 μL) containing 9M urea, 10 mM DTT and 50 mM triethylammonium bicarbonate (1/20 dilution of 1.0 M stock solution, pH 8.5) by thorough pipetting up and down. The resulting mixture was heated at 65° C. for 20 min. Sample was cooled to room temp, iodoacetamide (10 μL, 500 mM solution in H₂O) was added, and the samples were incubated at 37° C. for 30 min with shaking.

TMT-ABPP Trypsin Digestion and Streptavidin Enrichment

Following the labeling with iodoacetamide, samples were diluted with 305 μL of triethylammonium bicarbonate buffer (50 mM, 1/20 dilution of 1.0 M stock, pH 8.5; Final urea concentration: 2.0 M). Trypsin (4 μL of 0.25 μg/μL trypsin in trypsin buffer, containing 25 mM CaCl₂) was then added and the proteins were digested at 37° C. overnight. The following day, samples were diluted with wash buffer (400 IA, 25 mM Tris-HCl, pH 7.5, 150 mM NaCl, 0.2% NP-40), streptavidin-agarose beads (50% slurry in wash buffer) were added to each sample (40 μL/sample) and the bead mixture was rotated for 2 h at rt. Briefly, for a 10-plex sample, streptavidin-agarose bead slurry (440 μL, 50% slurry) was washed (2×1 mL, 25 mM Tris-HCl, pH 7.5, 150 mM NaCl, 0.1% NP-40) and brought up to the initial volume in the wash buffer prior to the addition to the sample. After incubation, the beads were pelleted by centrifugation (2,000 g, 1 min), transferred to a BioSpin column and washed extensively (3×1 mL wash buffer, 3×1 mL PBS, 3×1 mL H₂O). Peptides were eluted by the addition of 300 μL of 50% aqueous CH₃CN containing 0.1% FA. The eluate was then evaporated to dryness using SpeedVac vacuum concentrator.

TMT Tag Labeling

Peptides were resuspended in 100 μL EPPS buffer (200 mM, pH 8.0) with 30% dry CH₃CN, vortexed and spun down (2,000 g, 1 min). TMT tags (3 μL/tube in dry CH₃CN, 20 μg/μL) were added to the corresponding tubes and the reaction was allowed to proceed for 1 h 15 min. The reaction was quenched by the addition of 5% hydroxylamine (3 μL per sample), vortexed and left at room temperature for 15 min. FA (5 μL) was then added to each tube, the tubes were vortexed, spun down and combined in a low binding 1.5 mL Eppendorf tube. The final combined sample was dried in a SpeedVac vacuum concentrator and kept at −80° C. until the high pH fractionation step.

High pH Fractionation

The spin columns for high pH fractionation were pre-equilibrated prior to use. Briefly, the columns were placed in Eppendorf tubes (2 mL), spun down to remove the storage solution (5,000 g, 2 min), and washed with CH 3 CN (2×300 μL, 5,000 g, 2 min) and buffer A (2×300 μL, 95% H₂O, 5% CH 3 CN, 0.1% FA, 5,000 g, 2 min). TMT labeled peptides were re-dissolved in buffer A (300 μL, 95% H₂O, 5% CH₃CN, 0.1% FA) and loaded onto pre-equilibrated spin columns for high pH fractionation. The columns were spun down (2,000 g, 2 min) and the flow through was used to wash the original Eppendorf tube and passed through the spin column again (2,000 g, 2 min). The column was then washed with buffer A (300 μL, 2,000 g, 2 min) and 10 mM aqueous NH₄HCO₃ containing 5% CH 3 CN (300 μL, 2,000 g, 2 min), and the flow through was discarded. The peptides were eluted from the spin column into fresh Eppendorf tubes (2.0 mL) with a series of NH₄HCO₃/CH₃CN buffers (2000 g, 2 min). The following buffers were used for peptide elution:

| Fraction | Acetonitrile | Acetonitrile | 10 mM NH₄HCO₃ |
|---|---|---|---|
| Number | (%) | (μL) | (μL) |
| 1 | 7.5 | 75 | 925 |
| 2 | 10.0 | 100 | 900 |
| 3 | 12.5 | 125 | 875 |
| 4 | 15.0 | 150 | 850 |
| 5 | 17.5 | 175 | 825 |
| 6 | 20.0 | 200 | 800 |
| 7 | 22.5 | 225 | 775 |
| 8 | 25.0 | 250 | 750 |
| 9 | 27.5 | 275 | 725 |
| 10 | 30.0 | 300 | 700 |
| 11 | 32.5 | 325 | 675 |
| 12 | 35.0 | 350 | 650 |
| 13 | 37.5 | 375 | 625 |
| 14 | 40.0 | 400 | 600 |
| 15 | 42.5 | 425 | 575 |
| 16 | 45.0 | 450 | 550 |
| 17 | 47.5 | 475 | 525 |
| 18 | 50.0 | 500 | 500 |
| 19 | 52.5 | 525 | 475 |
| 20 | 55.0 | 550 | 450 |
| 21 | 75.0 | 750 | 250 |

Every 7th fraction was combined into a new clean Eppendorf tube (2 mL) and the solvent was removed using SpeedVac vacuum concentrator. The resulting 7 combined fractions were re-suspended in buffer A (10 μL) and analyzed on the Orbitrap Fusion mass-spectrometer (5 μL injection volume).

Whole Proteome TMT (TMT-Exp) Sample Preparation

Freshly isolated T cells (1.6×10⁷ cells, 2×10⁶ cells/mL in RPMI media) were treated with compound or DMSO for 24 h, pelleted (600 g, 5 min), and washed with PBS (1×10 mL). The cells were then transferred to an Eppendorf tube in additional PBS (1 mL), pelleted (600 g, 5 min), flash frozen, and kept at −80° C. until further analysis. On the first day of the whole proteome TMT protocol, the cells were thawed on ice and lysed in lysis buffer (150 μL, 1 tablet of Roche complete, mini, EDTA-free Protease Inhibitor Cocktail dissolved in 10 mL of PBS) using a probe sonicator (2×8 pulses). Protein concentration was adjusted to 2.0 mg/mL and the samples (100 μL) were transferred to new Eppendorf tubes (1.5 mL) containing urea (48 mg/tube, final urea concentration: 8 M). DTT (5 μL, 200 mM fresh stock in H₂O, final DTT concentration: 10 mM) was then added to the tubes and the samples were incubated at 65° C. for 15 min. Following this incubation, iodoacetamide (5 μL, 400 mM fresh stock in H₂O, final IA concentration: 20 mM) was added and the samples were incubated in the dark at 37° C. with shaking for 30 min. Ice-cold MeOH (600 μL), CHCl₃ (200 μL), and H₂O (500 μL) were then added, the mixture was vortexed and centrifuged (10,000 g, 10 min, 4° C.) to afford a protein disc at the interface of CHCl₃ and aqueous layers. The top layer was aspirated without perturbing the disk, additional MeOH (600 μL) was added and the proteins were pelleted (10,000 g, 10 min, 4° C.) and used in the next step or stored at −80° C. overnight.

Whole Proteome TMT LysC and Trypsin Digestion

The resulting protein pellets were resuspended in EPPS buffer (160 μL, 200 mM, pH 8) using a probe sonicator (2×6 pulses). LysC solution (4 μL/sample, 20 μg in 40 μL of HPLC grade water) was added and the samples were incubated at 37° C. with shaking for 2 h. Trypsin (10 μL, 0.5 μg/4 in trypsin buffer) and CaCl₂) (1.8 μL, 100 mM in H2O) were then added and the samples were incubated at 37° C. with shaking overnight.

Whole Proteome TMT Labeling with TMT Tags

The sample became clear after the overnight digestion. At this point, peptide concentration was determined using microBCA assay (Thermo Scientific) according to manufacturer's instructions. For each sample, a volume corresponding to 25 μg of peptides was transferred to a new Eppendorf tube and the total volume was brought up to 35 μL with EPPS buffer (200 mM, pH 8). The samples were diluted with CH₃CN (9 μL) and incubated with the corresponding TMT tags (3 μL/sample, 20 μg/μL) at rt for 30 min. The TMT tag treatment (3 μL/sample, 20 μg/μL, 30 min) was repeated, after which the tags were quenched by the addition of hydroxylamine (6 μL, 5% in H₂O). Following a 15 min incubation at it, formic acid was added (2.5 μL, final FA concentration: 5%) and the samples were stored at −80° C. until further analysis.

Whole Proteome TMT Ratio Check and High pH Fractionation

Small aliquots (2 μL) from each channel were combined in a separate Eppendorf tube and dried using SpeedVac vacuum concentrator. The residue was re-dissolved in Buffer A (20 μL) and desalted using C18 stage tips (made in-house using 200 μL pipette tips and C18 discs (3M Empore)). Briefly, the stage-tip was activated by passing MeOH (2×50 μL) through the stage tip and washed with Buffer B (2×50 μL, 5% H₂O, 95% CH₃CN, 0.1% FA), followed by Buffer A (2×50 μL, 5% CH₃CN/95% H₂O, 0.1% FA). The sample was then loaded and the stage-tip was washed with Buffer A. The sample was eluted into a new Eppendorf tube with Buffer B (2×50 μL) and dried using SpeedVac vacuum concentrator. The residue was re-dissolved in Buffer A (10 μL) and analyzed by mass-spectrometry using the following LC-MS gradient: 5% buffer B in buffer A from 0-15 min, 5-15% buffer B from 15-17.5 min, 15-35% buffer B from 17.5-92.5 min, 35-95% buffer B from 92.5-95 min, 95% buffer B from 95-105 min, 95-5% buffer B from 105-107 min, and 5% buffer B from 107-125 min (buffer A: 95% H₂O, 5% CH 3 CN, 0.1% FA; buffer B: 5% H₂O, 95% CH₃CN, 0.1% FA) and standard MS3-based quantification described below. Ratios were determined from the average peak intensities corresponding to each channel. For a ten-plex experiment, samples (20 μL/channel, final volumes adjusted based on the determined ratios) were combined in a new low binding Eppendorf tube (1.5 mL) and dried using SpeedVac. The residue was subjected to high pH fractionation as described above to yield 7 fractions which were re-suspended in buffer A (24 μL/sample) and analyzed by liquid chromatography tandem mass-spectrometry.

TMT-ABPP and Whole Proteome TMT Liquid Chromatography-Mass-Spectrometry (LC-MS) Analysis

Samples were analyzed by liquid chromatography tandem mass-spectrometry using an Orbitrap Fusion mass spectrometer (Thermo Scientific) coupled to an UltiMate 3000 Series Rapid Separation LC system and autosampler (Thermo Scientific Dionex). The peptides were eluted onto a capillary column (75 μm inner diameter fused silica, packed with C18 (Waters, Acquity BEH C18, 1.7 μm, 25 cm) and separated at a flow rate of 0.25 μL/min using the following gradient: 5% buffer B in buffer A from 0-15 min, 5-35% buffer B from 15-155 min, 35-95% buffer B from 155-160 min, 95% buffer B from 160-169 min, 95-5% buffer B from 169-170 min, and 5% buffer B from 170-200 min (buffer A: 95% H₂O, 5% acetonitrile, 0.1% FA; buffer B: 5% H₂O, 95% CH 3 CN, 0.1% FA). The voltage applied to the nano-LC electrospray ionization source was 1.9 kV. Data was acquired using an MS3-based TMT method adapted from (Wang, Y. et al., 2019) Briefly, the scan sequence began with an MS1 master scan (Orbitrap analysis, resolution 120,000, 400-1700 m/z, RF lens 60%, automatic gain control [ΔGC] target 2E5, maximum injection time 50 ms, centroid mode) with dynamic exclusion enabled (repeat count 1, duration 15 s). The top ten precursors were then selected for MS2/MS3 analysis. MS2 analysis consisted of: quadrupole isolation (isolation window 0.7) of precursor ion followed by collision-induced dissociation (CID) in the ion trap (ΔGC 1.8E4, normalized collision energy 35%, maximum injection time 120 ms). Following the acquisition of each MS2 spectrum, synchronous precursor selection (SPS) enabled the selection of up to 10 MS2 fragment ions for MS3 analysis. MS3 precursors were fragmented by HCD and analyzed using the Orbitrap (collision energy 55%, ΔGC 1.5E5, maximum injection time 120 ms, resolution was 50,000). For MS3 analysis, we used charge state—dependent isolation windows. For charge state z=2, the MS isolation window was set at 1.2; for z=3-6, the MS isolation window was set at 0.7. The MS2 and MS3 files were extracted from the raw files using RAW Converter (version 1.1.0.22; available at http://fields.scripps.edu/rawconv/), uploaded to Integrated Proteomics Pipeline (IP2), and searched using the ProLuCID algorithm (publicly available at http://fields.scripps.edu/downloads.php) using a reverse concatenated, non-redundant variant of the Human UniProt database (release-2012_11). Cysteine residues were searched with a static modification for carboxyamidomethylation (+57.02146) and up to one differential modification for the desthiobiotin (DTB) tag (+398.2529). N-terminus and lysine were also searched with a static modification corresponding to the TMT tag (+229.1629). Peptides were required to be at least 6 amino acids long, to have at least one tryptic terminus, and to contain the DTB modification. ProLuCID data was filtered through DTASelect (version 2.0) to achieve a peptide false-positive rate below 1%. The MS3-based peptide quantification was performed with reporter ion mass tolerance set to 20 ppm with Integrated Proteomics Pipeline (IP2).

TMT-ABPP R Value Calculation for Broad Ligandability Data

At individual TMT experiment level, the following filters were applied to remove low-quality peptides: removal of non-unique peptides; removal of half-tryptic peptides; removal of peptides with more than one internal miscleaved sites; removal of peptides with low (<20,000) sum of reporter ion intensities for either expanded or activated control channels; removal of peptides with high variation between the replicate control channels (coefficient of variance >0.5), and peptides corresponding to the lower average reporter ion intensity control channels (activated vs expanded) if the difference in the average reporter ion intensity between expanded and activated control channels was more than two-fold. R-value (DMSO-treated vs. KB02/KB05-treated) for each peptide entry was calculated using the reporter ion intensities of DMSO and KB02/KB05 treated TMT channels for each treatment group with a maximum ratio cap of 20. Once the R values were calculated, two types of grouping were performed to aggregate peptide quantification data: 1) overlapping peptides with the same modified cysteine (e.g., different charge states, high pH fractionation fractions, or tryptic termini) were grouped together, then their R values were averaged, and the shortest unique tryptic peptide was reported; 2) multiple modified cysteines on a tryptic peptide were grouped together, then the averaged R values were used for further data processing. Peptides with high donor variation (R>5 for one donor, while R<2 for the other donor) were discarded (<1%), then the R values of replicate channels of the same condition were averaged to obtain the final reported data. A cysteine was required to be quantified in at least two TMT channels for each proteomic fraction to be reported.

TMT-ABPP R Value Calculation for Elaborated Compounds Dataset

At individual TMT experiment level, the following filters were applied to remove low-quality peptides: removal of non-unique peptides; removal of half-tryptic peptides; removal of peptides with more than one internal miscleaved site; removal of peptides with low (<10,000) sum of reporter ion intensities for control channels, and peptides with high variation between the replicate control channels (coefficient of variance >0.5). R values (compound-treated vs. DMSO-treated) for each peptide entry were calculated using the reporter ion intensities of DMSO and compound treated TMT channels for each treatment group with a maximum ratio cap of 20. Once the R values for each peptide entry were calculated, two types of grouping were performed to aggregate peptide quantification data: 1) overlapping peptides with the same modified cysteine (e.g., different charge states, high pH fractionation fractions, or tryptic termini) were grouped together, then their R values were averaged, and the shortest unique tryptic peptide was reported; 2) multiple modified cysteines on a tryptic peptide were grouped together, then the averaged R values were used for further data processing. The R values of replicate channels of the same condition were averaged to obtain the final reported data with the requirement that all included peptides have been quantified in at least two individual experiments.

Whole Proteome Protein Ratios Calculation for Elaborated Compounds Dataset

At individual TMT experiment level, the following filters were applied to remove low-quality peptides: removal of non-unique peptides; removal of half-tryptic peptides; removal of peptides with more than one internal miscleaved site; removal of cysteine-containing peptides; removal of peptides with low (<10,000) sum of reporter ion intensities for control channels, and peptides with high variation between the replicate control channels (coefficient of variance >0.5). R values (compound-treated vs. DMSO-treated) were calculated using the reporter ion intensities of compound and DMSO treated TMT channels for each treatment group. Then the ratios of all peptides of a protein were averaged to be reported as the final protein ratio. Proteins were required to have at least two unique quantified peptides in each experiment and were quantified in at least two independent experiments.

Whole Proteome Protein Ratios Calculation for State-Dependent Dataset

The MS3-based peptide quantification was performed with reporter ion mass tolerance set to 20 ppm with Integrated Proteomics Pipeline (IP2). At individual TMT experiment level, the following filters were applied to remove low-quality peptides: removal of non-unique peptides; removal of half-tryptic peptides; removal of peptides with more than one internal miscleaved sites; removal of peptides with low (<10,000) sum of reporter ion intensities (5 channels/donor), and peptides with high variation between either of the replicate channels for expanded or activated T cells (coefficient of variance >0.5). R values (activated vs. expanded) for each peptide entry were calculated using the average reporter ion intensities of activated and expanded TMT channels. Then the ratios of all quantified peptides for a protein were averaged to obtain the final protein ratio. Proteins were required to have at least two unique quantified peptides in each experiment.

TMT-ABPP R Value Calculation for Cysteine State-Dependent Reactivity Dataset

At individual TMT experiment level, the following filters were applied to remove low-quality peptides: removal of non-unique peptides; removal of half-tryptic peptides; removal of peptides with more than one internal miscleaved site; removal of peptides with low (<10,000) sum of reporter ion intensities in both expanded or activated channels; removal of peptides with high variation (coefficient of variance >0.5) between the replicate expanded or activated channels if their sum of reporter ion intensities is greater than 5,000. R values (activated vs. expanded) for each peptide were calculated using the average reporter ion intensities of activated and expanded TMT channels. Once the R values were calculated, two types of grouping were performed to aggregate peptide quantification data: 1) overlapping peptides with the same modified cysteine (e.g., different charge states, high pH fractionation fractions, or tryptic termini) were grouped together, then their R values were averaged, and the shortest unique tryptic peptide was reported; 2) multiple modified cysteines on a tryptic peptide were grouped together, then the averaged R values were reported for further data processing. The median value derived from at least two biological replicates was reported as the final R value for each peptide with a maximum ratio cap of 20.

Data Processing and Analysis for IA-DTB Reactivity Dataset

Proteins must have at least three unique quantified peptides in either particulate or soluble fraction in the TMT-ABPP experiments within the state-dependent dataset to be analyzed. The fraction with the most quantified unique peptides was selected for analysis for each protein. If a protein had an equal number of unique quantified peptides in both fractions, the peptide R ratios (activated vs. expanded) from both fractions were averaged. To account for potential donor variations in protein expression level, proteins were required to have at least one peptide R ratio within 1.5-fold of the protein expression level measured in TMT-exp experiments (if available) and were excluded from the analysis if all peptide R ratios were greater than 2.0 or less than 0.5. For proteins with 5 or more quantified peptides, a cysteine was considered for potential change in reactivity if its peptide R value differed more than two-fold from both the median R value of all quantified cysteines on the same protein and from the protein expression level measured in TMT-exp experiments (if available). For proteins with three or four quantified peptides, a cysteine was considered for potential change in reactivity if its peptide R value differed more than two-fold from the protein expression level measured by TMT-exp data, with an additional requirement that the maximum peptide R ratio differed more than 2-fold from the minimum peptide R ratio. All the cysteines that passed the initial filters described above were manually curated to remove low quality profiles.

Generation of In Vitro Transcribed sgRNAs

DNA templates consisting of a T7 RNA Polymerase promoter, the ˜20 nt target-specific sequence, and the chimeric sgRNA scaffold were generated for each desired target by overlapping PCR using Q5 High Fidelity Master Mix (New England Biolabs) under the following conditions: 98° C. for 2 min; 50° C. for 10 min; 72° C. for 10 min. Guide RNA templates were used to transcribe guide RNAs using the HiScribe T7 High Yield RNA Synthesis kit (New England Biolabs) according to the manufacturer's instructions. Following in vitro transcription, guides were purified using Monarch RNA Cleanup Kit (New England Biolabs) following manufacturer's instructions.

Cas9 Ribonucleoprotein (RNP) Assembly and Electroporation

The Cas9 RNPs were assembled before transfection using the ArciTect™ Cas9-eGFP Nuclease (StemCell) with the T7 transcribed RNAs at a molar ratio of 1:3 in Buffer T. For each target of interest, the genome was tiled with 3 unique guide RNAs. Before the transfection, primary T cells were preactivated on αCD3/αCD28-precoated plates in complete RPMI medium supplemented with 100 U/mL IL2 for 48 h. The T cells were then washed with PBS and resuspended in Buffer T (10×10⁶ cells/mL) and the Cas9 RNP transfections were performed using the Neon Transfection system (ThermoFisher). Following the Cas9 RNP transfection, T cells were cultured in RPMI supplemented with 50 U/mL IL2 for 7 days.

FACS Analysis of T Cell Activation

Seven days post-transfection, the cells were stimulated for a second time using αCD3/αCD28-precoated plates in the presence of IL2 (100 U/mL) for 24 h. Cell surface staining for T cell activation was performed using αCD25-PE (Biolegend) and αCD69-APC (Biolegend) antibodies for 1 h at 4° C. Viable cells gating was performed using eBioscience™ Fixable Viability Dye eFluor™ 780 (ThermoFisher).

### Example 6

Molecular Modeling

Description of the Methods

Two different docking methods based on the software Autodock (Morris et al., 2009) were used: the reactive docking and the flexible side chain covalent docking. The reactive docking is a predictive method that allows to identify the residues most likely to be modified by covalent binding. This was accomplished in two steps: first, it performs a scanning of all solvent accessible residues of a given type (cysteines, in this case), then it applies conventional, untethered docking with a special potential to simulate the incipient reaction to identify the most likely ones to be modified by the ligands. Reactive docking was successfully applied in previous studies, where it was used to model electrophile reactions with cysteine, tyrosine and lysine, and serine. The flexible side chain covalent docking performs simulations in which the ligand is already attached to the covalent residue (via the newly formed covalent bond) and both ligand and residue are modeled as flexible. This method is used to analyze the non-covalent interactions of the bound ligands and target residues constituting the binding site.

Reactive Docking and Flexible Side Chain Covalent Docking on MYD88

isoTOP-ABPP and TMT-ABPP show that the TIR domain of MYD88 is covalently modified with different potency by BPK-25 and BPK-21 at C203, and by KB02 and KB05 at either C274 or C280 within the tryptic peptide (270-282). Consequently, two different docking techniques were applied to rationalize the different potencies of the first compounds on C203, and to attempt resolving the ambiguity between C274 and C280 modification. The first approach used the reactive docking method to sort the ambiguity between the labeling of the C274 and C280. Then the flexible side chain covalent docking was used to generate putative binding mode of all the compounds and provide structural insight for their different activities.

Reactive docking simulations on the entire domain (PDB 4DOM) were performed with ligands KB02 and KB05 and in addition, with BPK-25 and BPK-21, as a proof of concept, since experimental studies show direct labeling of C203 with BPK-25, but not BPK-21. Reactive docking analysis on BPK-25 and BPK-21 confirmed that the most favorable residue is C203, while C274 is the predicted residue for the covalent binding of KB02 and KB05. These results show that the position of C280 on the protein surface is less likely to be modified because it is largely solvent exposed, while on the contrary, C274 is located inside a cleft of the domain. Flexible side chain covalent docking was then used to refine the binding mode of compounds BPK-25 and BPK-21 (FIG. 5G). In particular, the predicted binding mode of BPK-25 shows that it could bind by establishing two hydrogen bonds with R188 and E183 via the amide moiety (FIG. 5G, bottom right), which is missing in BPK-21 (FIG. 5G, top tight). The lack of these interactions justifies the lower efficacy reported for BPK-21 in alkylating C203.

Flexible Side Chain Covalent Docking on ERCC3

isoTOP-ABPP and TMT-ABPP showed that compounds BPK-25 and BPK-21 bind to ERCC3 by alkylating C342. Flexible side chain docking simulations were performed to rationalize the higher efficacy of BPK-21 with respect to BPK-25, by modeling the two ligands bound to C342 on a low resolution Cryo-EM structure of the protein (PDB 5OF4, 4.4 Å resolution). Results showed that BPK-21 can form two hydrogen bonds with T469 and Q497 side chains, while its central aromatic ring establishes a π-π interaction with W493 (FIG. 5H, top right). None of these interactions are possible for BPK-25 (FIG. 5H, bottom right), which is reflected in a lower docking score.

Flexible Side Chain Covalent Docking on TMEM173

In order to provide a structural insight of the direct labeling of C91 of the protein TMEM173 with ligands BPK-21 and BPK-25, a flexible side chain covalent docking method was applied on a low-resolution Cryo-EM structure (PDB 6NT5, res 4.1 Å). Results show that both BPK-21 and BPK-25 engage the pocket (roughly delimited by residues L98 and P141) by placing their aromatic rings: 2,3-dichlorobenzene and chloropyridine for BPK-21 and BPK-25 (FIG. 12B), respectively, in proximity to P141. Despite their structural differences, both ligands occupy the pocket by establishing mostly hydrophobic interactions in a very similar manner, in agreement with the comparable reported efficacy.

General Methods

The crystal structures of the proteins were retrieved from the Protein Data Bank: TIR domain of MYD88 (PDB 4DOM), ERCC3 (PDB 5OF4), TMEM173 (PDB 6NT5). Hydrogens were added with Reduce, then were prepared using AutoDockTools following the standard AutoDock protocol. A grid box was defined for each cysteine: C168, C192, C216, C203, C247 and C280 (size x: 60, y: 60, z: 60 points).

For the flexible side chain covalent method, ligands were modelled attached to the alkylated residue via covalent bond, then processed following the covalent docking protocol (available online at http://autodock.scripps.edu/resources/covalentdocking) to be modeled as flexible during the docking. All dockings were performed using AutoDock 4.2.6, generating 100 poses using the default LGA parameters. Poses with the best energy score were selected and analyzed. Figures were generated using Pymol. (The PyMOL Molecular Graphics System, Version 2.0 Schrödinger, LLC.)

Reference Protein Tables

Immune-Relevant Genes

Immune-enriched genes were identified by analyzing microarray data from BioGPS (U133 Å and MOE430 datasets for human and mouse, respectively) and RNASeq data from GTex (release V7). Data were first filtered to restrict analyses to microarray signals above 150 and median RPKM values above 10. Samples from each transcriptomic dataset were grouped to identify immune related cells and tissues. Within each group the highest-expressing sample was chosen and group-level values were converted to Z-scores to identify genes showing immune enrichment within each dataset. Immune-enriched Z-scores above 3 or 4 (for RNA-Seq or microarray data, respectively) were summed across all probes and datasets and the summed Z-score was used to rank-order all genes. Genes with a summed Z-score above 11 were defined as “immune-enriched” as these represented the approximately 10% most-immune-enriched genes in the genome.

Genes with immune-related phenotypes were identified by parsing data in the Online Mendelian Inheritance of Man (OMIM) database (https://www.omim.org). OMIM associations were extracted from the human UniProt database downloaded in February 2019. From the 3925 genes for which human phenotypic associations could be identified, 654 genes with immune-related phenotypes were selected by querying phenotype titles and descriptions for immune-related substrings (‘*immun*’, ‘*inflam*’, ‘*rheum*’, ‘*psoria*’, etc).

T Cell Proliferation Gene List (SLICE) (Shifrut et al., 2018)

Hits (genes with FDR <0.2 and |Z score|>2, authors' criteria) were taken from the manuscript by Shifrut et al. (Shifrut et al., 2018) and cross-referenced with Table S5 based on UniProt accessions.

Immune Module Lists (Rieckmann et al., 2017)

Genes corresponding to immune modules were taken from Supplemental Table S2 from the manuscript by Rieckmann et al. (Rieckmann et al., 2017, Nat Immunol 18, 583-593) and cross-referenced with Table 55 based on UniProt accessions.

Transcription Factors

The list of putative transcription factors was adapted from the GSEA website (http://software.broadinstitute.org/gsea/msigdb/gene_families.jsp).

Adapters and Scaffolding Proteins

To generate a list of putative adapter and scaffolding proteins we combined data from several different sources including GO, Uniprot, (UniProt, 2019) the scaffold protein database ScaPD, manual literature review, and a reagent list from R&D Biosystems (Adaptor Proteins Research Areas: R&D Systems https://www.rndsystems.com/research-area/adaptor-proteins (accessed Sep. 4, 2019)). Proteins associated with following GO terms were included: GO:0035591 (signaling adaptor activity), GO:0060090 (molecular adaptor activity), GO:0008093 (cytoskeletal adaptor activity), GO:0035615 (clathrin adaptor activity). Lists of proteins for these GO terms were downloaded from the Gene Ontology project website using the AmiGO tool (http://amigo.geneontology.org/amigo/; version 2.5.12)(Carbon et al., 2009) with filters requiring that entries were of the type “protein” belonging to the “Homo sapiens” organism. Additionally, a keyword search was performed on a downloaded copy of SwissProt human data from Uniprot. Data was queried using BioPython and the following search terms were used: “adapter”, “adaptor”, and “scaffold” and the search was performed against the following columns: comments prefixed with “FUNCTION”, associated GO term cross-references, keywords, and entry descriptions. The data that was used from the ScaPD database consisted of experimentally verified scaffold proteins.

The resulting list was then cross-referenced with Table S5, and the categorization of every target protein was reviewed manually.

SwissPalm List

SwissPalm proteins and sites (Release 2 [Feb. 18, 2018]) were downloaded and cross-referenced with the Table S5 based on UniProt accessions. Sites were deemed a match if any of the cysteine residues in the detected tryptic peptide matched the SwissPalm reference (Table S18).

### Example 7

Chemical Proteomic Map of Cysteine Reactivity in Activated T Cells

Upon activation, T cells enter a growth phase associated with a number of biochemical changes that include alterations in cellular redox state, cytoskeletal rearrangements, and increased glycolytic and mitochondrial metabolism. The molecular pathways that both execute and are influenced by these changes have been studied by global gene and protein expression, as well as phosphoproteomic and metabolomic analyses, that compare resting versus activated T cells. Some of the discovered changes in activated T cells occur in general biochemical pathways associated with, for instance, cell proliferation, while others reflect immune-restricted processes. The extent to which these types of activation state-dependent changes in the biochemistry of T cells might also create a landscape of new targets for chemical probes that regulate T cell function remains largely unexplored. The global scale profiling was addressed by using activity-based protein profiling (ABPP) methods to quantify cysteine reactivity and electrophilic small-molecule interactions in primary human T cells activated by T cell receptor (TCR) stimulation.

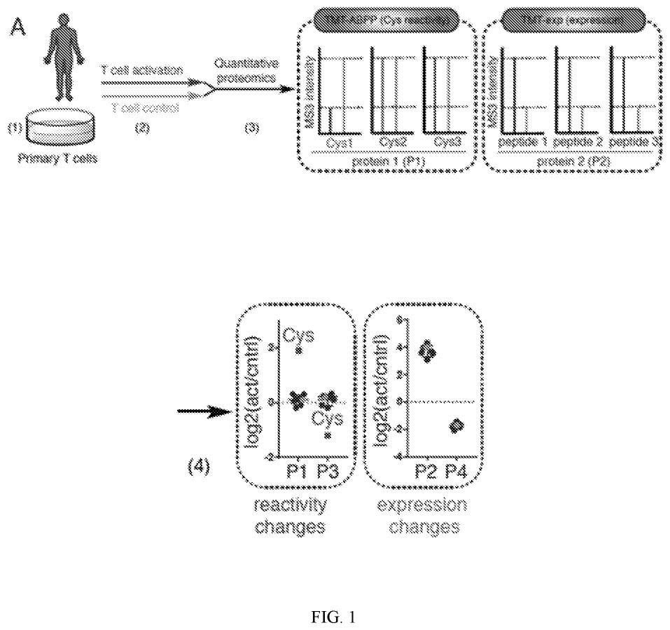

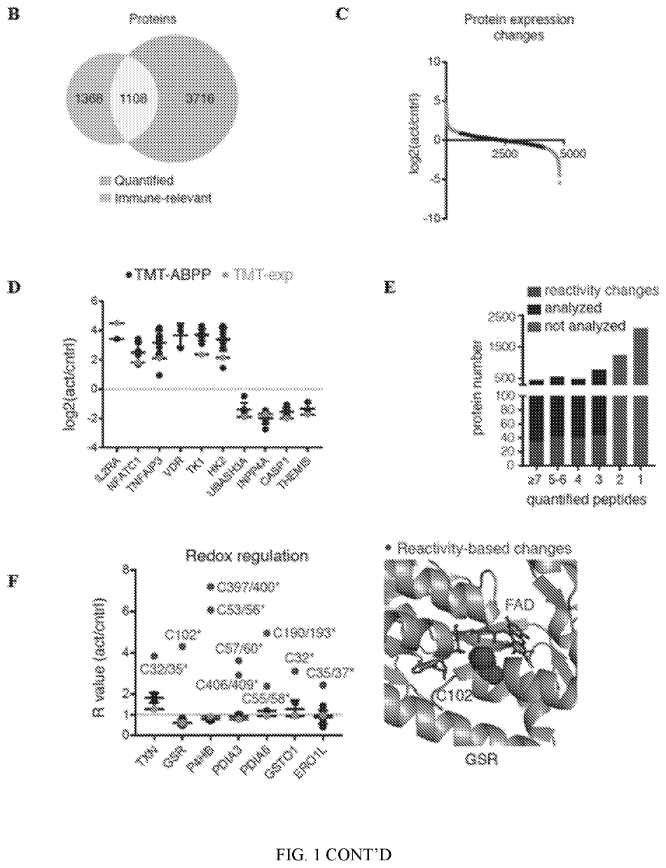

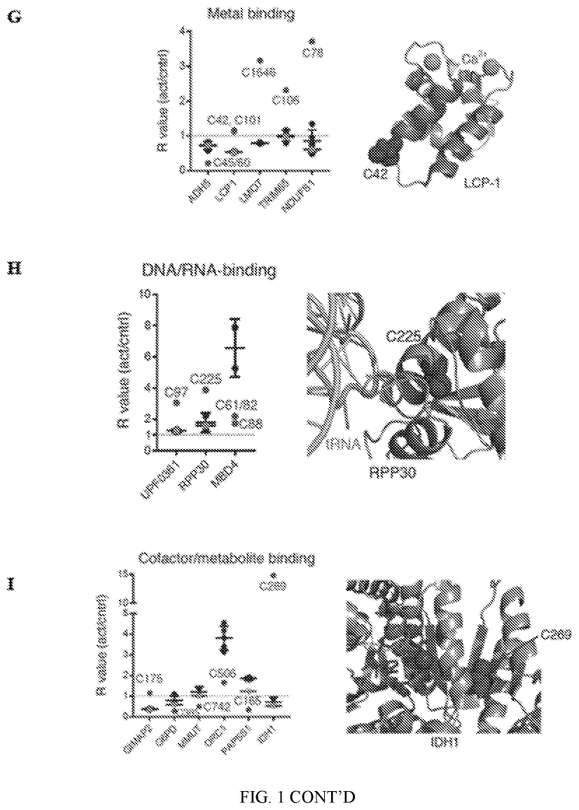

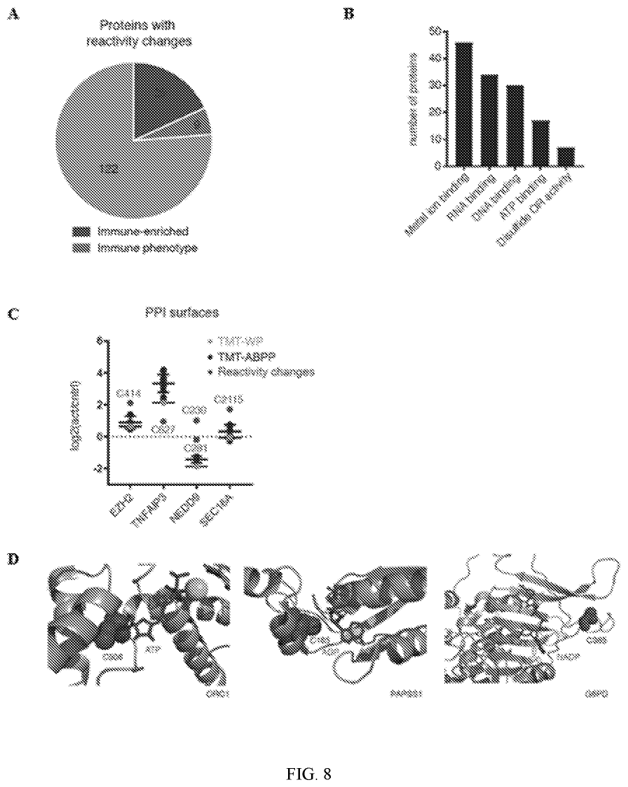

Primary human T cells were isolated from human blood, activated by exposure to anti-CD3 and anti-CD28 antibodies for three days, and then expanded in culture in the presence of IL2. Cysteine reactivity changes were measured by treating proteomic lysates from activated or expanded control T cells with a broad-spectrum, cysteine-directed iodoacetamide-desthiobiotin probe (1A-DTB) (Patricelli et al., 2016), protease digestion of the IA-DTB-treated proteomes, streptavidin enrichment of IA-DTB-labeled cysteine-containing peptides, and quantitative, multiplexed LC-MS-based proteomic analysis using tandem mass tags (TMT, 10-plex experiments); (FIGS. 1A and 8C, 8D). These cysteine reactivity profiles were then integrated with complementary proteomic experiments measuring protein expression changes in control versus activated T cells (FIGS. 1A and S1A, S1B). In total, more than 4800 proteins were quantified in expression-based (TMT-exp) proteomic experiments, the vast majority of which (˜80%) were also quantified by cysteine reactivity profiling (TMT-ABPP) (Table S1 and S2), and nearly a quarter of these proteins qualified as “immune-relevant” (FIG. 1B, Table S3), based on immune cell-enriched expression profiles derived from public databases and/or human genetic (OMIM) evidence of contributing to immune-related disorders. A protein was considered to show altered expression if its abundance was elevated or reduced by >two-fold in activated T cells, and −1100 proteins satisfied this requirement (FIG. 1C), including several immune-relevant proteins (e.g., IL2RA, TNFAIP3) (FIG. 1D), proteins involved in glycolysis, and proteins regulated by mTORC1 and MYC pathways that are known markers of T-cell activation and proliferation.

A distinct set of proteins (160 in total) harbored cysteine reactivity changes that differed substantially from the corresponding expression profiles for these proteins in activated T cells (FIG. 1E). These cysteine reactivity changes were found in immune-relevant proteins (FIG. 8A) and featured functional sub-groups that may reflect the diverse modulation of cellular biochemistry in activated T cells (FIG. 8B). For example, a number of catalytic and active-site cysteines in proteins involved in redox regulation showed much greater reactivity in activated T cells, possibly reflecting the higher intracellular reducing potential of these cells furnished, at least in part, by increases in glutathione production (FIG. 1F). Reactivity changes were also found for cysteines in the metal-binding domains of proteins (FIG. 1G), with one prominent example being the immune-relevant protein L-plastin (LCP1), which is a calcium-regulated actin-binding protein that participates in remodeling of the actin cytoskeleton during T cell activation. Calcium binding decreases the ability of LCP-1 to bind actin by inducing structural changes to the EF-hand motif of LCP-1, in particular, in α-helices 2 and 3 surrounding C42 (FIG. 1G), which showed increased reactivity in activated T cells (FIG. 1G). Additional reactivity changes were observed for cysteines in DNA or RNA-binding domains (FIG. 1H) and at the sites of protein-protein interactions (e.g., EZH2, NEDD9, TNFAIP3) (FIG. 8C), as well as cysteines proximal to cofactor- and metabolite-binding sites (FIG. 1I). These cysteine reactivity changes may reflect a landscape of dynamic intermolecular interactions occurring in activated T cells that, in turn, impinge upon the reactivity of cysteines. As one example, C269 in isocitrate dehydrogenase 1 (IDH1) undergoes a dramatic increase in reactivity in activated T cells (FIG. 1I), which could reflect changes in cofactor (NADP) and/or substrate (isocitrate) binding that promote a structural rearrangement in residues 271-277, which may, in turn, alter the reactivity of C269.

Chemical Proteomic Map of Cysteine Ligandability in Human T Cells

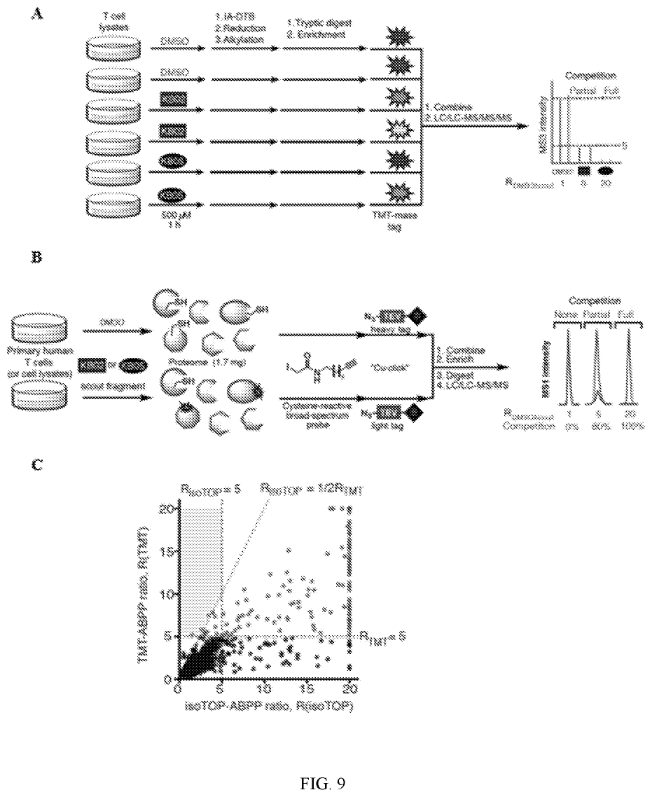

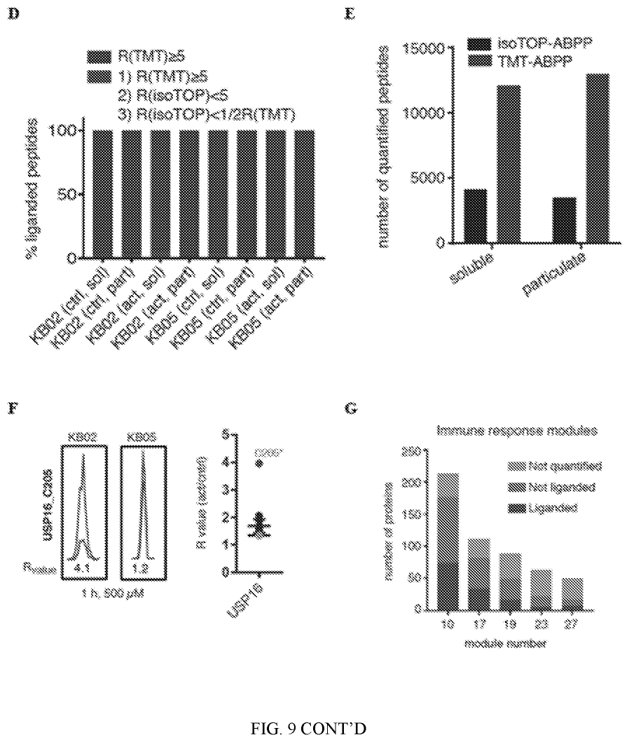

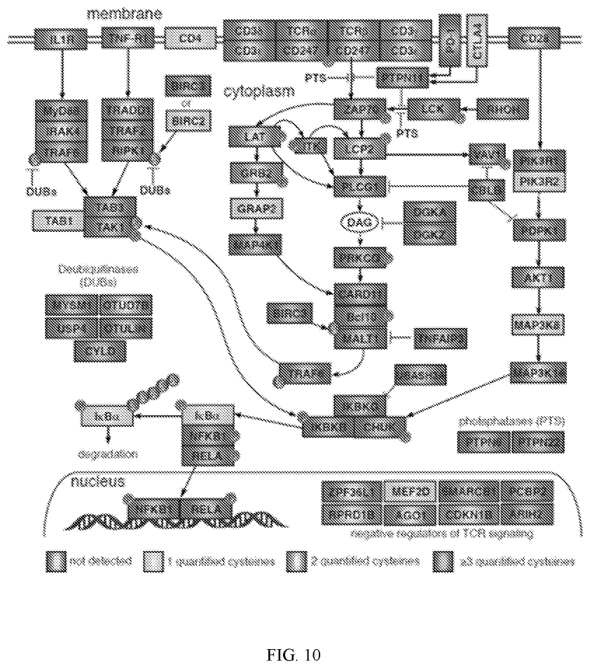

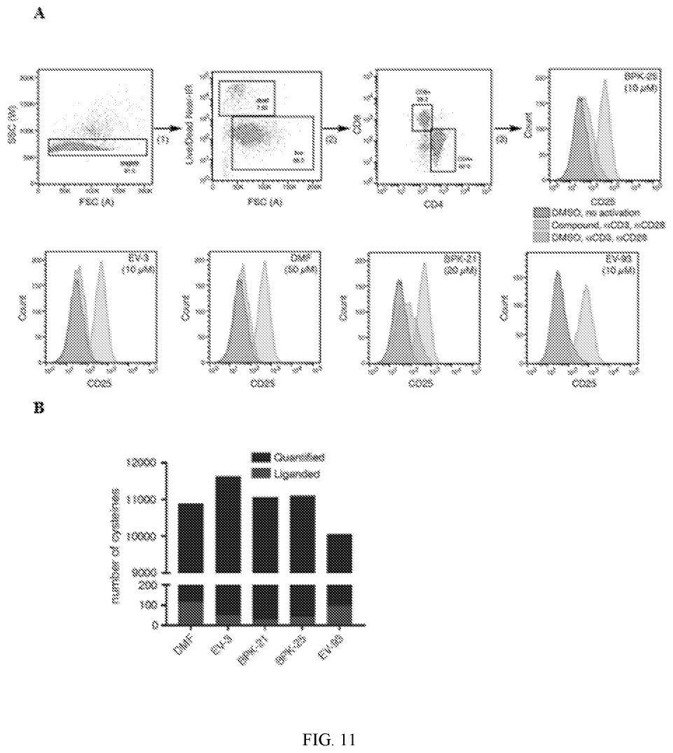

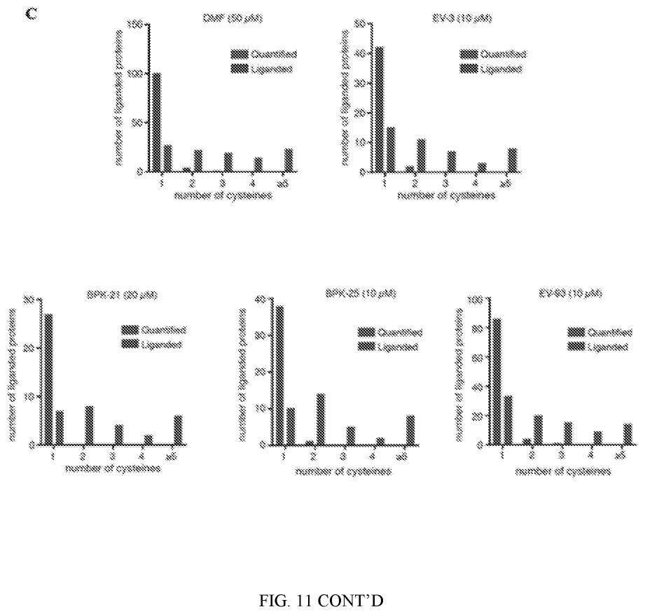

An efficient strategy to globally assess the ligandability of cysteines in native biological systems that leverages broad-reactivity was recently described. Electrophilic small-molecule fragments are referred to as “scouts”. Two scout fragments bearing a chloroacetamide (KB02) or acrylamide (KB05) (FIGS. 2A and 2B)—reactive groups frequently found in covalent chemical probes and drugs—were used to construct in-depth cysteine ligandability maps across primary human T cells in both resting and activated states. Scout fragment-cysteine interactions were analyzed using two complementary chemical proteomic methods that provided a balance of confidence in quantitative accuracy (isoTOP-ABPP) with greater multiplexing capacity (TMT-ABPP) (FIG. 2A). Both proteomic methods provided similar ratio (R) values (DMSO/scout fragment (500 μM, 1 h)) for cysteines in T cell proteomes, with the MS3-based quantification used in TMT-ABPP resulting in mild ratio compression (FIGS. S2C and S2D), which was countered by a substantial increase in proteomic coverage compared to isoTOP-ABPP (FIG. 9E). Designated cysteines were ligandable if they showed an R value of ≥5 as measured by either isoTOP-ABPP or TMT-ABPP. From a total of >18,000 cysteines and 6035 proteins quantified in human T cells, 3479 liganded cysteines in 2292 proteins were identified (FIGS. 2C, 2D and Table S5). These ligandability events were broadly distributed across cysteines with diverse intrinsic reactivities (Table S6, S7), underscoring contributions from both the electrophilic and binding groups of scout fragments in conferring strong engagement of cysteines in the T cell proteome (FIG. 2E).

Among the liganded cysteines were several targeted by existing covalent probes and drug candidates, including those being pursued for immunological disorders (e.g., C909 in JAK3, C528 in XPO1; Table S8), underscoring the potential for ABPP to “rediscover” established druggable sites on immune-relevant proteins. Ligandable cysteines were also well-represented within the subset of proteins showing expression and/or cysteine reactivity changes in activated T cells, where cysteines with altered reactivity showed a greater propensity for liganding by scout fragments (FIG. 2F). As one example, the discovery of a ligandable cysteine (C93) in programmed cell death protein 1 (PDCD1 or PD-1) was noted, which was only observed in activated T cells (FIG. 2G), and likely reflects the induced expression of this key immune checkpoint protein following T cell stimulation. Ligandable cysteines showing reactivity-based changes included the catalytic cysteine in the deubiqutinase USP16 (C205) (FIG. 9F), which has been shown to regulate hematopoietic stem cell differentiation.

Cross-referencing our ligandability map with the aforementioned database of immune-relevant proteins furnished >500 shared targets (FIG. 2H, and Table S5), of which >120 produce, upon mutation in humans, monogenic diseases with a strong immune phenotype (FIG. 2I). This point is emphasized because such Mendelian genetic relationships to immune disorders can be used to prioritize proteins for drug development programs aimed at treating autoimmune or autoinflammatory disorders (e.g., JAK3) as well as promoting immune responses to cancer (e.g., CTLA4). Integrating our ligandablity map with additional genomic and proteomic studies identified electrophilic fragment-sensitive cysteines in proteins: 1) found by genome-wide CRISPR screening to regulate T cell proliferation following stimulation (FIG. 2J and Table S9); and 2) that are part of immune-enriched modules (FIG. 2G), including those relevant for T cell activation (module 17), established in expression-based proteomics studies (Table S10).

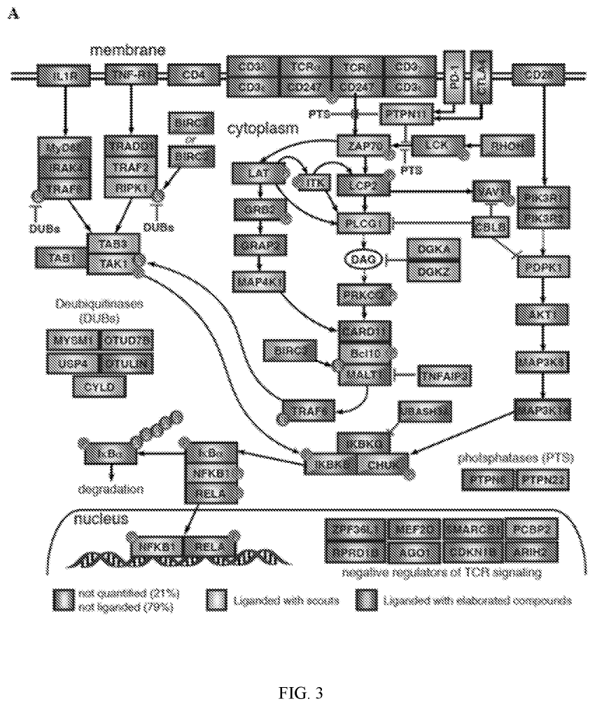

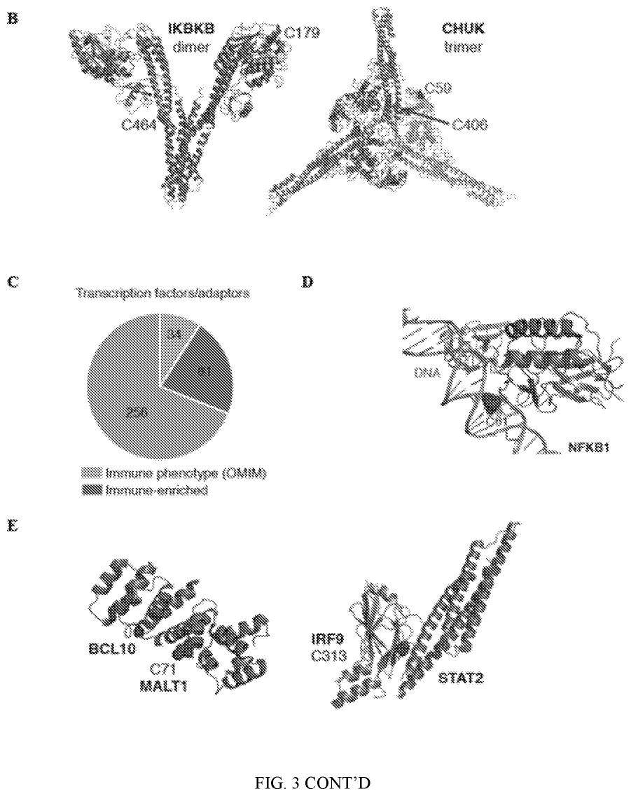

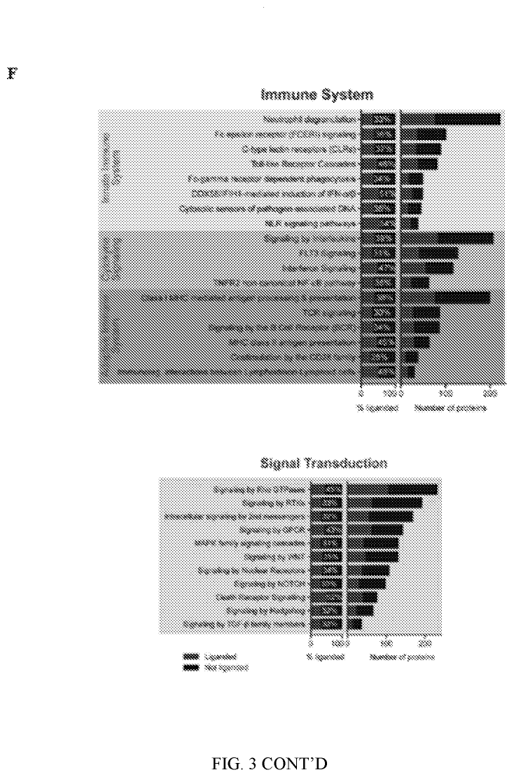

A more focused analysis revealed the striking breadth of ligandable cysteines in key immune signaling pathways, including those mediating T cell and TNF-receptor signaling and NF-κB activation (FIG. 3A). The proteins in these pathways harboring ligandable cysteines derived from diverse structural and functional classes, including not only enzymes (e.g., DGKA/Z, IKBKB), but also adaptor proteins (e.g., MYD88) and transcription factors (e.g. NFKB1) (FIG. 3A). Even for more classically druggable proteins like kinases, observed sites of cysteine ligandability were observed that were far removed from the ATP-binding pocket (FIG. 3B, IKBKB_C464, CHUK_C406), underscoring the potential for covalent ligands to engage non-canonical sites on proteins. Also supporting this conclusion was the large number of ligandable cysteines identified in historically challenging to drug proteins like transcription factors and adapter proteins, including a subset that have Mendelian links to immunological disorders (FIG. 3C, Table S11). Some of these cysteines reside in proximity to protein-protein interactions important for the assembly and function of immune-relevant protein complexes (FIG. 3D).

Scout fragment profiling results, taken together, present an extensive landscape of ligandable cysteines in immune-relevant proteins, pointing to a potentially broad opportunity to discover and develop covalent chemical probes that modulate T cell function. Next an experimental workflow was established that would illuminate the functional effects and tractability of covalent ligand-protein interactions, while also preserving the globality and biological integrity afforded by profiling these interactions in primary human T cells.

A Functional Screen of Elaborated Electrophilic Compounds in T Cells

Fragment-based screening, whether performed on a single protein of interest or more broadly across the proteome as described herein, offers advantages for discovering hit compounds for challenging protein classes. Nonetheless, progressing fragments to more advanced chemical probes and ultimately drugs can be confounded by a variety of factors, including the low-affinity and promiscuity of initial hits and the tractability of fragment-binding sites on proteins. These problems have been historically addressed by labor-intensive, structure-guided protocols that require purified protein and have limited throughput. An in cellulo strategy was created that integrates phenotypic screening with our chemical proteomic methods to furnish structure-activity relationships on many covalent ligand-cysteine interactions in parallel, such that the tractability and potential functional effects of these interactions could be comparatively evaluated.

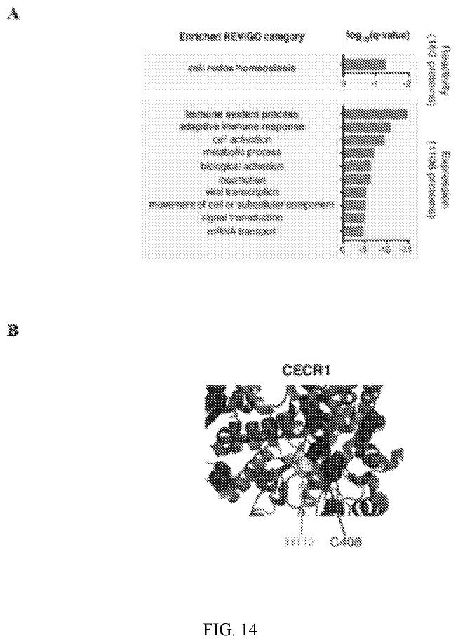

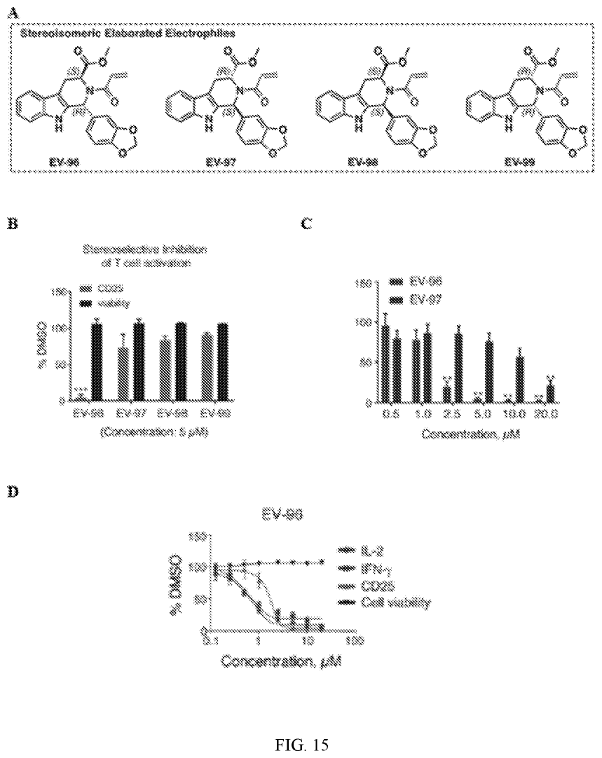

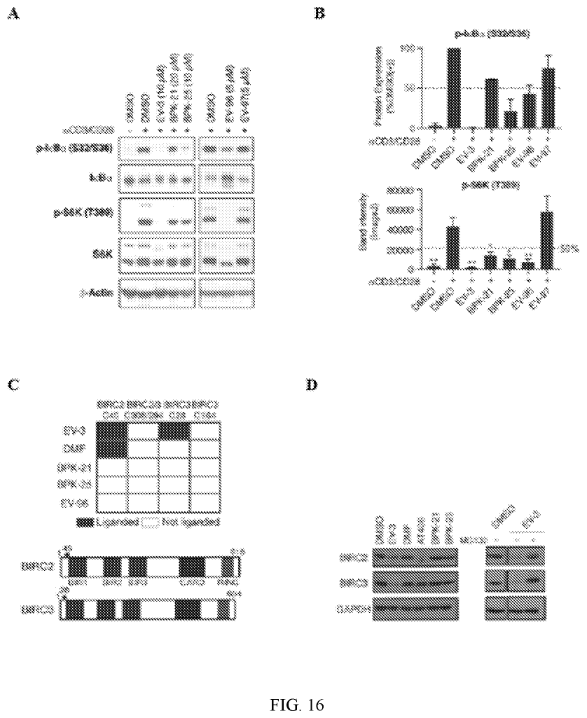

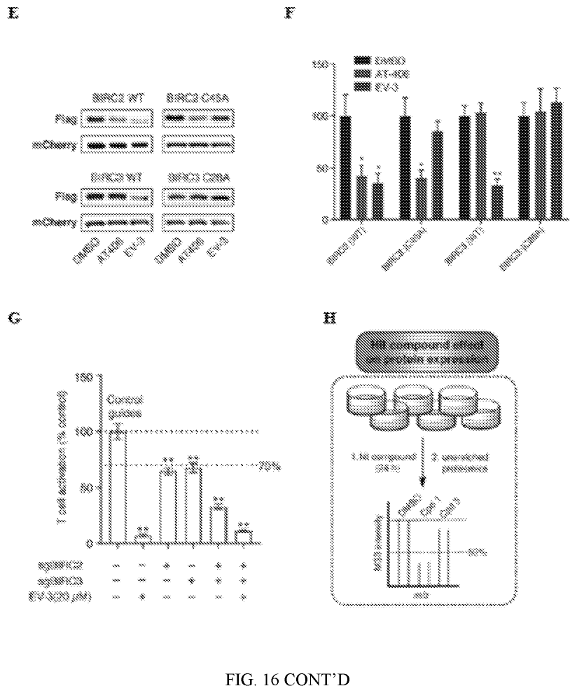

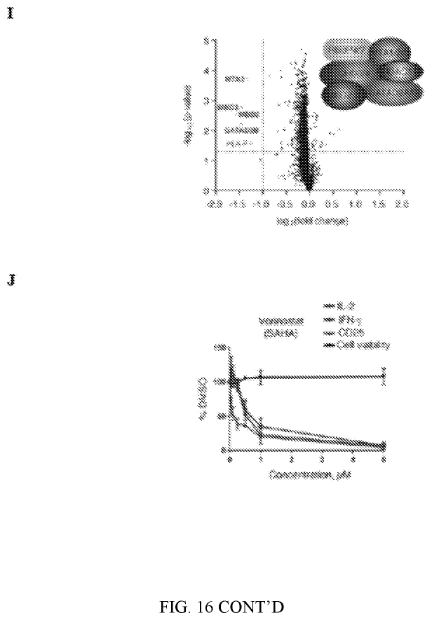

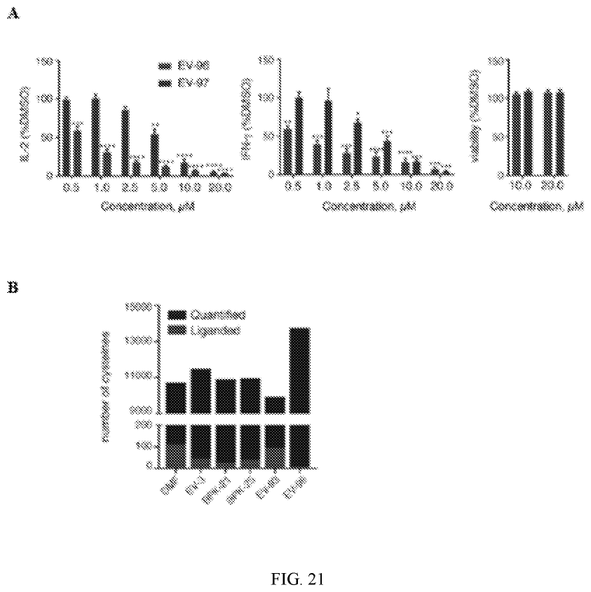

A multidimensional screen was preformed of a focused library of structurally elaborated electrophilic small molecules to identify compounds that suppress T cell activation at low-μM concentrations without causing cytotoxicity (FIG. 4A). Primary human T cells isolated from the blood of healthy donors were activated with anti-human antibodies against CD3 and CD28 antigens in the presence of an in-house collection of electrophilic compounds (10 μM each; ˜130 compounds tested in total; average compound MW=400 Da; Table S12), where suppression of T cell activation was measured 24 h later by reductions in secreted IL-2 and IFN-γ and in the expression of the cell surface markers CD69 and CD25 (FIG. 4A). The viability of T cells was monitored by flow cytometry using near-IR live-dead stain. 17 compounds, along with the immunosuppressive drug DMF, which was used as a positive control, were found to substantially suppress T cell activation (>65%) without causing cytotoxicity (viability >85%) (FIG. 4B-D, Table S13). The active compounds included different classes of electrophiles, of which representative acrylamides (BPK-21, BPK-25, EV-96) and chloroacetamides (EV-3, EV-93, EV-96) (FIG. 4C) were selected for further characterization based on their concentration-dependent profiles, which revealed near-complete blockade of T cell activation at <20 μM with negligible cytotoxicity (FIG. 4D). EV-96, which was part of a set of four stereoisomeric electrophiles (FIG. 4E), was found to stereoselectively block T-cell activation (FIG. 14A). EV-96 suppressed T-cell activation markers with an EC 50 of <2.5 μM (FIGS. 15B and 15C), while its enantiomeric analogue EV-97 showed an approximately 10-fold weaker activity (FIGS. 15B and 21A). These results highlight how the incorporation of stereochemistry into the electrophilic compound library enabled the discovery of physicochemically identical active (EV-96) and inactive (enantiomer EV-97) compounds for further mechanistic analysis.

Chemical Proteomic Analysis of Immunosuppressive Electrophilic Compounds

The targets of active compounds were mapped in primary human T cells by ABPP (Table S14). Cysteine reactivity profiles were acquired for T cells treated with active compounds (10-20 μM for elaborated compounds BKP-21/25 and EV-3/93; 50 μM for DMF) for 3 h. On average, 10,000-12,000 cysteines were quantified for each compound (aggregated across at least 6 biological replicates, where it was required that a cysteine was quantified in at least two replicates for interpretation), of which only a modest fraction (0.2-1.0%) was substantially altered in reactivity by compound treatment (RDMSO/compound ≥4) (FIGS. 5A and 8A). There is a distinct cysteine reactivity profiles of each hit compound, which engaged largely non-overlapping sets of targets (FIGS. 5A and 5B) that originated from diverse structural and functional classes (FIG. 5C and Table S15) and included several immune-relevant proteins (FIG. 5B). Most of the protein targets were engaged site-specifically in that only one of multiple quantified cysteines on these proteins was sensitive to the hit compounds (FIG. 9B).

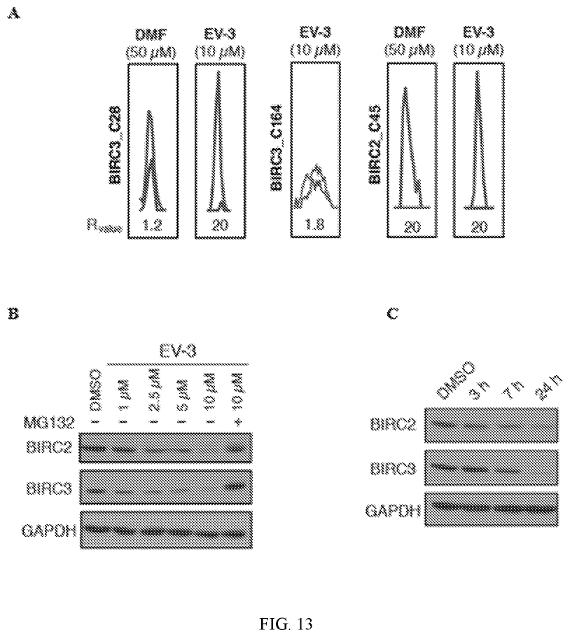

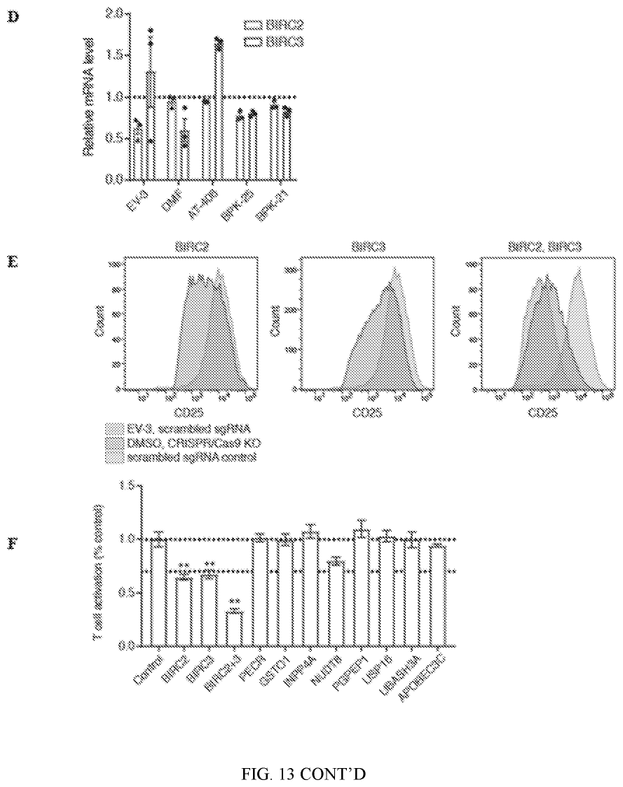

The vast majority of cysteines liganded by elaborated hit compounds (˜80%) were also engaged by scout fragments (FIGS. 5D, 5E, Table S16), underscoring the potential for fragment profiling to discover tractable sites of ligandability across the human proteome that can also be targeted by more elaborated electrophiles with improved potency (low-04) and credible SARs. In general support of this conclusion, molecular modeling was performed on the structures of proteins containing cysteines targeted by hit compounds (Table S17), which revealed, in −60% of the cases, predicted binding pockets within 5 Å of the liganded cysteines (FIGS. 5E, 5F, SSA). Docking studies on representative protein targets of the structurally related hit compounds BPK-21 and BPK-25 further supported the observed SAR profiles (e.g., selectivity of MyD88_C203 for BPK-25, ERCC3_C342 for BPK-21, and lack of observed selectivity for TMEM173_C91 FIGS. 5G, 5H, and Figs S6A, S6B). The liganded cysteine residue C91 in TMEM173, or STING, has been shown to be palmitoylated (Mukai et al., 2016) and targeted by other covalent ligands that antagonize TMEM173-dependent inflammatory cytokine production (Haag et al., 2018). Consistent with this past work, it was found that BPK-25 inhibited TMEM173 activation by the cyclic dinucleotide ligand cGAMP in both the human monocyte cell line THP1 and in primary human PBMCs (FIG. 13C-F). More global analysis of our scout fragment data sets revealed additional liganded cysteines subject to palmitoylation (Table S17), suggesting the broader potential to pharmacologically target these dynamic lipid modification sites on proteins.

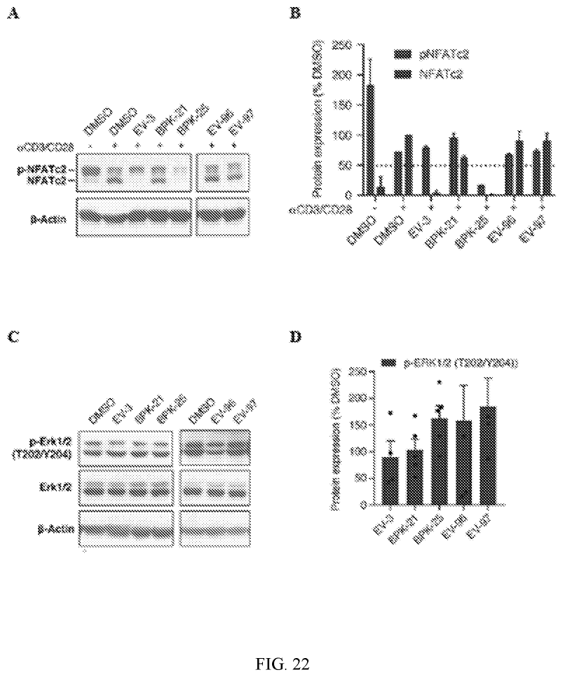

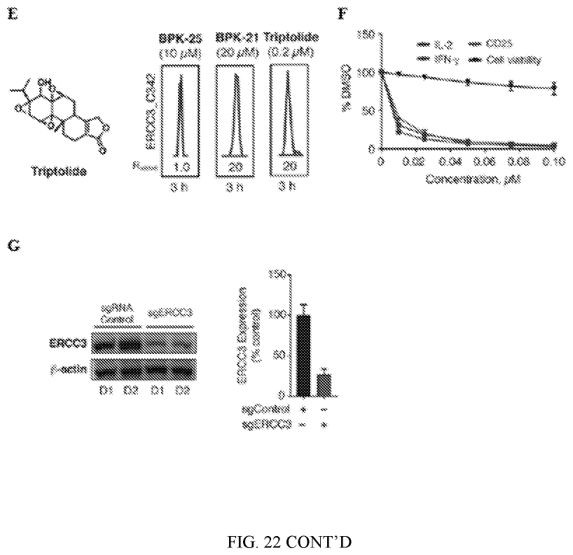

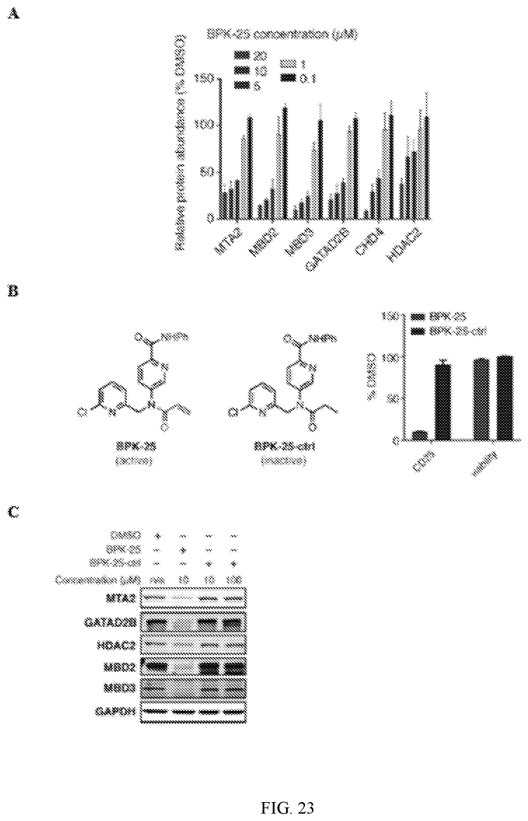

There appear to be a limited number of shared sites of engagement as potentially representing a common mechanism of action for immunosuppressive hit compounds, this model was undermined by several factors. First, the shared cysteines mostly reflected highly ligandable sites that have been observed in previous studies to interact broadly with diverse electrophilic compounds. It is therefore questioned whether such cysteines would show selective interaction with hit compounds over inactive compounds in the screened library. Second, very few of the shared targets had evidence of immune relevance (Table S13) and were instead mostly ubiquitously expressed proteins. The hit compounds also variably depleted glutathione (GSH) from T cells, but GSH reductions were excluded as a candidate mechanism because buthionine sulphoximine (BSO), an inhibitor of the GSH biosynthetic enzyme gamma-glutamyl cysteine ligase (GCLC), did not affect T cell activation (FIG. 6A), a result that is consistent with previous work showing that genetic deletion of GCLC does not impair T cell activation. It was alternatively hypothesized, based on the diverse target profiles of the hit compounds, combined with the greater body of literature documenting various ways that electrophiles can affect immune cell function, that the hit compounds acted by distinct mechanisms. In support of this hypothesis, it was found that hit compounds differentially impacted key transcriptional pathways involved in T cell activation, with DMF, EV-3, EV-96, and BPK-25 suppressing NF-id3 activation, as measured by >50% reductions in aphosphorylation (FIG. 6B), while only EV-3 and BPK-25 blocked NFAT activation, as measured by >50% reductions in NFATc2 dephosphorylation (FIGS. 22A and 22B). BPK-25 also reduced NFATc2 expression (FIGS. 22A and 22B). All of the hit compounds negatively affected mTOR pathway activation, as measured by >50% reductions in S6K phosphorylation (FIGS. 6A and 6B), while none of the active compounds substantially affected ERK phosphorylation, which was found to be a variable parameter in T cells (FIGS. 22C and 22D).

Among the active compounds, BPK-21 was unique in that it did not appear to impact the NF-κB or NFAT pathways. As noted above, a specific target of BPK-21, but not other active compounds, was C342 of ERCC3, a cysteine in the active site of this helicase that is also targeted by the electrophilic immunosuppressive natural product triptolide (FIGS. 6B and 6C). Like BPK-21, triptolide has been shown to impair T cell activation (FIG. 6B) without blocking NF-κB DNA binding activity. Using CRISPR/Cas9 technology that disruption of the ERCC3 gene (sgERCC3 cells), but not other representative targets of BPK-21, significantly impaired T cell activation to a similar degree as BPK-21 treatment (FIGS. 6D and 6E). Western blotting estimated an −80% loss of ERCC3 protein in sgERCC3 cells, which also showed only a modest further reduction in activation when treated with BPK-21 (FIGS. 6D and 6E). These data, taken together, indicate that BPK-21 likely suppresses T cell activation through blockade of ERCC3 function, which may in turn act downstream or separately from pathways involved in NFAT and NF-κB activation.

Electrophilic Compound-Dependent Degradation of BIRC2 and BIRC3

The NF-κB pathway is known to be regulated, both positively and negatively, by reactive oxygen species (ROS) and electrophilic compounds, and consistent with this, cysteines throughout this pathway were discovered that showed sensitivity to scout fragments and/or elaborated hit compounds (FIG. 3A). In reviewing these cysteines, C28 of BIRC3 was noted as a unique target of EV-3 compared to other hit compounds (FIGS. 5B and 6F), and the corresponding cysteine (C45) in BIRC2 was also engaged by EV-3, as well as by DMF, but not other hit compounds (FIGS. 5B, 6F, and 13A). Other quantified cysteines in BIRC2 and BIRC3 were not affected by EV-3 treatment (FIG. 6F). These proteins, also referred to as cellular inhibitor of apoptosis proteins C-IAP1 and C-IAP2, respectively, regulate both canonical and non-canonical NF-κB activation through ubiquitination of diverse substrates (FIG. 3A). C28 of BIRC3 (and C45 of BIRC2) is located in close proximity to the BIR1 domain, which interacts with TRAF2 (FIGS. 6G and 6H) to facilitate recruitment to the TNF receptor. This interaction has been suggested to stabilize BIRC2, preventing its autoubiquitination and subsequent degradation, and mutations in the BIR1 domain of BIRC2/3 have been found to abolish or significantly diminish interactions with TRAF2, but, chemical probes targeting this region of BIRC2/3 have not yet been described.

It was found that treatment of human T cells with EV-3, but not other hit compounds, including DMF, led to the time-dependent, and proteasome-sensitive degradation of both BIRC2 and BIRC3 (FIGS. 6I, 6J, 13B, and 13C). This profile differed from the described Smac mimetic inhibitor AT406, which targets the BIR3 domain and at tested concentrations selectively promoted the degradation of BIRC2, but not BIRC3 (FIGS. 61 and 6J). EV-3 caused minimal changes in mRNA content for BIRC2 or BIRC3 in T cells (FIG. 9D), supporting a direct effect on protein stability in T cells. Finally, genetic disruption of BIRC2 and BIRC3, either independently or in combination (BIRC2/BIRC3-null), using CRISPR/Cas-9 technology impaired T cell activation, with the combined disruption producing a more substantial effect (FIG. 6K). Treatment with EV-3, however, further decreased T cell activation in BIRC2/BIRC3-null cells, indicating that the genetic inactivation of BIRC2 and BIRC3 by CRISPR/Cas-9 may be incomplete (e.g., heterozygotic inactivation in a subset of cells) or additional targets of EV-3 contribute to its full suppressive effects in T cells. Several additional targets of B9 were evaluated by CRISPR/Cas-9 disruption, but none were found to substantially impair T cell activation (FIG. 13F).

The Acrylamide BPK-25 Promotes Degradation of the NuRD Complex

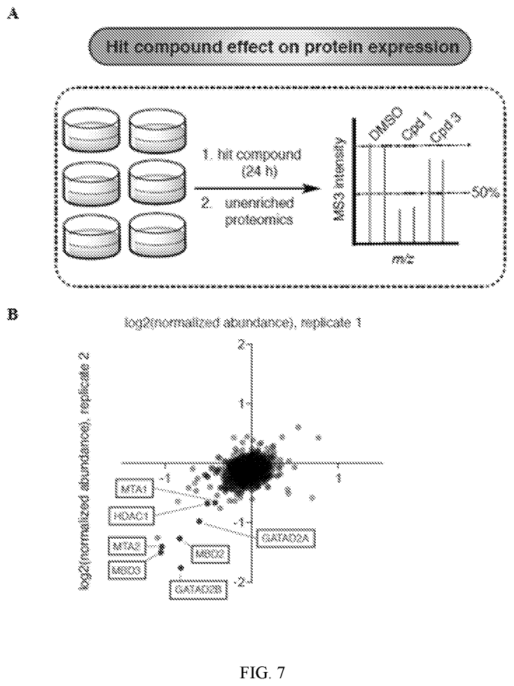

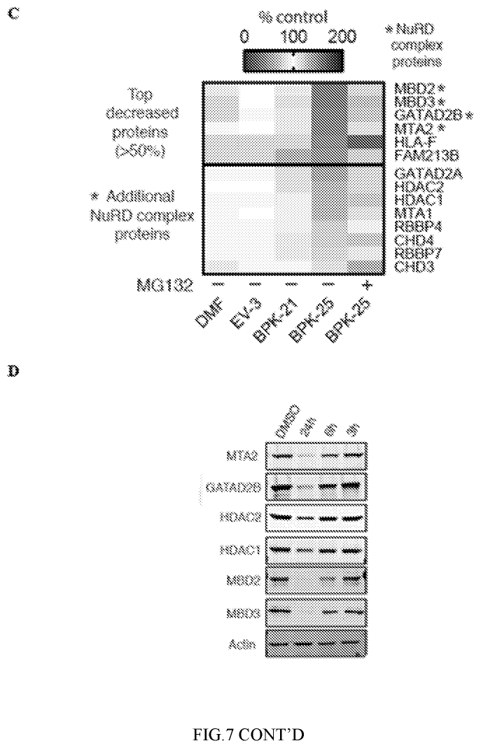

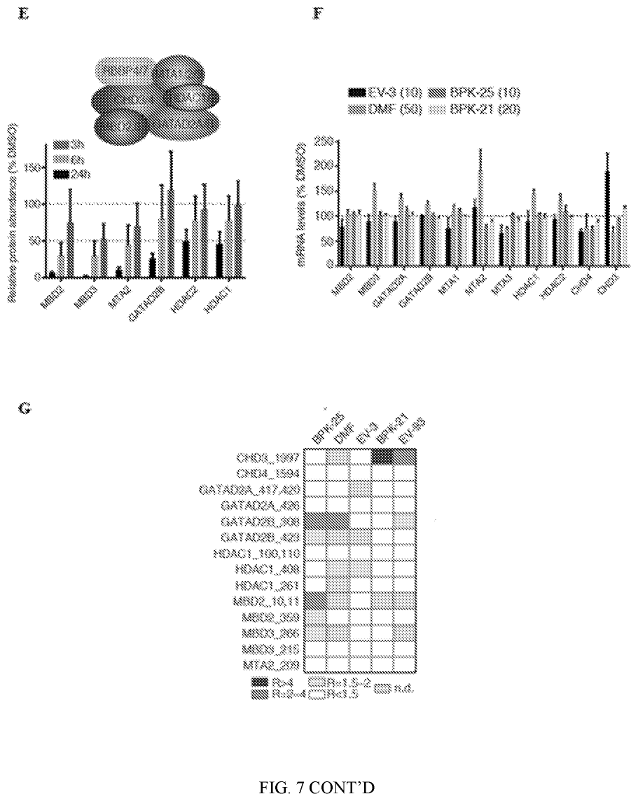

As noted previously, the structurally related acrylamides BPK-21 and BPK-25 both suppress T cell activation, but by apparently different mechanisms (FIGS. 6B and 6C). The data supported that BPK-21 impaired T cell activity by inhibiting ERCC3, while BPK-25 impaired NF-κB signaling (FIG. 6B). A survey of the cysteines preferentially engaged by BPK-25 over BPK-21 did not reveal obvious candidate proteins within the NF-κB pathway as potential targets of relevance for the former compound (Table S13). Motivated by the finding that EV-3 promoted the degradation of immunologically relevant BIRC proteins (FIGS. 61 and 6J), an expression-based proteomic analysis of primary human T cells treated with BPK-25 or other hit compounds was performed (FIG. 7A). This study revealed that BPK-25, but not other hit compounds, including BPK-21, led to the striking and selective reduction of several proteins in the Nucleasome Remodeling and Deacetylation Complex (NuRD), including a >50% decrease in GATAD2B, MBD2, MBD3, and MTA2, as well as directionally similar losses in GATAD2A, HDAC1, HDAC2, MTA1, and RBBP4 (FIGS. 7B, 7C, Table S18, S19). Only two other proteins across the >3000 total proteins quantified in our proteomic experiments showed substantial (>50%) reductions in BPK-25-treated cells (FAM213B and HLA-F), and these proteins were also affected by EV-3 and BPK-21) (FIG. 7C). BPK-25-mediated reductions in NuRD complex proteins were time-dependent, with initial changes observed as early as 6 h and maximal effects observed at 24 h (FIG. 7D) and blocked by treatment with the proteasome inhibitor MG132 (FIG. 7C). The reductions in NuRD complex members were not accompanied by corresponding changes in mRNA expression for the proteins (FIG. 7E), supporting that BPK-25 lowers NuRD complex by a post-transcriptional mechanism. While the cysteine reactivity profiles indicated an −50% reduction in C10/11 and C359 in MBD2, C266 in MBD3, and C308 of the GATAD2B in BPK-25-treated T cells (but not T cells treated with BPK-21 or EV-3 (FIG. 7F); 3 h treatment), these changes were difficult to assign with confidence as being reductions in cysteine reactivity versus protein expression. It was also note that DMF decreased the apparent reactivity of C308 of the GATAD2B (FIG. 7F), despite not leading to NuRD complex degradation (FIG. 7C). Whether one or more of the apparently BPK-25-sensitive cysteines in NuRD complex proteins is responsible for mediating complex loss in T cells remains to be determined. These studies have uncovered a remarkably selective, electrophile-mediated process for degradation of a key transcriptional regulatory complex in primary human T cells. Considering that HDAC inhibitors have been previously shown to block T cell activation, and HDACs can also support NF-κB function, it is possible that the BPK-25-mediated loss of the NuRD complex is relevant to the mechanism of action of this immunosuppressive compound.

Table S1 provides a summary of TMT-exp proteomic data showing changes in protein expression in control vs activated primary human T cells. Table S2 provides TMT-ABPP proteomic data showing changes in cysteine reactivity in control vs activated primary human T cells. Table 3 provides resource table for determination of immune-relevant genes. Multiple Z-scores were calculated from several transcriptomic tissue profiles, and these Z-scores were summed to generate the value in column H. Genes with a summed Z-score above 11.0 were defined as “immune-enriched” as these represented the approximately 10% most-immune-enriched genes in the genome. Annotation of immune phenotype (column T) was performed by parsing data in the Online Mendelian Inheritance of Man (OMIM) database and querying phenotype titles and descriptions for immune-related substrings (‘*immun*’, ‘*inflam*’, ‘*rheum*’, ‘*psoria*’, etc). In total, there were 2004 immune-enriched genes and 655 genes associated with immune phenotypes which, when combined, result in 2746 total immune-relevant genes (column U). Table S4 provides list of proteins with identified cysteine reactivity changes, along with designations of immune-relevance and GO-terms: “RNA-binding”, “Metal ion binding”, “DNA binding”, “disulfide oxidoreductase activity”, and “ATP binding”. The summary of GO-term analysis is provided in the figures. Table S5 provides a master table containing total cysteine reactivity proteomic data from combined isoTOP-ABPP and TMT-ABPP experiments with scout fragments (“broad ligandability”, KB02 and KB05) and active compounds (DMF, EV-3, EV-93, BPK-21, BPK-25), as well as isoTOP-ABPP hyperreactivity experiments. Values for each treatment group are final average values resulting from at least 2 replicate experiments described in Supplementary methods. Maximum values from all treatment groups (Columns J and P) for active compounds and scout fragments are used to determine “ligandability” of a select residue (related to FIG. 2). Table S5 illustrates an exemplary list of liganded cysteines which are identified from isoTOP-ABPP experiments performed in cell lysates (in vitro). Table S5 further shows the accession number (or the protein identifier) of the protein and the cysteine residue number. Table S6 shows the broad versus hyper-reactivity (expanded). Table S7 shows the broad versus hyper-reactivity (activated). Tables S6 and S7 show comparison of isoTOP ABPP and isoTOP-ABPP hyperreactivity R values for activated and control T cells (related to FIG. 2E). Table S8 shows the liganded cysteines in immune-relevant proteins that are also targeted by other electrophile compounds. Table S9 shows SLICE, Analysis of proteins liganded with scout fragments (Table S5) for assignment to T cell proliferation genes (related to FIG. 2). Table S10 shows a summary of the immune modules. Table S11 shows a summary of the immune-enriched versus hard to drug proteins (adapters and transcription factors). Table S12 shows the chemical structures screened. Table S13 shows the screening results of the compounds of Table S12 against T-cells. Table S14 shows elaborated target results. Table S14). isoTOP-ABPP and TMT-ABPP proteomic data for active compounds (DMF, EV-3, EV-93, BPK-21, BPK-25; related to FIG. 5). Values for each treatment group are final average values resulting from at least 2 replicate experiments. For each compound, maximum values across both methods and proteomic fractions are reported in the Master table (Table S5). Table S15: Distribution of protein classes containing cysteines liganded by active compounds. Table S16: Comparison of isoTOP-ABPP and TMT-ABPP R values for cysteines liganded by active compounds versus scout fragments in human T cells, as displayed in correlation plot (FIG. 5D) and pie chart (FIG. 5E) analyses. Table S17: Prediction of pocket volumes within the indicated distances from cysteines liganded by active compounds (related to FIG. 5F). Table S18: Liganded cysteine residues with scout fragments at known sites of palmitoylation (obtained by cross-referencing with SwissPalm proteins and sites). Table S19: Protein expression changes after BPK-25 (10 μM, 24 h) treatment as determined by TMT-exp (10 replicates from 5 donors, related to FIG. 7B). Table S20: Summary of protein expression changes after DMF (50 μM, 24 h), EV-3 (10 μM, 24 h), BPK-21 (20 μM, 24 h), BPK-25 (10 μM, 24 h), and BPK-25 (10 μM, 24 h)+MG132 (10 μM, 24 h) treatment as determined by TMT-exp (average values from 2-5 donors are reported, related to FIG. 7C). The tables can be found in “An activity-guided map of electrophile-cysteine interactions in primary human immune cells”, Vinogradova, et al., bioRxiv 808113; doi: https://doi.org/10.1101/808113 (accessible on Oct. 17, 2019), which is incorporated herein by reference in its entirety.

Stereoselective Degradation of Immune Kinases by EV-96

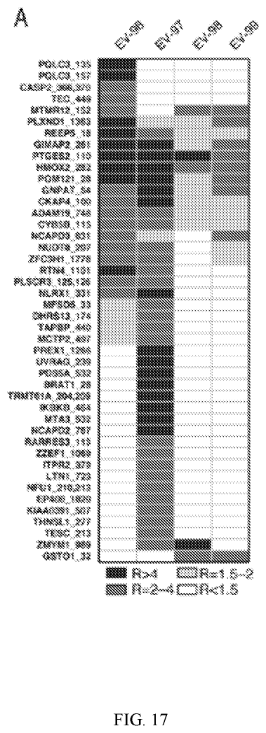

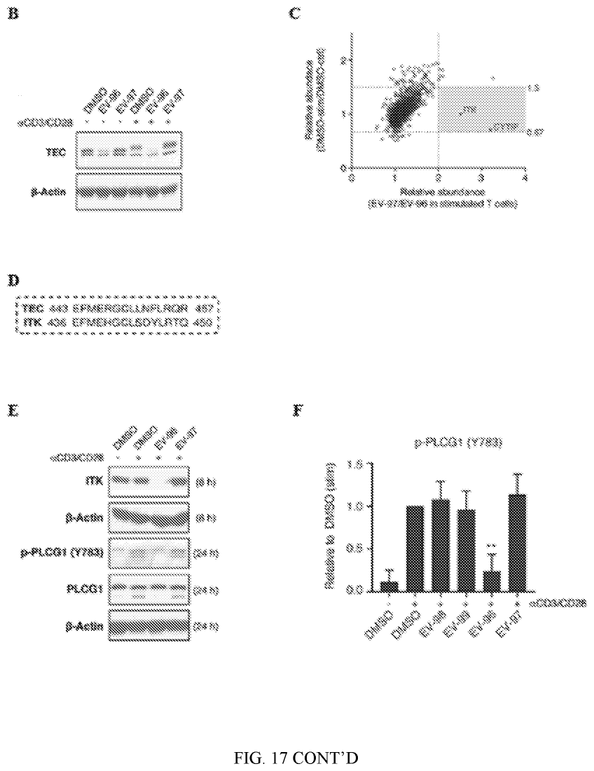

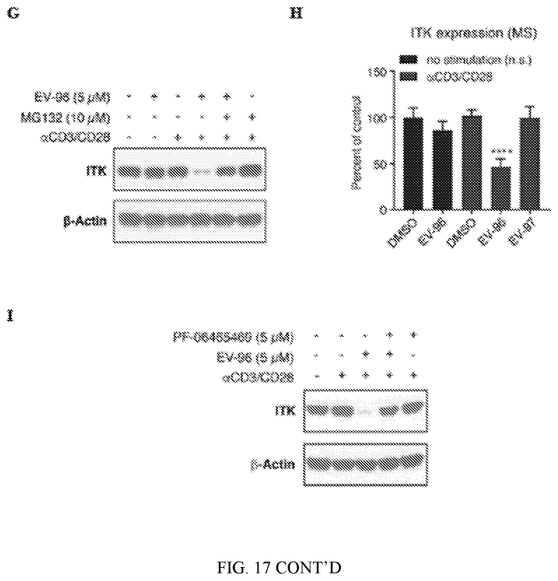

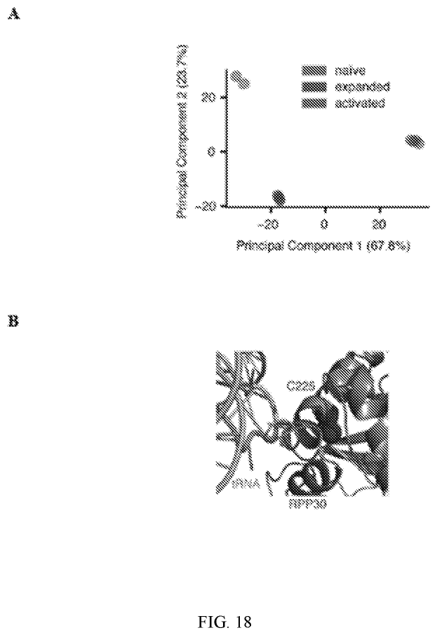

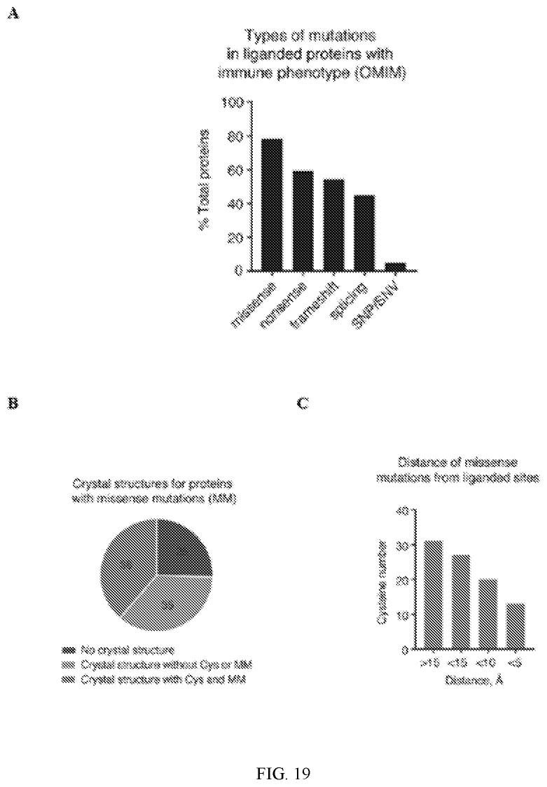

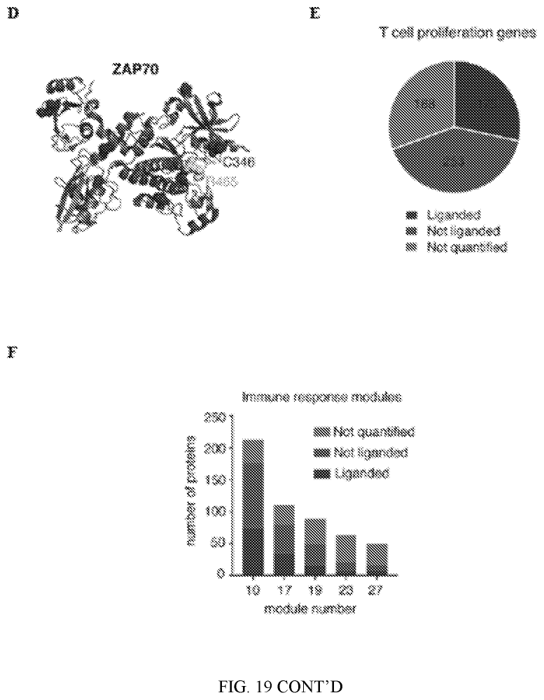

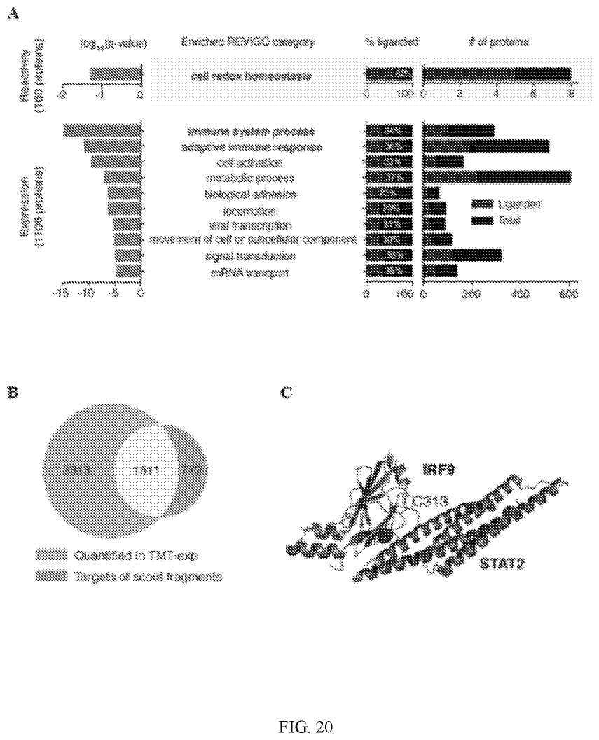

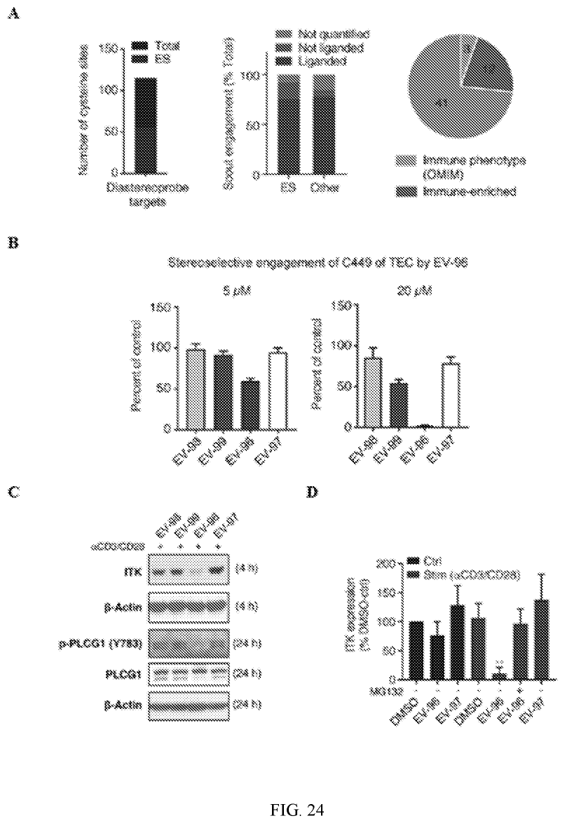

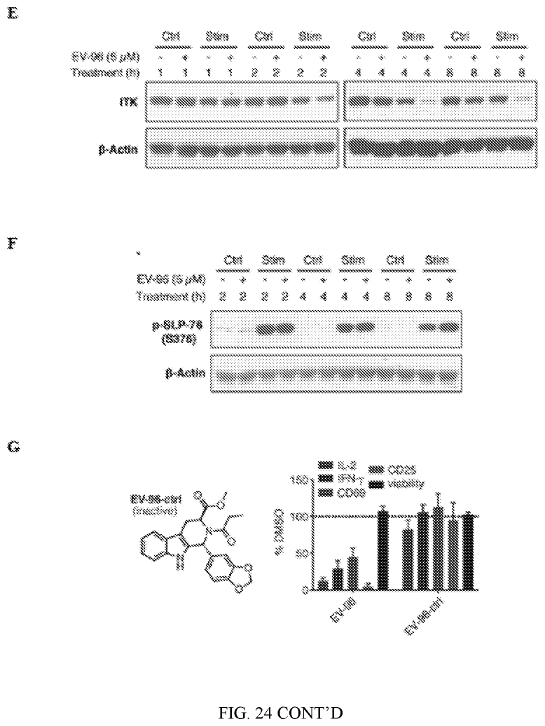

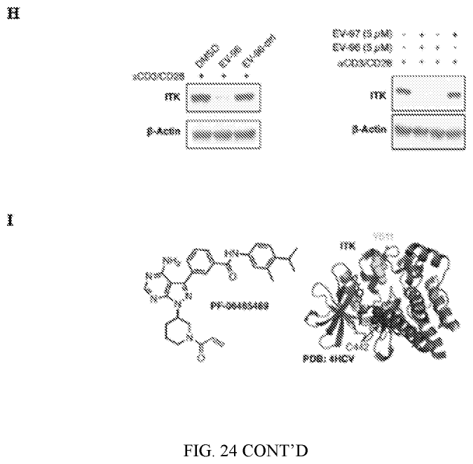

Among the active compounds, EV-96 showed a particularly interesting SAR, as its three stereoisomeric analogues were much less effective at suppressing T-cell activation (FIGS. 15B and 15C). The cysteine engagement profiles of this set of four stereoisomeric acrylamides revealed a striking number of stereoselective interactions at both 5 μM (FIG. 7A) and 20 μM (FIG. 24A) test concentrations, especially for the EV-96 and EV-97 pair of enantiomers. Most of these stereoselectively engaged cysteines were also liganded by scout fragments (FIG. 24A), and a number of them were found in immune-relevant proteins (FIG. 24A). To further investigate the mechanism of EV-96, we considered the relatively short list of protein targets harboring cysteines that were stereoselectively engaged by this compound at 5 μM (FIG. 7A). This group included a key immune kinase TEC, which was stereoselectively engaged at an active-site cysteine (C449) by EV-96 at both 5 and 20 μM (FIG. 24B). Western blotting revealed that EV-96, but not EV-97, also promoted the loss of TEC protein in T cells (FIG. 17B).

To better understand the global effects of EV-96 on the protein content of T cells, an unenriched proteomic experiment (TMT-exp) was performed on control versus activated T cells treated with DMSO, EV-96, or EV-97. To account for the immunosuppressive activity of EV-96, which is expected would indirectly block activation-dependent changes in protein expression in T cells, the profiles of DMSO-treated stimulated (DMSO-stim)-versus-control (DMSO-ctrl) T cells to EV-97-treated-versus-EV-96-treated stimulated T cells were compared. From a total of 3750 quantified proteins that displayed <2-fold expression changes between DMSO-stim and DMSO-ctrl T cells, two proteins were found to be substantially reduced in expression (>two-fold) in EV-96-treated, but not EV-97-treated stimulated T cells—the immune-relevant proteins ITK and CYTIP (FIG. 17C). Interestingly, ITK is a kinase that shares >55% identity with TEC kinase, including conservation of the active-site cysteine engaged by EV-96 (C442 in ITK; FIG. 17D). While TEC kinase was not detected in the unenriched proteomic experiment or C442 of ITK in TMT-ABPP experiments, the acquired data was interpreted to indicate that EV-96 may stereoselectively engage a shared active-site cysteine in both kinases, leading to their degradation.

ITK is a tyrosine kinase that plays a major role in T-cell signaling, undergoing recruitment to the plasma membrane following T-cell receptor (TCR) stimulation, where ITK is activated by LCK-mediated phosphorylation and in turn phosphorylates PLCG1 to promote downstream signaling pathways (Andreotti et al., 2010). It was verified by western blotting that EV-96, but not EV-97, caused the loss of ITK protein in stimulated T cells, and that this effect also led to a stereoselective blockade of PLCG1 phosphorylation (FIGS. 17E, F and 24C). Treatment with the proteasome inhibitor MG132 blocked EV-96-mediated loss of ITK (FIG. 17G). Remarkably, it was determined that EV-96 only caused the degradation of ITK in stimulated, but not naïve (FIGS. 17G, H and 24D) or expanded (FIG. 24E) control T cells, suggesting that upstream signaling events may be required to convert ITK into a form that is sensitive to EV-96-dependent degradation. Also consistent with this premise, EV-96 did not inhibit purified, recombinant ITK protein (FIG. 24F), which has been shown to behave differently from phosphorylated, activated ITK. It was determined that LCK-dependent phosphorylation of the upstream scaffolding protein SLP-76 was not affected by EV-96 (FIG. 24G), indicating the maintenance of early events in TCR signaling in cells treated with EV-96. Additionally, it was confirmed that a non-electrophilic propanamide analogue of EV-96 (EV-96-ctrl) did not suppress T cell activation (FIG. 24H) or induce ITK degradation (FIG. 24I) and pre-treatment with the inactive enantiomer EV-97 did not rescue ITK from EV-96-dependent degradation (FIG. 24J). Finally, pre-treatment with PF-064655469, a structurally distinct covalent inhibitor that engages C442 of ITK (Wang et al., 2020; Zapf et al., 2012) and blocks ITK enzymatic activity (FIG. 24F), rescued ITK from EV-96-induced degradation without affecting ITK stability on its own (FIGS. 171 and 24K).

Taken together, these studies indicate that EV-96 stereoselectively engages and promotes the degradation of key immune kinases, providing a plausible mechanistic basis for the compound's immunosuppressive effects. That the degradation of ITK was only observed in stimulated T cells further points to a provocative state-dependent pharmacological activity for EV-96, which should make this compound a uniquely useful chemical probe for studying T-cell signaling in various biological contexts.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods within the scope of these claims and their equivalents be covered thereby.

What is claimed is:

1. A method of modulating an immune response in a subject in need thereof, comprising administering to the subject a therapeutically effective amount of a small molecule fragment of Formula (I): 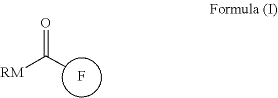
wherein:
RM is a reactive moiety selected from a Michael acceptor moiety, a leaving group moiety, or a moiety capable of forming a covalent bond with the thiol group of a cysteine residue; and
F is a small molecule fragment moiety.

2. The method of claim 1, wherein the small molecule fragment interacts with an endogenous cysteine-containing polypeptide expressed in the subject to form a cysteine-containing polypeptide-small molecule fragment adduct.

3. The method of claim 1 or 2, wherein the small molecule fragment is covalently bond to a cysteine residue of the cysteine-containing polypeptide.

4. The method of claim 2, wherein the cysteine-containing polypeptide-small molecule fragment adduct induces an immune response.

5. The method of claim 2 or 4, wherein the cysteine-containing polypeptide-small molecule fragment adduct induces a humoral immune response or a cell mediated immune response.

6. The method of claim 2, wherein the cysteine-containing polypeptide-small molecule fragment adduct increases an immune response relative to a control.

7. The method of claim 6, wherein the control is the level of an immune response in the subject prior to administration of the small molecule fragment or the level of an immune response in a subject who has not been exposed to the small molecule fragment.

8. The method of claim 2, wherein the cysteine-containing polypeptide is overexpressed in a disease or condition.

9. The method of claim 2, wherein the cysteine-containing polypeptide comprises one or more mutations, optionally overexpressed in a disease or condition.

10. The method of claim 8 or 9, wherein the disease or condition is cancer.

11. The method of claim 2, wherein the cysteine-containing polypeptide comprises a biologically active cysteine site, optionally located about 10 Å or less to an active-site ligand or residue.

12. The method of claim 2, wherein the cysteine-containing polypeptide is at most 50 amino acid residues in length.

13. The method of claim 1, wherein F is a small molecule fragment moiety illustrated in FIGS. 2B and 4C and Table S12.

14. The method of claim 1, wherein F is a fragment of the chloroacetamide compound in FIGS. 2B and 4C and Table S12 after the chlorine (Cl) has been removed, or a fragment of the acrylamide compound after the C═C has been converted to an ethylene.

15. The method of claim 1, wherein the method modulates T cell activation.

16. The method of claim 1, wherein the method suppresses T cell activation.

17. The method of claim 1, wherein F optionally comprises a second reactive moiety.

18. The method of claim 1, wherein the method is an in vivo method.
